SEQUENCE LISTING (92 sequences)

SEQ ID NO: 1 - DNA, 5431 bp, Artificial Sequence
  Description of Artificial Sequence Synthetic construct
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - DNA, 4479 bp, Artificial Sequence
  Description of Artificial Sequence Synthetic construct
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattcacgtgggcccggtaccgtatactctagagcggccgcggatccagatctttttccctcgccaaaaattatggggacatcatgaagccccttgagcatctgacttctggctaataaaggaaatttatttcattgcaatagtgtgttggaattttttgtgtctctcactcggaaggacatatgggagggcaaatcatttaaaacatcagaatcagtatttggtttagagtttggcaacatatgccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccagggcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 3 - DNA, 7648 bp, Artificial Sequence
  Description of Artificial Sequence Synthetic construct
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattccaccacactggactagtggatccatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcaaggatccatggctcgtgcggtcgggatcgacctcgggaccaccaactccgtcgtctcggttctggaaggtggcgacccggtcgtcgtcgccaactccgagggctccaggaccaccccgtcaattgtcgcgttcgcccgcaacggtgaggtgctggtcggccagcccgccaagaaccaggcagtgaccaacgtcgatcgcaccgtgcgctcggtcaagcgacacatgggcagcgactggtccatagagattgacggcaagaaatacaccgcgccggagatcagcgcccgcattctgatgaagctgaagcgcgacgccgaggcctacctcggtgaggacattaccgacgcggttatcacgacgcccgcctacttcaatgacgcccagcgtcaggccaccaaggacgccggccagatcgccggcctcaacgtgctgcggatcgtcaacgagccgaccgcggccgcgctggcctacggcctcgacaagggcgagaaggagcagcgaatcctggtcttcgacttgggtggtggcactttcgacgtttccctgctggagatcggcgagggtgtggttgaggtccgtgccacttcgggtgacaaccacctcggcggcgacgactgggaccagcgggtcgtcgattggctggtggacaagttcaagggcaccagcggcatcgatctgaccaaggacaagatggcgatgcagcggctgcgggaagccgccgagaaggcaaagatcgagctgagttcgagtcagtccacctcgatcaacctgccctacatcaccgtcgacgccgacaagaacccgttgttcttagacgagcagctgacccgcgcggagttccaacggatcactcaggacctgctggaccgcactcgcaagccgttccagtcggtgatcgctgacaccggcatttcggtgtcggagatcgatcacgttgtgctcgtgggtggttcgacccggatgcccgcggtgaccgatctggtcaaggaactcaccggcggcaaggaacccaacaagggcgtcaaccccgatgaggttgtcgcggtgggagccgctctgcaggccggcgtcctcaagggcgaggtgaaagacgttctgctgcttgatgttaccccgctgagcctgggtatcgagaccaagggcggggtgatgaccaggctcatcgagcgcaacaccacgatccccaccaagcggtcggagactttcaccaccgccgacgacaaccaaccgtcggtgcagatccaggtctatcagggggagcgtgagatcgccgcgcacaacaagttgctcgggtccttcgagctgaccggcatcccgccggcgccgcgggggattccgcagatcgaggtcactttcgacatcgacgccaacggcattgtgcacgtcaccgccaaggacaagggcaccggcaaggagaacacgatccgaatccaggaaggctcgggcctgtccaaggaagacattgaccgcatgatcaaggacgccgaagcgcacgccgaggaggatcgcaagcgtcgcgaggaggccgatgttcgtaatcaagccgagacattggtctaccagacggagaagttcgtcaaagaacagcgtgaggccgagggtggttcgaagttcgtaatcaagccgagacattggtctaccagacggagaagttcgtcaaagaacagcgtgaggccgagggtggttcgaaggtacctgaagacacgctgaacaaggttgatgccgcggtggcggaagcgaaggcggcacttggcggatcggatatttcggccatcaagtcggcgatggagaagctgggccaggagtcgcaggctctggggcaagcgatctacgaagcagctcaggctgcgtcacaggccactggcgctgcccaccccggctcggctgatgaaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaatgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 4 - DNA, 6221 bp, Artificial Sequence
  Description of Artificial Sequence Synthetic construct
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattcatgcgcctgcactttcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacgccgacgtggtgagcctgacctgcccggtcgccgccggtgaatgcgcgggcccggcggacagcggcgacgccctgctggagcgcaactatcccactggcgcggagttcctcggcgacggcggcgacgtcagcttcagcacccgcggcacgcagaacgaattcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcaaggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 5 - DNA, 5970 bp, Artificial Sequence
  Description of Artificial Sequence Synthetic construct
gctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccagggcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacatgctgctatccgtgccgctgctgctcggcctcctcggcctggccgtcgccgagcctgccgtctacttcaaggagcagtttctggacggggacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctggaattcatgcatggagatacacctacattgcatgaatatatgttagatttgcaaccagagacaactgatctctacggttatgggcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataaggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccatag

SEQ ID NO: 6 - DNA, 1257 bp, Artificial Sequence
  Description of Artificial Sequence Synthetic construct
atgacctctcgccgctccgtgaagtcgggtccgcgggaggttccgcgcgatgagtacgaggatctgtactacaccccgtcttcaggtatggcgagtcccgatagtccgcctgacacctcccgccgtggcgccctacagacacgctcgcgccagaggggcgaggtccgtttcgtccagtacgacgagtcggattatgccctctacgggggctcgtcttccgaagacgacgaacacccggaggtcccccggacgcggcgtcccgtttccggggcggttttgtccggcccggggcctgcgcgggcgcctccgccacccgctgggtccggaggggccggacgcacacccaccaccgccccccgggccccccgaacccagcgggtggcgtctaaggcccccgcggccccggcggcggagaccacccgcggcaggaaatcggcccagccagaatccgccgcactcccagacgcccccgcgtcgacggcgccaacccgatccaagacacccgcgcaggggctggccagaaagctgcactttagcaccgcccccccaaaccccgacgcgccatggaccccccgggtggccggctttaacaagcgcgtcttctgcgccgcggtcgggcgcctggcggccatgcatgcccggatggcggctgtccagctctgggacatgtcgcgtccgcgcacagacgaagacctcaacgaactccttggcatcaccaccatccgcgtgacggtctgcgagggcaaaaacctgcttcagcgcgccaacgagttggtgaatccagacgtggtgcaggacgtcgacgcggccacggcgactcgagggcgttctgcggcgtcgcgccccaccgagcgacctcgagccccagcccgctccgcttctcgccccagacggcccgtcgagggtaccgagctcggatccatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcaggataagcttaagtttaaaccgctgatcagcctcgactgtgccttctag

SEQ ID NO: 7 - DNA, 921 bp, Artificial Sequence
  Description of Artificial Sequence Synthetic construct
atgacctctcgccgctccgtgaagtcgggtccgcgggaggttccgcgcgatgagtacgaggatctgtactacaccccgtcttcaggtatggcgagtcccgatagtccgcctgacacctcccgccgtggcgccctacagacacgctcgcgccagaggggcgaggtccgtttcgtccagtacgacgagtcggattatgccctctacgggggctcgtcttccgaagacgacgaacacccggaggtcccccggacgcggcgtcccgtttccggggcggttttgtccggcccggggcctgcgcgggcgcctccgccacccgctgggtccggaggggccggacgcacacccaccaccgccccccgggccccccgaacccagcgggtggcgtctaaggcccccgcggccccggcggcggagaccacccgcggcaggaaatcggcccagccagaatccgccgcactcccagacgcccccgcgtcgacggcgccaacccgatccaagacacccgcgcaggggctggccagaaagctgcactttagcaccgcccccccaaaccccgacgcgccatggaccccccgggtggccggctttaacaagcgcgtcttctgcgccgcggtcgggcgcctggcggccatgcatgcccggatggcggctgtccagctctgggacatgtcgcgtccgcgcacagacgaagacctcaacgaactccttggcatcaccaccatccgcgtgacggtctgcgagggcaaaaacctgcttcagcgcgccaacgagttggtgaatccagacgtggtgcaggacgtcgacgcggccacggcgactcgagggcgttctgcggcgtcgcgccccaccgagcgacctcgagccccagcccgctccgcttctcgccccagacggcccgtcgagggtaccgagctcggatcc

SEQ ID NO: 8 - DNA, 297 bp, Human papillomavirus
  CDS (1)..(297)
atgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcaggataagctt

SEQ ID NO: 9 - protein, 99 aa, Human papillomavirus
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQDKL

SEQ ID NO: 10 - protein, 98 aa, Human papillomavirus
MHGDTPTLHEYMLDLQPETTDLYGYEGLNDSSEEEDEIDGPAGQAEPDRAHYNIVTFCCKCDSTLRLCVQSTHVDIRTLEDLLMGTLGIVCPICSQKP

SEQ ID NO: 11 - DNA, 477 bp, Human papillomavirus
  CDS (1)..(474)
atgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 12 - protein, 158 aa, Human papillomavirus
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 13 - protein, 151 aa, Human papillomavirus
MFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 14 - DNA, 1698 bp, Influenza virus
atgaaggcaaacctactggtcctgttaagtgcacttgcagctgcagatgcagacacaatatgtataggctaccatgcgaacaattcaaccgacactgttgacacagtactcgagaagaatgtgacagtgacacactctgttaacctgctcgaagacagccacaacggaaaactatgtagattaaaaggaatagccccactacaattggggaaatgtaacatcgccggatggctcttgggaaacccagaatgcgacccactgcttccagtgagatcatggtcctacattgtagaaacaccaaactctgagaatggaatatgttatccaggagatttcatcgactatgaggagctgagggagcaattgagctcagtgtcatcattcgaaagattcgaaatatttcccaaagaaagctcatggcccaaccacaacacaaacggagtaacggcagcatgctcccatgaggggaaaagcagtttttacagaaatttgctatggctgacggagaaggagggctcatacccaaagctgaaaaattcttatgtgaacaaaaaagggaaagaagtccttgtactgtggggtattcatcacccgcctaacagtaaggaacaacagaatatctatcagaatgaaaatgcttatgtctctgtagtgacttcaaattataacaggagatttaccccggaaatagcagaaagacccaaagtaagagatcaagctgggaggatgaactattactggaccttgctaaaacccggagacacaataatatttgaggcaaatggaaatctaatagcaccaatgtatgctttcgcactgagtagaggctttgggtccggcatcatcacctcaaacgcatcaatgcatgagtgtaacacgaagtgtcaaacacccctgggagctataaacagcagtctcccttaccagaatatacacccagtcacaataggagagtgcccaaaatacgtcaggagtgccaaattgaggatggttacaggactaaggaacactccgtccattcaatccagaggtctatttggagccattgccggttttattgaagggggatggactggaatgatagatggatggtatggttatcatcatcagaatgaacagggatcaggctatgcagcggatcaaaaaagcacacaaaatgccattaacgggattacaaacaaggtgaacactgttatcgagaaaatgaacattcaattcacagctgtgggtaaagaattcaacaaattagaaaaaaggatggaaaatttaaataaaaaagttgatgatggatttctggacatttggacatataatgcagaattgttagttctactggaaaatgaaaggactctggatttccatgactcaaatgtgaagaatctgtatgagaaagtaaaaagccaattaaagaataatgccaaagaaatcggaaatggatgttttgagttctaccacaagtgtgacaatgaatgcatggaaagtgtaagaaatgggacttatgattatcccaaatattcagaagagtcaaagttgaacagggaaaaggtagatggagtgaaattggaatcaatggggatctatcagattctggcgatctactcaactgtcgccagttcactggtgcttttggtctccctgggggcaatcagtttctggatgtgttctaatggatctttgcagtgcagaatatgcatctga

SEQ ID NO: 15 - protein, 565 aa, Influenza virus
MKANLLVLLSALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNIYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNTPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 16 - DNA, 501 bp, Artificial Sequence
  Description of Artificial Sequence Synthetic construct
atggcggcccccggcgcccggcggccgctgctcctgctgctgctggcaggccttgcacatggcgcctcagcactctttgaggatctaatcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgttaccttttgttgcaagtgtgactctacgcttcggttgtgcgtacaaagcacacacgtagacattcgtactttggaagacctgttaatgggcacactaggaattgtgtgccccatctgttctcaggatcttaacaacatgttgatccccattgctgtgggcggtgccctggcagggctggtcctcatcgtcctcattgcctacctcattggcaggaagaggagtcacgccggctatcagaccatctag

SEQ ID NO: 17 - protein, 166 aa, Artificial Sequence
  Description of Artificial Sequence Synthetic construct
MAAPGARRPLLLLLLAGLAHGASALFEDLIMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQDLNNMLIPIAVGGALAGLVLIVLIAYLIGRKRSHAGYQTI

SEQ ID NO: 18 - DNA, 5915 bp, Artificial Sequence
  Description of Artificial Sequence Synthetic construct
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattcatggcggcccccggcgcccggcggccgctgctcctgctgctgctggcaggccttgcacatggcgcctcagcactctttgaggatctaatcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgttaccttttgttgcaagtgtgactctacgcttcggttgtgcgtacaaagcacacacgtagacattcgtactttggaagacctgttaatgggcacactaggaattgtgtgccccatctgttctcaggatcttaacaacatgttgatccccattgctgtgggcggtgccctggcagggctggtcctcatcgtcctcattgcctacctcattggcaggaagaggagtcacgccggctatcagaccatctagggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 19 - DNA, 1878 bp, Mycobacterium tuberculosis
atggctcgtgcggtcgggatcgacctcgggaccaccaactccgtcgtctcggttctggaaggtggcgacccggtcgtcgtcgccaactccgagggctccaggaccaccccgtcaattgtcgcgttcgcccgcaacggtgaggtgctggtcggccagcccgccaagaaccaggcagtgaccaacgtcgatcgcaccgtgcgctcggtcaagcgacacatgggcagcgactggtccatagagattgacggcaagaaatacaccgcgccggagatcagcgcccgcattctgatgaagctgaagcgcgacgccgaggcctacctcggtgaggacattaccgacgcggttatcacgacgcccgcctacttcaatgacgcccagcgtcaggccaccaaggacgccggccagatcgccggcctcaacgtgctgcggatcgtcaacgagccgaccgcggccgcgctggcctacggcctcgacaagggcgagaaggagcagcgaatcctggtcttcgacttgggtggtggcactttcgacgtttccctgctggagatcggcgagggtgtggttgaggtccgtgccacttcgggtgacaaccacctcggcggcgacgactgggaccagcgggtcgtcgattggctggtggacaagttcaagggcaccagcggcatcgatctgaccaaggacaagatggcgatgcagcggctgcgggaagccgccgagaaggcaaagatcgagctgagttcgagtcagtccacctcgatcaacctgccctacatcaccgtcgacgccgacaagaacccgttgttcttagacgagcagctgacccgcgcggagttccaacggatcactcaggacctgctggaccgcactcgcaagccgttccagtcggtgatcgctgacaccggcatttcggtgtcggagatcgatcacgttgtgctcgtgggtggttcgacccggatgcccgcggtgaccgatctggtcaaggaactcaccggcggcaaggaacccaacaagggcgtcaaccccgatgaggttgtcgcggtgggagccgctctgcaggccggcgtcctcaagggcgaggtgaaagacgttctgctgcttgatgttaccccgctgagcctgggtatcgagaccaagggcggggtgatgaccaggctcatcgagcgcaacaccacgatccccaccaagcggtcggagactttcaccaccgccgacgacaaccaaccgtcggtgcagatccaggtctatcagggggagcgtgagatcgccgcgcacaacaagttgctcgggtccttcgagctgaccggcatcccgccggcgccgcgggggattccgcagatcgaggtcactttcgacatcgacgccaacggcattgtgcacgtcaccgccaaggacaagggcaccggcaaggagaacacgatccgaatccaggaaggctcgggcctgtccaaggaagacattgaccgcatgatcaaggacgccgaagcgcacgccgaggaggatcgcaagcgtcgcgaggaggccgatgttcgtaatcaagccgagacattggtctaccagacggagaagttcgtcaaagaacagcgtgaggccgagggtggttcgaaggtacctgaagacacgctgaacaaggttgatgccgcggtggcggaagcgaaggcggcacttggcggatcggatatttcggccatcaagtcggcgatggagaagctgggccaggagtcgcaggctctggggcaagcgatctacgaagcagctcaggctgcgtcacaggccactggcgctgcccaccccggcggcgagccgggcggtgcccaccccggctcggctgatgacgttgtggacgcggaggtggtcgacgacggccgggaggccaagtga

SEQ ID NO: 20 - protein, 625 aa, Mycobacterium tuberculosis
MARAVGIDLGTTNSVVSVLEGGDPVVVANSEGSRTTPSIVAFARNGEVLVGQPAKNQAVTNVDRTVRSVKRHMGSDWSIEIDGKKYTAPEISARILMKLKRDAEAYLGEDITDAVITTPAYFNDAQRQATKDAGQIAGLNVLRIVNEPTAAALAYGLDKGEKEQRILVFDLGGGTFDVSLLEIGEGVVEVRATSGDNHLGGDDWDQRVVDWLVDKFKGTSGIDLTKDKMAMQRLREAAEKAKIELSSSQSTSINLPYITVDADKNPLFLDEQLTRAEFQRITQDLLDRTRKPFQSVIADTGISVSEIDHVVLVGGSTRMPAVTDLVKELTGGKEPNKGVNPDEVVAVGAALQAGVLKGEVKDVLLLDVTPLSLGIETKGGVMTRLIERNTTIPTKRSETFTTADDNQPSVQIQVYQGEREIAAHNKLLGSFELTGIPPAPRGIPQIEVTFDIDANGIVHVTAKDKGTGKENTIRIQEGSGLSKEDIDRMIKDAEAHAEEDRKRREEADVRNQAETLVYQTEKFVKEQREAEGGSKVPEDTLNKVDAAVAEAKAALGGSDISAIKSAMEKLGQESQALGQAIYEAAQAASQATGAAHPGGEPGGAHPGSADDVVDAEVVDDGREAK

SEQ ID NO: 21 - DNA, 2104 bp, Artificial Sequence
  Description of Artificial Sequence Synthetic construct
atgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcaaggatccatggctcgtgcggtcgggatcgacctcgggaccaccaactccgtcgtctcggttctggaaggtggcgacccggtcgtcgtcgccaactccgagggctccaggaccaccccgtcaattgtcgcgttcgcccgcaacggtgaggtgctggtcggccagcccgccaagaaccaggcagtgaccaacgtcgatcgcaccgtgcgctcggtcaagcgacacatgggcagcgactggtccatagagattgacggcaagaaatacaccgcgccggagatcagcgcccgcattctgatgaagctgaagcgcgacgccgaggcctacctcggtgaggacattaccgacgcggttatcacgacgcccgcctacttcaatgacgcccagcgtcaggccaccaaggacgccggccagatcgccggcctcaacgtgctgcggatcgtcaacgagccgaccgcggccgcgctggcctacggcctcgacaagggcgagaaggagcagcgaatcctggtcttcgacttgggtggtggcactttcgacgtttccctgctggagatcggcgagggtgtggttgaggtccgtgccacttcgggtgacaaccacctcggcggcgacgactgggaccagcgggtcgtcgattggctggtggacaagttcaagggcaccagcggcatcgatctgaccaaggacaagatggcgatgcagcggctgcgggaagccgccgagaaggcaaagatcgagctgagttcgagtcagtccacctcgatcaacctgccctacatcaccgtcgacgccgacaagaacccgttgttcttagacgagcagctgacccgcgcggagttccaacggatcactcaggacctgctggaccgcactcgcaagccgttccagtcggtgatcgctgacaccggcatttcggtgtcggagatcgatcacgttgtgctcgtgggtggttcgacccggatgcccgcggtgaccgatctggtcaaggaactcaccggcggcaaggaacccaacaagggcgtcaaccccgatgaggttgtcgcggtgggagccgctctgcaggccggcgtcctcaagggcgaggtgaaagacgttctgctgcttgatgttaccccgctgagcctgggtatcgagaccaagggcggggtgatgaccaggctcatcgagcgcaacaccacgatccccaccaagcggtcggagactttcaccaccgccgacgacaaccaaccgtcggtgcagatccaggtctatcagggggagcgtgagatcgccgcgcacaacaagttgctcgggtccttcgagctgaccggcatcccgccggcgccgcgggggattccgcagatcgaggtcactttcgacatcgacgccaacggcattgtgcacgtcaccgccaaggacaagggcaccggcaaggagaacacgatccgaatccaggaaggctcgggcctgtccaaggaagacattgaccgcatgatcaaggacgccgaagcgcacgccgaggaggatcgcaagcgtcgcgaggaggccgatgttcgtaatcaagccgagacattggtctaccagacggagaagttcgtcaaagaacagcgtgaggccgagggtggttcgaaggtacctgaagacacgctgaacaaggttgatgccgcggtggcggaagcgaaggcggcacttggcggatcggatatttcggccatcaagtcggcgatggagaagctgggccaggagtcgcaggctctggggcaagcgatctacgaagcagctcaggctgcgtcacaggccactggcgctgcccaccccggctcggctgatgaaagca

SEQ ID NO: 22 - protein, 701 aa, Artificial Sequence
  Description of Artificial Sequence Synthetic construct
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQGSMARAVGIDLGTTNSVVSVLEGGDPVVVANSEGSRTTPSIVAFARNGEVLVGQPAKNQAVTNVDRTVRSVKRHMGSDWSIEIDGKKYTAPEISARILMKLKRDAEAYLGEDITDAVITTPAYFNDAQRQATKDAGQIAGLNVLRIVNEPTAAALAYGLDKGEKEQRILVFDLGGGTFDVSLLEIGEGVVEVRATSGDNHLGGDDWDQRVVDWLVDKFKGTSGIDLTKDKMAMQRLREAAEKAKIELSSSQSTSINLPYITVDADKNPLFLDEQLTRAEFQRITQDLLDRTRKPFQSVIADTGISVSEIDHVVLVGGSTRMPAVTDLVKELTGGKEPNKGVNPDEVVAVGAALQAGVLKGEVKDVLLLDVTPLSLGIETKGGVMTRLIERNTTIPTKRSETFTTADDNQPSVQIQVYQGEREIAAHNKLLGSFELTGIPPAPRGIPQIEVTFDIDANGIVHVTAKDKGTGKENTIRIQEGSGLSKEDIDRMIKDAEAHAEEDRKRREEADVRNQAETLVYQTEKFVKEQREAEGGSKVPEDTLNKVDAAVAEAKAALGGSDISAIKSAMEKLGQESQALGQAIYEAAQAASQATGAAHPGSADES

SEQ ID NO: 23 - DNA, 2760 bp, Pseudomonas aeruginosa
ctgcagctggtcaggccgtttccgcaacgcttgaagtcctggccgatataccggcagggccagccatcgttcgacgaataaagccacctcagccatgatgccctttccatccccagcggaaccccgacatggacgccaaagccctgctcctcggcagcctctgcctggccgccccattcgccgacgcggcgacgctcgacaatgctctctccgcctgcctcgccgcccggctcggtgcaccgcacacggcggagggccagttgcacctgccactcacccttgaggcccggcgctccaccggcgaatgcggctgtacctcggcgctggtgcgatatcggctgctggccaggggcgccagcgccgacagcctcgtgcttcaagagggctgctcgatagtcgccaggacacgccgcgcacgctgaccctggcggcggacgccggcttggcgagcggccgcgaactggtcgtcaccctgggttgtcaggcgcctgactgacaggccgggctgccaccaccaggccgagatggacgccctgcatgtatcctccgatcggcaagcctcccgttcgcacattcaccactctgcaatccagttcataaatcccataaaagccctcttccgctccccgccagcctccccgcatcccgcaccctagacgccccgccgctctccgccggctcgcccgacaagaaaaaccaaccgctcgatcagcctcatccttcacccatcacaggagccatcgcgatgcacctgataccccattggatccccctggtcgccagcctcggcctgctcgccggcggctcgtccgcgtccgccgccgaggaagccttcgacctctggaacgaatgcgccaaagcctgcgtgctcgacctcaaggacggcgtgcgttccagccgcatgagcgtcgacccggccatcgccgacaccaacggccagggcgtgctgcactactccatggtcctggagggcggcaacgacgcgctcaagctggccatcgacaacgccctcagcatcaccagcgacggcctgaccatccgcctcgaaggcggcgtcgagccgaacaagccggtgcgctacagctacacgcgccaggcgcgcggcagttggtcgctgaactggctggtaccgatcggccacgagaagccctcgaacatcaaggtgttcatccacgaactgaacgccggcaaccagctcagccacatgtcgccgatctacaccatcgagatgggcgacgagttgctggcgaagctggcgcgcgatgccaccttcttcgtcagggcgcacgagagcaacgagatgcagccgacgctcgccatcagccatgccggggtcagcgtggtcatggcccagacccagccgcgccgggaaaagcgctggagcgaatgggccagcggcaaggtgttgtgcctgctcgacccgctggacggggtctacaactacctcgcccagcaacgctgcaacctcgacgatacctgggaaggcaagatctaccgggtgctcgccggcaacccggcgaagcatgacctggacatcaaacccacggtcatcagtcatcgcctgcactttcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacgccgacgtggtgagcctgacctgcccggtcgccgccggtgaatgcgcgggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgcgcgaggacctgaagtaactgccgcgaccggccggctcccttcgcaggagccggccttctcggggcctggccatacatcaggttttcctgatgccagcccaatcgaatatgaattc

SEQ ID NO: 24 - protein, 638 aa, Pseudomonas aeruginosa
MHLIPHWIPLVASLGLLAGGSSASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQTQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 25 - protein, 171 aa, Pseudomonas aeruginosa
RLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNW

SEQ ID NO: 26 - DNA, 870 bp, Artificial Sequence
  Description of Artificial Sequence Synthetic construct
atgcgcctgcactttcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacgccgacgtggtgagcctgacctgcccggtcgccgccggtgaatgcgcgggcccggcggacagcggcgacgccctgctggagcgcaactatcccactggcgcggagttcctcggcgacggcggcgacgtcagcttcagcacccgcggcacgcagaacgaattcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcaaggatccgagctcggtaccaagcttaagtttaaaccgctgatcagcctcgactgtgccttctag

SEQ ID NO: 27 - protein, 289 aa, Artificial Sequence
  Description of Artificial Sequence Synthetic construct
MRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNEFMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQGSELGTKLKFKPLISLDCAF

SEQ ID NO: 28 - DNA, 1254 bp, Homo sapiens
atgctgctatccgtgccgctgctgctcggcctcctcggcctggccgtcgccgagcccgccgtctacttcaaggagcagtttctggacggagacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctgtag

SEQ ID NO: 29 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 30 - protein, 170 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTH

SEQ ID NO: 31 - protein, 109 aa, Homo sapiens
LYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQ

SEQ ID NO: 32 - protein, 138 aa, Homo sapiens
IDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 33 - DNA, 540 bp, Homo sapiens
atgctgctatccgtgccgctgctgctcggcctcctcggcctggccgtcgccgagcccgccgtctacttcaaggagcagtttctggacggagacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaac

SEQ ID NO: 34 - DNA, 267 bp, Homo sapiens
acctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccct

SEQ ID NO: 35 - DNA, 444 bp, Homo sapiens
gagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctg

SEQ ID NO: 36 - DNA, 5970 bp, Artificial Sequence; identical to SEQ ID NO: 5
  Description of Artificial Sequence Synthetic construct

SEQ ID NO: 37 - DNA, 750 bp, Artificial Sequence
  Description of Artificial Sequence Synthetic construct
atgggggattctgaaaggcggaaatcggaacggcgtcgttcccttggatatccctctgcatatgatgacgtctcgattcctgctcgcagaccatcaacacgtactcagcgaaatttaaaccaggatgatttgtcaaaacatggaccatttaccgaccatccaacacaaaaacataaatcggcgaaagccgtatcggaagacgtttcgtctaccacccggggtggctttacaaacaaaccccgtaccaagcccggggtcagagctgtacaaagtaataaattcgctttcagtacggctccttcatcagcatctagcacttggagatcaaatacagtggcatttaatcagcgtatgttttgcggagcggttgcaactgtggctcaatatcacgcataccaaggcgcgctcgccctttggcgtcaagatcctccgcgaacaaatgaagaattagatgcatttctttccagagctgtcattaaaattaccattcaagagggtccaaatttgatgggggaagccgaaacctgtgcccgcaaactattggaagagtctggattatcccaggggaacgagaacgtaaagtccaaatctgaacgtacaaccaaatctgaacgtacaagacgcggcggtgaaattgaaatcaaatcgccagatccgggatctcatcgtacacataaccctcgcactcccgcaacttcgcgtcgccatcattcatccgcccgcggatatcgtagcagtgatagcgaataa

SEQ ID NO: 38 - protein, 301 aa, Artificial Sequence
  Description of Artificial Sequence Synthetic construct
MTSRRSVKSGPREVPRDEYEDLYYTPSSGMASPDSPPDTSRRGALQTRSRQRGEVRFVQYDESDYALYGGSSSEDDEHPEVPRTRRPVSGAVLSGPGPARAPPPPAGSGGAGRTPTTAPRAPRTQRVASKAPAAPAAETTRGRKSAQPESAALPDAPASTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVE

SEQ ID NO: 39 - protein, 418 aa, Artificial Sequence
  Description of Artificial Sequence Synthetic construct
MTSRRSVKSGPREVPRDEYEDLYYTPSSGMASPDSPPDTSRRGALQTRSRQRGEVRFVQYDESDYALYGGSSSEDDEHPEVPRTRRPVSGAVLSGPGPARAPPPPAGSGGAGRTPTTAPRAPRTQRVASKAPAAPAAETTRGRKSAQPESAALPDAPASTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVEGTELGSMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQDKLKFKPLISLDCAF

SEQ ID NO: 40 - protein, 249 aa, Artificial Sequence
  Description of Artificial Sequence Synthetic construct
MGDSERRKSERRRSLGYPSAYDDVSIPARRPSTRTQRNLNQDDLSKHGPFTDHPTQKHKSAKAVSEDVSSTTRGGFTNKPRTKPGVRAVQSNKFAFSTAPSSASSTWRSNTVAFNQRMFCGAVATVAQYHAYQGALALWRQDPPRTNEELDAFLSRAVIKITIQEGPNLMGEAETCARKLLEESGLSQGNENVKSKSERTTKSERTRRGGEIEIKSPDPGSHRTHNPRTPATSRRHHSSARGYRSSDSE

SEQ ID NO: 41 - protein, 96 aa, Artificial Sequence
  Description of Artificial Sequence Synthetic construct
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQ

SEQ ID NO: 42 - DNA, 21 bp, Artificial Sequence
  Description of Combined DNA/RNA Molecule Synthetic oligonucleotide
ugccuacgaacucuucacctt

SEQ ID NO: 43 - DNA, 21 bp, Artificial Sequence
  Description of Combined DNA/RNA Molecule Synthetic oligonucleotide
ggugaagaguucguaggcatt

SEQ ID NO: 44 - DNA, 627 bp, Mus musculus
atggcatctggacaaggaccaggtcccccgaaggtgggctgcgatgagtccccgtccccttctgaacagcaggttgcccaggacacagaggaggtctttcgaagctacgttttttacctccaccagcaggaacaggagacccaggggcggccgcctgccaaccccgagatggacaacttgcccctggaacccaacagcatcttgggtcaggtgggtcggcagcttgctctcatcggagatgatattaaccggcgctacgacacagagttccagaatttactagaacagcttcagcccacagccgggaatgcctacgaactcttcaccaagatcgcctccagcctatttaagagtggcatcagctggggccgcgtggtggctctcctgggctttggctaccgtctggccctgtacgtctaccagcgtggtttgaccggcttcctgggccaggtgacctgctttttggctgatatcatactgcatcattacatcgccagatggatcgcacagagaggcggttgggtggcagccctgaatttgcgtagagaccccatcctgaccgtaatggtgatttttggtgtggttctgttgggccaattcgtggtacacagattcttcagatcatga

SEQ ID NO: 45 - DNA, 19 bp, Artificial Sequence
  Description of Artificial Sequence Synthetic oligonucleotide
tgcctacgaactcttcacc

SEQ ID NO: 46 - DNA, 21 bp, Artificial Sequence
  Description of Combined DNA/RNA Molecule Synthetic oligonucleotide
uauggagcugcagaggaugtt

SEQ ID NO: 47 - DNA, 21 bp, Artificial Sequence
  Description of Combined DNA/RNA Molecule Synthetic oligonucleotide
cauccucugcagcuccauatt

SEQ ID NO: 48 - DNA, 579 bp, Mus musculus
atggacgggtccggggagcagcttgggagcggcgggcccaccagctctgaacagatcatgaagacaggggcctttttgctacagggtttcatccaggatcgagcagggaggatggctggggagacacctgagctgaccttggagcagccgccccaggatgcgtccaccaagaagctgagcgagtgtctccggcgaattggagatgaactggatagcaatatggagctgcagaggatgattgctgacgtggacacggactccccccgagaggtcttcttccgggtggcagctgacatgtttgctgatggcaacttcaactggggccgcgtggttgccctcttctactttgctagcaaactggtgctcaaggccctgtgcactaaagtgcccgagctgatcagaaccatcatgggctggacactggacttcctccgtgagcggctgcttgtctggatccaagaccagggtggctgggaaggcctcctctcctacttcgggacccccacatggcagacagtgaccatctttgtggctggagtcctcaccgcctcgctcaccatctggaagaagatgggctga

SEQ ID NO: 49 - DNA, 19 bp, Artificial Sequence
  Description of Artificial Sequence Synthetic oligonucleotide
tatggagctgcagaggatg

SEQ ID NO: 50 - DNA, 1491 bp, Homo sapiens
atggacttcagcagaaatctttatgatattggggaacaactggacagtgaagatctggcctccctcaagttcctgagcctggactacattccgcaaaggaagcaagaacccatcaaggatgccttgatgttattccagagactccaggaaaagagaatgttggaggaaagcaatctgtccttcctgaaggagctgctcttccgaattaatagactggatttgctgattacctacctaaacactagaaaggaggagatggaaagggaacttcagacaccaggcagggctcaaatttctgcctacaggttccacttctgccgcatgagctgggctgaagcaaacagccagtgccagacacagtctgtacctttctggcggagggtcgatcatctattaataagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaaggggaggagttgtgtggggtaatgacaatctcggactctccaagagaacaggatagtgaatcacagactttggacaaagtttaccaaatgaaaagcaaacctcggggatactgtctgatcatcaacaatcacaattttgcaaaagcacgggagaaagtgcccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgctgtatcctctcccatggagacaagggcatcatctatggcactgatggacaggaggcccccatctatgagctgacatctcagttcactggtttgaagtgcccttcccttgctggaaaacccaaagtgttttttattcaggcttgtcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattga

SEQ ID NO: 51 - DNA, 23 bp, Artificial Sequence
  Description of Combined DNA/RNA Molecule Synthetic oligonucleotide
aaccucggggauacugucugatt

SEQ ID NO: 52 - DNA, 23 bp, Artificial Sequence
  Description of Combined DNA/RNA Molecule Synthetic oligonucleotide
ucagacaguauccccgagguutt

SEQ ID NO: 53 - DNA, 1251 bp, Homo sapiens
atggacgaagcggatcggcggctcctgcggcggtgccggctgcggctggtggaagagctgcaggtggaccagctctgggacgccctgctgagccgcgagctgttcaggccccatatgatcgaggacatccagcgggcaggctctggatctcggcgggatcaggccaggcagctgatcatagatctggagactcgagggagtcaggctcttcctttgttcatctcctgcttagaggacacaggccaggacatgctggcttcgtttctgcgaactaacaggcaagcagcaaagttgtcgaagccaaccctagaaaaccttaccccagtggtgctcagaccagagattcgcaaaccagaggttctcagaccggaaacacccagaccagtggacattggttctggaggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcataa

SEQ ID NO: 54 - DNA, 834 bp, Homo sapiens
atggagaacactgaaaactcagtggattcaaaatccattaaaaatttggaaccaaagatcatacatggaagcgaatcaatggactctggaatatccctggacaacagttataaaatggattatcctgagatgggtttatgtataataattaataataagaattttcataaaagcactggaatgacatctcggtctggtacagatgtcgatgcagcaaacctcagggaaacattcagaaacttgaaatatgaagtcaggaataaaaatgatcttacacgtgaagaaattgtggaattgatgcgtgatgtttctaaagaagatcacagcaaaaggagcagttttgtttgtgtgcttctgagccatggtgaagaaggaataatttttggaacaaatggacctgttgacctgaaaaaaataacaaactttttcagaggggatcgttgtagaagtctaactggaaaacccaaacttttcattattcaggcctgccgtggtacagaactggactgtggcattgagacagacagtggtgttgatgatgacatggcgtgtcataaaataccagtggaggccgacttcttgtatgcatactccacagcacctggttattattcttggcgaaattcaaaggatggctcctggttcatccagtcgctttgtgccatgctgaaacagtatgccgacaagcttgaatttatgcacattcttacccgggttaaccgaaaggtggcaacagaatttgagtccttttcctttgacgctacttttcatgcaaagaaacagattccatgtattgtttccatgctcacaaaagaactctatttttatcactaa

SEQ ID NO: 55 - DNA, 750 bp, Homo sapiens
atggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatga

SEQ ID NO: 56 - protein, 249 aa, Homo sapiens
MAYPYDVPDYASLRSTMSQSNRELVVDFLSYKLSQKGYSWSQFSDVEENRTEAPEGTESEMETPSAINGNPSWHLADSPAVNGATAHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 57 - DNA, 6187 bp, Artificial Sequence
  Description of Artificial Sequence Synthetic construct
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattccaccacactggactagtggatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaagatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaaatgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 58 - DNA, 6452 bp, Artificial Sequence
  Description of Artificial Sequence Synthetic construct
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccaggatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctactgggctcactcttcagtcggaaatgaagatccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 59 - protein, 349 aa, Artificial Sequence
  Description of Artificial Sequence Synthetic construct
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPGSMAYPYDVPDYASLRSTMSQSNRELVVDFLSYKLSQKGYSWSQFSDVEENRTEAPEGTESEMETPSAINGNPSWHLADSPAVNGATAHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 60 - DNA, 750 bp, Artificial Sequence
  Description of Artificial Sequence Synthetic construct
atggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtagccattcttcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatga

SEQ ID NO: 61 - protein, 249 aa, Artificial Sequence
  Description of Artificial Sequence Synthetic construct
MAYPYDVPDYASLRSTMSQSNRELVVDFLSYKLSQKGYSWSQFSDVEENRTEAPEGTESEMETPSAINGNPSWHLADSPAVNGATAHSSSLDAREVIPMAAVKQALREAGDEFELRYRRAFSDLTSQLHITPGTAYQSFEQVVNELFRDGVAILRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 62 - protein, 349 aa, Artificial Sequence
  Description of Artificial Sequence Synthetic construct
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPGSMAYPYDVPDYASLRSTMSQSNRELVVDFLSYKLSQKGYSWSQFSDVEENRTEAPEGTESEMETPSAINGNPSWHLADSPAVNGATAHSSSLDAREVIPMAAVKQALREAGDEFELRYRRAFSDLTSQLHITPGTAYQSFEQVVNELFRDGVAILRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 63 - DNA, 6187 bp, Artificial Sequence; identical to SEQ ID NO: 57
  Description of Artificial Sequence Synthetic construct

SEQ ID NO: 64 - DNA, 6452 bp, Artificial Sequence; identical to SEQ ID NO: 58
  Description of Artificial Sequence Synthetic construct

SEQ ID NO: 65 - DNA, 12347 bp, Artificial Sequence
  Description of Artificial Sequence Synthetic construct
atggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgctagccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattggatcccaaacgggccctctagactcgagcggccgccactgtgctggatatctgcagaattccaccacactggactagtggatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcatggttctactgggctcactcttcagtcggaaatgaagatccgagctcggtaccaagcttaagtttgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgtatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaaactagtgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctagtctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcgcgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggcattctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttctgtctaagcggatgccgggagcagacaagcccgtcagggcgcgtcagcgggtgttggcgggtgtcggggctggcttaactatgcggcatcagagcagattgtactgagagtgcaccatatcgacgctctcccttatgcgactcctgcattaggaagcagcccagtactaggttgaggccgttgagcaccgccgccgcaaggaatggtgcatgcgtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccggaagcttgaattc

SEQ ID NO: 66 - DNA, 12612 bp, Artificial Sequence
  Description of Artificial Sequence Synthetic construct
atggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgctagccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattggatcccaaacgggccctctagactcgagcggccgccactgtgctggatatctgcagaattcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccaggatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaagatccaagcttaagtttgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgtatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaaactagtgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctagtctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcgcgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggcattctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttctgtctaagcggatgccgggagcagacaagcccgtcagggcgcgtcagcgggtgttggcgggtgtcggggctggcttaactatgcggcatcagagcagattgtactgagagtgcaccatatcgacgctctcccttatgcgactcctgcattaggaagcagcccagtactaggttgaggccgttgagcaccgccgccgcaaggaatggtgcatgcgtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccggaagcttgaattc

SEQ ID NO: 67 - DNA, 12347 bp, Artificial Sequence; identical to SEQ ID NO: 65
  Description of Artificial Sequence Synthetic construct

SEQ ID NO: 68 - DNA, 12612 bp, Artificial Sequence; identical to SEQ ID NO: 66
  Description of Artificial Sequence Synthetic construct

SEQ ID NO: 69 - DNA, 4832 bp, Artificial Sequence
  Description of Artificial Sequence Synthetic construct
gtcgacttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcggatccagatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaggccagcaaaaggccaggaaccgtaaaaaggccgcgttgctggcgttttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcttttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaatt

SEQ ID NO: 70 - DNA, 4832 bp, Artificial Sequence; identical to SEQ ID NO: 69
  Description of Artificial Sequence Synthetic construct

SEQ ID NO: 71 - DNA, 1499 bp, Artificial Sequence
  Description of Artificial Sequence Synthetic construct
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagatagatggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtattgtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtcttaatctaa

SEQ ID NO: 72 - protein, 497 aa, Artificial Sequence [sequence omitted]

SEQ ID NO: 73 - DNA, 5575 bp, Artificial Sequence
  Description of Artificial Sequence Synthetic construct
gtcgacttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaataatcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcggatccatgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagatagatggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtattgtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtcttaatctaaagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcttttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaatt

SEQ ID NO: 74 - DNA, 1395 bp, Artificial Sequence
  Description of Artificial Sequence Synthetic construct
atggacttcagcagaaatctttatgatattggggaacaactggacagtgaagatctggcctccctcaagttcctgagcctggactacattccgcaaaggaagcaagaacccatcaaggatgccttgatgttattccagagactccaggaaaagagaatgttggaggaaagcaatctgtccttcctgaaggagctgctcttccgaattaatagactggatttgctgattacctacctaaacactagaaaggaggagatggaaagggaacttcagacaccaggcagggctcaaatttctgcctacagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaaggggaggagttgtgtggggtaatgacaatctcggactctccaagagaacaggatagtgaatcacagactttggacaaagtttaccaaatgaaaagcaaacctcggggatactgtctgatcatcaacaatcacaattttgcaaaagcacgggagaaagtgcccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgctgtatcctctcccatggagacaagggcatcatctatggcactgatggacaggaggcccccatctatgagctgacatctcagttcactggtttgaagtgcccttcccttgctggaaaacccaaagtgttttttattcaggcttgtcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattga

SEQ ID NO: 75 - protein, 464 aa, Artificial Sequence
  Description of Artificial Sequence Synthetic construct
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 76 - DNA, 5471 bp, Artificial Sequence
  Description of Artificial Sequence Synthetic construct
gtcgacttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcatggacttcagcagaaatctttatgatattggggaacaactggacagtgaagatctggcctccctcaagttcctgagcctggactacattccgcaaaggaagcaagaacccatcaaggatgccttgatgttattccagagactccaggaaaagagaatgttggaggaaagcaatctgtccttcctgaaggagctgctcttccgaattaatagactggatttgctgattacctacctaaacactagaaaggaggagatggaaagggaacttcagacaccaggcagggctcaaatttctgcctacagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaaggggaggagttgtgtggggtaatgacaatctcggactctccaagagaacaggatagtgaatcacagactttggacaaagtttaccaaatgaaaagcaaacctcgggatactgtctgatcatcaacaatcacaattttgcaaaagcacgggagaaagtgccccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgctgtatcctctcccatggagacaagggcatcatctatggcactgatggacaggaggcccccatctatgagctgacatctcagttcactggtttgaagtgcccttcccttgctggaaaacccaaagtgttttttattcaggcttgtcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattgaggatccagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcttttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaatt

SEQ ID NO: 77 - DNA, 618 bp, Artificial Sequence
  Description of Artificial Sequence Synthetic construct
atggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggtaggtgcacttggtgatgtgagtctgggctga

SEQ ID NO: 78 - protein, 205 aa, Artificial Sequence
  Description of Artificial Sequence Synthetic construct
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWVGALGDVSLG

SEQ ID NO: 79 - DNA, 4699 bp, Artificial Sequence
  Description of Artificial Sequence Synthetic construct
gtcgacttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcggatccagatctatggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggtaggtgcacttggtgatgtgagtctgggctgaagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcttttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaatt

SEQ ID NO: 80 - DNA, 5471 bp, Artificial Sequence
  Description of Artificial Sequence Synthetic construct
gtcgacttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcggatccatggacttcagcagaaatctttatgatattggggaacaactggacagtgaagatctggcctccctcaagttcctgagcctggactacattccgcaaaggaagcaagaacccatcaaggatgccttgatgttattccagagactccaggaaaagagaatgttggaggaaagcaatctgtccttcctgaaggagctgctcttccgaattaatagactggatttgctgattacctacctaaacactagaaaggaggagatggaaagggaacttcagacaccaggcagggctcaaatttctgcctacagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaaggggaggagttgtgtggggtaatgacaatctcggactctccaagagaacaggatagtgaatcacagactttggacaaagtttaccaaatgaaaagcaaacctcggggatactgtctgatcatcaacaatcacaattttgcaaaagcacgggagaaagtgcccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgctgtatcctctcccatggagacaagggcatcatctatggcactgatggacaggaggcccccatctatgagctgacatctcagttcactggtttgaagtgcccttcccttgctggaaaacccaaagtgttttttattcaggcttctcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattgaagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcttttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaatt

SEQ ID NO: 81 - protein, 464 aa, Artificial Sequence
  Description of Artificial Sequence Synthetic construct
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQASQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 82 - DNA, 5327 bp, Artificial Sequence
  Description of Artificial Sequence Synthetic construct
gtcgacttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcggatccatggacgaagcggatcggcggctcctgcggcggtgccggctgcggctggtggaagagctgcaggtggaccagctctgggacgccctgctgagccgcgagctgttcaggccccatatgatcgaggacatccagcgggcaggctctggatctcggcgggatcaggccaggcagctgatcatagatctggagactcgagggagtcaggctcttcctttgttcatctcctgcttagaggacacaggccaggacatgctggcttcgtttctgcgaactaacaggcaagcagcaaagttgtcgaagccaaccctagaaaaccttaccccagtggtgctcagaccagagattcgcaaaccagaggttctcagaccggaaacacccagaccagtggacattggttctggaggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctctggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcataaagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcttttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaatt

SEQ ID NO: 83 - protein, 416 aa, Artificial Sequence
  Description of Artificial Sequence Synthetic construct
MDEADRRLLRRCRLRLVEELQVDQLWDALLSRELFRPHMIEDIQRAGSGSRRDQARQLIIDLETRGSQALPLFISCLEDTGQDMLASFLRTNRQAAKLSKPTLENLTPVVLRPEIRKPEVLRPETPRPVDIGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQASGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 84 - DNA, 1819 bp, Artificial Sequence
  Description of Artificial Sequence Synthetic construct
gaattccgggctggattgagaagccgcaactgtgactctgcatcatgaatactctgtctgaaggaaatggcacctttgccatccatcttttgaagatgctatgtcaaagcaacccttccaaaaatgtatgttattctcctgcgagcatctcctctgctctagctatggttctcttgggtgcaaagggacagacggcagtccagatatctcaggcacttggtttgaataaagaggaaggcatccatcagggtttccagttgcttctcaggaagctgaacaagccagacagaaagtactctcttagagtggccaacaggctctttgcagacaaaacttgtgaagtcctccaaacctttaaggagtcctctcttcacttctatgactcagagatggagcagctctcctttgctgaagaagcagaggtgtccaggcaacacataaacacatgggtctccaaacaaactgaaggtaaaattccagagttgttgtcaggtggctccgtcgattcagaaaccaggctggttctcatcaatgccttatattttaaaggaaagtggcatcaaccatttaacaaagagtacacaatggacatgccctttaaaataaacaaggatgagaaaaggccagtgcagatgatgtgtcgtgaagacacatataacctcgcctatgtgaaggaggtgcaggcgcaagtgctggtgatgccatatgaaggaatggagctgagcttggtggttctgctcccagatgagggtgtggacctcagcaaggtggaaaacaatctcacttttgagaagttaacagcctggatggaagcagattttatgaagagcactgatgttgaggttttccttccaaaatttaaactccaagaggattatgacatggagtctctgtttcagcgcttgggagtggtggatgtcttccaagaggacaaggctgacttatcaggaatgtctccagagagaaacctgtgtgtgtccaagtttgttcaccagagtgtagtggagatcaatgaggaaggcacagaggctgcagcagcctctgccatcatagaattttgctgtgcctcttctgtcccaacattctgtgctgaccaccccttccttttcttcatcaggcacaacaaagcaaacagcatcctgttctgtggcaggttctcatctccataaagacacatatactacacagggagagttctctcttcagtatccctaccactcctacagctctgtcaagatgggcaagtagggggaagtcatgttctaagatgaagacactttccttctctgtcagcctgatcttataatgcctgcattcaactctccctgtcttgaatgcatctatgccctttaccaggttatgtctaatgatgccaaataccttctgctatgctattgattgatagcctagccagtaatttatagccagttagaactgacttgactgtgcaagaatgctataatggagctagagagaaggcacaaacactaggaaaggttgctgtttttgcagaggacacagggacatttcccaccactcacatggctgcttacaacctctggaaattccagtttctgtccatgacttgattcctttctttggcttctactggctccagcatcctgcacatacatgtatcgtcattcagttacacacaaacaagtaaaattttaaaaataaataaaaatttaaagagagagtctaaaattttagtaatggttagataatagctgctattgtgcctttttcaggttttaatgtcattattcttgtgtataaagtcaataatttataggaaaacatcagtgccccggaattc

SEQ ID NO: 85 - protein, 374 aa, Artificial Sequence
  Description of Artificial Sequence Synthetic construct
MNTLSEGNGTFAIHLLKMLCQSNPSKNVCYSPASISSALAMVLLGAKGQTAVQISQALGLNKEEGIHQGFQLLLRKLNKPDRKYSLRVANRLFADKTCEVLQTFKESSLHFYDSEMEQLSFAEEAEVSRQHINTWVSKQTEGKIPELLSGGSVDSETRLVLINALYFKGKWHQPFMKEYTMDMPFKINKDEKRPVQMMCREDTYNLAYVKEVQAQVLVMPYEGMELSLVVLLPDEGVDLSKVENNLTFEKLTAWMEADFMKSTDVEVFLPKFKLQEDYDMESLFQRLGVVDVFQEDKADLSGMSPERNLCVSKFVHQSVVEINEEGTEAAAASAIIEFCCASSVPTFCADHPFLFFIRHNKANSILFCGRFSSP

SEQ ID NO: 86 - DNA, 1125 bp, Artificial Sequence
  Description of Artificial Sequence Synthetic construct
atgaatactctgtctgaaggaaatggcacctttgccatccatcttttgaagatgctatgtcaaagcaacccttccaaaaatgtatgttattctcctgcgagcatctcctctgctctagctatggttctcttgggtgcaaagggacagacggcagtccagatatctcaggcacttggtttgaataaagaggaaggcatccatcagggtttccagttgcttctcaggaagctgaacaagccagacagaaagtactctcttagagtggccaacaggctctttgcagacaaaacttgtgaagtcctccaaacctttaaggagtcctctcttcacttctatgactcagagatggagcagctctcctttgctgaagaagcagaggtgtccaggcaacacataaacacatgggtctccaaacaaactgaaggtaaaattccagagttgttgtcaggtggctccgtcgattcagaaaccaggctggttctcatcaatgccttatattttaaaggaaagtggcatcaaccatttaacaaagagtacacaatggacatgccctttaaaataaacaaggatgagaaaaggccagtgcagatgatgtgtcgtgaagacacatataacctcgcctatgtgaaggaggtgcaggcgcaagtgctggtgatgccatatgaaggaatggagctgagcttggtggttctgctcccagatgagggtgtggacctcagcaaggtggaaaacaatctcacttttgagaagttaacagcctggatggaagcagattttatgaagagcactgatgttgaggttttccttccaaaatttaaactccaagaggattatgacatggagtctctgtttcagcgcttgggagtggtggatgtcttccaagaggacaaggctgacttatcaggaatgtctccagagagaaacctgtgtgtgtccaagtttgttcaccagagtgtagtggagatcaatgaggaaggcagagaggctgcagcagcctctgccatcatagaattttgctgtgcctcttctgtcccaacattctgtgctgaccaccccttccttttcttcatcaggcacaacaaagcaaacagcatcctgttctgtggcaggttctcatctccataa

SEQ ID NO: 87 - protein, 374 aa, Artificial Sequence
  Description of Artificial Sequence Synthetic construct
MNTLSEGNGTFAIHLLKMLCQSNPSKNVCYSPASISSALAMVLLGAKGQTAVQISQALGLNKEEGIHQGFQLLLRKLNKPDRKYSLRVANRLFADKTCEVLQTFKESSLHFYDSEMEQLSFAEEAEVSRQHINTWVSKQTEGKIPELLSGGSVDSETRLVLINALYFKGKWHQPFNKEYTMDMPFKINKDEKRPVQMMCREDTYNLAYVKEVQAQVLVMPYEGMELSLVVLLPDEGVDLSKVENNLTFEKLTAWMEADFMKSTDVEVFLPKFKLQEDYDMESLFQRLGVVDVFQEDKADLSGMSPERNLCVSKFVHQSVVEINEEGREAAAASAIIEFCCASSVPTFCADHPFLFFIRHNKANSILFCGRFSSP

SEQ ID NO: 88 - DNA, 6536 bp, Artificial Sequence
  Description of Artificial Sequence Synthetic construct
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattcatgaatactctgtctgaaggaaatggcacctttgccatccatcttttgaagatgctatgtcaaagcaacccttccaaaaatgtatgttattctcctgcgagcatctcctctgctctagctatggttctcttgggtgcaaagggacagacggcagtccagatatctcaggcacttggtttgaataaagaggaaggcatccatcagggtttccagttgcttctcaggaagctgaacaagccagacagaaagtactctcttagagtggccaacaggctctttgcagacaaaacttgtgaagtcctccaaacctttaaggagtcctctcttcacttctatgactcagagatggagcagctctcctttgctgaagaagcagaggtgtccaggcaacacataaacacatgggtctccaaacaaactgaaggtaaaattccagagttgttgtcaggtggctccgtcgattcagaaaccaggctggttctcatcaatgccttatattttaaaggaaagtggcatcaaccatttaacaaagagtacacaatggacatgccctttaaaataaacaaggatgagaaaaggccagtgcagatgatgtgtcgtgaagacacatataacctcgcctatgtgaaggaggtgcaggcgcaagtgctggtgatgccatatgaaggaatggagctgagcttggtggttctgctcccagatgagggtgtggacctcagcaaggtggaaaacaatctcacttttgagaagttaacagcctggatggaagcagattttatgaagagcactgatgttgaggttttccttccaaaatttaaactccaagaggattatgacatggagtctctgtttcagcgcttgggagtggtggatgtcttccaagaggacaaggctgacttatcaggaatgtctccagagagaaacctgtgtgtgtccaagtttgttcaccagagtgtagtggagatcaatgaggaaggcacagaggctgcagcagcctctgccatcatagaattttgctgtgcctcttctgtcccaacattctgtgctgaccaccccttccttttcttcatcaggcacaacaaagcaaacagcatcctgttctgtggcaggttctcatctccaggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 89 - DNA, 6536 bp, Artificial Sequence; identical to SEQ ID NO: 88
  Description of Artificial Sequence Synthetic construct

SEQ ID NO: 90 - DNA, 31 bp, Artificial Sequence
  Description of Artificial Sequence Synthetic primer
aaagtcgacatgctgctatccgtgccgctgc

SEQ ID NO: 91 - DNA, 26 bp, Artificial Sequence
  Description of Artificial Sequence Synthetic primer
gaattcgttgtctggccgcacaatca

SEQ ID NO: 92 - protein, 9 aa, Artificial Sequence
  Description of Artificial Sequence Synthetic peptide
RAHYNIVTF

(19) United States
(12) Patent Application Publication
(10) Pub. No.: US 2010/0330105 A1
(43) Pub. Date: Dec. 30, 2010

(54) Anticancer Combination Therapies

(75) Inventors: Tzyy-Choou Wu, Stevenson, MD (US); Chien-Fu Hung, Timonium, MD (US)

Correspondence Address: Foley Hoag, LLP (w/JHV), World Trade Center West, 155 Seaport Blvd, Boston, MA, 02210-2600, US

(73) Assignee: John Hopkins University, Baltimore, MD (US)

(21) Appl. No.: 12/438,300

(22) PCT Filed: Aug. 22, 2007
(86) PCT No.: PCT/US07/76525

§ 371 Date: Jun. 7, 2010

Related U.S. Application Data
(60) Provisional application No. 60/839,254, filed on Aug. 22, 2006.

(51) Int. Cl. A61K 39/12; A61K 39/00; A61K 39/395; A61P 35/00; A61P 37/04

(52) U.S. Cl. 424/174.1; 424/277.1; 424/204.1; 424/192.1

(57) ABSTRACT

Methods for treating or preventing hyperproliferating diseases, e.g., cancer, are described. A method may comprise administering to a subject in need thereof a therapeutically effective amount of a chemotherapeutic agent and a DNA vaccine.

### CROSS-REFERENCE TO RELATED APPLICATIONS

This application claims the benefit of U.S. Provisional Application No. 60/839,254, filed on Aug. 22, 2006, the content of which is specifically incorporated by reference herein in its entirety.

### GOVERNMENTAL SUPPORT

This invention was made with government support under grant numbers P50 CA098252 and RO1 CA114425, awarded by the U.S. National Institutes of Health. The government has certain rights in this invention.

### BACKGROUND

Although chemotherapeutic regimens have been useful in treating cancer, their success is limited by the often severe systemic toxicity frequently associated with their use. Similarly, cancer immunotherapeutics have shown promise for the treatment of a number of tumors and hyperproliferiative diseases, but their utility is limited in situations where the tumor is relatively large or rapidly growing.

The present inventors have developed a number of DNA vaccine systems for HPV-associated cervical neoplasia as well as HPV-associated head and neck cancers (3-5). Cervical cancer can serve as a model of how a viral infection can progress through a multistep process from initial infection to premalignant dysplasia, called cervical intraepithelial neoplasia (CIN), to invasive cancer. Human papilloma virus (HPV), particularly HPV-16, is associated with a majority of cervical cancers and a subset of head and neck cancers (for review, see (6)). HPV-16 E7, one of its oncoproteins, is essential for the induction and maintenance of cellular transformation (6). Thus, HPV-16 E7 is an ideal target for developing vaccine and immunotherapeutic strategies for the control of HPV infections and HPV-associated lesions (for review, see (7, 8)). However, the antigen-specific immune responses and antitumor effects generated by DNA vaccines encoding wild type E7 is weak and not enough to be effective in controlling tumor growth. To overcome the weak antigenicity of E7, the present inventors have previously created a DNA vaccine encoding HPV-16 E7 linked to the sorting signal of the lysosome-associated membrane protein 1 (LAMP-1) (9-11). The encoded chimeric protein (Sig/E7/LAMP-1) also includes the signal peptide derived from LAMP-1 protein. Vaccination with Sig/E7/LAMP-1 DNA led to a significantly enhanced E7-specific CD4⁺ and CD8⁺ T cell-mediated immune responses, resulting in potent antitumor effects against E7-expressing tumors in vaccinated mice (9-11).

In addition to the Sig/E7/LAMP-1 construct described above, the present inventors and their colleagues have also previously developed several additional intracellular targeting and intercellular spreading strategies to enhance DNA vaccine potency using various immunogenicity-potentiating polypeptides (IPPs), described in further detail below. See for example, publications of the present inventors and their colleagues: Hung, C F et al., J Virol 76:2676-82, 2002; Cheng, W F et al., J Clin Invest 108:669-78, 2001; Hung, C F et al., J Immunol 166:5733-40, 2001; Chen, C H et al., Gene Ther 6:1972-81, 1999; Ji, H et al., Hum Gene Ther 10:2727-40, 1999; Chen, C H et al., Cancer Res 60:1035-42, 2000; U.S. Pat. No. 6,734,173, WO 01/29233; WO03/085085; WO 02/012281; WO 02/061113).

Among these strategies was the linkage of antigen to the intracellular targeting moiety calreticulin (CRT). The present inventors and their colleagues were the first to provide naked DNA and self-replicating RNA vaccines that incorporated CRT (or other IPPs). The present inventors and their colleagues also demonstrated that linking antigen to Mycobacterium tuberculosis heat shock protein 70 (HSP70) or its C-terminal domain, domain II of Pseudomonas aeruginosa exotoxin A (ETA(dII)) enhanced DNA vaccine potency compared to compositions comprising only DNA encoding the antigen of interest. As discussed above, to enhance MHC class II antigen processing, the present inventors' colleagues (Lin, K Y et al., Cancer Res 56: 21-6, 1996) linked the sorting signals of the lysosome-associated membrane protein (LAMP-1) to the cytoplasmic/nuclear human papilloma virus (HPV-16) E7 antigen, creating a chimera (Sig/E7/LAMP-1). These findings point to the importance of adding an additional “element” to an antigenic composition at the DNA level to enhance in vivo potency of a recombinant DNA vaccine.

Intradermal administration of DNA vaccines via gene gun in vivo has proven to be an effective means to deliver such vaccines into professional antigen-presenting cells (APCs), primarily dendritic cells (DCs), which function in the uptake, processing, and presentation of antigen to T cells. The interaction between APCs and T cells is crucial for developing a potent specific immune response.

Even if current cancer therapies are effective, there remains a need for anticancer therapies that are yet more effective.

### SUMMARY OF THE INVENTION

Although antigen-specific DNA vaccines may be effective against small tumors inpreclinical models, many tumors can grow rapidly, resulting in bulky tumors which present a challenge to immunotherapeutic strategies alone. The present invention is directed at overcoming this challenge through multi-modality treatment regimens which combine immunotherapy, such as DNA vaccination, with an apoptosis-inducing chemotherapeutic drugs, such as epigallocatechin-3-gallate (EGCG), 5,6 di-methylxanthenone-4-acetic acid (DMXAA), cisplatin, apigenin, doxorubicin, an anti-death receptor 5 antibody, a proteasome inhibitor, an inhibitor of DNA methylation, genistein, celecoxib and biologically active analogs thereof. As shown in the current invention, a combination of cancer immunotherapy with a tumor-killing cancer drug is a plausible approach for the control of bulky tumors.

Provided herein are methods and kits for inhibiting tumor growth or treating a hyperproliferative disease using combinations of chemotherapeutic drugs, or their derivatives, and DNA vaccines. A hyperproliferative disease may be a cancer, such as cervical cancer, ano-genital cancer, prostate cancer, head and neck cancer, or a skin cancer, or a non-cancerous cellular growth. In some embodiments, the methods and kits disclosed herein may be used to induce apoptosis in tumors or cells involved in hyperproliferative disease. In certain embodiments, the methods and kits may be used to induce an immune response against a tumor or cells involved in a hyperproliferative disease. The methods and kits disclosed in this application may lead to both increased apoptotic cell death and an increase in the antigen-specific CD8+ and CD4+ T cell-mediated immune responses toward tumor cells, or other cells involved in hyperproliferative diseases.

In some embodiments, the present invention includes the use of DNA vaccines encoding IPPs, e.g., comprising lysosomal associated membrane protein 1 (LAMP-1), heat shock protein 70 (HSP70) from M. tuberculosis, ETA(dIII) from P. aeruginosa, calreticulin (CRT), VP22 or a biologically active homolog thereof. In certain embodiments, the methods and kits of the present invention may include a self-replicating RNA vector. One of skill in the art will readily recognize that other IPPs and vectors can be used with the methods and kits disclosed in the present invention.

The present invention may include the use of DNA sequences encoding antigenic peptides, e.g., those derived from human pailloma virus (HPV), HPV-16 E7, HPV-16 E6, Influenza hemagglutinin, Mycobacterium, Listeria, Bordetella, Ehrlichia, Staphylococcus, Toxoplasma, Legionella, Brucella, Salmonella, Chlamydia, Rickettsia, hepatitis B virus (HBV), hepatitis C virus (HCV), human immunodeficiency virus (HCV), herpesviruses, and antigens associated with parasitic pathogens, including Plasmodium and biologically active homologs thereof. In some embodiments, the methods and kits disclosed herein may also be used for the treatment of fungal infections, such as Paracoccidioides. One of skill in the art will readily recognize that other antigenic peptides can be used with the methods and kits disclosed in the present invention.

The methods and kits disclosed herein may also be used with siRNA sequences directed at modulating apoptotic signaling pathways in immune cells. Representative siRNA targets include Bax, Bak, caspase 8, caspase 9, and caspase 3. One of skill in the art will readily recognize that other siRNA targets in apoptotic signaling pathways can be used with the methods and kits disclosed in the present invention.

The methods and kits disclosed herein may also be used with DNA encoding anti-apoptotic proteins. Representative anti-apoptotic proteins include Bcl-2, Bcl-XL, XIAP, dominant negative mutants of caspase 8 and caspase 9, serine protease inhibitor 6 (SPI-6), and FLICEc-s. One of skill in the art will readily recognize that other anti-apoptotic proteins can be used with the methods and kits disclosed in the present invention.

Provided herein are methods for treating cancer in a subject, comprising administering to a subject in need thereof a DNA vaccine encoding a tumor antigen or a biologically active homolog thereof and an apoptosis-inducing chemotherapeutic drug. The chemotherapeutic drug may be selected from the group consisting of epigallocatechin-3-gallate (EGCG), 5,6 di-methylxanthenone-4-acetic acid (DMXAA), cisplatin, apigenin, doxorubicin, an anti-death receptor 5 antibody, a proteasome inhibitor, an inhibitor of DNA methylation, genistein, celecoxib and biologically active analogs thereof. The tumor antigen may be an antigen from a pathogenic organism, such as a viral antigen, e.g., an antigen from a human papilloma virus (HPV). The tumor antigen may be E6 or E7. HPV may be HPV-16.

The tumor antigen may be a protein that comprises an amino acid sequence that is at least about 90% identical to the amino acid sequence of an antigen from HPV or a biologically active fragment thereof. The tumor antigen may be a protein that comprises an amino acid sequence that is at least about 90% identical to the amino acid sequence of a detox E6 or detox E7 protein and comprising the amino acid substitutions that are specific to detox E6 or E7, respectively, or a biologically active fragment thereof.

The DNA vaccine may comprise a nucleotide sequence encoding a fusion protein comprising the tumor antigen or a biologically active homolog thereof and an immunogenicity-potentiating polypeptide (IPP). The IPP may comprise one or more of the translocation domain of a bacterial toxin, an endoplasmic reticulumn chaperone polypeptide, and an intercellular spreading protein or a biologically active homolog thereof. The IPP may comprise ETA(dII), HSP70, calreticulin, LAMP-1 or VP22 or a biologically active homolog thereof. The fusion protein may further comprise a linker linking the tumor antigen or the biologically active homolog thereof to the IPP.

In one embodiment, the chemotherapeutic drug is EGCG and at least one dose of EGCG is administered before the first dose of the DNA vaccine. In one embodiment, the chemotherapeutic drug is DMXAA and at least one dose of the DNA vaccine is administered before the first dose of DMXAA. In one embodiment, the chemotherapeutic drug is cisplatin and at least one dose of cisplatin is administered before the first dose of DNA vaccine.

A method may further comprise administering to the subject a nucleic acid that inhibits the expression of a pro-apoptotic protein and/or a nucleic acid that encoding an anti-apoptotic protein.

Also provided herein are compositions comprising a DNA vaccine encoding a tumor antigen or a biologically active homolog thereof and an apoptosis-inducing chemotherapeutic drug. Also provided are kits, e.g., for treating cancer, comprising a DNA vaccine encoding a tumor antigen or a biologically active homolog thereof and an apoptosis-inducing chemotherapeutic drug.

### BRIEF DESCRIPTION OF THE DRAWINGS

FIGS. 1A, 1B, 1C, and 1D. Tumor treated with EGCG induced apoptosis, generated HPV-16 E7-specific CD8⁺ T cells and inhibited tumor growth of E7-expressing tumors.

FIGS. 2A and 2B. TC-1 Tumor treated with EGCG generated higher levels of E7-peptide-loaded dendritic cells in the draining lymph nodes of tumor-bearing mice.

FIGS. 3A, 3B, and 3C. Combined DNA vaccination and EGCG treatment in the presence of tumor generated an enhanced E7-specific CD8⁺ T cell immune response as compared to monotherapy alone.

FIGS. 4A, 4B, 4C, and 4D. Characterization of E7-specific CD8⁺ T cell immune responses and anti-tumor effects generated by the Sig/E7/LAMP-1 DNA vaccine combined with EGCG.

FIGS. 5A and 5B. Combined DNA vaccination and EGCG treatment generated an enhanced Th1 E7-specific CD4⁺ T cell immune response.

FIGS. 6A, 6B, and 6C. Combined DNA vaccination and oral EGCG treatment generated a significant long-term immune response and antitumor protection in cured mice.

FIG. 7. Combined DNA vaccination and oral EGCG treatment generated synergistic anti-tumor therapeutic effects as compared to monotherapy alone.

FIG. 8. Schema for vaccination with DMXAA and DNA vaccination in naïve mice. Diagram showing the time lines of vaccination regimens.

FIG. 9. Flow cytometry analysis of the E7-specific CD8+ T cell response in mice vaccinated with CRT/E7 DNA and/or DMXAA showing that DMXAA enhances HPV16 E7-specific CD8+T cell response induced by CRT/E7 DNA vaccine in vaccinated mice.

FIG. 10. Flow cytometry analysis of the E6-specific CD8+ T cell response in mice vaccinated with CRT/E6 DNA and/or DMXAA showing that DMXAA enhances HPV16 E7-specific CD8+T cell response induced by CRT/E6 DNA vaccine in vaccinated mice.

FIG. 11. Schema for vaccination with DMXAA and DNA vaccination in TC-1 bearing mice. Diagram showing the time lines of vaccination regimens.

FIG. 12. Flow cytometry analysis of the E7-specific CD8+ T cell response in tumor challenged mice treated with CRT/E7 DNA and/or DMXAA showing that DMXAA enhances HPV16 E7-specific CD8+T cell response induced by CRT/E7 DNA vaccine in tumor bearing mice.

FIGS. 13A, 13B, 13C, and 13D. Immunohistochemical staining of tumor cells in tumor challenged mice treated with CRT/E7 DNA and/or DMXAA showing that DMXAA causes extensive tumor necrosis.

FIGS. 14A, 14B, 14C, and 14D. Immunohistochemical staining of tumor infiltrating immune cells in tumor challenged mice treated with CRT/E7 DNA and/or DMXAA, showing infiltration of inflammatory cells into the tumor.

FIG. 15. Characterization of HPV-16 E7-Specific Tumor Infiltrating CD8+ T Cells by E7 Peptide-Loaded MHC Class I Tetramer Staining.

FIG. 16. In vivo tumor treatment experiment. C57BL/6 tumor challenged mice were treated with CRT/E7 DNA vaccine and/or DMXAA as illustrated in FIG. 11, showing synergistic antitumor effects generated by combination of CRT/E7 vaccine with DMXAA.

FIG. 17. Schematic diagram of the treatment regimens of cisplatin and/or DNA vaccine. Diagrammatic representation of the different treatment regimens of cisplatin and/or DNA vaccine.

FIGS. 18A and 18B. In vivo tumor treatment experiments.

FIGS. 19A and 19B. Intracellular cytokine staining followed by flow cytometry analysis to determine the number of E7-specific CD8+ T cells in tumor challenged mice treated with cisplatin and/or DNA vaccine.

FIGS. 20A and 20B. Intracellular cytokine staining followed by flow cytometry analysis to determine the number of E7-specific CD8+ T cells in tumor challenged mice treated with or without cisplatin.

FIGS. 21A and 21B. In vitro cytotoxicity assay.

FIG. 22. Sequence of the pcDNA3 plasmid vector (SEQ ID NO: 1).

FIG. 23. Sequence of the pNGVL4a plasmid vector (SEQ ID NO: 2).

FIG. 24. Sequence of the pcDNA3-E7-Hsp70 plasmid (SEQ ID NO: 3).

FIG. 25. Sequence of the pcDNA3-ETA(dII)/E7 plasmid (SEQ ID NO: 4).

FIG. 26. Sequence of the pNGVL4a-CRT/E7(detox) plasmid (SEQ ID NO: 5).

FIG. 27. Nucleotide sequence of VP22/E7 DNA as it appears in the pcDNA3 vector (SEQ ID NO: 6) which is 1254 nucleotides (+stop codon). SEQ ID NO: 6 includes nucleotides 1-903 (upper case) encoding VP22 (SEQ ID NO: 7). Nucleotides 904-921 and the corresponding amino acids 302-307 are a “linker” sequence. Nucleotides 922-1209 (lower case) encode 96 of the 98 amino acids of wild-type E7 protein. Also shown is a stretch of vector sequence (underscored) from nucleotides 1210-1257 (including stop codon).

FIG. 28. Regimen for treatment with doxorubicin and a DNA vaccine in vaccinated mice.

FIGS. 29A and 29B. Anti-tumor effects generated by treatment with the mouse DR5 antibody and/or CRT/E7(detox) DNA vaccine in vaccinated mice.

FIGS. 30A and 30B. Anti-tumor effects generated by treatment with bortezomib and/or CRT/E7(detox) DNA vaccine in vaccinated mice.

FIGS. 31A and 31B. Anti-tumor effects generated by treatment with 5-aza-2-deoxycytidin and/or CRT/E7(detox) DNA vaccine in vaccinated mice.

FIGS. 32A and 32B. Anti-tumor effects generated by treatment with genistein and/or CRT/E7(detox) DNA vaccine in vaccinated mice.

FIGS. 33A and 33B. Anti-tumor effects generated by treatment with celecoxib and/or CRT/E7(detox) DNA vaccine in vaccinated mice.

FIGS. 34A and 34B. Anti-tumor effects generated by treatment with apigenin and/or E7-HSP70 DNA vaccine in vaccinated mice.

### DETAILED DESCRIPTION

### Partial List of Abbreviations

APC, antigen presenting cell; CRT, calreticulin; CTL, cytotoxic T lymphocyte; DC, dendritic cell; ECD, extracellular domain; EGCG, epigallocatechin-3-gallate; E6, HPV oncoprotein E6; E7, HPV oncoproteinE7; ELISA, enzyme-linked immunosorbent assay; HPV, human papillomavirus; HSP, heat shock protein; Hsp70, mycobacterial heat shock protein 70; IFN γ, interferon-γ; i.m., intramuscular(ly); i.v., intravenous(ly); MHC, major histocompatibility complex; PBS, phosphate-buffered saline; PCR, polymerase chain reaction; β-gal, β-galactosidase

### General

Provided herein are methods for treating a hyperproliferating disease, e.g., cancer, comprising administering to a subject in need thereof (i) a vaccine, e.g., a DNA vaccine, encoding an antigen or a biologically active homolog thereof and (ii) a drug such as a chemotherapeutic drug, e.g., an apoptosis-inducing chemotherapeutic drug. An antigen may be an antigen from a hyperproliferating, e.g., cancer, cell. A subject in need thereof may be a subject having been diagnosed with cancer. Also provided are methods for enhancing the efficacy of a vaccine, e.g., DNA vaccine, in a subject, comprising administering a chemotherapeutic drug to a subject who is treated with the vaccine. Further provided are methods for enhancing the efficacy of a chemotherapeutic drug in a subject, comprising administering a vaccine, e.g., DNA vaccine, to a subject who is treated with the chemotherapeutic drug.

### Chemotherapeutic Drugs

Generally, any drug that reduces the growth of cells without significantly affecting the immune system may be used, or at least not suppressing the immune system to the extent of eliminating the positive effects of a DNA vaccine that is administered to the subject. Preferred drugs are chemotherapeutic drugs.

A wide variety of chemotherapeutic drugs may be used, provided that the drug stimulates the effect of a vaccine, e.g., DNA vaccine. In certain embodiments, a chemotherapeutic drug may be a drug that (a) induces apoptosis of cells, in particular, cancer cells, when contacted therewith; (b) reduces tumor burden; and/or (c) enhances CD8+ T cell-mediated antitumor immunity. In certain embodiments, the drug must also be on that does not inhibit the immune system, or at least not at certain concentrations.

In one embodiment, the chemotherapeutic drug is epigallocatechin-3-gallate (EGCG) or a chemical derivative or pharmaceutically acceptable salt thereof. Epigallocatechin gallate (EGCG) is the major polyphenol component found in green tea (for reviews, see (12-17)). EGCG has demonstrated antitumor effects in various human and animal models, including cancers of the breast, prostate, stomach, esophagus, colon, pancreas, skin, lung, and other sites (for reviews, see (18, 19, 12)). EGCG has been shown to act on different pathways to regulate cancer cell growth, survival, angiogenesis and metastasis (for review see (12, 13, 20)). For example, some studies suggest that EGCG protects against cancer by causing cell cycle arrest and inducing apoptosis (21). It is also reported that telomerase inhibition might be one of the major mechanisms underlying the anticancer effects of EGCG (22, 23). In comparison with commonly-used antitumor agents, including retinoids and doxorubicin, EGCG has a relatively low toxicity and is convenient to administer due to its oral bioavailability (24, 25). Thus, EGCG has been used in clinical trials (26) and appears to be a potentially ideal antitumor agent (27, 28).

Exemplary analogs or derivatives of EGCG include (−)-EGCG, (+)-EGCG, (−)-EGCG-amide, (−)-GCG, (+)-GCG, (+)-EGCG-amide, (−)-ECG, (−)-CG, genistein, GTP-1, GTP-2, GTP-3, GTP-4, GTP-5, Bn-(+)-epigallocatechin gallate (US 2004/0186167), and dideoxy-epigallocatechin gallate (Furuta, et al., Bioorg. Med. Chem. Letters, 2007, 11: 3095-3098), For additional examples, see US 2004/0186167 (incorporated by reference in its entirety); Waleh, et al., Anticancer Res., 2005, 25: 397-402; Wai, et al., Bioorg. Med. Chem., 2004, 12: 5587-5593; Smith, et al., Proteins: Struc. Func. & Bioinform., 2003, 54: 58-70; U.S. Pat. No. 7,109,236 (incorporated by reference in its entirety); Landis-Piwowar, et al., Int. J. Mol. Med., 2005, 15: 735-742; Landis-Piwowar, et al., J. Cell. Phys., 2007, 213: 252-260; Daniel, et al., Int. J. Mol. Med., 2006, 18: 625-632; Tanaka, et al., Ang. Chemie Int., 2007, 46: 5934-5937.

Another chemotherapeutic drug that may be used is (a) 5,6 di-methylxanthenone-4-acetic acid (DMXAA), or a chemical derivative or analog thereof or a pharmaceutically acceptable salt thereof. Exemplary analogs or derivatives include xanthenone-4-acetic acid, flavone-8-acetic acid, xanthen-9-one-4-acetic acid, methyl (2,2-dimethyl-6-oxo-1,2-dihydro-6H-3,11-dioxacyclopentaManthracen-10-yl)acetate, methyl (2-methyl-6-oxo-1,2-dihydro-6H-3,11-dioxacyclopenta[α]anthracen-10-yl)acetate, methyl (3,3-dimethyl-7-oxo-3H,7H-4,12-dioxabenzo[α]anthracen-10-yl)acetate, methyl-6-alkyloxyxanthen-9-one-4-acetates (Gobbi, et al., 2002, J. Med. Chem., 45: 4931) or a. For additional examples, see WO 2007/023302 A1, WO 2007/023307 A1, US 2006/9505, WO 2004/39363 A1, WO 2003/80044 A1, AU 2003/217035 A1, and AU 2003/282215 A1, each incorporated by reference in their entirety.

A chemotherapeutic drug may also be cisplatin, or a chemical derivative or analog thereof or a pharmaceutically acceptable salt thereof. Exemplary analogs or derivatives include dichloro[4,4′-bis(4,4,4-trifluorobutyl)-2,2′-bipyridine]platinum (Kyler et al., Bioorganic & Medicinal Chemistry, 2006, 14: 8692-8700), cis-[Rh2(-O2CCH3)2(CH3CN)6]2+ (Lutterman et al., J. Am. Chem. Soc., 2006, 128: 738-739), (+)-cis-(1,1-Cyclobutanedicarboxylato)((2R)-2-methyl-1,4-butanediamine-N,N′)platinum (O'Brien et al., Cancer Res., 1992, 52: 4130-4134), cis-bisneodecanoato-trans-R,R-1,2-diaminocyclohexane platinum(II) (Lu et al., J. of Clin. Oncol., 2005, 23: 3495-3501), carboplatin (Woloschuk, Drug Intell. Clin. Pharm., 1988, 22: 843-849), sebriplatin (Kanazawa et al., Head & Neck, 2006, 14: 38-43), satraplatin (Amorino et al., Cancer Chemother. and Pharmacol., 2000, 46: 423-426), azane (dichloroplatinum) (CID: 11961987), azanide (CID: 6712951), platinol (CID: 5702198), lopac-P-4394 (CID: 5460033), MOLI001226 (CID: 450696), trichloroplatinum (CID: 420479), platinate(1-), amminetrichloro-, ammonium (CID: 160995), triammineplatinum (CID: 119232), biocisplatinum (CID: 84691), platiblastin (CID: 2767) and pharmaceutically acceptable salts thereof. For additional examples, see U.S. Pat. No. 5,922,689, U.S. Pat. No. 4,996,337, U.S. Pat. No. 4,937,358, U.S. Pat. No. 4,808,730, U.S. Pat. No. 6,130,245, U.S. Pat. No. 7,232,919, and U.S. Pat. No. 7,038,071, each incorporated by reference in their entirety.

Another chemotherapeutic drug that may be used is apigenin, or a chemical derivative or analog thereof or a pharmaceutically acceptable salt thereof. Exemplary analogs or derivatives include acacetin, chrysin, kampherol, luteolin, myricetin, naringenin, quercetin (Wang et al., Nutrition and Cancer, 2004, 48: 106-114), puerarin (US 2006/0276458, incorporated by reference in its entirety) and pharmaceutically acceptable salts thereof. For additional examples, see US 2006/189680 A1, incorporated by reference in its entirety).

Another chemotherapeutic drug that may be used is doxorubicin, or a chemical derivative or analog thereof or a pharmaceutically acceptable salt thereof. Exemplary analogs or derivatives include anthracyclines, 3′-deamino-3′-(3-cyano-4-morpholinyl)doxorubicin, WP744 (Faderl, et al., Cancer Res., 2001, 21: 3777-3784), annamycin (Zou, et al., Cancer Chemother. Pharmacol., 1993, 32:190-196), 5-imino-daunorubicin, 2-pyrrolinodoxorubicin, DA-125 (Lim, et al., Cancer Chemother. Pharmacol., 1997, 40: 23-30), 4-demethoxy-4′-O-methyldoxorubicin, PNU 152243 and pharmaceutically acceptable salts thereof (Yuan, et al., Anti-Cancer Drugs, 2004, 15: 641-646). For additional examples, see EP 1242438 B1, U.S. Pat. No. 6,630,579, AU 2001/29066 B2, U.S. Pat. No. 4,826,964, U.S. Pat. No. 4,672,057, U.S. Pat. No. 4,314,054, AU 2002/358298 A1, and U.S. Pat. No. 4,301,277, each incorporated by reference in their entirety);

Other chemotherapeutic drugs that may be used are anti-death receptor 5 antibodies and binding proteins, and their derivatives, including antibody fragments, single-chain antibodies (scFvs), Avimers, chimeric antibodies, humanized antibodies, human antibodies and peptides binding death receptor 5. For examples, see US 2007/31414 and US 2006/269554, each incorporated by reference in their entirety.

Another chemotherapeutic drug that may be used is bortezomib, or a chemical derivative or analog thereof or a pharmaceutically acceptable salt thereof. Exemplary analogs or derivatives include MLN-273 and pharmaceutically acceptable salts thereof (Witola, et al., Eukaryotic Cell, 2007, doi:10.1128/EC.00229-07). For additional possibilities, see Groll, et al., Structure, 14:451.

Another chemotherapeutic drug that may be used is 5-aza-2-deoxycytidine, or a chemical derivative or analog thereof or a pharmaceutically acceptable salt thereof. Exemplary analogs or derivatives include other deoxycytidine derivatives and other nucleotide derivatives, such as deoxyadenine derivatives, deoxyguanine derivatives, deoxythymidine derivatives and pharmaceutically acceptable salts thereof.

Another chemotherapeutic drug that may be used is genistein, or a chemical derivative or analog thereof or a pharmaceutically acceptable salt thereof. Exemplary analogs or derivatives include 7-O-modified genistein derivatives (Zhang, et al., Chem. & Biodiv., 2007, 4: 248-255), 4′,5,7-tri[3-(2-hydroxyethylthio)propoxy]isoflavone, genistein glycosides (Polkowski, Cancer Letters, 2004, 203: 59-69), other genistein derivatives (Li, et al., Chem & Biodiv., 2006, 4: 463-472; Sarkar, et al., Mini. Rev. Med. Chem., 2006, 6: 401-407) or pharmaceutically acceptable salts thereof. For additional examples, see U.S. Pat. No. 6,541,613, U.S. Pat. No. 6,958,156, and WO/2002/081491, each incorporated by reference in their entirety.

Another chemotherapeutic drug that may be used is celecoxib, or a chemical derivative or analog thereof or a pharmaceutically acceptable salt thereof. Exemplary analogs or derivatives include N-(2-aminoethyl)-4-[5-(4-tolyl)-3-(trifluoromethyl)-1H-pyrazol-1-yl]benzenesulfonamide, 4-[5-(4-aminophenyl)-3-(trifluoromethyl)-1H-pyrazol-1-yl]benzenesulfonamide, OSU03012 (Johnson, et al., Blood, 2005, 105: 2504-2509), OSU03013 (Tong, et. al, Lung Cancer, 2006, 52: 117-124), dimethyl celecoxib (Backhus, et al., J. Thorac. and Cardiovasc. Surg., 2005, 130: 1406-1412), and other derivatives or pharmaceutically acceptable salts thereof (Ding, et al., Int. J. Cancer, 2005, 113: 803-810; Zhu, et al., Cancer Res., 2004, 64: 4309-4318; Song, et al., J. Natl. Cancer Inst., 2002, 94: 585-591). For additional examples, see U.S. Pat. No. 7,026,346, incorporated by reference in its entirety.

One of skill in the art will readily recognize that other chemotherapeutics can be used with the methods and kits disclosed in the present invention, including proteasome inhibitors (in addition to bortezomib) and inhibitors of DNA methylation. Other drugs that may be used include Paclitaxel; selenium compounds; SN38, etoposide, 5-Fluorouracil; VP-16, cox-2 inhibitors, Vioxx, cyclooxygenase-2 inhibitors, curcumin, MPC-6827, tamoxifen or flutamide, etoposide, PG490, 2-methoxyestradiol, AEE-788, aglycon protopanaxadiol, aplidine, ARQ-501, arsenic trioxide, BMS-387032, canertinib dihydrochloride, canfosfamide hydrochloride, combretastatin A-4 prodrug, idronoxil, indisulam, INGN-201, mapatumumab, motexafin gadolinium, oblimersen sodium, OGX-011, patupilone, PXD-101, rubitecan, tipifarnib, trabectedin PXD-101, methotrexate, Zerumbone, camptothecin, MG-98, VX-680, Ceflatonin, Oblimersen sodium, motexafin gadolinium, 1D09C3, PCK-3145, ME-2 and apoptosis-inducing-ligand (TRAIL/Apo-2 ligand). Others are provided in a report entitled “competitive outlook on apoptosis in oncology, December 2006, published by Bioseeker, and available, e.g., at http://bizwiz.bioseeker.com/bw/Archives/Files/TOC_BSG0612193.pdf.

Generally, any drug that affects an apoptosis target may also be used. Apoptosis targets include the tumour-necrosis factor (TNF)-related apoptosis-inducing ligand (TRAIL) receptors, the BCL2 family of anti-apoptotic proteins (such as Bcl-2), inhibitor of apoptosis (IAP) proteins, MDM2, p53, TRAIL and caspases. Exemplary targets include B-cell CLL/lymphoma 2, Caspase 3, CD4 molecule, Cytosolic ovarian carcinoma antigen 1, Eukaryotic translation elongation factor 2, Farnesyltransferase, CAAX box, alpha; Fc fragment of IgE; Histone deacetylase 1; Histone deacetylase 2; Interleukin 13 receptor, alpha 1; Phosphodiesterase 2A, cGMP-stimulatedPhosphodiesterase 5A, cGMP-specific; Protein kinase C, beta 1; Steroid 5-alpha-reductase, alpha polypeptide 1; 8.1.15 Topoisomerase (DNA) I; Topoisomerase (DNA) II alpha; Tubulin, beta polypeptide; and p53 protein.

In certain embodiments, the compounds described herein, e.g., EGCG, are naturally-occurring and may, e.g., be isolated from nature. Accordingly, in certain embodiments, a compound is used in an isolated or purified form, i.e., it is not in a form in which it is naturally occurring. For example, an isolated compound may contain less than about 50%, 30%, 10%, 1%, 0.1% or 0.01% of a molecule that is associated with the compound in nature. A purified preparation of a compound may comprise at least about 50%, 70%, 80%, 90%, 95%, 97%, 98% or 99% of the compound, by molecule number or by weight. Compositions may comprise, consist essentially of consist of one or more compounds described herein. Some compounds that are naturally occurring may also be synthesized in a laboratory and may be referred to as “synthetic.” Yet other compounds described herein are non-naturally occurring.

In certain embodiments, the chemotherapeutic drug is in a preparation from a natural source, e.g., a preparation from green tea.

Pharmaceutical compositions comprising 1, 2, 3, 4, 5 or more chemotherapeutic drugs or pharmaceutically acceptable salts thereof are also provided herein. A pharmaceutical composition may comprise a pharmaceutically acceptable carrier. A composition, e.g., a pharmaceutical composition, may also comprise a vaccine, e.g., a DNA vaccine, and optionally 1, 2, 3, 4, 5 or more vectors, e.g., other DNA vaccines or other constructs, e.g., described herein.

Compounds may be provided with a pharmaceutically acceptable salt. The term “pharmaceutically acceptable salts” is art-recognized, and includes relatively non-toxic, inorganic and organic acid addition salts of compositions, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66:1-19 (1977).

DNA Vaccines

Any vaccine, e.g., protein or DNA vaccine, may be used as described herein. In a preferred embodiment, a vaccine is a nucleic acid vaccine, e.g., a DNA vaccine. Any type of nucleic acid vaccine may be used, provided that its effect is increased by administration of a chemotherapeutic drug, as described herein. A DNA vaccine may encode one or more antigens (e.g., 1, 2, 3, 4, 5 or more).

The experiments described herein demonstrate that the methods of the invention can enhance a cellular immune response, particularly, tumor-destructive CTL reactivity, induced by a DNA vaccine encoding an epitope of a human pathogen. Human HPV-16 E7 was used as a model antigen for vaccine development because human papillomaviruses (HPVs), particularly HPV-16, are associated with most human cervical cancers. The oncogenic HPV proteins E7 and E6 are important in the induction and maintenance of cellular transformation and co-expressed in most HPV-containing cervical cancers and their precursor lesions. Therefore, cancer vaccines, such as the compositions of the invention, that target E7 or E6 can be used to control of HPV-associated neoplasms (Wu, T-C, Curr Opin Immunol. 6:746-54, 1994).

However, as noted, the present invention is not limited to the exemplified antigen(s). Rather, one of skill in the art will appreciate that the same results are expected for any antigen (and epitopes thereof) for which a T cell-mediated response is desired. The response so generated will be effective in providing protective or therapeutic immunity, or both, directed to an organism or disease in which the epitope or antigenic determinant is involved—for example as a cell surface antigen of a pathogenic cell or an envelope or other antigen of a pathogenic virus, or a bacterial antigen, or an antigen expressed as or as part of a pathogenic molecule.

Exemplary antigens and their sequences are set forth below.

E7 Protein from HPV-16

The E7 nucleic acid sequence (SEQ ID NO: 8) and amino acid sequence (SEQ ID NO: 9) from HPV-16 are shown below (see GenBank Accession No. NC_{—}001526)

| | |
|---|---|
| atg cat gga gat aca cct aca ttg cat gaa tat atg tta gat ttg caa cca gag aca act | 60 |
| Met His Gly Asp Thr Pro Thr Leu His Glu Tyr Met Leu Asp Leu Gln Pro Glu Thr Thr | 20 |
| gat ctc tac tgt tat gag caa tta aat gac agc tca gag gag gag gat gaa ata gat ggt | 120 |
| Asp Leu Tyr Cys Tyr Glu Gln Leu Asn Asp Ser Ser Glu Glu Glu Asp Glu Ile Asp Gly | 40 |
| cca gct gga caa gca gaa ccg gac aga gcc cat tac aat att gta acc ttt tgt tgc aag | 180 |
| Pro Ala Gly Gln Ala Glu Pro Asp Arg Ala His Tyr Asn Ile Val Thr Phe Cys Cys Lys | 60 |
| tgt gac tct acg ctt cgg ttg tgc gta caa agc aca cac gta gac att cgt act ttg gaa | 240 |
| Cys Asp Ser Thr Leu Arg Leu Cys Val Gln Ser Thr His Val Asp Ile Arg Thr Leu Glu | 80 |
| gac ctg tta atg ggc aca cta gga att gtg tgc ccc atc tgt tct cag gat aag ctt | 297 |
| Asp Leu Leu Met Gly Thr Leu Gly Ile Val Cys Pro Ile Cys Ser Gln Asp Lys Leu | 99 |

In single letter code, the wild type E7 amino acid sequence is:

(SEQ ID NO: 9 above)

| MHGDTPTLHE YMLDLQPETT DLYCYEQLND SSEEEDEIDG | |
|---|---|
| PAGQAEPDRA HYNIVTFCCK CDSTLRLCVQ STHVDIRTLE | |
| DLLMGTLGIV CPICSQDKL | 99 |

In another embodiment (See GenBank Accession No. AF125673, nucleotides 562-858 and the E7 amino acid sequence), the C-terminal four amino acids QDKL (and their codons) above are replaced with the three amino acids QKP (and the codons cag aaa cca), yielding a protein of 98 residues.

When an oncoprotein or an epitope thereof is the immunizing moiety, it is preferable to reduce the tumorigenic risk of the vaccine itself. Because of the potential oncogenicity of the HPV E7 protein, the E7 protein is preferably used in a “detoxified” form.

To reduce oncogenic potential of E7 in a construct of this invention, one or more of the following positions of E7 is mutated:

| | | | | |
|---|---|---|---|---|
| Original | Mutant | Preferred | nt Position | Amino acid (in |
| residue | residue | codon mutation | (in SEQ ID NO: 8) | SEQ ID NO: 9) |

| | | | | |
|---|---|---|---|---|
| Cys | Gly (or Ala) | TGT→GGT | 70 | 24 |
| Glu | Gly (or Ala) | GAG→GGG | 77 | 26 |
| | | (or GCG) | | |
| Cys | Gly (or Ala) | TGC→GGC | 271 | 91 |

The preferred E7 (detox) mutant sequence has the following two mutations: a TGT→GGT mutation resulting in a Cys→Gly substitution at position 24 of SEQ ID NO: 9 a and GAG→GGG mutation resulting in a Glu→Gly substitution at position 26 of SEQ ID NO: 9. This mutated amino acid sequence is shown below with the replacement residues underscored:

(SEQ ID NO: 10)

MHGDTPTLHE YMLDLQPETT DLYGYEGLND SSEEEDEIDG
PAGQAEPDRA HYNIVTFCCK CDSTLRLCVQ STHVDIRTLE
DLLMGTLGIV CPICSQKP 97

These substitutions completely eliminate the capacity of the E7 to bind to Rb, and thereby nullify its transforming activity. Any nucleotide sequence that encodes the above E7 or E7(detox) polypeptide, or an antigenic fragment or epitope thereof, can be used in the present compositions and methods, though the preferred E7 and E7(detox) sequences are shown above.
E6 Protein from HPV-16

The wild type E6 nucleotide (SEQ ID NO: 11) and amino acid (SEQ ID NO: 12) sequences are shown below (see GenBank accession Nos. K02718 and NC_{—}001526)):

| | |
|---|---|
| atg cac caa aag aga act gca atg ttt cag gac cca cag gag cga ccc aga aag tta cca | 60 |
| Met His Gln Lys Arg Thr Ala Met Phe Gln Asp Pro Gln Glu Arg Pro Arg Lys Leu Pro | 20 |
| cag tta tgc aca gag ctg caa aca act ata cat gat ata ata tta gaa tgt gtg tac tgc | 120 |
| Gln Leu Cys Thr Glu Leu Gln Thr Thr Ile His Asp Ile Ile Leu Glu Cys Val Tyr Cys | 40 |
| aag caa cag tta ctg cga cgt gag gta tat gac ttt gct ttt cgg gat tta tgc ata gta | 180 |
| Lys Gln Gln Leu Leu Arg Arg Glu Val Tyr Asp Phe Ala Phe Arg Asp Leu Cys Ile Val | 60 |
| tat aga gat ggg aat cca tat gct gta tgt gat aaa tgt tta aag ttt tat tct aaa att | 240 |
| Tyr Arg Asp Gly Asn Pro Tyr Ala Val Cys Asp Lys Cys Leu Lys Phe Tyr Ser Lys Ile | 80 |
| agt gag tat aga cat tat tgt tat agt ttg tat gga aca aca tta gaa cag caa tac aac | 300 |
| Ser Glu Tyr Arg His Tyr Cys Tyr Ser Leu Tyr Gly Thr Thr Leu Glu Gln Gln Tyr Asn | 100 |
| aaa ccg ttg tgt gat ttg tta att agg tgt att aac tgt caa aag cca ctg tgt cct gaa | 360 |
| Lys Pro Leu Cys Asp Leu Leu Ile Arg Cys Ile Asn Cys Gln Lys Pro Leu Cys Pro Glu | 120 |
| gaa aag caa aga cat ctg gac aaa aag caa aga ttc cat aat ata agg ggt cgg tgg acc | 420 |
| Glu Lys Gln Arg His Leu Asp Lys Lys Gln Arg Phe His Asn Ile Arg Gly Arg Trp Thr | 140 |
| ggt cga tgt atg tct tgt tgc aga tca tca aga aca cgt aga gaa acc cag ctg taa | 474 |
| Gly Arg Cys Met Ser Cys Cys Arg Ser Ser Arg Thr Arg Arg Glu Thr Gln Leu stop | 158 |

This polypeptide has 158 amino acids and is shown below in single letter code:

[SEQ ID NO: 12, above]

MHQKRTAMFQ DPQERPRKLP QLCTELQTTI HDIILECVYC
KQQLLRREVY DFAFRDLCIV YRDGNPYAVC DKCLKFYSKI
SEYRHYCYSL YGTTLEQQYN KPLCDLLIRC INCQKPLCPE
EKQRHLDKKQ RFHNIRGRWT GRCMSCCRSS RTRRETQL 158

E6 proteins from cervical cancer-associated HPV types such as HPV-16 induce proteolysis of the p53 tumor suppressor protein through interaction with E6-AP. Human mammary epithelial cells (MECs) immortalized by E6 display low levels of p53. HPV-16 E6, as well as other cancer-related papillomavirus E6 proteins, also binds the cellular protein E6BP (ERC-55). As with E7, described below, it is preferred to used a non-oncogenic mutated form of E6, referred to as “E6(detox).” Several different E6 mutations and publications describing them are discussed below.

The preferred amino acid residues to be mutated are underscored in the E6 amino acid sequence above. Some studies of E6 mutants are based upon a shorter E6 protein of 151 nucleic acids, wherein the N-terminal residue was considered to be the Met at position 8 in SEQ ID NO: 12 above. That shorter version of E6 is shown below as SEQ ID NO: 13.

MFQDPQERPR KLPQLCTELQ TTIHDIILEC VYCKQQLLRR
EVYDFAFRDL CIVYRDGNPY AVCDKCLKFY
SKISEYRHYC YSLYGTTLEQ QYNKPLCDLL IRCINCQKPL
CPEEKQRHLD KKQRFHNIRG RWTGRCMSCC
RSSRTRRETQ L

To reduce oncogenic potential of E6 in a construct of this invention, one or more of the following positions of E6 is mutated:

| Original | Mutant | aa position in | aa position in |
|---|---|---|---|
| residue | residue | SEQ ID NO: 12 | SEQ ID NO: 13 |
| Cys | Gly (or Ala) | 70 | 63 |
| Cys | Gly (or Ala) | 113 | 106 |
| Ile | Thr | 135 | 128 |

Nguyen et al., J virol. 6:13039-48, 2002, described a mutant of HPV-16 E6 deficient in binding α-helix partners which displays reduced oncogenic potential in vivo. This mutant, which includes a replacement of Ile with Thr as position 128 (of SEQ ID NO: 13), may be used in accordance with the present invention to make an E6 DNA vaccine that has a lower risk of being oncogenic. This E6(I¹²⁸T) mutant is defective in its ability to bind at least a subset of α-helix partners, including E6AP, the ubiquitin ligase that mediates E6-dependent degradation of the p53 protein,

Cassetti M C et al., Vaccine 22:520-52, 2004, examined the effects of mutations four or five amino acid positions in E6 and E7 to inactivate their oncogenic potential. The following mutations were examined: E6-C⁶³G and E6 C¹⁰⁶G (positions based on SEQ ID NO: 13); E7-C²⁴G, E7-E²⁶G, and E7 C⁹¹G (positions based on SEQ ID NO: 9). Venezuelan equine encephalitis virus replicon particle (VRP) vaccines encoding mutant or wild type E6 and E7 proteins elicited comparable CTL responses and generated comparable antitumor responses in several HPV16 E6(+)E7(+) tumor challenge models: protection from either C3 or TC-1 tumor challenge was observed in 100% of vaccinated mice. Eradication of C3 tumors was observed in approximately 90% of the mice. The predicted inactivation of E6 and E7 oncogenic potential was confirmed by demonstrating normal levels of both p53 and Rb proteins in human mammary epithelial cells infected with VRPs expressing mutant E6 and E7 genes.

The HPV16 E6 protein contains two zinc fingers important for structure and function; one cysteine (C) amino acid position in each pair of C-X-X-C (where X is any amino acid) zinc finger motifs are preferably was mutated at E6 positions 63 and 106 (based on SEQ ID NO: 13). Mutants are created, for example, using the Quick Change Site-Directed Mutagenesis Kit (Stratagene, La Jolla, Calif.). HPV16 E6 containing a single point mutation in the codon for Cys¹⁰⁶ in SEQ ID NO: 13 (=Cys 113 in SEQ ID NO: 12). Cys¹⁰⁶ neither binds nor facilitates degradation of p53 and is incapable of immortalizing human mammary epithelial cells (MEC), a phenotype dependent upon p53 degradation. A single amino acid substitution at position Cys⁶³ of SEQ ID NO: 13 (=Cys⁷⁰ in SEQ ID NO: 12) destroys several HPV16 E6 functions: p53 degradation, E6TP-1 degradation, activation of telomerase, and, consequently, immortalization of primary epithelial cells.

Any nucleotide sequence that encodes these E6 polypeptides, or preferably, one of the mutants thereof, or an antigenic fragment or epitope thereof, can be used in the present invention. Other mutations can be tested and used in accordance with the methods described herein including those described in Cassetti et al., supra. These mutations can be produced from any appropriate starting sequences by mutation of the coding DNA.

The present invention also includes the use of a tandem E6-E7 vaccine, using one or more of the mutations described herein to render the oncoproteins inactive with respect to their oncogenic potential in vivo. VRP vaccines (described in Cassetti et al., supra) comprised fused E6 and E7 genes in one open reading frame which were mutated at four or five amino acid positions (see below). Thus, the present constructs may include one or more epitopes of E6 and E7, which may be arranged in their native order or shuffled in any way that permits the expressed protein to bear the E6 and E7 antigenic epitopes in an immunogenic form. DNA encoding amino acid spacers between E6 and E7 or between individual epitopes of these proteins may be introduced into the vector, provided again, that the spacers permit the expression or presentation of the epitopes in an immunogenic manner after they have been expressed by transduced host cells.

### Influenza Hemagglutinin (HA)

A nucleic acid sequence encoding HA [SEQ ID NO: 14] is shown below.

atgaaggcaaacctactggtcctgttaagtgcacttgcagctgcagatgcagacacaatatgtataggctaccatgcgaacaat
tcaaccgacactgttgacacagtactcgagaagaatgtgacagtgacacactctgttaacctgctcgaagacagccacaacgga
aaactatgtagattaaaaggaatagccccactacaattggggaaatgtaacatcgccggatggctcttgggaaacccagaatgc
gacccactgcttccagtgagatcatggtcctacattgtagaaacaccaaactctgagaatggaatatgttatccaggagatttc
atcgactatgaggagctgagggagcaattgagctcagtgtcatcattcgaaagattcgaaatatttcccaaagaaagctcatgg
cccaaccacaacacaaacggagtaacggcagcatgctcccatgaggggaaaagcagtttttacagaaatttgctatggctgacg
gagaaggagggctcatacccaaagctgaaaaattcttatgtgaacaaaaaagggaaagaagtccttgtactgtggggtattcat
cacccgcctaacagtaaggaacaacagaatatctatcagaatgaaaatgcttatgtctctgtagtgacttcaaattataacagg
agatttaccccggaaatagcagaaagacccaaagtaagagatcaagctgggaggatgaactattactggaccttgctaaaaccc
ggagacacaataatatttgaggcaaatggaaatctaatagcaccaatgtatgctttcgcactgagtagaggctttgggtccggc
atcatcacctcaaacgcatcaatgcatgagtgtaacacgaagtgtcaaacacccctgggagctataaacagcagtctcccttac
cagaatatacacccagtcacaataggagagtgcccaaaatacgtcaggagtgccaaattgaggatggttacaggactaaggaac
actccgtccattcaatccagaggtctatttggagccattgccggttttattgaagggggatggactggaatgatagatggatgg
tatggttatcatcatcagaatgaacagggatcaggctatgcagcggatcaaaaaagcacacaaaatgccattaacgggattaca
aacaaggtgaacactgttatcgagaaaatgaacattcaattcacagctgtgggtaaagaattcaacaaattagaaaaaaggatg
gaaaatttaaataaaaaagttgatgatggatttctggacatttggacatataatgcagaattgttagttctactggaaaatgaa
aggactctggatttccatgactcaaatgtgaagaatctgtatgagaaagtaaaaagccaattaaagaataatgccaaagaaatc
ggaaatggatgttttgagttctaccacaagtgtgacaatgaatgcatggaaagtgtaagaaatgggacttatgattatcccaaa
tattcagaagagtcaaagttgaacagggaaaaggtagatggagtgaaattggaatcaatggggatctatcagattctggcgatc
tactcaactgtcgccagttcactggtgcttttggtctccctgggggcaatcagtttctggatgtgttctaatggatctttgcag
tgcagaatatgcatctga

The amino acid sequence of HA [SEQ ID NO: 15; immunodominant epitope underscored, is:

MKANLLVLLS ALAAADADTI CIGYHANNST DTVDTVLEKN VTVTHSVNLL EDSHNGKLCR LKGIAPLQLG
KCNIAGWLLG NPECDPLLPV RSWSYIVETP NSENGICYPG DFIDYEELRE QLSSVSSFER FEIFPKESSW
PNHNTNGVTA ACSHEGKSSF YRNLLWLTEK EGSYPKLKNS YVNKKGKEVL VLWGIHHPPN SKEQQNIYQN
ENAYVSVVTS NYNRRFTPEI AERPKVRDQA GRMNYYWTLL KPGDTIIFEA NGNLIAPMYA FALSRGFGSG
IITSNASMHE CNTKCQTPLG AINSSLPYQN IHPVTIGECP KYVRSAKLRM VTGLRNTPSI QSRGLFGAIA
GFIEGGWTGM IDGWYGYHHQ NEQGSGYAAD QKSTQNAING ITNKVNTVIE KMNIQFTAVG KEFNKLEKRM
ENLNKKVDDG FLDIWTYNAE LLVLLENERT LDFHDSNVKN LYEKVKSQLK NNAKEIGNGC FEFYHKCDNE
CMESVRNGTY DYPKYSEESK LNREKVDGVK LESMGIYQIL AIYSTVASSL VLLVSLGAIS FWMCSNGSLQ
CRICI

### Other Exemplary Antigens

Exemplary antigens are epitopes of pathogenic microorganisms against which the host is defended by effector T cells responses, including CTL and delayed type hypersensitivity. These typically include viruses, intracellular parasites such as malaria, and bacteria that grow intracellularly such as Mycobacterium and Listeria species. Thus, the types of antigens included in the vaccine compositions of this invention may be any of those associated with such pathogens as well as tumor-specific antigens. It is noteworthy that some viral antigens are also tumor antigens in the case where the virus is a causative factor in the tumor.

In fact, the two most common cancers worldwide, hepatoma and cervical cancer, are associated with viral infection. Hepatitis B virus (HBV) (Beasley, R. P. et al., Lancet 2:1129-1133 (1981) has been implicated as etiologic agent of hepatomas. About 80-90% of cervical cancers express the E6 and E7 antigens (discussed above and exemplified herein) from one of four “high risk” human papillomavirus types: HPV-16, HPV-18, HPV-31 and HPV-45 (Gissmann, L. et al., Ciba Found Symp. 120:190-207, 1986; Beaudenon, S., et al. Nature 321:246-9, 1986). The HPV E6 and E7 antigens are the most promising targets for virus associated cancers in immunocompetent individuals because of their ubiquitous expression in cervical cancer. In addition to their importance as targets for therapeutic cancer vaccines, virus-associated tumor antigens are also ideal candidates for prophylactic vaccines. Indeed, introduction of prophylactic HBV vaccines in Asia have decreased the incidence of hepatoma (Chang, M H et al. New Engl. J. Med. 336, 1855-1859 (1997), representing a great impact on cancer prevention.

Among the most important viruses in chronic human viral infections are HPV, HBV, hepatitis C Virus (HCV), retroviruses such as human immunodeficiency virus (HIV-1 and HIV-2), herpesviruses such as Epstein Barr Virus (EBV), cytomegalovirus (CMV), HSV-1 and HSV-2, and influenza virus. Useful antigens include HBV surface antigen or HBV core antigen; ppUL83 or pp 89 of CMV; antigens of gp120, gp41 or p24 proteins of HIV-1; ICP27, gD2, gB of HSV; or influenza hemagglutinin or nucleoprotein (Anthony, L S et al., Vaccine 1999; 17:373-83). Other antigens associated with pathogens that can be utilized as described herein are antigens of various parasites, includes malaria, preferably malaria peptide based on repeats of NANP.

In alternative embodiments, the antigen is from a pathogen that is a bacterium, such as Bordetella pertussis; Ehrlichia chaffeensis; Staphylococcus aureus; Toxoplasma gondii; Legionella pneumophila; Brucella suis; Salmonella enterica; Mycobacterium avium; Mycobacterium tuberculosis; Listeria monocytogenes; Chlamydia trachomatis; Chlamydia pneumoniae; Rickettsia rickettsii; or, a fungus, such as, e.g., Paracoccidioides brasiliensis; or other pathogen, e.g., Plasmodium falciparum.

In addition to its applicability to human cancer and infectious diseases, the present invention is also intended for use in treating animal diseases in the veterinary medicine context. Thus, the approaches described herein may be readily applied by one skilled in the art to treatment of veterinary herpesvirus infections including equine herpesviruses, bovine viruses such as bovine viral diarrhea virus (for example, the E2 antigen), bovine herpesviruses, Marek's disease virus in chickens and other fowl; animal retroviral and lentiviral diseases (e.g., feline leukemia, feline immunodeficiency, simian immunodeficiency viruses, etc.); pseudorabies and rabies; and the like.

As for tumor antigens, any tumor-associated or tumor-specific antigen (or tumor cell derived epitope) that can be recognized by T cells, preferably by CTL, can be used. These include, without limitation, mutant p53, HER2/neu or a peptide thereof, or any of a number of melanoma-associated antigens such as MAGE-1, MAGE-3, MART-1/Melan-A, tyrosinase, gp75, gp100, BAGE, GAGE-1, GAGE-2, GnT-V, and p15 (see, for example, U.S. Pat. No. 6,187,306).

It is not necessary to include a full length antigen in a DNA vaccine; it suffices to include a fragment that will be presented by MHC class I.

### Approaches for Mutagenesis of E6, E7, and Other Antigens

Mutants of the antigens described here may be created, for example, using the Quick Change Site-Directed Mutagenesis Kit (Stratagene, La Jolla, Calif.). Generally, antigens that may be used herein may be proteins or peptides that differ from the naturally-occurring proteins or peptides but yet retain the necessary epitopes for functional activity. An antigen may comprise, consist essentially of, or consist of an amino acid sequence that is at least about 50%, 55%, 60%, 65%, 70%, 75%, 80%, 85%, 90%, 95%, 96%, 97%, 98%, or 99% identical to that of the naturally-occurring antigen or a fragment thereof. An antigen may also comprise, consist essentially of, or consist of an amino acid sequence that is encoded by a nucleotide sequence that is at least about 50%, 55%, 60%, 65%, 70%, 75%, 80%, 85%, 90%, 95%, 96%, 97%, 98%, or 99% identical to a nucleotide sequence encoding the naturally-occurring antigen or a fragment thereof. An antigen may also comprise, consist essentially of, or consist of an amino acid sequence that is encoded by a nucleic acid that hybridizes under high stringency conditions to a nucleic acid encoding the naturally-occurring antigen or a fragment thereof. Hybridization conditions are further described herein.

An exemplary protein may comprise, consist essentially of, or consist of, an amino acid sequence that is at least about 50%, 55%, 60%, 65%, 70%, 75%, 80%, 85%, 90%, 95%, 96%, 97%, 98%, or 99% identical to that of a viral protein, such as E6 or E7, such as an E6 or E7 sequence provided herein. Where the E6 or E7 protein is a detox E6 or E7 protein, the amino acid sequence of the protein may comprise, consist essentially of, or consist of an amino acid sequence that is at least about 50%, 55%, 60%, 65%, 70%, 75%, 80%, 85%, 90%, 95%, 96%, 97%, 98%, or 99% identical to that of an E6 or E7 protein, wherein the amino acids that render the protein a “detox” protein are present.

### Exemplary DNA Vaccines Encoding an Immunogenicity-Potentiating Polypeptide (IPP) and an Antigen

In one embodiment, a DNA vaccine encodes a fusion protein comprising an antigen and an IPP. An IPP preferably may act in potentiating an immune response by promoting: processing of the linked antigenic polypeptide via the MHC class I pathway or targeting of a cellular compartment that increases the processing. This basic strategy may be combined with an additional strategy pioneered by the present inventors and colleagues, that involve linking DNA encoding another protein, generically termed a “targeting polypeptide,” to the antigen-encoding DNA. Again, for the sake of simplicity, the DNA encoding such a targeting polypeptide will be referred to herein as a “targeting DNA.” That strategy has been shown to be effective in enhancing the potency of the vectors carrying only antigen-encoding DNA. See for example, the following PCT publications by Wu et al: WO 01/29233; WO 02/009645; WO 02/061113; WO 02/074920; and WO 02/12281, all of which are incorporated by reference in their entirety. The other strategies include the use of DNA encoding polypeptides that promote or enhance:
(a) development, accumulation or activity of antigen presenting cells or targeting of antigen to compartments of the antigen presenting cells leading to enhanced antigen presentation;
(b) intercellular transport and spreading of the antigen; or
(c) any combination of (a) and (b).
(d) sorting of the lysosome-associated membrane protein type 1 (Sig/LAMP-1).
   The strategy includes use of:
(e) a viral intercellular spreading protein selected from the group of herpes simplex virus-1 VP22 protein, Marek's disease virus UL49 (see WO 02/09645), protein or a functional homologue or derivative thereof;
(f) other endoplasmic reticulum chaperone polypeptides selected from the group of CRT-like molecules ER60, GRP94, gp96, or a functional homologue or derivative thereof (see WO 02/12281, hereby incorporated by reference;
(g) a cytoplasmic translocation polypeptide domains of a pathogen toxin selected from the group of domain II of Pseudomonas exotoxin ETA or a functional homologue or derivative thereof;
(h) a polypeptide that targets the centrosome compartment of a cell selected from γ-tubulin or a functional homologue or derivative thereof; or
(i) a polypeptide that stimulates dendritic cell precursors or activates dendritic cell activity selected from the group of GM-CSF, Flt3-ligand extracellular domain, or a functional homologue or derivative thereof; or.
(j) a costimulatory signal, such as a B7 family protein, including B7-DC (see U.S. Ser. No. 09/794,210), B7.1, B7.2, soluble CD40, etc.).
(k) an anti-apoptotic polypeptide preferably selected from the group consisting of (1) BCL-xL, (2) BCL2, (3) XIAP, (4) FLICEc-s, (5) dominant-negative caspase-8, (6) dominant negative caspase-9, (7) SPI-6, and (8) a functional homologue or derivative of any of (1)-(7). (See WO 2005/047501).

The following publications, all of which are incorporated by reference in their entirety, describe IPPs: Kim T W et al., J Clin Invest 112: 109-117, 2003; Cheng W F et al., J Clin Invest 108: 669-678, 2001; Hung C F et al., Cancer Res 61:3698-3703, 2001; Chen C H et al., 2000, supra; U.S. Pat. No. 6,734,173; published patent applications WO05/081716, WO05/047501, WO03/085085, WO02/12281, WO02/074920, WO02/061113, WO02/09645, and WO01/29233. Comparative studies of these IPPs using HPV E6 as the antigen are described in Peng, S. et al., J Biomed Sci. 12:689-700 2005.

An antigen may be linked N-terminally or C-terminally to an IPP. Exemplary IPPs and fusion constructs encoding such are described below.

### Lysosomal Associated Membrane Protein 1 (LAMP-1)

The DNA sequence encoding the E7 protein fused to the translocation signal sequence and LAMP-1 domain (Sig-E7-LAMP-1) [SEQ ID NO: 16] is:

ATGGCGGCCCCCGGCGCCCGGCGGCCGCTGCTCCTGCTGCTGCTGGCAGG
CCTTGCACATGGCGCCTCAGCACTCTTTGAGGATCTAATCATGCATGGA
GATACACCTACATTGCATGAATATATGTTAGATTTGCAACCAGAGACAAC
TGATCTCTACTGTTATGAGCAATTAAATGACAGCTCAGAGGAGGAGGATG
AAATAGATGGTCCAGCTGGACAAGCAGAACCGGACAGAGCCCATTACAA
TATTGTTACCTTTTGTTGCAAGTGTGACTCTACGCTTCGGTTGTGCGTAC
AAAGCACACACGTAGACATTCGTACTTTGGAAGACCTGTTAATGGGCA
CACTAGGAATTGTGTGCCCCATCTGTTCTCAGGATCTTAACAACATGTT
GATCCCCATTGCTGTGGGCGGTGCCCTGGCAGGGCTGGTCCTCATCG
TCCTCATTGCCTACCTCATTGGCAGGAAGAGGAGTCACGCCGGCTATC
AGACCATCTAG

The amino acid sequence of Sig/E7/LAMP-1 [SEQ ID NO: 17] is:

MAAPGARRPL LLLLLAGLAH GASALFEDLI MHGDTPTLHE
YMLDLQPETT DLYCYEQLND SSEEEDEIDG PAGQAEPDRA
HYNIVTFCCK CDSTLRLCVQ STHVDIRTLE DLLMGTLGIV
CPICSQDLNN MLIPIAVGGA LAGLVLIVLI AYLIGRKRSH
AGYQTI

The nucleotide sequence of the immunogenic vector pcDNA3-Sig/E7/LAMP-1 [SEQ ID NO: 18] is shown below with the SigE7-LAMP-1 coding sequence in lower case and underscored:

GACGGATCGGGAGATCTCCCGATCCCCTATGGTCGACTCTCAGTACAATCTGCTCTGATGCCGCATAGTTAAGCCAGTAT
CTGCTCCCTGCTTGTGTGTTGGAGGTCGCTGAGTAGTGCGCGAGCAAAATTTAAGCTACAACAAGGCAAGGCTTGACCGA
CAATTGCATGAAGAATCTGCTTAGGGTTAGGCGTTTTGCGCTGCTTCGCGATGTACGGGCCAGATATACGCGTTGACATT
GATTATTGACTAGTTATTAATAGTAATCAATTACGGGGTCATTAGTTCATAGCCCATATATGGAGTTCCGCGTTACATAA
CTTACGGTAAATGGCCCGCCTGGCTGACCGCCCAACGACCCCCGCCCATTGACGTCAATAATGACGTATGTTCCCATAGT
AACGCCAATAGGGACTTTCCATTGACGTCAATGGGTGGACTATTTACGGTAAACTGCCCACTTGGCAGTACATCAAGTGT
ATCATATGCCAAGTACGCCCCCTATTGACGTCAATGACGGTAAATGGCCCGCCTGGCATTATGCCCAGTACATGACCTTA
TGGGACTTTCCTACTTGGCAGTACATCTACGTATTAGTCATCGCTATTACCATGGTGATGCGGTTTTGGCAGTACATCAA
TGGGCGTGGATAGCGGTTTGACTCACGGGGATTTCCAAGTCTCCACCCCATTGACGTCAATGGGAGTTTGTTTTGGCACC
AAAATCAACGGGACTTTCCAAAATGTCGTAACAACTCCGCCCCATTGACGCAAATGGGCGGTAGGCGTGTACGGTGGGAG
GTCTATATAAGCAGAGCTCTCTGGCTAACTAGAGAACCCACTGCTTACTGGCTTATCGAAATTAATACGACTCACTATAG
GGAGACCCAAGCTGGCTAGCGTTTAAACGGGCCCTCTAGACTCGAGCGGCCGCCACTGTGCTGGATATCTGCAGAATTCa
tggcggcccccggcgcccggcggccgctgctcctgctgctgctggcaggccttgcacatggcgcctcagcactctttgag
gatctaatcatgcatggagatacacctacattgcatgaatatatgttagatttgcaaccagagacaactgatctctactg
ttatgagcaattaaatgacagctcagaggaggaggatgaaatagatggtccagctggacaagcagaaccggacagagccc
attacaatattgttaccttttgttgcaagtgtgactctacgcttcggttgtgcgtacaaagcacacacgtagacattcgt
actttggaagacctgttaatgggcacactaggaattgtgtgccccatctgttctcaggatcttaacaacatgttgatccc
cattgctgtgggcggtgccctggcagggctggtcctcatcgtcctcattgcctacctcattggcaggaagaggagtcacg
ccggctatcagaccatctagGGATCCGAGCTCGGTACCAAGCTTAAGTTTAAACCGCTGATCAGCCTCGACTGTGCCTTC
TAGTTGCCAGCCATCTGTTGTTTGCCCCTCCCCCGTGCCTTCCTTGACCCTGGAAGGTGCCACTCCCACTGTCCTTTCCT
AATAAAATGAGGAAATTGCATCGCATTGTCTGAGTAGGTGTCATTCTATTCTGGGGGGTGGGGTGGGGCAGGACAGCAAG
GGGGAGGATTGGGAAGACAATAGCAGGCATGCTGGGGATGCGGTGGGCTCTATGGCTTCTGAGGCGGAAAGAACCAGCTG
GGGCTCTAGGGGGTATCCCCACGCGCCCTGTAGCGGCGCATTAAGCGCGGCGGGTGTGGTGGTTACGCGCAGCGTGACCG
CTACACTTGCCAGCGCCCTAGCGCCCGCTCCTTTCGCTTTCTTCCCTTCCTTTCTCGCCACGTTCGCCGGCTTTCCCCGT
CAAGCTCTAAATCGGGGCATCCCTTTAGGGTTCCGATTTAGTGCTTTACGGCACCTCGACCCCAAAAAACTTGATTAGGG
TGATGGTTCACGTAGTGGGCCATCGCCCTGATAGACGGTTTTTCGCCCTTTGACGTTGGAGTCCACGTTCTTTAATAGTG
GACTCTTGTTCCAAACTGGAACAACACTCAACCCTATCTCGGTCTATTCTTTTGATTTATAAGGGATTTTGGGGATTTCG
GCCTATTGGTTAAAAAATGAGCTGATTTAACAAAAATTTAACGCGAATTAATTCTGTGGAATGTGTGTCAGTTAGGGTGT
GGAAAGTCCCCAGGCTCCCCAGGCAGGCAGAAGTATGCAAAGCATGCATCTCAATTAGTCAGCAACCAGGTGTGGAAAGT
CCCCAGGCTCCCCAGCAGGCAGAAGTATGCAAAGCATGCATCTCAATTAGTCAGCAACCATAGTCCCGCCCCTAACTCCG
CCCATCCCGCCCCTAACTCCGCCCAGTTCCGCCCATTCTCCGCCCCATGGCTGACTAATTTTTTTTATTTATGCAGAGGC
CGAGGCCGCCTCTGCCTCTGAGCTATTCCAGAAGTAGTGAGGAGGCTTTTTTGGAGGCCTAGGCTTTTGCAAAAAGCTCC
CGGGAGCTTGTATATCCATTTTCGGATCTGATCAAGAGACAGGATGAGGATCGTTTCGCATGATTGAACAAGATGGATTG
CACGCAGGTTCTCCGGCCGCTTGGGTGGAGAGGCTATTCGGCTATGACTGGGCACAACAGACAATCGGCTGCTCTGATGC
CGCCGTGTTCCGGCTGTCAGCGCAGGGGCGCCCGGTTCTTTTTGTCAAGACCGACCTGTCCGGTGCCCTGAATGAACTGC
AGGACGAGGCAGCGCGGCTATCGTGGCTGGCCACGACGGGCGTTCCTTGCGCAGCTGTGCTCGACGTTGTCACTGAAGCG
GGAAGGGACTGGCTGCTATTGGGCGAAGTGCCGGGGCAGGATCTCCTGTCATCTCACCTTGCTCCTGCCGAGAAAGTATC
CATCATGGCTGATGCAATGCGGCGGCTGCATACGCTTGATCCGGCTACCTGCCCATTCGACCACCAAGCGAAACATCGCA
TCGAGCGAGCACGTACTCGGATGGAAGCCGGTCTTGTCGATCAGGATGATCTGGACGAAGAGCATCAGGGGCTCGCGCCA
GCCGAACTGTTCGCCAGGCTCAAGGCGCGCATGCCCGACGGCGAGGATCTCGTCGTGACCCATGGCGATGCCTGCTTGCC
GAATATCATGGTGGAAAATGGCCGCTTTTCTGGATTCATCGACTGTGGCCGGCTGGGTGTGGCGGACCGCTATCAGGACA
TAGCGTTGGCTACCCGTGATATTGCTGAAGAGCTTGGCGGCGAATGGGCTGACCGCTTCCTCGTGCTTTACGGTATCGCC
GCTCCCGATTCGCAGCGCATCGCCTTCTATCGCCTTCTTGACGAGTTCTTCTGAGCGGGACTCTGGGGTTCGAAATGACC
GACCAAGCGACGCCCAACCTGCCATCACGAGATTTCGATTCCACCGCCGCCTTCTATGAAAGGTTGGGCTTCGGAATCGT
TTTCCGGGACGCCGGCTGGATGATCCTCCAGCGCGGGGATCTCATGCTGGAGTTCTTCGCCCACCCCAACTTGTTTATTG
CAGCTTATAATGGTTACAAATAAAGCAATAGCATCACAAATTTCACAAATAAAGCATTTTTTTCACTGCATTCTAGTTGT
GGTTTGTCCAAACTCATCAATGTATCTTATCATGTCTGTATACCGTCGACCTCTAGCTAGAGCTTGGCGTAATCATGGTC
ATAGCTGTTTCCTGTGTGAAATTGTTATCCGCTCACAATTCCACACAACATACGAGCCGGAAGCATAAAGTGTAAAGCCT
GGGGTGCCTAATGAGTGAGCTAACTCACATTAATTGCGTTGCGCTCACTGCCCGCTTTCCAGTCGGGAAACCTGTCGTGC
CAGCTGCATTAATGAATCGGCCAACGCGCGGGGAGAGGCGGTTTGCGTATTGGGCGCTCTTCCGCTTCCTCGCTCACTGA
CTCGCTGCGCTCGGTCGTTCGGCTGCGGCGAGCGGTATCAGCTCACTCAAAGGCGGTAATACGGTTATCCACAGAATCAG
GGGATAACGCAGGAAAGAACATGTGAGCAAAAGGCCAGCAAAAGGCCAGGAACCGTAAAAAGGCCGCGTTGCTGGCGTTT
TTCCATAGGCTCCGCCCCCCTGACGAGCATCACAAAAATCGACGCTCAAGTCAGAGGTGGCGAAACCCGACAGGACTATA
AAGATACCAGGCGTTTCCCCCTGGAAGCTCCCTCGTGCGCTCTCCTGTTCCGACCCTGCCGCTTACCGGATACCTGTCCG
CCTTTCTCCCTTCGGGAAGCGTGGCGCTTTCTCAATGCTCACGCTGTAGGTATCTCAGTTCGGTGTAGGTCGTTCGCTCC
AAGCTGGGCTGTGTGCACGAACCCCCCGTTCAGCCCGACCGCTGCGCCTTATCCGGTAACTATCGTCTTGAGTCCAACCC
GGTAAGACACGACTTATCGCCACTGGCAGCAGCCACTGGTAACAGGATTAGCAGAGCGAGGTATGTAGGCGGTGCTACAG
AGTTCTTGAAGTGGTGGCCTAACTACGGCTACACTAGAAGGACAGTATTTGGTATCTGCGCTCTGCTGAAGCCAGTTACC
TTCGGAAAAAGAGTTGGTAGCTCTTGATCCGGCAAACAAACCACCGCTGGTAGCGGTGGTTTTTTTGTTTGCAAGCAGCA
GATTACGCGCAGAAAAAAAGGATCTCAAGAAGATCCTTTGATCTTTTCTACGGGGTCTGACGCTCAGTGGAACGAAAACT
CACGTTAAGGGATTTTGGTCATGAGATTATCAAAAAGGATCTTCACCTAGATCCTTTTAAATTAAAAATGAAGTTTTAAA
TCAATCTAAAGTATATATGAGTAAACTTGGTCTGACAGTTACCAATGCTTAATCAGTGAGGCACCTATCTCAGCGATCTG
TCTATTTCGTTCATCCATAGTTGCCTGACTCCCCGTCGTGTAGATAACTACGATACGGGAGGGCTTACCATCTGGCCCCA
GTGCTGCAATGATACCGCGAGACCCACGCTCACCGGCTCCAGATTTATCAGCAATAAACCAGCCAGCCGGAAGGGCCGAG
CGCAGAAGTGGTCCTGCAACTTTATCCGCCTCCATCCAGTCTATTAATTGTTGCCGGGAAGCTAGAGTAAGTAGTTCGCC
AGTTAATAGTTTGCGCAACGTTGTTGCCATTGCTACAGGCATCGTGGTGTCACGCTCGTCGTTTGGTATGGCTTCATTCA
GCTCCGGTTCCCAACGATCAAGGCGAGTTACATGATCCCCCATGTTGTGCAAAAAAGCGGTTAGCTCCTTCGGTCCTCCG
ATCGTTGTCAGAAGTAAGTTGGCCGCAGTGTTATCACTCATGGTTATGGCAGCACTGCATAATTCTCTTACTGTCATGCC
ATCCGTAAGATGCTTTTCTGTGACTGGTGAGTACTCAACCAAGTCATTCTGAGAATAGTGTATGCGGCGACCGAGTTGCT
CTTGCCCGGCGTCAATACGGGATAATACCGCGCCACATAGCAGAACTTTAAAAGTGCTCATCATTGGAAAACGTTCTTCG
GGGCGAAAACTCTCAAGGATCTTACCGCTGTTGAGATCCAGTTCGATGTAACCCACTCGTGCACCCAACTGATCTTCAGC
ATCTTTTACTTTCACCAGCGTTTCTGGGTGAGCAAAAACAGGAAGGCAAAATGCCGCAAAAAAGGGAATAAGGGCGACAC
GGAAATGTTGAATACTCATACTCTTCCTTTTTCAATATTATTGAAGCATTTATCAGGGTTATTGTCTCATGAGCGGATAC
ATATTTGAATGTATTTAGAAAAATAAACAAATAGGGGTTCCGCGCACATTTCCCCGAAAAGTGCCACCTGACGTC

The nucleotide sequence encoding HSP70 (SEQ ID NO: 19) is (nucleotides 10633-12510 of the M. tuberculosis genome in GenBank NC_{—}000962):

atggctcg tgcggtcggg atcgacctcg ggaccaccaa ctccgtcgtc tcggttctgg aaggtggcga
cccggtcgtc gtcgccaact ccgagggctc caggaccacc ccgtcaattg tcgcgttcgc ccgcaacggt
gaggtgctgg tcggccagcc cgccaagaac caggcagtga ccaacgtcga tcgcaccgtg cgctcggtca
agcgacacat gggcagcgac tggtccatag agattgacgg caagaaatac accgcgccgg agatcagcgc
ccgcattctg atgaagctga agcgcgacgc cgaggcctac ctcggtgagg acattaccga cgcggttatc
acgacgcccg cctacttcaa tgacgcccag cgtcaggcca ccaaggacgc cggccagatc gccggcctca
acgtgctgcg gatcgtcaac gagccgaccg cggccgcgct ggcctacggc ctcgacaagg gcgagaagga
gcagcgaatc ctggtcttcg acttgggtgg tggcactttc gacgtttccc tgctggagat cggcgagggt
gtggttgagg tccgtgccac ttcgggtgac aaccacctcg gcggcgacga ctgggaccag cgggtcgtcg
attggctggt ggacaagttc aagggcacca gcggcatcga tctgaccaag gacaagatgg cgatgcagcg
gctgcgggaa gccgccgaga aggcaaagat cgagctgagt tcgagtcagt ccacctcgat caacctgccc
tacatcaccg tcgacgccga caagaacccg ttgttcttag acgagcagct gacccgcgcg gagttccaac
ggatcactca ggacctgctg gaccgcactc gcaagccgtt ccagtcggtg atcgctgaca ccggcatttc
ggtgtcggag atcgatcacg ttgtgctcgt gggtggttcg acccggatgc ccgcggtgac cgatctggtc
aaggaactca ccggcggcaa ggaacccaac aagggcgtca accccgatga ggttgtcgcg gtgggagccg
ctctgcaggc cggcgtcctc aagggcgagg tgaaagacgt tctgctgctt gatgttaccc cgctgagcct
gggtatcgag accaagggcg gggtgatgac caggctcatc gagcgcaaca ccacgatccc caccaagcgg
tcggagactt tcaccaccgc cgacgacaac caaccgtcgg tgcagatcca ggtctatcag ggggagcgtg
agatcgccgc gcacaacaag ttgctcgggt ccttcgagct gaccggcatc ccgccggcgc cgcgggggat
tccgcagatc gaggtcactt tcgacatcga cgccaacggc attgtgcacg tcaccgccaa ggacaagggc
accggcaagg agaacacgat ccgaatccag gaaggctcgg gcctgtccaa ggaagacatt gaccgcatga
tcaaggacgc cgaagcgcac gccgaggagg atcgcaagcg tcgcgaggag gccgatgttc gtaatcaagc
cgagacattg gtctaccaga cggagaagtt cgtcaaagaa cagcgtgagg ccgagggtgg ttcgaaggta
cctgaagaca cgctgaacaa ggttgatgcc gcggtggcgg aagcgaaggc ggcacttggc ggatcggata
tttcggccat caagtcggcg atggagaagc tgggccagga gtcgcaggct ctggggcaag cgatctacga
agcagctcag gctgcgtcac aggccactgg cgctgcccac cccggcggcg agccgggcgg tgcccacccc
ggctcggctg atgacgttgt ggacgcggag gtggtcgacg acggccggga ggccaagtga

The amino acid sequence of HSP70 [SEQ ID NO: 20] is:

MARAVGIDLG TTNSVVSVLE GGDPVVVANS EGSRTTPSIV AFARNGEVLV GQPAKNQAVT NVDRTVRSVK
RHMGSDWSIE IDGKKYTAPE ISARILMKLK RDAEAYLGED ITDAVITTPA YFNDAQRQAT KDAGQIAGLN
VLRIVNEPTA AALAYGLDKG EKEQRILVFD LGGGTFDVSL LEIGEGVVEV RATSGDNHLG GDDWDQRVVD
WLVDKFKGTS GIDLTKDKMA MQRLREAAEK AKIELSSSQS TSINLPYITV DADKNPLFLD EQLTRAEFQR
ITQDLLDRTR KPFQSVIADT GISVSEIDHV VLVGGSTRMP AVTDLVKELT GGKEPNKGVN PDEVVAVGAA
LQAGVLKGEV KDVLLLDVTP LSLGIETKGG VMTRLIERNT TIPTKRSETF TTADDNQPSV QIQVYQGERE
IAAHNKLLGS FELTGIPPAP RGIPQIEVTF DIDANGIVHV TAKDKGTGKE NTIRIQEGSG LSKEDIDRMI
KDAEAHAEED RKRREEADVR NQAETLVYQT EKFVKEQREA EGGSKVPEDT LNKVDAAVAE AKAALGGSDI
SAIKSAMEKL GQESQALGQA IYEAAQAASQ ATGAAHPGGE PGGAHPGSAD DVVDAEVVDD GREAK

The E7-Hsp70 chimera/fusion polypeptide sequences (Nucleotide sequence SEQ ID NO: 21 and amino acid sequence SEQ ID NO: 22) are provided below. The E7 coding sequence is shown in upper case and underscored.

1/1 31/11
ATG CAT GGA GAT ACA CCT ACA TTG CAT GAA TAT ATG TTA GAT TTG CAA CCA GAG ACA ACT
Met His Gly Asp Thr Pro Thr Leu His Glu Tyr Met Leu Asp Leu Gln Pro Glu Thr Thr
61/21 91/31
GAT CTC TAC TGT TAT GAG CAA TTA AAT GAC AGC TCA GAG GAG GAG GAT GAA ATA GAT GGT
Asp Leu Tyr Cys Tyr Glu Gln Leu Asn Asp Ser Ser Glu Glu Glu Asp Glu Ile Asp Gly
121/41 151/51
CCA GCT GGA CAA GCA GAA CCG GAC AGA GCC CAT TAC AAT ATT GTA ACC TTT TGT TGC AAG
Pro Ala Gly Gln Ala Glu Pro Asp Arg Ala His Tyr Asn Ile Val Thr Phe Cys Cys Lys
181/61 211/71
TGT GAC TCT ACG CTT CGG TTG TGC GTA CAA AGC ACA CAC GTA GAC ATT CGT ACT TTG GAA
Cys Asp Ser Thr Leu Arg Leu Cys Val Gln Ser Thr His Val Asp Ile Arg Thr Leu Glu
241/81 271/91
GAC CTG TTA ATG GGC ACA CTA GGA ATT GTG TGC CCC ATC TGT TCT CAA GGA TCC atg gc
Asp Leu Leu Met Gly Thr Leu Gly Ile Val Cys Pro Ile Cys Ser Gln Gly Ser Met Ala
301/101 331/111
cgt gcg gtc ggg atc gac ctc ggg acc acc aac tcc gtc gtc tcg gtt ctg gaa ggt ggc
Arg Ala Val Gly Ile Asp Leu Gly Thr Thr Asn Ser Val Val Ser Val Leu Glu Gly Gly
361/121 391/131
gac ccg gtc gtc gtc gcc aac tcc gag ggc tcc agg acc acc ccg tca att gtc gcg ttc
Asp Pro Val Val Val Ala Asn Ser Glu Gly Ser Arg Thr Thr Pro Ser Ile Val Ala Phe
421/141 451/151
gcc cgc aac ggt gag gtg ctg gtc ggc cag ccc gcc aag aac cag gca gtg acc aac gtc
Ala Arg Asn Gly Glu Val Leu Val Gly Gln Pro Ala Lys Asn Gln Ala Val Thr Asn Val
481/161 511/171
gat cgc acc gtg cgc tcg gtc aag cga cac atg ggc agc gac tgg tcc ata gag att gac
Asp Arg Thr Val Arg Ser Val Lys Arg His Met Gly Ser Asp Trp Ser Ile Glu Ile Asp
541/181 571/191
ggc aag aaa tac acc gcg ccg gag atc agc gcc cgc att ctg atg aag ctg aag cgc gac
Gly Lys Lys Tyr Thr Ala Pro Glu Ile Ser Ala Arg Ile Leu Met Lys Leu Lys Arg Asp
601/201 631/211
gcc gag gcc tac ctc ggt gag gac att acc gac gcg gtt atc acg acg ccc gcc tac ttc
Ala Glu Ala Tyr Leu Gly Glu Asp Ile Thr Asp Ala Val Ile Thr Thr Pro Ala Tyr Phe
661/221 691/231
aat gac gcc cag cgt cag gcc acc aag gac gcc ggc cag atc gcc ggc ctc aac gtg ctg
Asn Asp Ala Gln Arg Gln Ala Thr Lys Asp Ala Gly Gln Ile Ala Gly Leu Asn Val Leu
721/241 751/251
cgg atc gtc aac gag ccg acc gcg gcc gcg ctg gcc tac ggc ctc gac aag ggc gag aag
Arg Ile Val Asn Glu Pro Thr Ala Ala Ala Leu Ala Tyr Gly Leu Asp Lys Gly Glu Lys
781/261 811/271
gag cag cga atc ctg gtc ttc gac ttg ggt ggt ggc act ttc gac gtt tcc ctg ctg gag
Glu Gln Arg Ile Leu Val Phe Asp Leu Gly Gly Gly Thr Phe Asp Val Ser Leu Leu Glu
841/281 871/291
atc ggc gag ggt gtg gtt gag gtc cgt gcc act tcg ggt gac aac cac ctc ggc ggc gac
Ile Gly Glu Gly Val Val Glu Val Arg Ala Thr Ser Gly Asp Asn His Leu Gly Gly Asp
901/301 931/311
gac tgg gac cag cgg gtc gtc gat tgg ctg gtg gac aag ttc aag ggc acc agc ggc atc
Asp Trp Asp Gln Arg Val Val Asp Trp Leu Val Asp Lys Phe Lys Gly Thr Ser Gly Ile
961/321 991/331
gat ctg acc aag gac aag atg gcg atg cag cgg ctg cgg gaa gcc gcc gag aag gca aag
Asp Leu Thr Lys Asp Lys Met Ala Met Gln Arg Leu Arg Glu Ala Ala Glu Lys Ala Lys
1021/341 1051/351
atc gag ctg agt tcg agt cag tcc acc tcg atc aac ctg ccc tac atc acc gtc gac gcc
Ile Glu Leu Ser Ser Ser Gln Ser Thr Ser Ile Asn Leu Pro Tyr Ile Thr Val Asp Ala
1081/361 1111/371
gac aag aac ccg ttg ttc tta gac gag cag ctg acc cgc gcg gag ttc caa cgg atc act
Asp Lys Asn Pro Leu Phe Leu Asp Glu Gln Leu Thr Arg Ala Glu Phe Gln Arg Ile Thr
1141/381 1171/391
cag gac ctg ctg gac cgc act cgc aag ccg ttc cag tcg gtg atc gct gac acc ggc att
Gln Asp Leu Leu Asp Arg Thr Arg Lys Pro Phe Gln Ser Val Ile Ala Asp Thr Gly Ile
1201/401 1231/411
tcg gtg tcg gag atc gat cac gtt gtg ctc gtg ggt ggt tcg acc cgg atg ccc gcg gtg
Ser Val Ser Glu Ile Asp His Val Val Leu Val Gly Gly Ser Thr Arg Met Pro Ala Val
1261/421 1291/431
acc gat ctg gtc aag gaa ctc acc ggc ggc aag gaa ccc aac aag ggc gtc aac ccc gat
Thr Asp Leu Val Lys Glu Leu Thr Gly Gly Lys Glu Pro Asn Lys Gly Val Asn Pro Asp
1321/441 1351/451
gag gtt gtc gcg gtg gga gcc gct ctg cag gcc ggc gtc ctc aag ggc gag gtg aaa gac
Glu Val Val Ala Val Gly Ala Ala Leu Gln Ala Gly Val Leu Lys Gly Glu Val Lys Asp
1381/461 1411/471
gtt ctg ctg ctt gat gtt acc ccg ctg agc ctg ggt atc gag acc aag ggc ggg gtg atg
Val Leu Leu Leu Asp Val Thr Pro Leu Ser Leu Gly Ile Glu Thr Lys Gly Gly Val Met
1441/481 1471/491
acc agg ctc atc gag cgc aac acc acg atc ccc acc aag cgg tcg gag act ttc acc acc
Thr Arg Leu Ile Glu Arg Asn Thr Thr Ile Pro Thr Lys Arg Ser Glu Thr Phe Thr Thr
1501/501 1531/511
gcc gac gac aac caa ccg tcg gtg cag atc cag gtc tat cag ggg gag cgt gag atc gcc
Ala Asp Asp Asn Gln Pro Ser Val Gln Ile Gln Val Tyr Gln Gly Glu Arg Glu Ile Ala
1561/521 1591/531
gcg cac aac aag ttg ctc ggg tcc ttc gag ctg acc ggc atc ccg ccg gcg ccg cgg ggg
Ala His Asn Lys Leu Leu Gly Ser Phe Glu Leu Thr Gly Ile Pro Pro Ala Pro Arg Gly
1621/541 1651/551
att ccg cag atc gag gtc act ttc gac atc gac gcc aac ggc att gtg cac gtc acc gcc
Ile Pro Gln Ile Glu Val Thr Phe Asp Ile Asp Ala Asn Gly Ile Val His Val Thr Ala
1681/561 1711/571
aag gac aag ggc acc ggc aag gag aac acg atc cga atc cag gaa ggc tcg ggc ctg tcc
Lys Asp Lys Gly Thr Gly Lys Glu Asn Thr Ile Arg Ile Gln Glu Gly Ser Gly Leu Ser
1741/581 1771/591
aag gaa gac att gac cgc atg atc aag gac gcc gaa gcg cac gcc gag gag gat cgc aag
Lys Glu Asp Ile Asp Arg Met Ile Lys Asp Ala Glu Ala His Ala Glu Glu Asp Arg Lys
1801/601 1831/611
cgt cgc gag gag gcc gat gtt cgt aat caa gcc gag aca ttg gtc tac cag acg gag aag
Arg Arg Glu Glu Ala Asp Val Arg Asn Gln Ala Glu Thr Leu Val Tyr Gln Thr Glu Lys
1861/621 1891/631
ttc gtc aaa gaa cag cgt gag gcc gag ggt ggt tcg aag gta cct gaa gac acg ctg aac
Phe Val Lys Glu Gln Arg Glu Ala Glu Gly Gly Ser Lys Val Pro Glu Asp Thr Leu Asn
1921/641 1951/651
aag gtt gat gcc gcg gtg gcg gaa gcg aag gcg gca ctt ggc gga tcg gat att tcg gcc
Lys Val Asp Ala Ala Val Ala Glu Ala Lys Ala Ala Leu Gly Gly Ser Asp Ile Ser Ala
1981/661 2011/671
atc aag tcg gcg atg gag aag ctg ggc cag gag tcg cag gct ctg ggg caa gcg atc tac
Ile Lys Ser Ala Met Glu Lys Leu Gly Gln Glu Ser Gln Ala Leu Gly Gln Ala Ile Tyr
2041/681 2071/691
gaa gca gct cag gct gcg tca cag gcc act ggc gct gcc cac ccc ggc tcg gct gat gaA
GLU ALA ALA GLN ALA ALA SER GLN ALA THR GLY ALA ALA HIS PRO GLY SER ALA ASP GLU
2101/701
AGC a
Ser

ETA(dII) from Pseudomonas aeruginosa

The complete coding sequence for Pseudomonas aeruginosa exotoxin type A (ETA)—SEQ ID NO: 23—GenBank Accession No. K01397, is shown below:

ctgcagctgg tcaggccgtt tccgcaacgc ttgaagtcct ggccgatata ccggcagggc cagccatcgt tcgacgaata
aagccacctc agccatgatg ccctttccat ccccagcgga accccgacat ggacgccaaa gccctgctcc tcggcagcct
ctgcctggcc gccccattcg ccgacgcggc gacgctcgac aatgctctct ccgcctgcct cgccgcccgg ctcggtgcac
cgcacacggc ggagggccag ttgcacctgc cactcaccct tgaggcccgg cgctccaccg gcgaatgcgg ctgtacctcg
gcgctggtgc gatatcggct gctggccagg ggcgccagcg ccgacagcct cgtgcttcaa gagggctgct cgatagtcgc
caggacacgc cgcgcacgct gaccctggcg gcggacgccg gcttggcgag cggccgcgaa ctggtcgtca ccctgggttg
tcaggcgcct gactgacagg ccgggctgcc accaccaggc cgagatggac gccctgcatg tatcctccga tcggcaagcc
tcccgttcgc acattcacca ctctgcaatc cagttcataa atcccataaa agccctcttc cgctccccgc cagcctcccc
gcatcccgca ccctagacgc cccgccgctc tccgccggct cgcccgacaa gaaaaaccaa ccgctcgatc agcctcatcc
ttcacccatc acaggagcca tcgcgatgca cctgataccc cattggatcc ccctggtcgc cagcctcggc ctgctcgccg
gcggctcgtc cgcgtccgcc gccgaggaag ccttcgacct ctggaacgaa tgcgccaaag cctgcgtgct cgacctcaag
gacggcgtgc gttccagccg catgagcgtc gacccggcca tcgccgacac caacggccag ggcgtgctgc actactccat
ggtcctggag ggcggcaacg acgcgctcaa gctggccatc gacaacgccc tcagcatcac cagcgacggc ctgaccatcc
gcctcgaagg cggcgtcgag ccgaacaagc cggtgcgcta cagctacacg cgccaggcgc gcggcagttg gtcgctgaac
tggctggtac cgatcggcca cgagaagccc tcgaacatca aggtgttcat ccacgaactg aacgccggca accagctcag
ccacatgtcg ccgatctaca ccatcgagat gggcgacgag ttgctggcga agctggcgcg cgatgccacc ttcttcgtca
gggcgcacga gagcaacgag atgcagccga cgctcgccat cagccatgcc ggggtcagcg tggtcatggc ccagacccag
ccgcgccggg aaaagcgctg gagcgaatgg gccagcggca aggtgttgtg cctgctcgac ccgctggacg gggtctacaa
ctacctcgcc cagcaacgct gcaacctcga cgatacctgg gaaggcaaga tctaccgggt gctcgccggc aacccggcga
agcatgacct ggacatcaaa cccacggtca tcagtcatcg cctgcacttt cccgagggcg gcagcctggc cgcgctgacc
gcgcaccagg cttgccacct gccgctggag actttcaccc atcatcgcca gccgcgcggc tgggaacaac tggagcagtg
cggctatccg gtgcagcggc tggtcgccct ctacctggcg gcgcggctgt cgtggaacca ggtcgaccag gtgatccgca
acgccctggc cagccccggc agcggcggcg acctgggcga agcgatccgc gagcagccgg agcaggcccg tctggccctg
accctggccg ccgccgagag cgagcgcttc gtccggcagg gcaccggcaa cgacgaggcc ggcgcggcca acgccgacgt
ggtgagcctg acctgcccgg tcgccgccgg tgaatgcgcg ggcccggcgg acagcggcga cgccctgctg gagcgcaact
atcccactgg cgcggagttc ctcggcgacg gcggcgacgt cagcttcagc acccgcggca cgcagaactg gacggtggag
cggctgctcc aggcgcaccg ccaactggag gagcgcggct atgtgttcgt cggctaccac ggcaccttcc tcgaagcggc
gcaaagcatc gtcttcggcg gggtgcgcgc gcgcagccag gacctcgacg cgatctggcg cggtttctat atcgccggcg
atccggcgct ggcctacggc tacgcccagg accaggaacc cgacgcacgc ggccggatcc gcaacggtgc cctgctgcgg
gtctatgtgc cgcgctcgag cctgccgggc ttctaccgca ccagcctgac cctggccgcg ccggaggcgg cgggcgaggt
cgaacggctg atcggccatc cgctgccgct gcgcctggac gccatcaccg gccccgagga ggaaggcggg cgcctggaga
ccattctcgg ctggccgctg gccgagcgca ccgtggtgat tccctcggcg atccccaccg acccgcgcaa cgtcggcggc
gacctcgacc cgtccagcat ccccgacaag gaacaggcga tcagcgccct gccggactac gccagccagc ccggcaaacc
gccgcgcgag gacctgaagt aactgccgcg accggccggc tcccttcgca ggagccggcc ttctcggggc ctggccatac
atcaggtttt cctgatgcca gcccaatcga atatgaattc 2760

The amino acid sequence of ETA (SEQ ID NO: 24), GenBank Accession No. K01397, is:

MHLIPHWIPL VASLGLLAGG SSASAAEEAF DLWNECAKAC VLDLKDGVRS SRMSVDPAIA DTNGQGVLHY
SMVLEGGNDA LKLAIDNALS ITSDGLTIRL EGGVEPNKPV RYSYTRQARG SWSLNWLVPI GHEKPSNIKV
FIHELNAGNQ LSHMSPIYTI EMGDELLAKL ARDATFFVRA HESNEMQPTL AISHAGVSVV MAQTQPRREK
RWSEWASGKV LCLLDPLDGV YNYLAQQRCN LDDTWEGKIY RVLAGNPAKH DLDIKPTVIS HRLHFPEGGS
LAALTAHQAC HLPLETFTRH RQPRGWEQLE QCGYPVQRLV ALYLAARLSW NQVDQVIRNA LASPGSGGDL
GEAIREQPEQ ARLALTLAAA ESERFVRQGT GNDEAGAANA DVVSLTCPVA AGECAGPADS GDALLERNYP
TGAEFLGDGG DVSFSTRGTQ NWTVERLLQA HRQLEERGYV FVGYHGTFLE AAQSIVFGGV RARSQDLDAI
WRGFYIAGDP ALAYGYAQDQ EPDARGRIRN GALLRVYVPR SSLPGFYRTS LTLAAPEAAG EVERLIGHPL
PLRLDAITGP EEEGGRLETI LGWPLAERTV VIPSAIPTDP RNVGGDLDPS SIPDKEQAIS ALPDYASQPG
KPPREDLK 638

Residues 1-25 (italicized) above represent the signal peptide. The first residue of the mature polypeptide, Ala, is bolded/underscored. The mature polypeptide is residues 26-638 of SEQ ID NO: 24.

Domain II (ETA(II)), translocation domain (underscored above) spans residues 247-417 of the mature polypeptide (corresponding to residues 272-442 of SEQ ID NO: 24) and is presented below separately as SEQ ID NO: 25.

RLHFPEGGSL AALTAHQACH LPLETFTRHR QPRGWEQLEQ
CGYPVQRLVA LYLAARLSWN QVDQVIRNAL ASPGSGGDLG
EAIREQPEQA RLALTLAAAE SERFVRQGTG NDEAGAANAD
VVSLTCPVAA GECAGPADSG DALLERNYPT GAEFLGDGGD
VSFSTRGTQN W 171

The construct in which ETA(dII) is fused to HPV-16 E7 is shown below (nucleotides; SEQ ID NO: 26 and amino acids; SEQ ID NO: 27). The ETA(dII) sequence appears in plain font, extra codons from plasmid pcDNA3 are italicized. Nucleotides between ETA(dII) and E7 are also bolded (and result in the interposition of two amino acids between ETA(dII) and E7). The E7 amino acid sequence is underscored (ends with Gln at position 269).

1/1 31/11
atg cgc ctg cac ttt ccc gag ggc ggc agc ctg gcc gcg ctg acc gcg cac cag get tgc
Met arg leu his phe pro glu gly gly ser leu ala ala leu thr ala his gln ala cys
61/21 91/31
cac ctg ccg ctg gag act ttc acc cgt cat cgc cag ccg cgc ggc tgg gaa caa ctg gag
His Leu Pro Leu Glu Thr Phe Thr Arg His Arg Gln Pro Arg Gly Trp Glu Gln Leu Glu
121/41 151/51
cag tgc ggc tat ccg gtg cag cgg ctg gtc gcc ctc tac ctg gcg gcg cgg ctg tcg tgg
Gln Cys Gly Tyr Pro Val Gln Arg Leu Val Ala Leu Tyr Leu Ala Ala Arg Leu Ser Trp
181/61 211/71
aac cag gtc gac cag gtg atc cgc aac gcc ctg gcc agc ccc ggc agc ggc ggc gac ctg
Asn Gln Val Asp Gln Val Ile Arg Asn Ala Leu Ala Ser Pro Gly Ser Gly Gly Asp Leu
241/81 271/91
ggc gaa gcg atc cgc gag cag ccg gag cag gcc cgt ctg gcc ctg acc ctg gcc gcc gcc
Gly Glu Ala Ile Arg Glu Gln Pro Glu Gln Ala Arg Leu Ala Leu Thr Leu Ala Ala Ala
301/101 331/111
gag agc gag cgc ttc gtc cgg cag ggc acc ggc aac gac gag gcc ggc gcg gcc aac gcc
Glu Ser Glu Arg Phe Val Arg Gln Gly Thr Gly Asn Asp Glu Ala Gly Ala Ala Asn Ala
361/121 391/131
gac gtg gtg agc ctg acc tgc ccg gtc gcc gcc ggt gaa tgc gcg ggc ccg gcg gac agc
Asp Val Val Ser Leu Thr Cys Pro Val Ala Ala Gly Glu Cys Ala Gly Pro Ala Asp Ser
421/141 451/151
ggc gac gcc ctg ctg gag cgc aac tat ccc act ggc gcg gag ttc ctc ggc gac ggc ggc
Gly Asp Ala Leu Leu Glu Arg Asn Tyr Pro Thr Gly Ala Glu Phe Leu Gly Asp Gly Gly
481/161 511/171
gac gtc agc ttc agc acc cgc ggc acg cag [symbol] [symbol] [symbol] atg cat gga gat aca cct aca
Asp Val Ser Phe Ser Thr Arg Gly Thr Gln [symbol] [symbol] [symbol] Met His Gly Asp Thr Pro Thr
541/181 571/191
ttg cat gaa tat atg tta gat ttg caa cca gag aca act gat ctc tac tgt tat gag caa
Leu His Glu Tyr Met Leu Asp Leu Gln Pro Glu Thr Thr Asp Leu Tyr Cys Tyr Glu Gln
601/201 631/211
tta aat gac agc tca gag gag gag gat gaa ata gat ggt cca gct gga caa gca gaa ccg
Leu Asn Asp Ser Ser Glu Glu Glu Asp Glu Ile Asp Gly Pro Ala Gly Gln Ala Glu Pro
661/221 691/231
gac aga gcc cat tac aat att gta acc ttt tgt tgc aag tgt gac tct acg ctt cgg ttg
Asp Arg Ala His Tyr Asn Ile Val Thr Phe Cys Cys Lys Cys Asp Ser Thr Leu Arg Leu
721/241 751/251
tgc gta caa agc aca cac gta gac att cgt act ttg gaa gac ctg tta atg ggc aca cta
Cys Val Gln Ser Thr His Val Asp Ile Arg Thr Leu Glu Asp Leu Leu Met Gly Thr Leu
781/261 811/271
gga att gtg tgc ccc atc tgt tct caa gga tcc gag ctc ggt acc aag ctt aag ttt aaa
Gly Ile Val Cys Pro Ile Cys Ser Gln Gly Ser Glu Leu Gly Thr Lys Leu Lys Phe Lys
841/281
ccg ctg atc agc ctc gac tgt gcc ttc tag
Pro Leu Ile Ser Leu Asp Cys Ala Phe AMB

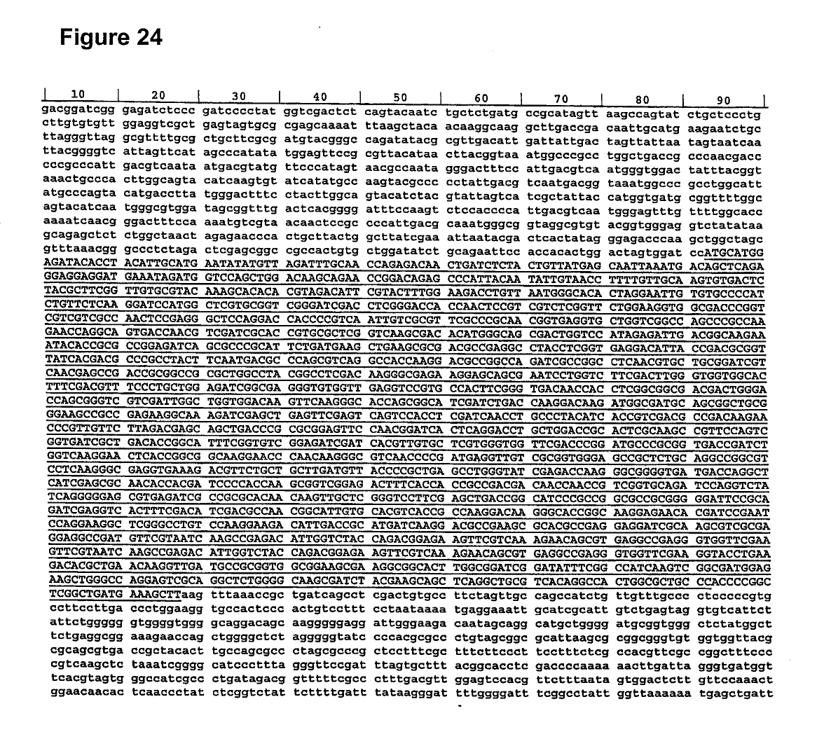

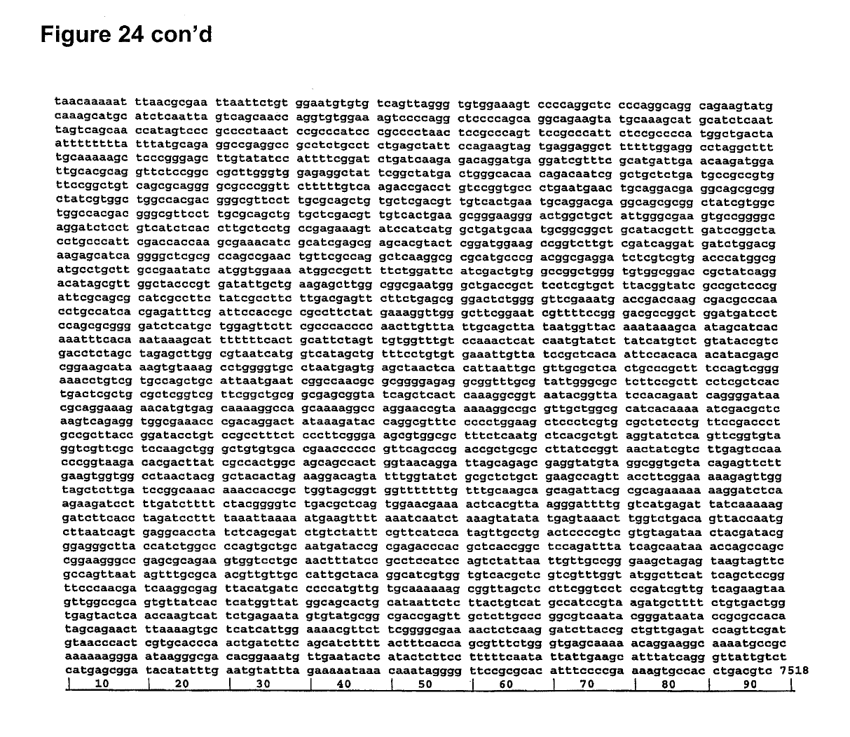

The nucleotide sequence of the pcDNA3 vector encoding E7 and HSP70 (pcDNA3-E7-Hsp70) (SEQ ID NO: 3) is shown in FIG. 24. The E7-Hsp70 fusion sequence is shown in upper case, underscored. Plasmid sequences are in lower case.

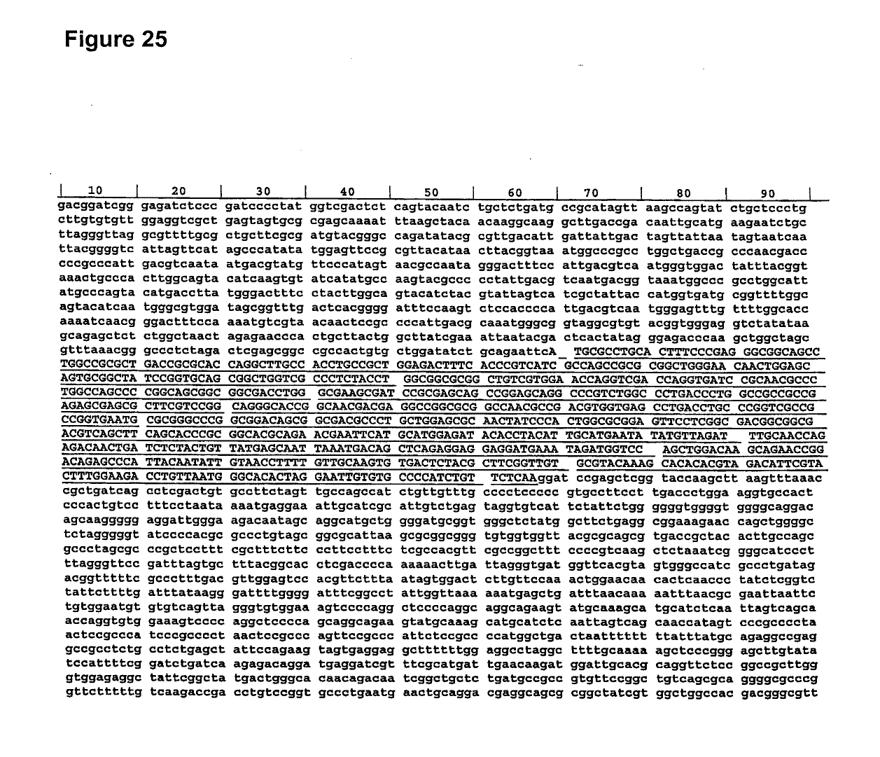

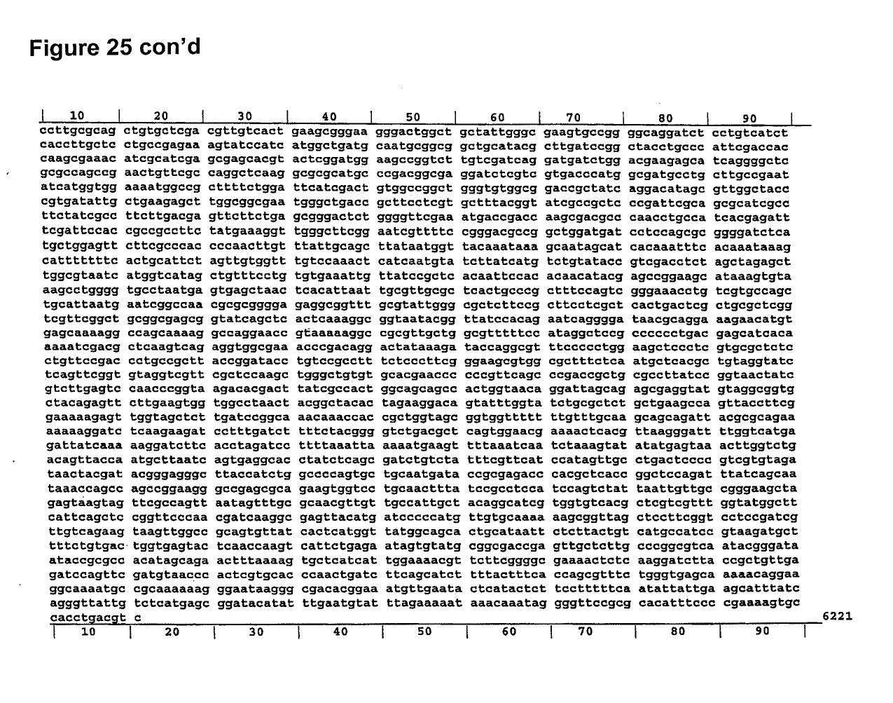

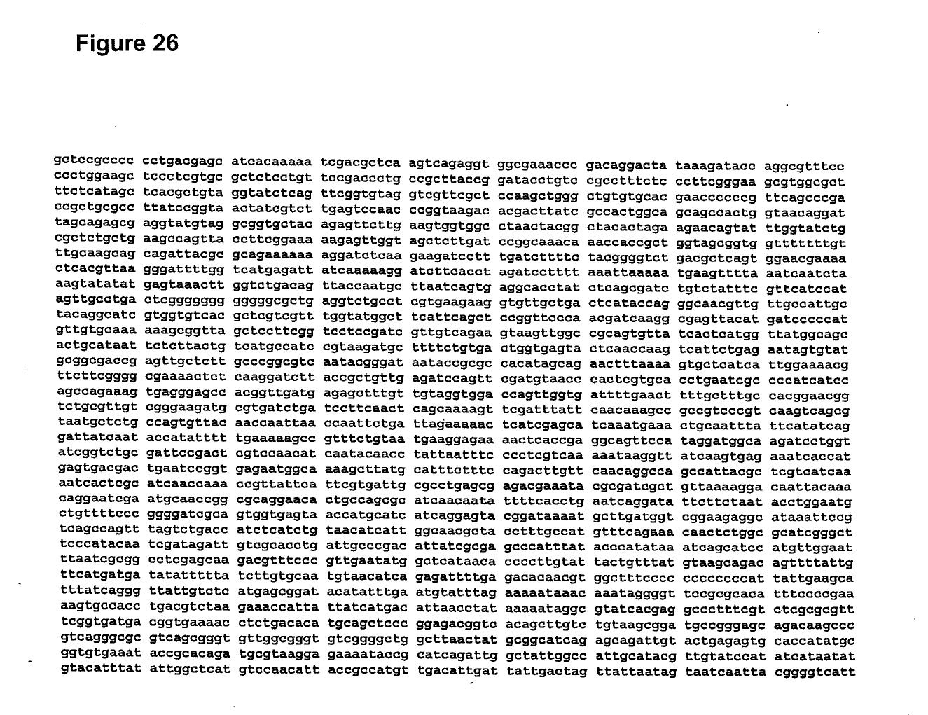

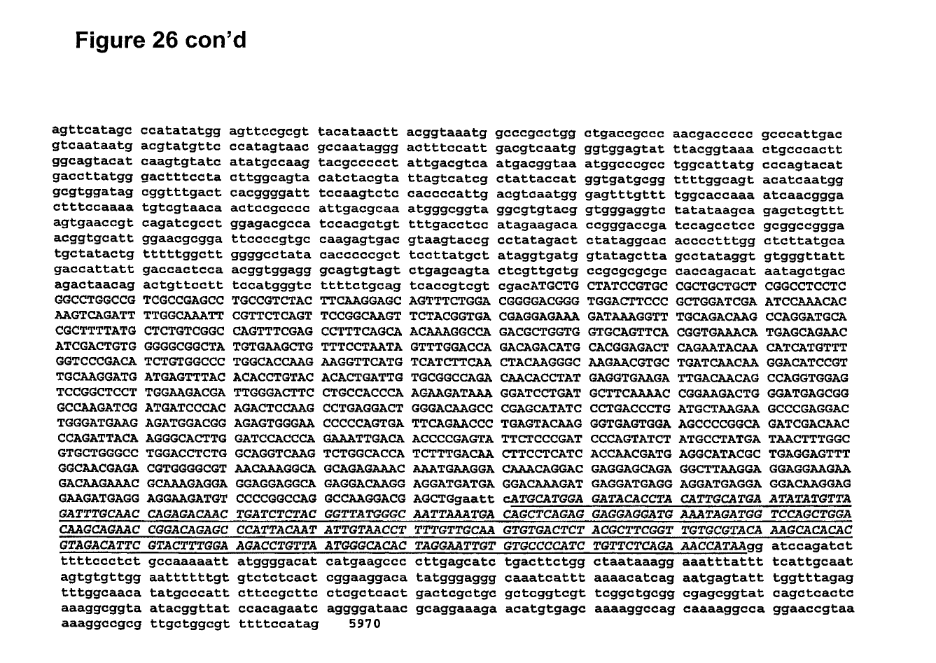

The nucleic acid sequence of plasmid construct pcDNA3-ETA(dII)/E7 (SEQ ID NO: 4) is shown in FIG. 25. ETA(dII)/E7 is ligated into the EcoRI/BamHI sites of pcDNA3 vector. The nucleotides encoding ETA(dII)/E7 are shown in upper case and underscored. Plasmid sequence is lower case.

### Calreticulin (CRT)

Calreticulin (CRT), a well-characterized ˜46 kDa protein was described briefly above, as were a number of its biological and biochemical activities. As used herein, “calreticulin” or “CRT” refers to polypeptides and nucleic acids molecules having substantial identity (defined herein) to the exemplary human CRT sequences as described herein or homologues thereof, such as rabbit and rat CRT—well-known in the art. A CRT polypeptide is a polypeptides comprising a sequence identical to or substantially identical (defined herein) to the amino acid sequence of CRT. An exemplary nucleotide and amino acid sequence for a CRT used in the present compositions and methods are presented below. The terms “calreticulin” or “CRT” encompass native proteins as well as recombinantly produced modified proteins that, when fused with an antigen (at the DNA or protein level) promote the induction of induce immune responses and, promote angiogenesis., including a CTL response. Thus, the terms “calreticulin” or “CRT” encompass homologues and allelic variants of human CRT, including variants of native proteins constructed by in vitro techniques, and proteins isolated from natural sources. The CRT polypeptides of the invention, and sequences encoding them, also include fusion proteins comprising non-CRT sequences, particularly MHC class I-binding peptides; and also further comprising other domains, e.g., epitope tags, enzyme cleavage recognition sequences, signal sequences, secretion signals and the like.

A human CRT coding sequence is shown below (SEQ ID NO: 28):

| | |
|---|---|
| 1 | atgctgctat ccgtgccgct gctgctcggc ctcctcggcc tggccgtcgc cgagcccgcc |
| 61 | gtctacttca aggagcagtt tctggacgga gacgggtgga cttcccgctg gatcgaatcc |
| 121 | aaacacaagt cagattttgg caaattcgtt ctcagttccg gcaagttcta cggtgacgag |
| 181 | gagaaagata aaggtttgca gacaagccag gatgcacgct tttatgctct gtcggccagt |
| 241 | ttcgagcctt tcagcaacaa aggccagacg ctggtggtgc agttcacggt gaaacatgag |
| 301 | cagaacatcg actgtggggg cggctatgtg aagctgtttc ctaatagttt ggaccagaca |
| 361 | gacatgcacg gagactcaga atacaacatc atgtttggtc ccgacatctg tggccctggc |
| 421 | accaagaagg ttcatgtcat cttcaactac aagggcaaga acgtgctgat caacaaggac |
| 481 | atccgttgca aggatgatga gtttacacac ctgtacacac tgattgtgcg gccagacaac |
| 541 | acctatgagg tgaagattga caacagccag gtggagtccg gctccttgga agacgattgg |
| 601 | gacttcctgc cacccaagaa gataaaggat cctgatgctt caaaaccgga agactgggat |
| 661 | gagcgggcca agatcgatga tcccacagac tccaagcctg aggactggga caagcccgag |
| 721 | catatccctg accctgatgc taagaagccc gaggactggg atgaagagat ggacggagag |
| 781 | tgggaacccc cagtgattca gaaccctgag tacaagggtg agtggaagcc ccggcagatc |
| 841 | gacaacccag attacaaggg cacttggatc cacccagaaa ttgacaaccc cgagtattct |
| 901 | cccgatccca gtatctatgc ctatgataac tttggcgtgc tgggcctgga cctctggcag |
| 961 | gtcaagtctg gcaccatctt tgacaacttc ctcatcacca acgatgaggc atacgctgag |
| 1021 | gagtttggca acgagacgtg gggcgtaaca aaggcagcag agaaacaaat gaaggacaaa |
| 1081 | caggacgagg agcagaggct taaggaggag gaagaagaca agaaacgcaa agaggaggag |
| 1141 | gaggcagagg acaaggagga tgatgaggac aaagatgagg atgaggagga tgaggaggac |
| 1201 | aaggaggaag atgaggagga agatgtcccc ggccaggcca aggacgagct gtag 1251 |

The amino acid sequence of the human CRT protein encoded by SEQ ID NO: 28 is set forth below (SEQ ID NO: 29). This amino acid sequence is highly homologous to GenBank Accession No. NM 004343.

| | | |
|---|---|---|
| 1 | MLLSVPLLLG LLGLAVAEPA VYFKEQFLDG DGWTSRWIES KHKSDFGKFV LSSGKFYGDE | |
| 61 | EKDKGLQTSQ DARFYALSAS FEPFSNKGQT LVVQFTVKHE QNIDCGGGYV KLFPNSLDQT | |
| 121 | DMHGDSEYNI MFGPDICGPG TKKVHVIFNY KGKNVLINKD IRCKDDEFTH LYTLIVRPDN | |
| 181 | TYEVKIDNSQ VESGSLEDDW DFLPPKKIKD PDASKPEDWD ERAKIDDPTD SKPEDWDKPE | |
| 241 | HIPDPDAKKP EDWDEEMDGE WEPPVIQNPE YKGEWKPRQI DNPDYKGTWI HPEIDNPEYS | |
| 301 | PDPSIYAYDN FGVLGLDLWQ VKSGTIFDNF LITNDEAYAE EFGNETWGVT KAAEKQMKDK | |
| 361 | QDEEQRLKEE EEDKKRKEEE EAEDKEDDED KDEDEEDEED KEEDEEEDVP GQAKDEL | 417 |

The amino acid sequence of the rabbit and rat CRT proteins are set forth in GenBank Accession Nos. P15253 and NM 022399, respectively). An alignment of human, rabbit and rat CRT shows that these proteins are highly conserved, and most of the amino acid differences between species are conservative in nature. Most of the variation is found in the alignment of the approximately 36 C-terminal residues. Thus, for the present invention, although human CRT is preferred, DNA encoding any homologue of CRT from any species that has the requisite biological activity (as an IPP) or any active domain or fragment thereof, may be used in place of human CRT or a domain thereof.

The present inventors and colleagues (Cheng et al., supra; incorporated by reference in its entirety) that DNA vaccines encoding each of the N, P, and C domains of CRT chimerically linked to HPV-16 E7 elicited potent antigen-specific CD8+ T cell responses and antitumor immunity in mice vaccinated i.d., by gene gun administration. N-CRT/E7, P-CRT/E7 or C-CRT/E7 DNA each exhibited significantly increased numbers of E7-specific CD8⁺ T cell precursors and impressive antitumor effects against E7-expressing tumors when compared with mice vaccinated with E7 DNA (antigen only). N-CRT DNA administration also resulted in anti-angiogenic antitumor effects. Thus, cancer therapy using DNA encoding N-CRT linked to a tumor antigen may be used for treating tumors through a combination of antigen-specific immunotherapy and inhibition of angiogenesis.

The constructs comprising CRT or one of its domains linked to E7 is illustrated schematically below.

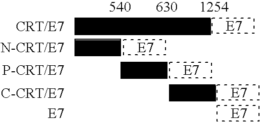

The amino acid sequences of the 3 human CRT domains are shown as annotations of the full length protein (SEQ ID NO: 29). The N domain comprises residues 1-170 (normal text); the P domain comprises residues 171-269 (underscored); and the C domain comprises residues 270-417 (bold/italic)

| | | |
|---|---|---|
| 1 | MLLSVPLLLG LLGLAVAEPA VYFKEQFLDG DGWTSRWIES KHKSDFGKFV LSSGKFYGDE | |
| 61 | EKDKGLQTSQ DARFYALSAS FEPFSNKGQT LVVQFTVKHE QNIDCGGGYV KLFPNSLDQT | |
| 121 | DMHGDSEYNI MFGPDICGPG TKKVHVIFNY KGKNVLINKD IRCKDDEFTH LYTLIVRPDN | |
| 181 | TYEVKIDNSQ VESGSLEDDW DFLPPKKIKD PDASKPEDWD ERAKIDDPTD SKPEDWDKPE | |
| 241 | HIPDPDAKKP EDWDEEMDGE WEPPVIQNPE YKGEWKPRQ [symbol] [symbol] [symbol] I [symbol] D [symbol] | |
| 301 | [symbol] [symbol] [symbol] [symbol] [symbol] [symbol] [symbol] E [symbol] [symbol] [symbol] [symbol] [symbol] | |
| 361 | [symbol] [symbol] [symbol] [symbol] [symbol] [symbol] [symbol] [symbol] [symbol] [symbol] [symbol] [symbol] | 417 |

The sequences of the three domains are shown as separate polypeptides below:

| Human N-CRT | | |
|---|---|---|
| (SEQ ID NO: 30) | | |
| 1 | MLLSVPLLLG LLGLAVAEPA VYFKEQFLDG DGWTSRWIES KHKSDFGKFV LSSGKFYGDE | |
| 61 | EKDKGLQTSQ DARFYALSAS FEPFSNKGQT LVVQFTVKHE QNIDCGGGYV KLFPNSLDQT | |
| 121 | DMHGDSEYNI MFGPDICGPG TKKVHVIFNY KGKNVLINKD IRCKDDEFTH | 170 |
| Human P-CRT | | |
| (SEQ ID NO: 31) | | |
| 1 | LYTLIVRPDN TYEVKIDNSQ VESGSLEDDW DFLPPKKIKD PDASKPEDWD ERAKIDDPTD | |
| 61 | SKPEDWDKPE HIPDPDAKKP EDWDEEMDGE WEPPVIQNPE YKGEWKPRQ | 109 |
| Human C-CRT | | |
| (SEQ ID NO: 32) | | |
| 1 | IDNPDYKGTW IHPEIDNPEY SPDPSIYAYD NFGVLGLDLW QVKSGTIFDN FLITNDEAYA | |
| 61 | EEFGNETWGV TKAAEKQMKD KQDEEQRLKE EEEDKKRKEE EEAEDKEDDE DKDEDEEDEE | |
| 121 | DKEEDEEEDV PGQAKDEL | 138 |

The present vectors may comprises DNA encoding one or more of these domain sequences, which are shown by annotation of SEQ ID NO: 28, below, wherein the N-domain sequence is upper case, the P-domain sequence is lower case/italic/underscored, and the C domain sequence is lower case. The stop codon is also shown but not counted.

| 1 | ATGCTGCTAT CCGTGCCGCT GCTGCTCGGC CTCCTCGGCC TGGCCGTCGC CGAGCCCGCC | |
|---|---|---|
| 61 | GTCTACTTCA AGGAGCAGTT TCTGGACGGA GACGGGTGGA CTTCCCGCTG GATCGAATCC | |
| 121 | AAACACAAGT CAGATTTTGG CAAATTCGTT CTCAGTTCCG GCAAGTTCTA CGGTGACGAG | |
| 181 | GAGAAAGATA AAGGTTTGCA GACAAGCCAG GATGCACGCT TTTATGCTCT GTCGGCCAGT | |
| 241 | TTCGAGCCTT TCAGCAACAA AGGCCAGACG CTGGTGGTGC AGTTCACGGT GAAACATGAG | |
| 301 | CAGAACATCG ACTGTGGGGG CGGCTATGTG AAGCTGTTTC CTAATAGTTT GGACCAGACA | |
| 361 | GACATGCACG GAGACTCAGA ATACAACATC ATGTTTGGTC CCGACATCTG TGGCCCTGGC | |
| 421 | ACCAAGAAGG TTCATGTCAT CTTCAACTAC AAGGGCAAGA ACGTGCTGAT CAACAAGGAC | |
| 481 | ATCCGTTGCA AGGATGATGA GTTTACACAC CTGTACACAC TGATTGTGCG GCCAGACAAC | |
| 541 | acctatgagg tgaagattga caacagccag gtggagtccg gctccttgga agacgattgg | |
| 601 | gacttcctgc cacccaagaa gataaaggat cctgatgctt caaaaccgga agactgggat | |
| 661 | gagcgggcca agatcgatga tcccacagac tccaagcctg aggactggga caagcccgag | |
| 721 | catatccctg accctgatgc taagaagccc gaggactggg atgaagagat ggacggagag | |
| 781 | tgggaacccc cagtgattca gaaccctgag tacaagggtg agtggaagcc ccggcagatc | |
| 841 | gacaacccag attacaaggg cacttggatc cacccagaaa ttgacaaccc cgagtattct | |
| 901 | cccgatccca gtatctatgc ctatgataac tttggcgtgc tgggcctgga cctctggcag | |
| 961 | gtcaagtctg gcaccatctt tgacaacttc ctcatcacca acgatgaggc atacgctgag | |
| 1021 | gagtttggca acgagacgtg gggcgtaaca aaggcagcag agaaacaaat gaaggacaaa | |
| 1081 | caggacgagg agcagaggct taaggaggag gaagaagaca agaaacgcaa agaggaggag | |
| 1141 | gaggcagagg acaaggagga tgatgaggac aaagatgagg atgaggagga tgaggaggac | |
| 1201 | aaggaggaag atgaggagga agatgtcccc ggccaggcca aggacgagct gtag | 1251 |
| The coding sequence for each separate domain is provided below: | | |
| Human N-CRT DNA | | |
| (SEQ ID NO: 33) | | |
| 1 | ATGCTGCTAT CCGTGCCGCT GCTGCTCGGC CTCCTCGGCC TGGCCGTCGC CGAGCCCGCC | |
| 61 | GTCTACTTCA AGGAGCAGTT TCTGGACGGA GACGGGTGGA CTTCCCGCTG GATCGAATCC | |
| 121 | AAACACAAGT CAGATTTTGG CAAATTCGTT CTCAGTTCCG GCAAGTTCTA CGGTGACGAG | |
| 181 | GAGAAAGATA AAGGTTTGCA GACAAGCCAG GATGCACGCT TTTATGCTCT GTCGGCCAGT | |
| 241 | TTCGAGCCTT TCAGCAACAA AGGCCAGACG CTGGTGGTGC AGTTCACGGT GAAACATGAG | |
| 301 | CAGAACATCG ACTGTGGGGG CGGCTATGTG AAGCTGTTTC CTAATAGTTT GGACCAGACA | |
| 361 | GACATGCACG GAGACTCAGA ATACAACATC ATGTTTGGTC CCGACATCTG TGGCCCTGGC | |
| 421 | ACCAAGAAGG TTCATGTCAT CTTCAACTAC AAGGGCAAGA ACGTGCTGAT CAACAAGGAC | |
| 481 | ATCCGTTGCA AGGATGATGA GTTTACACAC CTGTACACAC TGATTGTGCG GCCAGACAAC | |
| Human P-CRT DNA | | |
| (SEQ ID NO: 34) | | |
| 1 | acctatgagg tgaagattga caacagccag gtggagtccg gctccttgga agacgattgg | |
| 61 | gacttcctgc cacccaagaa gataaaggat cctgatgctt caaaaccgga agactgggat | |
| 121 | gagcgggcca agatcgatga tcccacagac tccaagcctg aggactggga caagcccgag | |
| 181 | catatccctg accctgatgc taagaagccc gaggactggg atgaagagat ggacggagag | |
| 241 | tgggaacccc cagtgattca gaaccct | 267 |
| Human C-CRT DNA | | |
| (SEQ ID NO: 35) | | |
| 1 | gagtacaagg gtgagtggaa gccccggcag atcgacaacc cagattacaa gggcacttgg | |
| 61 | atccacccag aaattgacaa ccccgagtat tctcccgatc ccagtatcta tgcctatgat | |
| 121 | aactttggcg tgctgggcct ggacctctgg caggtcaagt ctggcaccat ctttgacaac | |
| 181 | ttcctcatca ccaacgatga ggcatacgct gaggagtttg gcaacgagac gtggggcgta | |
| 241 | acaaaggcag cagagaaaca aatgaaggac aaacaggacg aggagcagag gcttaaggag | |
| 301 | gaggaagaag acaagaaacg caaagaggag gaggaggcag aggacaagga ggatgatgag | |
| 361 | gacaaagatg aggatgagga ggatgaggag gacaaggagg aagatgagga ggaagatgtc | |
| 421 | cccggccagg ccaaggacga gctg | 444 |

Alternatively, any nucleotide sequences that encodes these domains may be used in the present constructs. Thus, for use in humans, the sequences may be further codon-optimized

The present construct may employ combinations of one or more CRT domains, in any of a number of orientations. Using the designations N^{CRT}, P^{CRT} and C^{CRT} to designate the domains, the following are but a few examples of the combinations that may be used in the DNA vaccine vectors of the present invention (where it is understood that Ag can be any antigen, preferably E7(detox) or E6 (detox).

| | | | |
|---|---|---|---|
| N^{CRT}-P^{CRT}-Ag; | N^{CRT}-P^{CRT}-Ag; | N^{CRT}-C^{CRT}-Ag; | N^{CRT}-N^{CRT}-Ag; |
| N^{CRT}-N^{CRT}-N^{CRT}-Ag; | P^{CRT}-P^{CRT}-Ag; | P^{CRT}-C^{CRT}-Ag; | P^{CRT}-N^{CRT}-Ag; |
| C^{CRT}-P^{CRT}-Ag; | N^{CRT}-P^{CRT}-Ag; | etc. | |

The present invention may employ shorter polypeptide fragments of CRT or CRT domains provided such fragments can enhance the immune response to an antigen with which they are paired. Shorter peptides from the CRT or domain sequences shown above that have the ability to promote protein processing via the MHC-1 class I pathway are also included, and may be defined by routine experimentation.

The present invention may also employ shorter nucleic acid fragments that encode CRT or CRT domains provided such fragments are functional, e.g., encode polypeptides that can enhance the immune response to an antigen with which they are paired (e.g., linked). Nucleic acids that encode shorter peptides from the CRT or domain sequences shown above and are functional, e.g., have the ability to promote protein processing via the MHC-1 class I pathway, are also included, and may be defined by routine experimentation.

A polypeptide fragment of CRT may include at least or about 50, 100, 200, 300, or 400 amino acids. A polypeptide fragment of CRT may also include at least or about 25, 50, 75, 100, 25-50, 50-100, or 75-125 amino acids from a CRT domain selected from the group consisting of the N-CRT, P-CRT, and C-CRT. A polypeptide fragment of CRT may include residues 1-50, 50-75, 75-100, 100-125, 125-150, 150-170 of the N-domain (e.g., of SEQ ID NO: 30). A polypeptide fragment of CRT may include residues 1-50, 50-75, 75-100, 100-109 of the P-domain (e.g., of SEQ ID NO: 31). A polypeptide fragment of CRT may include residues 1-50, 50-75, 75-100, 100-125, 125-138 of the C-domain (e.g., of SEQ ID NO: 32).

A nucleic acid fragment of CRT may encode at least or about 50, 100, 200, 300, or 400 amino acids. A nucleic acid fragment of CRT may also encode at least or about 25, 50, 75, 100, 25-50, 50-100, or 75-125 amino acids from a CRT domain selected from the group consisting of the N-CRT, P-CRT, and C-CRT. A nucleic acid fragment of CRT may encode residues 1-50, 50-75, 75-100, 100-125, 125-150, 150-170 of the N-domain (e.g., of SEQ ID NO: 30). A nucleic acid fragment of CRT may encode residues 1-50, 50-75, 75-100, 100-109 of the P-domain (e.g., of SEQ ID NO: 31). A nucleic acid fragment of CRT may encode residues 1-50, 50-75, 75-100, 100-125, 125-138 of the C-domain (e.g., of SEQ ID NO: 32).

Polypeptide “fragments” of CRT, as provided herein, do not include full-length CRT. Likewise, nucleic acid “fragments” of CRT, as provided herein, do not include a full-length CRT nucleic acid sequence and do not encode a full-length CRT polypeptide.

A most preferred vector construct of a complete chimeric nucleic acid of the invention, is shown below (SEQ ID NO: 36). The sequence is annotated to show plasmid-derived nucleotides (lower case letters), CRT-derived nucleotides (upper case bold letters), and HPV-E7-derived nucleotides (upper case, italicized/underlined letters). Note that 5 plasmid nucleotides are found between the CRT and E7 coding sequences and that the stop codon for the E7 sequence is double underscored. This plasmid is also referred to as pNGVL4a-CRT/E7(detox).

```
      1 gctccgcccc cctgacgagc atcacaaaaa tcgacgctca agtcagaggt ggcgaaaccc
    61 gacaggacta taaagatacc aggcgtttcc ccctggaagc tccctcgtgc gctctcctgt
  121 tccgaccctg ccgcttaccg gatacctgtc cgcctttctc ccttcgggaa gcgtggcgct
  181 ttctcatagc tcacgctgta ggtatctcag ttcggtgtag gtcgttcgct ccaagctggg
  241 ctgtgtgcac gaaccccccg ttcagcccga ccgctgcgcc ttatccggta actatcgtct
  301 tgagtccaac ccggtaagac acgacttatc gccactggca gcagccactg gtaacaggat
  361 tagcagagcg aggtatgtag gcggtgctac agagttcttg aagtggtggc ctaactacgg
  421 ctacactaga agaacagtat ttggtatctg cgctctgctg aagccagtta ccttcggaaa
  481 aagagttggt agctcttgat ccggcaaaca aaccaccgct ggtagcggtg gtttttttgt
  541 ttgcaagcag cagattacgc gcagaaaaaa aggatctcaa gaagatcctt tgatcttttc
  601 tacggggtct gacgctcagt ggaacgaaaa ctcacgttaa gggattttgg tcatgagatt
  661 atcaaaaagg atcttcacct agatcctttt aaattaaaaa tgaagtttta aatcaatcta
  721 aagtatatat gagtaaactt ggtctgacag ttaccaatgc ttaatcagtg aggcacctat
  781 ctcagcgatc tgtctatttc gttcatccat agttgcctga ctcggggggg gggggcgctg
  841 aggtctgcct cgtgaagaag gtgttgctga ctcataccag ggcaacgttg ttgccattgc
  901 tacaggcatc gtggtgtcac gctcgtcgtt tggtatggct tcattcagct ccggttccca
  961 acgatcaagg cgagttacat gatcccccat gttgtgcaaa aaagcggtta gctccttcgg
1021 tcctccgatc gttgtcagaa gtaagttggc cgcagtgtta tcactcatgg ttatggcagc
1081 actgcataat tctcttactg tcatgccatc cgtaagatgc ttttctgtga ctggtgagta
1141 ctcaaccaag tcattctgag aatagtgtat gcggcgaccg agttgctctt gcccggcgtc
1201 aatacgggat aataccgcgc cacatagcag aactttaaaa gtgctcatca ttggaaaacg
1261 ttcttcgggg cgaaaactct caaggatctt accgctgttg agatccagtt cgatgtaacc
1321 cactcgtgca cctgaatcgc cccatcatcc agccagaaag tgagggagcc acggttgatg
1381 agagctttgt tgtaggtgga ccagttggtg attttgaact tttgctttgc cacggaacgg
1441 tctgcgttgt cgggaagatg cgtgatctga tccttcaact cagcaaaagt tcgatttatt
1501 caacaaagcc gccgtcccgt caagtcagcg taatgctctg ccagtgttac aaccaattaa
1561 ccaattctga ttagaaaaac tcatcgagca tcaaatgaaa ctgcaattta ttcatatcag
1621 gattatcaat accatatttt tgaaaaagcc gtttctgtaa tgaaggagaa aactcaccga
1681 ggcagttcca taggatggca agatcctggt atcggtctgc gattccgact cgtccaacat
1741 caatacaacc tattaatttc ccctcgtcaa aaataaggtt atcaagtgag aaatcaccat
1801 gagtgacgac tgaatccggt gagaatggca aaagcttatg catttctttc cagacttgtt
1861 caacaggcca gccattacgc tcgtcatcaa aatcactcgc atcaaccaaa ccgttattca
1921 ttcgtgattg cgcctgagcg agacgaaata cgcgatcgct gttaaaagga caattacaaa
1981 caggaatcga atgcaaccgg cgcaggaaca ctgccagcgc atcaacaata ttttcacctg
2041 aatcaggata ttcttctaat acctggaatg ctgttttccc ggggatcgca gtggtgagta
2101 accatgcatc atcaggagta cggataaaat gcttgatggt cggaagaggc ataaattccg
2161 tcagccagtt tagtctgacc atctcatctg taacatcatt ggcaacgcta cctttgccat
2221 gtttcagaaa caactctggc gcatcgggct tcccatacaa tcgatagatt gtcgcacctg
2281 attgcccgac attatcgcga gcccatttat acccatataa atcagcatcc atgttggaat
2341 ttaatcgcgg cctcgagcaa gacgtttccc gttgaatatg gctcataaca ccccttgtat
2401 tactgtttat gtaagcagac agttttattg ttcatgatga tatattttta tcttgtgcaa
2461 tgtaacatca gagattttga gacacaacgt ggctttcccc ccccccccat tattgaagca
2521 tttatcaggg ttattgtctc atgagcggat acatatttga atgtatttag aaaaataaac
2581 aaataggggt tccgcgcaca tttccccgaa aagtgccacc tgacgtctaa gaaaccatta
2641 ttatcatgac attaacctat aaaaataggc gtatcacgag gccctttcgt ctcgcgcgtt
2701 tcggtgatga cggtgaaaac ctctgacaca tgcagctccc ggagacggtc acagcttgtc
2761 tgtaagcgga tgccgggagc agacaagccc gtcagggcgc gtcagcgggt gttggcgggt
2821 gtcggggctg gcttaactat gcggcatcag agcagattgt actgagagtg caccatatgc
2881 ggtgtgaaat accgcacaga tgcgtaagga gaaaataccg catcagattg gctattggcc
2941 attgcatacg ttgtatccat atcataatat gtacatttat attggctcat gtccaacatt
3001 accgccatgt tgacattgat tattgactag ttattaatag taatcaatta cggggtcatt
3061 agttcatagc ccatatatgg agttccgcgt tacataactt acggtaaatg gcccgcctgg
3121 ctgaccgccc aacgaccccc gcccattgac gtcaataatg acgtatgttc ccatagtaac
3181 gccaataggg actttccatt gacgtcaatg ggtggagtat ttacggtaaa ctgcccactt
3241 ggcagtacat caagtgtatc atatgccaag tacgccccct attgacgtca atgacggtaa
3301 atggcccgcc tggcattatg cccagtacat gaccttatgg gactttccta cttggcagta
3361 catctacgta ttagtcatcg ctattaccat ggtgatgcgg ttttggcagt acatcaatgg
3421 gcgtggatag cggtttgact cacggggatt tccaagtctc caccccattg acgtcaatgg
3481 gagtttgttt tggcaccaaa atcaacggga ctttccaaaa tgtcgtaaca actccgcccc
3541 attgacgcaa atgggcggta ggcgtgtacg gtgggaggtc tatataagca gagctcgttt
3601 agtgaaccgt cagatcgcct ggagacgcca tccacgctgt tttgacctcc atagaagaca
3661 ccgggaccga tccagcctcc gcggccggga acggtgcatt ggaacgcgga ttccccgtgc
3721 caagagtgac gtaagtaccg cctatagact ctataggcac acccctttgg ctcttatgca
3781 tgctatactg tttttggctt ggggcctata cacccccgct tccttatgct ataggtgatg
3841 gtatagctta gcctataggt gtgggttatt gaccattatt gaccactcca acggtggagg
3901 gcagtgtagt ctgagcagta ctcgttgctg ccgcgcgcgc caccagacat aatagctgac
3961 agactaacag actgttcctt tccatgggtc ttttctgcag tcaccgtcgt cgacATGCTG
4021 CTATCCGTGC CGCTGCTGCT CGGCCTCCTC GGCCTGGCCG TCGCCGAGCC TGCCGTCTAC
4081 TTCAAGGAGC AGTTTCTGGA CGGGGACGGG TGGACTTCCC GCTGGATCGA ATCCAAACAC
4141 AAGTCAGATT TTGGCAAATT CGTTCTCAGT TCCGGCAAGT TCTACGGTGA CGAGGAGAAA
4201 GATAAAGGTT TGCAGACAAG CCAGGATGCA CGCTTTTATG CTCTGTCGGC CAGTTTCGAG
4261 CCTTTCAGCA ACAAAGGCCA GACGCTGGTG GTGCAGTTCA CGGTGAAACA TGAGCAGAAC
4321 ATCGACTGTG GGGGCGGCTA TGTGAAGCTG TTTCCTAATA GTTTGGACCA GACAGACATG
4381 CACGGAGACT CAGAATACAA CATCATGTTT GGTCCCGACA TCTGTGGCCC TGGCACCAAG
4441 AAGGTTCATG TCATCTTCAA CTACAAGGGC AAGAACGTGC TGATCAACAA GGACATCCGT
4501 TGCAAGGATG ATGAGTTTAC ACACCTGTAC ACACTGATTG TGCGGCCAGA CAACACCTAT
4561 GAGGTGAAGA TTGACAACAG CCAGGTGGAG TCCGGCTCCT TGGAAGACGA TTGGGACTTC
4621 CTGCCACCCA AGAAGATAAA GGATCCTGAT GCTTCAAAAC CGGAAGACTG GGATGAGCGG
4681 GCCAAGATCG ATGATCCCAC AGACTCCAAG CCTGAGGACT GGGACAAGCC CGAGCATATC
4741 CCTGACCCTG ATGCTAAGAA GCCCGAGGAC TGGGATGAAG AGATGGACGG AGAGTGGGAA
4801 CCCCCAGTGA TTCAGAACCC TGAGTACAAG GGTGAGTGGA AGCCCCGGCA GATCGACAAC
4861 CCAGATTACA AGGGCACTTG GATCCACCCA GAAATTGACA ACCCCGAGTA TTCTCCCGAT
4921 CCCAGTATCT ATGCCTATGA TAACTTTGGC GTGCTGGGCC TGGACCTCTG GCAGGTCAAG
4981 TCTGGCACCA TCTTTGACAA CTTCCTCATC ACCAACGATG AGGCATACGC TGAGGAGTTT
5041 GGCAACGAGA CGTGGGGCGT AACAAAGGCA GCAGAGAAAC AAATGAAGGA CAAACAGGAC
5101 GAGGAGCAGA GGCTTAAGGA GGAGGAAGAA GACAAGAAAC GCAAAGAGGA GGAGGAGGCA
5161 GAGGACAAGG AGGATGATGA GGACAAAGAT GAGGATGAGG AGGATGAGGA GGACAAGGAG

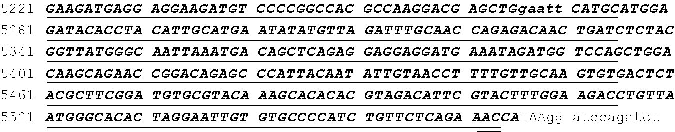

5581 ttttccctct gccaaaaatt atggggacat catgaagccc cttgagcatc tgacttctgg
5641 ctaataaagg aaatttattt tcattgcaat agtgtgttgg aattttttgt gtctctcact
5701 cggaaggaca tatgggaggg caaatcattt aaaacatcag aatgagtatt tggtttagag
5761 tttggcaaca tatgcccatt cttccgcttc ctcgctcact gactcgctgc gctcggtcgt
5821 tcggctgcgg cgagcggtat cagctcactc aaaggcggta atacggttat ccacagaatc
5881 aggggataac gcaggaaaga acatgtgagc aaaaggccag caaaaggcca ggaaccgtaa
5941 aaaggccgcg ttgctggcgt ttttccatag 5970
```

Table 2 below describes the structure of the above plasmid.

TABLE 2

| Plasmid Position | Genetic Construct | Source of Construct |
|---|---|---|
| 5970-0823 | E. coli ORI (ColE1) | pBR/E. coli-derived |
| 0837-0881 | portion of transposase (tpnA) | Common plasmid |
| | | sequence Tn5/Tn903 |
| 0882-1332 | β-Lactamase (Amp^{R}) | pBRpUC derived |
| | | plasmid |
| 1331-2496 | AphA (Kan^{R}) | Tn903 |
| 2509-2691 | P3 Promoter DNA binding | Tn3/pBR322 |
| | site | |
| 2692-2926 | pUC backbone | Common plasmid |
| | | sequence pBR322- |
| | | derived |
| 2931-4009 | NF1 binding and promoter | HHV-5(HCMV UL-10 |
| | | lE1 gene) |
| 4010-4014 | Poly-cloning site | Common plasmid |
| | | sequence |
| 4015-5265 | Calreticulin (CRT) | Human Calreticulin |
| 5266-5271 | GAATTC plasmid sequence | Remain after cloning |
| 5272-5568 | dE7 gene (detoxified partial) | HPV-16 (E7 gene) |
| | | incl. stop codon |
| 5569-5580 | Poly-cloning site | Common plasmid |
| | | sequence |
| 551-5970 | Poly-Adenylation site | Mammalian signal, |
| | | pHCMV-derived |

In some embodiments, an alternative to CRT is one the other ER chaperone polypeptide exemplified by ER60, GRP94 or gp96, well-characterized ER chaperone polypeptide that representatives of the HSP90 family of stress-induced proteins (see WO 02/012281). The term “endoplasmic reticulum chaperone polypeptide” as used herein means any polypeptide having substantially the same ER chaperone function as the exemplary chaperone proteins CRT, tapasin, ER60 or calnexin. Thus, the term includes all functional fragments or variants or mimics thereof A polypeptide or peptide can be routinely screened for its activity as an ER chaperone using assays known in the art. While the invention is not limited by any particular mechanism of action, in vivo chaperones promote the correct folding and oligomerization of many glycoproteins in the ER, including the assembly of the MHC class I heterotrimeric molecule (heavy (H) chain, β2m, and peptide). They also retain incompletely assembled MHC class I heterotrimeric complexes in the ER (Hauri FEBS Lett. 476:32-37, 2000).

### Intercellular Spreading Proteins

The potency of naked DNA vaccines may be enhanced by their ability to amplify and spread in vivo. VP22, a herpes simplex virus type 1 (HSV-1) protein and its “homologues” in other herpes viruses, such as the avian Marek's Disease Virus (MDV) have the property of intercellular transport that provide an approach for enhancing vaccine potency. The present inventors have previously created novel fusions of VP22 with a model antigen, human papillomavirus type 16 (HPV-16) E7, in a DNA vaccine which generated enhanced spreading and MHC class I presentation of antigen. These properties led to a dramatic increase in the number of E7-specific CD8+ T cell precursors in vaccinated mice (at least 50-fold) and converted a less effective DNA vaccine into one with significant potency against E7-expressing tumors. In comparison, a non-spreading mutant, VP22(1-267), failed to enhance vaccine potency. Results presented in U.S. Patent Application publication No. 20040028693, hereby incorporated by reference in its entirety, show that the potency of DNA vaccines is dramatically improved through enhanced intercellular spreading and MHC class I presentation of the antigen.

A similar study linking MDV-1 UL49 to E7 also led to a dramatic increase in the number of E7-specific CD8+ T cell precursors and potency response against E7-expressing tumors in vaccinated mice. Mice vaccinated with a MDV-1 UL49 DNA vaccine stimulated E7-specific CD8+ T cell precursor at a level comparable to that induced by HSV-1 VP22/E7. Thus, fusion of MDV-1UL49 DNA to DNA encoding a target antigen gene significantly enhances the DNA vaccine potency.

The spreading protein is preferably a viral spreading protein, most preferably a herpesvirus VP22 protein. Exemplified herein are fusion constructs that comprise herpes simplex virus-1 (HSV-1) VP22 (abbreviated HVP22) and its homologue from Marek's disease virus (MDV) termed MDV-VP22 or MVP-22). Also included in the invention are homologues of VP22 from other members of the herpesviridae or polypeptides from nonviral sources that are considered to be homologous and share the functional characteristic of promoting intercellular spreading of a polypeptide or peptide that is fused or chemically conjugated thereto.

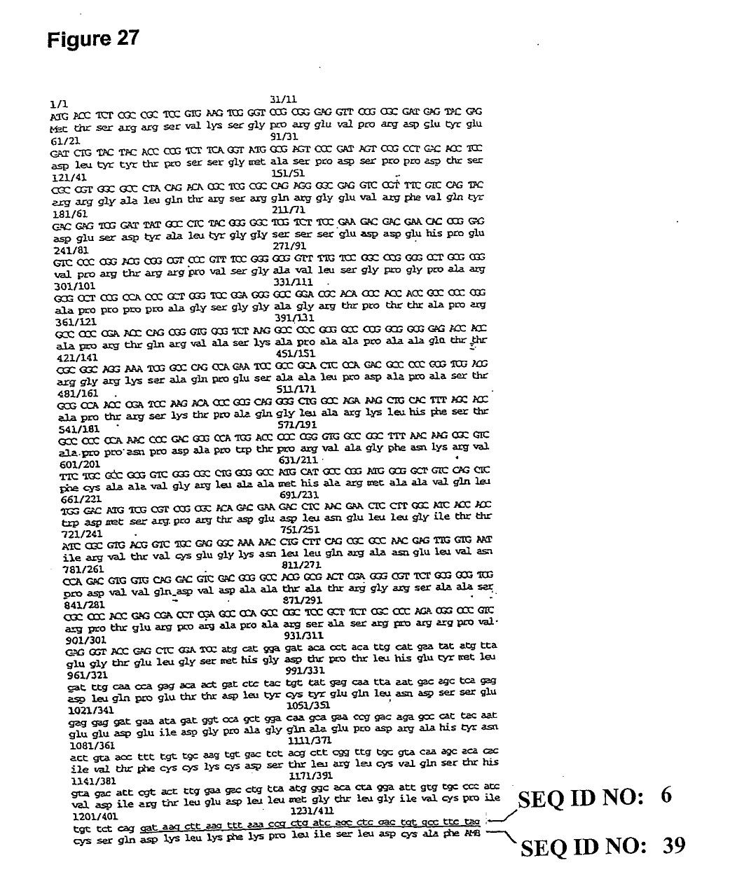

DNA encoding HVP22 has the sequence SEQ ID NO: 7 which is shown in FIG. 27 as nucleotides 1-921 of the longer sequence SEQ ID NO: 6 (which is the full length nucleotide sequence of a vector that comprises HVP22). DNA encoding MDV-VP22 is SEQ ID NO: 37 shown below:

| 1 atg ggg gat tct gaa agg cgg aaa tcg gaa cgg | |
|---|---|
| cgt cgt tcc ctt gga 48 tat ccc tct gca tat gat | |
| gac gtc tcg att cct gct cgc aga cca tca 96 aca | |
| cgt act cag cga aat tta aac cag gat gat ttg tca | |
| aaa cat gga 144 cca ttt acc gac cat cca aca caa | |
| aaa cat aaa tcg gcg aaa gcc gta 192 tcg gaa gac | |
| gtt tcg tct acc acc cgg ggt ggc ttt aca aac aaa | |
| ccc 240 cgt acc aag ccc ggg gtc aga gct gta caa | |
| agt aat aaa ttc gct ttc 288 agt acg gct cct tca | |
| tca gca tct agc act tgg aga tca aat aca gtg 336 | |
| gca ttt aat cag cgt atg ttt tgc gga gcg gtt gca | |
| act gtg gct caa 384 tat cac gca tac caa ggc gcg | |
| ctc gcc ctt tgg cgt caa gat cct ccg 432 cga aca | |
| aat gaa gaa tta gat gca ttt ctt tcc aga gct gtc | |
| att aaa 480 att acc att caa gag ggt cca aat ttg | |
| atg ggg gaa gcc gaa acc tgt 528 gcc cgc aaa cta | |
| ttg gaa gag tct gga tta tcc cag ggg aac gag aac | |
| 576 gta aag tcc aaa tot gaa cgt aca acc aaa tct | |
| gaa cgt aca aga cgc 624 ggc ggt gaa att gaa atc | |
| aaa tcg cca gat ccg gga tct cat cgt aca 672 cat | |
| aac cct cgc act ccc gca act tcg cgt cgc cat cat | |
| tca tcc gcc 720 cgc gga tat cgt agc agt gat agc | |
| gaa taa | 747 |

The amino acid sequence of HVP22 polypeptide is SEQ ID NO: 38 which is shown in FIG. 27 as amino acid residues 1-301 of SEQ ID NO: 39 (the full length amino acid encoded by the vector).

The amino acid sequence of the MDV-VP22, SEQ ID NO: 40, is below:

2 Met Gly Asp Ser Glu Arg Arg Lys Ser Glu Arg Arg
Arg Ser Leu Gly 16 Tyr Pro Ser Ala Tyr Asp Asp Val
Ser Ile Pro Ala Arg Arg Pro Ser 32 Thr Arg Thr Gln
Arg Asn Leu Asn Gln Asp Asp Leu Ser Lys His Gly
48 Pro Phe Thr Asp His Pro Thr Gln Lys His Lys
Ser Ala Lys Ala Val 64 Ser Glu Asp Val Ser Ser Thr
Thr Arg Gly Gly Phe Thr Asn Lys Pro 80 Arg Thr Lys
Pro Gly Val Arg Ala Val Gln Ser Asn Lys Phe Ala
Phe 96 Ser Thr Ala Pro Ser Ser Ala Ser Ser Thr Trp
Arg Ser Asn Thr Val 112 Ala Phe Asn Gln Arg Met
Phe Cys Gly Ala Val Ala Thr Val Ala Gln 128 Tyr
His Ala Tyr Gln Gly Ala Leu Ala Leu Trp Arg Gln
Asp Pro Pro 144 Arg Thr Asn Glu Glu Leu Asp Ala
Phe Leu Ser Arg Ala Val Ile Lys 160 Ile Thr Ile
Gln Glu Gly Pro Asn Leu Met Gly Glu Ala Glu Thr
Cys 176 Ala Arg Lys Leu Leu Glu Glu Ser Gly Leu
Ser Gln Gly Asn Glu Asn 192 Val Lys Ser Lys Ser
Glu Arg Thr Thr Lys Ser Glu Arg Thr Arg Arg 208
Gly Gly Glu Ile Glu Ile Lys Ser Pro Asp Pro Gly
Ser His Arg Thr 224 His Asn Pro Arg Thr Pro Ala
Thr Ser Arg Arg His His Ser Ser Ala 240 Arg Gly
Tyr Arg Ser Ser Asp Ser Glu -- 249

A DNA clone pcDNA3 VP22/E7, that includes the coding sequence for HVP22 and the HPV-16 protein, E7 (plus some additional vector sequence) is SEQ ID NO: 6.

The amino acid sequence of E7 (SEQ ID NO: 41) is residues 308-403 of SEQ ID NO: 39. This particular clone has only 96 of the 98 residues present in E7. The C-terminal residues of wild-type E7, Lys and Pro, are absent from this construct. This is an example of a deletion variant as the term is described below. Such deletion variants (e.g., terminal truncation of two or a small number of amino acids) of other antigenic polypeptides are examples of the embodiments intended within the scope of the fusion polypeptides of this invention.

### Homologues of IPPs

Homologues or variants of IPPs described herein, may also be used, provided that they have the requisite biological activity. These include various substitutions, deletions, or additions of the amino acid or nucleic acid sequences. Due to code degeneracy, for example, there may be considerable variation in nucleotide sequences encoding the same amino acid sequence.

A functional derivative of an IPP retains measurable IPP-like activity, preferably that of promoting immunogenicity of one or more antigenic epitopes fused thereto by promoting presentation by class I pathways. “Functional derivatives” encompass “variants” and “fragments” regardless of whether the terms are used in the conjunctive or the alternative herein.

The term “chimeric” or “fusion” polypeptide or protein refers to a composition comprising at least one polypeptide or peptide sequence or domain that is chemically bound in a linear fashion with a second polypeptide or peptide domain. One embodiment of this invention is an isolated or recombinant nucleic acid molecule encoding a fusion protein comprising at least two domains, wherein the first domain comprises an IPP and the second domain comprises an antigenic epitope, e.g., an MHC class I-binding peptide epitope. The “fusion” can be an association generated by a peptide bond, a chemical linking, a charge interaction (e.g., electrostatic attractions, such as salt bridges, H-bonding, etc.) or the like. If the polypeptides are recombinant, the “fusion protein” can be translated from a common mRNA. Alternatively, the compositions of the domains can be linked by any chemical or electrostatic means. The chimeric molecules of the invention (e.g., targeting polypeptide fusion proteins) can also include additional sequences, e.g., linkers, epitope tags, enzyme cleavage recognition sequences, signal sequences, secretion signals, and the like. Alternatively, a peptide can be linked to a carrier simply to facilitate manipulation or identification/location of the peptide.

Also included is a “functional derivative” of an IPP, which refers to an amino acid substitution variant, a “fragment,” etc., of the protein, which terms are defined below. A functional derivative of an IPP retains measurable activity, preferably that is manifest as promoting immunogenicity of one or more antigenic epitopes fused thereto or co-administered therewith. “Functional derivatives” encompass “variants” and “fragments” regardless of whether the terms are used in the conjunctive or the alternative herein.

A functional homologue must possess the above biochemical and biological activity. In view of this functional characterization, use of homologous proteins including proteins not yet discovered, fall within the scope of the invention if these proteins have sequence similarity and the recited biochemical and biological activity.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred method of alignment, Cys residues are aligned.

In a preferred embodiment, the length of a sequence being compared is at least 30%, preferably at least 40%, more preferably at least 50%, even more preferably at least 60%, and even more preferably at least 70%, 80%, 90%, 95%, 96%, 97%, 98%, or 99% of the length of the IPP reference sequence. The amino acid residues (or nucleotides) at corresponding amino acid (or nucleotide) positions are then compared. When a position in the first sequence is occupied by the same amino acid residue (or nucleotide) as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases, for example, to identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to IPP nucleic acid molecules. BLAST protein searches can be performed with the XBLAST program, score=50, wordlength=3 to obtain amino acid sequences homologous to IPP protein molecules. To obtain gapped alignments for comparison purposes, Gapped BLAST can be utilized as described in Altschul et al. (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm nih gov.

Thus, a homologue of an IPP or of an IPP domain described above is characterized as having (a) functional activity of native IPP or domain thereof and (b) amino acid sequence similarity to a native IPP protein or domain thereof when determined as above, of at least about 50%, 55%, 60%, 65%, 70%, 75%, 80%, 85%, 90%, 95%, 96%, 97%, 98%, or 99%.

It is within the skill in the art to obtain and express such a protein using DNA probes based on the disclosed sequences of an IPP. Then, the fusion protein's biochemical and biological activity can be tested readily using art-recognized methods such as those described herein, for example, a T cell proliferation, cytokine secretion or a cytolytic assay, or an in vivo assay of tumor protection or tumor therapy. A biological assay of the stimulation of antigen-specific T cell reactivity will indicate whether the homologue has the requisite activity to qualify as a “functional” homologue.

A “variant” refers to a molecule substantially identical to either the full protein or to a fragment thereof in which one or more amino acid residues have been replaced (substitution variant) or which has one or several residues deleted (deletion variant) or added (addition variant). A “fragment” of an IPP refers to any subset of the molecule, that is, a shorter polypeptide of the full-length protein.

A number of processes can be used to generate fragments, mutants and variants of the isolated DNA sequence. Small subregions or fragments of the nucleic acid encoding the spreading protein, for example 1-30 bases in length, can be prepared by standard, chemical synthesis. Antisense oligonucleotides and primers for use in the generation of larger synthetic fragment.

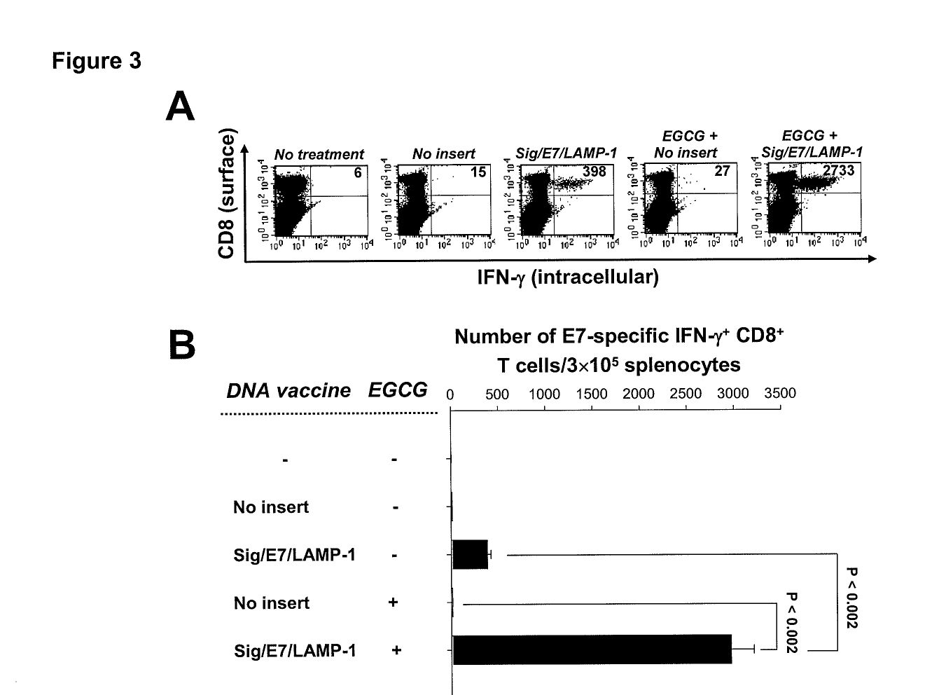

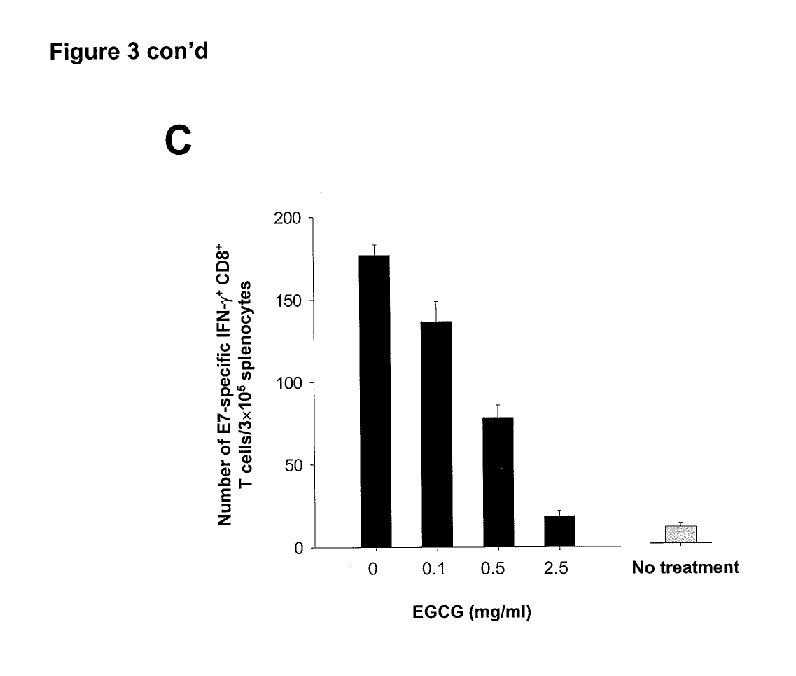

A preferred group of variants are those in which at least one amino acid residue and preferably, only one, has been substituted by different residue. For a detailed description of protein chemistry and structure, see Schulz, G E et al., Principles of Protein Structure, Springer-Verlag, New York, 1978, and Creighton, T. E., Proteins: Structure and Molecular Properties, W.H. Freeman & Co., San Francisco, 1983, which are hereby incorporated by reference. The types of substitutions that may be made in the protein molecule may be based on analysis of the frequencies of amino acid changes between a homologous protein of different species, such as those presented in Table 1-2 of Schulz et al. (supra) and FIG. 3-9 of Creighton (supra). Based on such an analysis, conservative substitutions are defined herein as exchanges within one of the following five groups:

| | | |
|---|---|---|
| 1. | Small aliphatic, nonpolar or slightly polar | Ala, Ser, Thr (Pro, Gly); |
| | residues | |
| 2. | Polar, negatively charged residues and | Asp, Asn, Glu, Gln; |
| | their amides | |
| 3. | Polar, positively charged residues | His, Arg, Lys; |
| 4. | Large aliphatic, nonpolar residues | Met, Leu, Ile, Val (Cys) |
| 5. | Large aromatic residues | Phe, Tyr, Trp. |

The three amino acid residues in parentheses above have special roles in protein architecture. Gly is the only residue lacking a side chain and thus imparts flexibility to the chain. Pro, because of its unusual geometry, tightly constrains the chain. Cys can participate in disulfide bond formation, which is important in protein folding.

More substantial changes in biochemical, functional (or immunological) properties are made by selecting substitutions that are less conservative, such as between, rather than within, the above five groups. Such changes will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain. Examples of such substitutions are (i) substitution of Gly and/or Pro by another amino acid or deletion or insertion of Gly or Pro; (ii) substitution of a hydrophilic residue, e.g., Ser or Thr, for (or by) a hydrophobic residue, e.g., Leu, Ile, Phe, Val or Ala; (iii) substitution of a Cys residue for (or by) any other residue; (iv) substitution of a residue having an electropositive side chain, e.g., Lys, Arg or His, for (or by) a residue having an electronegative charge, e.g., Glu or Asp; or (v) substitution of a residue having a bulky side chain, e.g., Phe, for (or by) a residue not having such a side chain, e.g., Gly.

Most acceptable deletions, insertions and substitutions according to the present invention are those that do not produce radical changes in the characteristics of the wild-type or native protein in terms of its relevant biological activity, e.g., its ability to stimulate antigen specific T cell reactivity to an antigenic epitope or epitopes that are fused to the protein. However, when it is difficult to predict the exact effect of the substitution, deletion or insertion in advance of doing so, one skilled in the art will appreciate that the effect can be evaluated by routine screening assays such as those described here, without requiring undue experimentation.

Exemplary fusion proteins provided herein comprise an IPP protein or homolog thereof and an antigen. For example, a fusion protein may comprise, consists essentially of, or consists of an IPP or a an IPP fragment, e.g., N-CRT, P-CRT and/or C-CRT, or an amino acid sequence that is at least about 50%, 55%, 60%, 65%, 70%, 75%, 80%, 85%, 90%, 95%, 96%, 97%, 98%, or 99% identical to the amino acid sequence of the IPP or IPP fragment, wherein the IPP fragment is functionally active as further described herein, linked to an antigen. A fusion protein may also comprise an IPP or an IPP fragment and at least 1, 2, 3, 4, 5, 6, 7, 8, 9, 10 or more amino acids, or about 1-5, 1-10, 1-15, 1-20, 1-25, 1-30, 1-50 amino acids, at the N- and/or C-terminus of the IPP fragment. These additional amino acids may have an amino acid sequence that is unrelated to the amino acid sequence at the corresponding position in the IPP protein.

Homologs of an IPP or an IPP fragments may also comprise, consist essentially of, or consist of an amino acid sequence that differs from that of an IPP or IPP fragment by the addition, deletion, or substitution, e.g., conservative substitution, of at least about 1, 2, 3, 4, 5, 6, 7, 8, 9 or 10 amino acids, or from about 1-5, 1-10, 1-15 or 1-20 amino acids. Homologs of an IPP or IPP fragments may be encoded by nucleotide sequences that are at least about 50%, 55%, 60%, 65%, 70%, 75%, 80%, 85%, 90%, 95%, 96%, 97%, 98%, or 99% identical to the nucleotide sequence encoding an IPP or IPP fragment, such as those described herein.

Yet other homologs of an IPP or IPP fragments are encoded by nucleic acids that hybridize under stringent hybridization conditions to a nucleic acid that encodes an IPP or IPP fragment. For example, homologs may be encoded by nucleic acids that hybridize under high stringency conditions of 0.2 to 1×SSC at 65° C. followed by a wash at 0.2×SSC at 65° C. to a nucleic acid consisting of a sequence described herein. Nucleic acids that hybridize under low stringency conditions of 6×SSC at room temperature followed by a wash at 2×SSC at room temperature to nucleic acid consisting of a sequence described herein or a portion thereof can be used. Other hybridization conditions include 3×SSC at 40 or 50° C., followed by a wash in 1 or 2×SSC at 20, 30, 40, 50, 60, or 65° C. Hybridizations can be conducted in the presence of formaldehyde, e.g., 10%, 20%, 30% 40% or 50%, which further increases the stringency of hybridization. Theory and practice of nucleic acid hybridization is described, e.g., in S. Agrawal (ed.) Methods in Molecular Biology, volume 20; and Tijssen (1993) Laboratory Techniques in biochemistry and molecular biology-hybridization with nucleic acid probes, e.g., part I chapter 2 “Overview of principles of hybridization and the strategy of nucleic acid probe assays,” Elsevier, N.Y. provide a basic guide to nucleic acid hybridization.

A fragment of a nucleic acid sequence is defined as a nucleotide sequence having fewer nucleotides than the nucleotide sequence encoding the full length CRT polypeptide, antigenic polypeptide, or the fusion thereof. This invention includes such nucleic acid fragments that encode polypeptides which retain (1) the ability of the fusion polypeptide to induce increases in frequency or reactivity of T cells, preferably CD8+ T cells, that are specific for the antigen part of the fusion polypeptide.

Nucleic acid sequences of this invention may also include linker sequences, natural or modified restriction endonuclease sites and other sequences that are useful for manipulations related to cloning, expression or purification of encoded protein or fragments. For example, a fusion protein may comprise a linked between the antigen and the IPP protein.

### Backbone of DNA Vaccine

The DNA vaccine may comprise an “expression vector” or “expression cassette,” i.e., a nucleotide sequence which is capable of affecting expression of a protein coding sequence in a host compatible with such sequences. Expression cassettes include at least a promoter operably linked with the polypeptide coding sequence; and, optionally, with other sequences, e.g., transcription termination signals. Additional factors necessary or helpful in effecting expression may also be included, e.g., enhancers.

“Operably linked” means that the coding sequence is linked to a regulatory sequence in a manner that allows expression of the coding sequence. Known regulatory sequences are selected to direct expression of the desired protein in an appropriate host cell. Accordingly, the term “regulatory sequence” includes promoters, enhancers and other expression control elements. Such regulatory sequences are described in, for example, Goeddel, Gene Expression Technology. Methods in Enzymology, vol. 185, Academic Press, San Diego, Calif. (1990)).

A promoter region of a DNA or RNA molecule binds RNA polymerase and promotes the transcription of an “operably linked” nucleic acid sequence. As used herein, a “promoter sequence” is the nucleotide sequence of the promoter which is found on that strand of the DNA or RNA which is transcribed by the RNA polymerase. Two sequences of a nucleic acid molecule, such as a promoter and a coding sequence, are “operably linked” when they are linked to each other in a manner which permits both sequences to be transcribed onto the same RNA transcript or permits an RNA transcript begun in one sequence to be extended into the second sequence. Thus, two sequences, such as a promoter sequence and a coding sequence of DNA or RNA are operably linked if transcription commencing in the promoter sequence will produce an RNA transcript of the operably linked coding sequence. In order to be “operably linked” it is not necessary that two sequences be immediately adjacent to one another in the linear sequence.

The preferred promoter sequences of the present invention must be operable in mammalian cells and may be either eukaryotic or viral promoters. Although preferred promoters are described in the Examples, other useful promoters and regulatory elements are discussed below. Suitable promoters may be inducible, repressible or constitutive. A “constitutive” promoter is one which is active under most conditions encountered in the cell's environmental and throughout development. An “inducible” promoter is one which is under environmental or developmental regulation. A “tissue specific” promoter is active in certain tissue types of an organism. An example of a constitutive promoter is the viral promoter MSV-LTR, which is efficient and active in a variety of cell types, and, in contrast to most other promoters, has the same enhancing activity in arrested and growing cells. Other preferred viral promoters include that present in the CMV-LTR (from cytomegalovirus) (Bashart, M. et al., Cell 41:521, 1985) or in the RSV-LTR (from Rous sarcoma virus) (Gorman, C M, Proc. Natl. Acad. Sci. USA 79:6777, 1982). Also useful are the promoter of the mouse metallothionein I gene (Hamer, D, et al., J. Mol. Appl. Gen. 1:273-88, 1982; the TK promoter of Herpes virus (McKnight, S, Cell 31:355-65, 1982); the SV40 early promoter (Benoist, C., et al., Nature 290:304-10, 1981); and the yeast gal4 gene promoter (Johnston, S A et al., Proc. Natl. Acad. Sci. USA 79:6971-5, 1982); Silver, P A, et al., Proc. Natl. Acad. Sci. (USA) 81:5951-5, 1984)). Other illustrative descriptions of transcriptional factor association with promoter regions and the separate activation and DNA binding of transcription factors include: Keegan et al., Nature 231:699, 1986; Fields et al., Nature 340:245, 1989; Jones, Cell 61:9, 1990; Lewin, Cell 61:1161, 1990; Ptashne et al., Nature 346:329, 1990; Adams et al., Cell 72:306, 1993.

The promoter region may further include an octamer region which may also function as a tissue specific enhancer, by interacting with certain proteins found in the specific tissue. The enhancer domain of the DNA construct of the present invention is one which is specific for the target cells to be transfected, or is highly activated by cellular factors of such target cells. Examples of vectors (plasmid or retrovirus) are disclosed, e.g., in Roy-Burman et al., U.S. Pat. No. 5,112,767. For a general discussion of enhancers and their actions in transcription, see, Lewin, B M, Genes IV, Oxford University Press pp. 552-576, 1990 (or later edition). Particularly useful are retroviral enhancers (e.g., viral LTR) that is preferably placed upstream from the promoter with which it interacts to stimulate gene expression. For use with retroviral vectors, the endogenous viral LTR may be rendered enhancer-less and substituted with other desired enhancer sequences which confer tissue specificity or other desirable properties such as transcriptional efficiency.

Thus, expression cassettes include plasmids, recombinant viruses, any form of a recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include replicons (e.g., RNA replicons), bacteriophages) to which fragments of DNA may be attached and become replicated. Vectors thus include, but are not limited to RNA, autonomous self-replicating circular or linear DNA or RNA, e.g., plasmids, viruses, and the like (U.S. Pat. No. 5,217,879), and includes both the expression and nonexpression plasmids. Where a recombinant cell or culture is described as hosting an “expression vector” this includes both extrachromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

Exemplary virus vectors that may be used include recombinant adenoviruses (Horowitz, M S, In: Virology, Fields, B N et al., eds, Raven Press, NY, 1990, p. 1679; Berkner, K L, Biotechniques 6:616-29, 1988; Strauss, S E, In: The Adenoviruses, Ginsberg, H S, ed., Plenum Press, NY, 1984, chapter 11) and herpes simplex virus (HSV). Advantages of adenovirus vectors for human gene delivery include the fact that recombination is rare, no human malignancies are known to be associated with such viruses, the adenovirus genome is double stranded DNA which can be manipulated to accept foreign genes of up to 7.5 kb in size, and live adenovirus is a safe human vaccine organisms. Adeno-associated virus is also useful for human therapy (Samulski, R J et al., EMBO J. 10:3941, 1991) according to the present invention.

Another vector which can express the DNA molecule of the present invention, and is useful in the present therapeutic setting is vaccinia virus, which can be rendered non-replicating (U.S. Pat. Nos. 5,225,336; 5,204,243; 5,155,020; 4,769,330; Fuerst, T R et al., Proc. Natl. Acad. Sci. USA 86:2549-53, 1992; Chakrabarti, S et al., Mol Cell Biol 5:3403-9, 1985). Descriptions of recombinant vaccinia viruses and other viruses containing heterologous DNA and their uses in immunization and DNA therapy are reviewed in: Moss, B, Curr Opin Genet Dev 3:86-90, 1993; Moss, B, Biotechnol. 20:345-62, 1992).

Other viral vectors that may be used include viral or non-viral vectors, including adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus).

A DNA vaccine may also use a replicon, e.g., an RNA replicon, a self-replicating RNA vector. A preferred replicon is one based on a Sindbis virus RNA replicon, e.g., SINrepS. The present inventors tested E7 in the context of such a vaccine and showed (see Wu et al, U.S. patent application Ser. No. 10/343,719) that a Sindbis virus RNA vaccine encoding HSV-1 VP22 linked to E7 significantly increased activation of E7-specific CD8 T cells, resulting in potent antitumor immunity against E7-expressing tumors. The Sindbis virus RNA replicon vector used in these studies, SlNrep5, has been described (Bredenbeek, P J et al., 1993, J. Virol. 67:6439-6446).

Generally, RNA replicon vaccines may be derived from alphavirus vectors, such as Sindbis virus (Hariharan, M J et al., 1998. J Virol 72:950-8.), Semliki Forest virus (Berglund, P M et al., 1997. AIDS Res Hum Retroviruses 13:1487-95; Ying, H T et al., 1999. Nat Med 5:823-7) or Venezuelan equine encephalitis virus (Pushko, P M et al., 1997. Virology 239:389-401). These self-replicating and self-limiting vaccines may be administered as either (1) RNA or (2) DNA which is then transcribed into RNA replicons in cells transfected in vitro or in vivo (Berglund, P C et al., 1998. Nat Biotechnol 16:562-5; Leitner, W W et al., 2000. Cancer Res 60:51-5). An exemplary Semliki Forest virus is pSCA1 (DiCiommo, D P et al., J Biol Chem 1998; 273:18060-6).

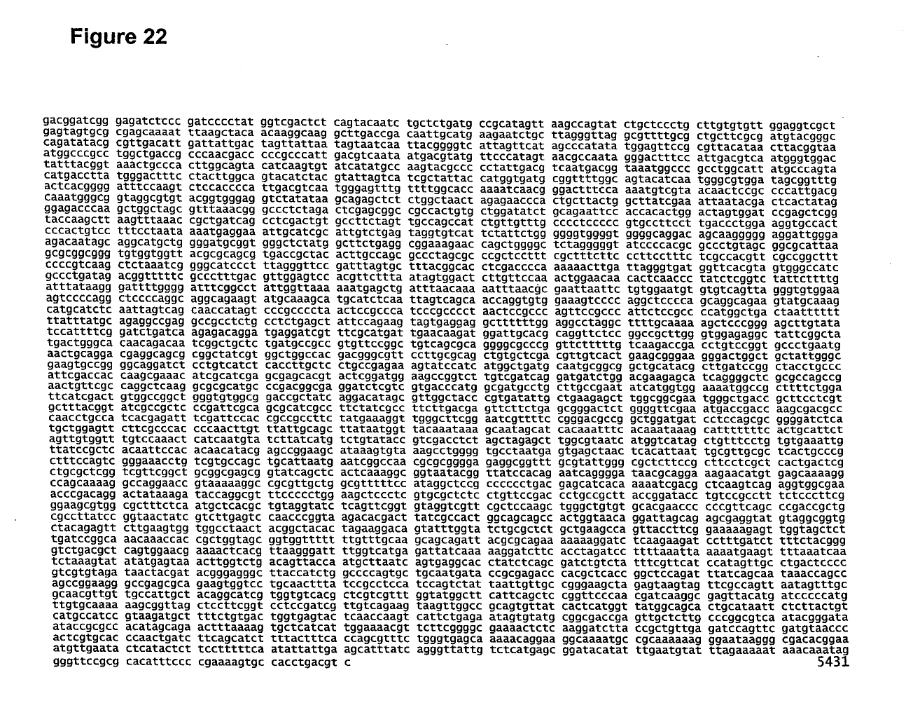

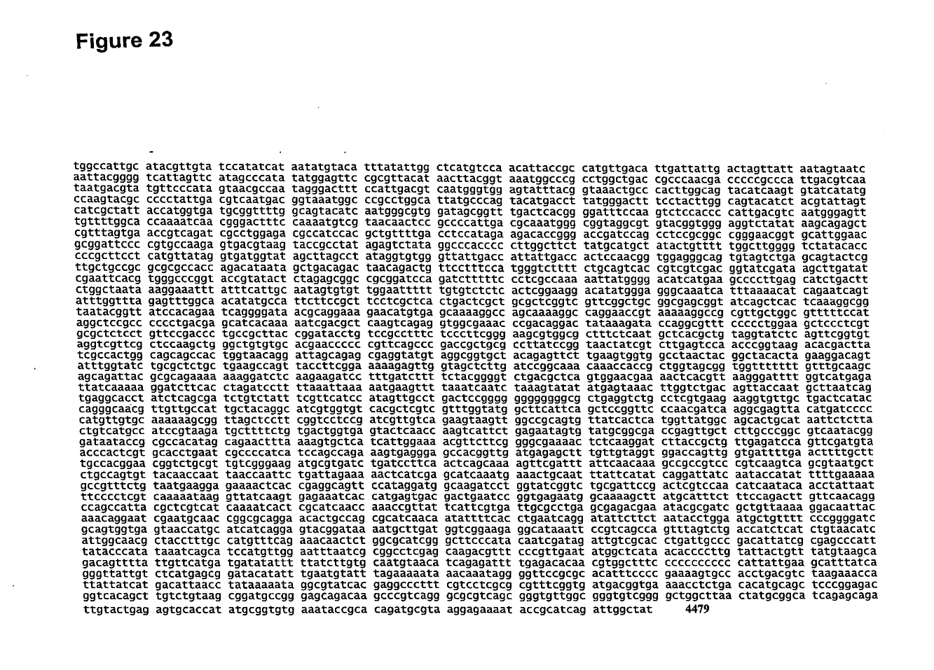

The plasmid vector pcDNA3 or a functional homolog thereof, which is shown in FIG. 22 (SEQ ID NO: 1) may be used in a DNA vaccine. In other embodiments, pNGVL4a, shown in FIG. 23 (SEQ ID NO: 2) is used.

pNGVL4a, one preferred plasmid backbone for the present invention was originally derived from the pNGVL3 vector, which has been approved for human vaccine trials. The pNGVL4a vector includes two immunostimulatory sequences (tandem repeats of CpG dinucleotides) in the noncoding region. Whereas any other plasmid DNA that can transform either APCs, preferably DC's or other cells which, via cross-priming, transfer the antigenic moiety to DCs, is useful in the present invention, pNGFVLA4a is preferred because of the fact that it has already been approved for human therapeutic use.

The following references set forth principles and current information in the field of basic, medical and veterinary virology and are incorporated by reference: Fields Virology, Fields, B N et al., eds., Lippincott Williams & Wilkins, N.Y., 1996; Principles of Virology: Molecular Biology, Pathogenesis, and Control, Flint, S. J. et al., eds., Amer Soc Microbiol, Washington D.C., 1999; Principles and Practice of Clinical Virology, 4th Edition, Zuckerman A. J. et al., eds, John Wiley & Sons, NY, 1999; The Hepatitis C Viruses, by Hagedorn, C H et al., eds., Springer Verlag, 1999; Hepatitis B Virus: Molecular Mechanisms in Disease and Novel Strategies for Therapy, Koshy, R. et al., eds, World Scientific Pub Co, 1998; Veterinary Virology, Murphy, F. A. et al., eds., Academic Press, NY, 1999; Avian Viruses: Function and Control, Ritchie, B. W., Iowa State University Press, Ames, 2000; Virus Taxonomy: Classification and Nomenclature of Viruses: Seventh Report of the International Committee on Taxonomy of Viruses, by M. H. V. Van Regenmortel, M H V et al., eds., Academic Press; NY, 2000.

In addition to naked DNA or viral vectors, engineered bacteria may be used as vectors. A number of bacterial strains including Salmonella, BCG and Listeria monocytogenes (LM) (Hoiseth et al., Nature 291:238-9, 1981; Poirier, T P et al., J Exp Med 168:25-32, 1988); Sadoff, J C et al., Science 240:336-8, 1988; Stover, C K et al., Nature 351:456-60, 1991; Aldovini, A et al., Nature 351:479-82, 1991). These organisms display two promising characteristics for use as vaccine vectors: (1) enteric routes of infection, providing the possibility of oral vaccine delivery; and (2) infection of monocytes/macrophages thereby targeting antigens to professional APCs.

In addition to virus-mediated gene transfer in vivo, physical means well-known in the art can be used for direct transfer of DNA, including administration of plasmid DNA (Wolff et al., 1990, supra) and particle-bombardment mediated gene transfer (Yang, N-S, et al., Proc Natl Acad Sci USA 87:9568, 1990; Williams, R S et al., Proc Natl Acad Sci USA 88:2726, 1991; Zelenin, A V et al., FEBS Lett 280:94, 1991; Zelenin, A V et al., FEBS Lett 244:65, 1989); Johnston, S A et al., In Vitro Cell Dev Biol 27:11, 1991). Furthermore, electroporation, a well-known means to transfer genes into cell in vitro, can be used to transfer DNA molecules according to the present invention to tissues in vivo (Titomirov, A V et al., Biochim Biophys Acta 1088:131, 1991).

“Carrier mediated gene transfer” has also been described (Wu, C H et al., J Biol Chem 264:16985, 1989; Wu, G Y et al., J Biol Chem 263:14621, 1988; Soriano, P et al., Proc Nat. Acad Sci USA 80:7128, 1983; Wang, C-Y et al., Pro. Natl Acad Sci USA 84:7851, 1982; Wilson, J M et al., J Biol Chem 267:963, 1992). Preferred carriers are targeted liposomes (Nicolau, C et al., Proc Natl Acad Sci USA 80:1068, 1983; Soriano et al., supra) such as immunoliposomes, which can incorporate acylated mAbs into the lipid bilayer (Wang et al., supra). Polycations such as asialoglycoprotein/polylysine (Wu et al., 1989, supra) may be used, where the conjugate includes a target tissue-recognizing molecule (e.g., asialo-orosomucoid for liver) and a DNA binding compound to bind to the DNA to be transfected without causing damage, such as polylysine. This conjugate is then complexed with plasmid DNA of the present invention.

Plasmid DNA used for transfection or microinjection may be prepared using methods well-known in the art, for example using the Quiagen procedure (Quiagen), followed by DNA purification using known methods, such as the methods exemplified herein.

Such expression vectors may be used to transfect host cells (in vitro, ex vivo or in vivo) for expression of the DNA and production of the encoded proteins which include fusion proteins or peptides. In one embodiment, a DNA vaccine is administered to or contacted with a cell, e.g., a cell obtained from a subject (e.g., an antigen presenting cell), and administered to a subject, wherein the subject is treated before, after or at the same time as the cells are administered to the subject.

The term “isolated” as used herein, when referring to a molecule or composition, such as a translocation polypeptide or a nucleic acid coding therefor, means that the molecule or composition is separated from at least one other compound (protein, other nucleic acid, etc.) or from other contaminants with which it is natively associated or becomes associated during processing. An isolated composition can also be substantially pure. An isolated composition can be in a homogeneous state and can be dry or in aqueous solution. Purity and homogeneity can be determined, for example, using analytical chemical techniques such as polyacrylamide gel electrophoresis (PAGE) or high performance liquid chromatography (HPLC). Even where a protein has been isolated so as to appear as a homogenous or dominant band in a gel pattern, there are trace contaminants which co-purify with it.

Host cells transformed or transfected to express the fusion polypeptide or a homologue or functional derivative thereof are within the scope of the invention. For example, the fusion polypeptide may be expressed in yeast, or mammalian cells such as Chinese hamster ovary cells (CHO) or, preferably human cells. Preferred cells for expression according to the present invention are APCs most preferably, DCs. Other suitable host cells are known to those skilled in the art.

Therapeutic Compositions and their Administration

A vaccine composition comprising a nucleic acid, a particle comprising the nucleic acid or a cell expressing this nucleic acid, is administered to a mammalian subject. The vaccine composition is administered in a pharmaceutically acceptable carrier in a biologically-effective and/or a therapeutically-effective amount.

Certain preferred conditions are disclosed in the Examples. The composition may be given alone or in combination with another protein or peptide such as an immunostimulatory molecule. Treatment may include administration of an adjuvant, used in its broadest sense to include any nonspecific immune stimulating compound such as an interferon. Adjuvants contemplated herein include resorcinols, non-ionic surfactants such as polyoxyethylene oleyl ether and n-hexadecyl polyethylene ether.

A therapeutically effective amount is a dosage that, when given for an effective period of time, achieves the desired immunological or clinical effect.

A therapeutically active amount of a nucleic acid encoding the fusion polypeptide may vary according to factors such as the disease state, age, sex, and weight of the individual, and the ability of the peptide to elicit a desired response in the individual. Dosage regimes may be adjusted to provide the optimum therapeutic response. For example, several divided doses may be administered daily or the dose may be proportionally reduced as indicated by the exigencies of the therapeutic situation. A therapeutically effective amounts of the protein, in cell associated form may be stated in terms of the protein or cell equivalents.

Thus an effective amount of the vaccine may be between about 1 nanogram and about 1 gram per kilogram of body weight of the recipient, more preferably between about 0.1 mg/kg and about 10 mg/kg, more preferably between about 1 mg/kg and about 1 mg/kg. Dosage forms suitable for internal administration preferably contain (for the latter dose range) from about 0.1 mg to 100 mg of active ingredient per unit. The active ingredient may vary from 0.5 to 95% by weight based on the total weight of the composition. Alternatively, an effective dose of cells transfected with the DNA vaccine constructs of the present invention is between about 10⁴ and 10⁸ cells. Those skilled in the art of immunotherapy will be able to adjust these doses without undue experimentation.

Preferred routes of administration of the DNA include (a) intradermal “gene gun” delivery wherein DNA-coated gold particles in an effective amount are delivered using a helium-driven gene gun (BioRad, Hercules, Calif.) with a discharge pressure set at a known level, e.g., of 400 p.s.i.; (b) intramuscularly (i.m.) injection using a conventional syringe needle; and (c) use of a needle-free biojector such as the Biojector 2000 (Bioject Inc., Portland, Oreg.) which is an injection device consisting of an injector and a disposable syringe. The orifice size controls the depth of penetration. For example, 50 mg of DNA may be delivered using the Biojector with no. 2 syringe nozzle.

Other routes of administration include the following. The term “systemic administration” refers to administration of a composition or agent such as a DNA vaccine as described herein, in a manner that results in the introduction of the composition into the subject's circulatory system or otherwise permits its spread throughout the body. “Regional” administration refers to administration into a specific, and somewhat more limited, anatomical space, such as intraperitoneal, intrathecal, subdural, or to a specific organ. “Local administration” refers to administration of a composition or drug into a limited, or circumscribed, anatomic space, such as intratumoral injection into a tumor mass, subcutaneous injections, intradermal or intramuscular injections. Those of skill in the art will understand that local administration or regional administration may also result in entry of a composition into the circulatory system—i.e., rendering it systemic to one degree or another. Other routes of administration include oral, intranasal or rectal or any other route known in the art.

For accomplishing the objectives of the present invention, nucleic acid therapy may be accomplished by direct transfer of a functionally active DNA into mammalian somatic tissue or organ in vivo. DNA transfer can be achieved using a number of approaches described below. These systems can be tested for successful expression in vitro by use of a selectable marker (e.g., G418 resistance) to select transfected clones expressing the DNA, followed by detection of the presence of the antigen-containing expression product (after treatment with the inducer in the case of an inducible system) using an antibody to the product in an appropriate immunoassay.

The DNA molecules, e.g., encoding a fusion polypeptides, may also be packaged into retrovirus vectors using packaging cell lines that produce replication-defective retroviruses, as is well-known in the art (e.g., Cone, R. D. et al., Proc Natl Acad Sci USA 81:6349-53, 1984; Mann, R F et al., Cell 33:153-9, 1983; Miller, A D et al., Molec Cell Biol 5:431-7, 1985; Sorge, J, et al., Molec Cell Biol 4:1730-7, 1984; Hock, R A et al., Nature 320:257, 1986; Miller, A D et al., Molec Cell Biol 6:2895-2902 (1986). Newer packaging cell lines which are efficient an safe for gene transfer have also been described (Bank et al., U.S. Pat. No. 5,278,056).

The above approach can be utilized in a site specific manner to deliver the retroviral vector to the tissue or organ of choice. Thus, for example, a catheter delivery system can be used (Nabel, E G et al., Science 244:1342 (1989)). Such methods, using either a retroviral vector or a liposome vector, are particularly useful to deliver the nucleic acid to be expressed to a blood vessel wall, or into the blood circulation of a tumor.

Depending on the route of administration, the composition may be coated in a material to protect the compound from the action of enzymes, acids and other natural conditions which may inactivate the compound. Thus it may be necessary to coat the composition with, or co-administer the composition with, a material to prevent its inactivation. For example, an enzyme inhibitors of nucleases or proteases (e.g., pancreatic trypsin inhibitor, diisopropylfluorophosphate and trasylol).or in an appropriate carrier such as liposomes (including water-in-oil-in-water emulsions as well as conventional liposomes (Strejan et al., J. Neuroimmunol 7:27, 1984).

Other pharmaceutically acceptable carriers for the nucleic acid vaccine compositions according to the present invention are liposomes, pharmaceutical compositions in which the active protein is contained either dispersed or variously present in corpuscles consisting of aqueous concentric layers adherent to lipidic layers. The active protein is preferably present in the aqueous layer and in the lipidic layer, inside or outside, or, in any event, in the non-homogeneous system generally known as a liposomic suspension. The hydrophobic layer, or lipidic layer, generally, but not exclusively, comprises phospholipids such as lecithin and sphingomyelin, steroids such as cholesterol, more or less ionic surface active substances such as dicetylphosphate, stearylamine or phosphatidic acid, and/or other materials of a hydrophobic nature. Those skilled in the art will appreciate other suitable embodiments of the present liposomal formulations.

A chemotherapeutic drug may be administered in doses that are similar to the doses that the chemotherapeutic drug is used to be administered for cancer therapy. Alternatively, it may be possible to use lower doses, e.g., doses that are lower by 10%, 30%, 50%, or 2, 5, or 10 fold lower. Generally, the dose of chemotherapeutic agent is a dose that is effective to increase the effectiveness of a DNA vaccine, but less than a dose that results in significant immunosuppression or immunosuppression that essentially cancels out the effect of the DNA vaccine.

The route of administration of chemotherapeutic drugs may depend on the drug. For use in the methods described herein, a chemotherapeutic drug may be used as it is commonly used in known methods. Generally, the drugs will be administered orally or they may be injected. The regimen of administration of the drugs may be the same as it is commonly used in known methods. For example, certain drugs are administered one time, other drugs are administered every third day for a set period of time, yet other drugs are administered every other day or every third, fourth, fifth, sixth day or weekly. The Examples provide examplary regimens for administrating the drugs, as well as DNA vaccines.

The DNA vaccine and the chemotherapeutic drug may be administered simultaneously or subsequently. In a preferred embodiment, a subject first receives one or more doses of chemotherapeutic drug and then one or more doses of DNA vaccine. In the case of DMXAA, it is preferable to administer to the subject a dose of DNA vaccine first and then a dose of chemotherapeutic drug.

One may administer 1, 2, 3, 4, 5 or more doses of DNA vaccine and 1, 2, 3, 4, 5 or more doses of chemotherapeutic agent. Exemplary regimes are provided in the examples.

A method may further comprise subjecting a subject to another cancer treatment, e.g., radiotherapy, an anti-angiogenesis agent and/or a hydrogel-based system.

As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

Preferred pharmaceutically acceptable diluents include saline and aqueous buffer solutions. Pharmaceutical compositions suitable for injection include sterile aqueous solutions (where water soluble) or dispersions and sterile powders for the extemporaneous preparation of sterile injectable solutions or dispersion. Isotonic agents, for example, sugars, polyalcohols such as mannitol, sorbitol, sodium chloride may be included in the pharmaceutical composition. In all cases, the composition should be sterile and should be fluid. It should be stable under the conditions of manufacture and storage and must include preservatives that prevent contamination with microorganisms such as bacteria and fungi. Dispersions can also be prepared in glycerol, liquid polyethylene glycols, and mixtures thereof and in oils. Under ordinary conditions of storage and use, these preparations may contain a preservative to prevent the growth of microorganisms.

The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms in the pharmaceutical composition can be achieved by various antibacterial and antifungal agents, for example, parabens, chlorobutanol, phenol, ascorbic acid, thimerosal, and the like.

Compositions are preferably formulated in dosage unit form for ease of administration and uniformity of dosage. Dosage unit form refers to physically discrete units suited as unitary dosages for a mammalian subject; each unit contains a predetermined quantity of active material (e.g., the nucleic acid vaccine) calculated to produce the desired therapeutic effect, in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active compound for the treatment of, and sensitivity of, individual subjects

For lung instillation, aerosolized solutions are used. In a sprayable aerosol preparations, the active protein may be in combination with a solid or liquid inert carrier material. This may also be packaged in a squeeze bottle or in admixture with a pressurized volatile, normally gaseous propellant. The aerosol preparations can contain solvents, buffers, surfactants, and antioxidants in addition to the protein of the invention.

Methods of administrating a chemotherapeutic drug and a vaccine may further comprise administration of one or more other constructs, e.g., to prolong the life of antigen presenting cells. Exemplary constructs are described in the following two sections. Such constructs may be administered simultaneously or at the same time as a DNA vaccine. Alternatively, they may be administered before or after administration of the DNA vaccine or chemotherapeutic drug.

Diseases that may be treated as described herein include hyperproliferative diseases, e.g., cancer, whether localized or having metastasized. Exemplary cancers include head and neck cancers and cervical cancer. Any cancer can be treated provided that there is a tumor associated antigen that is associated with the particular cancer. Other cancers include skin cancer, lung cancer, colon cancer, kidney cancer, breast cancer, prostate cancer, pancreatic cancer, bone cancer, brain cancer, as well as blood cancers, e.g., myeloma, leukemia and lymphoma. Generally, any cell growth can be treated provided that there is an antigen associated with the cell growth, which antigen or homolog thereof can be encoded by a DNA vaccine.

Treating a subject includes curing a subject or improving at least one symptom of the disease or preventing or reducing the likelihood of the disease to return. For example, treating a subject having cancer could be reducing the tumor mass of a subject, e.g., by about 10%, 30%, 50%, 75%, 90% or more, eliminating the tumor, preventing or reducing the likelihood of the tumor to return, or partial or complete remission.

Potentiation of Immune Responses Using siRNA Directed at Apoptotic Pathways

Administration to a subject of a DNA vaccine and a chemotherapeutic drug may accompanied by administration of one or more other agents, e.g., constructs. In one embodiment, a method comprises further administering to a subject an siRNA directed at an apoptotic pathway, such as described in WO 2006/073970, which is incorporated herein in its entirety.

The present inventors have previously designed siRNA sequences that hybridize to, and block expression of the activation of Bak and Bax proteins that are central players in the apoptosis signalling pathway. The present invention is also directed to the methods of treating tumors or hyperproliferative disease involving the administration of siRNA molecules (sequences), vectors containing or encoding the siRNA, expression vectors with a promoter operably linked to the siRNA coding sequence that drives transcription of siRNA sequences that are “specific” for sequences Bak and Bax nucleic acid. siRNAs may include single stranded “hairpin” sequences because of their stability and binding to the target mRNA.

Since Bak and Bax are involved, among other death proteins, in apoptosis of APCs, particularly DCs, the present siRNA sequences may be used in conjunction with a broad range of DNA vaccine constructs encoding antigens to enhance and promote the immune response induced by such DNA vaccine constructs, particularly CD8+ T cell mediated immune responses typified by CTL activation and action. This is believed to occur as a result of the effect of the siRNA in prolonging the life of antigen-presenting DCs which may otherwise be killed in the course of a developing immune response by the very same CTLs that the DCs are responsible for inducing.

In addition to Bak and Bax, additional targets for siRNAs designed in an analogous manner include caspase 8, caspase 9 and caspase 3. The present invention includes compositions and methods in which siRNAs targeting any two or more of Bak, Bax, caspase 8, caspase 9 and caspase 3 are used in combination, optionally simultaneously (along with a DNA immunogen that encodes an antigen), to administer to a subject. Such combinations of siRNAs may also be used to transfect DCs (along with antigen loading) to improve the immunogenicity of the DCs as cellular vaccines by rendering them resistant to apoptosis.

siRNAs suppress gene expression through a highly regulated enzyme-mediated process called RNA interference (RNAi) (Sharp, P. A., Genes Dev. 15:485-90, 2001; Bernstein, E et al., Nature 409:363-66, 2001; Nykanen, A et al., Cell 107:309-21, 2001; Elbashir et al., Genes Dev. 15:188-200, 2001). RNA interference is the sequence-specific degradation of homologues in an mRNA of a targeting sequence in an siNA. As used herein, the term siNA (small, or short, interfering nucleic acid) is meant to be equivalent to other terms used to describe nucleic acid molecules that are capable of mediating sequence specific RNAi (RNA interference), for example short (or small) interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), translational silencing, and others. RNAi involves multiple RNA-protein interactions characterized by four major steps: assembly of siRNA with the RNA-induced silencing complex (RISC), activation of the RISC, target recognition and target cleavage. These interactions may bias strand selection during siRNA-RISC assembly and activation, and contribute to the overall efficiency of RNAi (Khvorova, A et al., Cell 115:209-216 (2003); Schwarz, D S et al. 115:199-208 (2003)))

Considerations to be taken into account when designing an RNAi molecule include, among others, the sequence to be targeted, secondary structure of the RNA target and binding of RNA binding proteins. Methods of optimizing siRNA sequences will be evident to the skilled worker. Typical algorithms and methods are described in Vickers et al. (2003) J Biol Chem 278:7108-7118; Yang et al. (2003) Proc Natl Acad Sci USA 99:9942-9947; Far et al. (2003) Nuc. Acids Res. 31:4417-4424; and Reynolds et al. (2004) Nature Biotechnology 22:326-330, all of which are incorporated by reference in their entirety.

The methods described in Far et al., supra, and Reynolds et al., supra, may be used by those of ordinary skill in the art to select targeted sequences and design siRNA sequences that are effective at silencing the transcription of the relevant mRNA. Far et al. suggests options for assessing target accessibility for siRNA and supports the design of active siRNA constructs. This approach can be automated, adapted to high throughput and is open to include additional parameters relevant to the biological activity of siRNA. To identify siRNA-specific features likely to contribute to efficient processing at each of the steps of RNAi noted above. Reynolds et al., supra, present a systematic analysis of 180 siRNAs targeting the mRNA of two genes. Eight characteristics associated with siRNA functionality were identified: low G/C content, a bias towards low internal stability at the sense strand 3′-terminus, lack of inverted repeats, and sense strand base preferences (positions 3, 10, 13 and 19). Application of an algorithm incorporating all eight criteria significantly improves potent siRNA selection. This highlights the utility of rational design for selecting potent siRNAs that facilitate functional gene knockdown.

Candidate siRNA sequences against mouse and human Bax and Bak are selected using a process that involves running a BLAST search against the sequence of Bax or Bak (or any other target) and selecting sequences that “survive” to ensure that these sequences will not be cross matched with any other genes.

siRNA sequences selected according to such a process and algorithm may be cloned into an expression plasmid and tested for their activity in abrogating Bak/Bax function cells of the appropriate animal species. Those sequences that show RNAi activity may be used by direct administration bound to particles, or recloned into a viral vector such as a replication-defective human adenovirus serotype 5 (Ad5).

One advantage of this viral vector is the high titer obtainable (in the range of 10¹⁰) and therefore the high multiplicities-of infection that can be attained. For example, infection with 100 infectious units/cell ensures all cells are infected. Another advantage of this virus is the high susceptibility and infectivity and the host range (with respect to cell types). Even if expression is transient, cells would survive, possibly replicate, and continue to function before Bak/Bax activity would recover and lead to cell death. Preferred constructs include the following:

For Bak:

(SEQ ID NO: 42)

5′P-UGCCUACGAACUCUUCACCdTdT-3′ (sense)

(SEQ ID NO: 43)

5′P-GGUGAAGAGUUCGUAGGCAdTdT-3′ (antisense),

The nucleotide sequence encoding the Bak protein (including the stop codon) (GenBank accession No. NM_{—}007523 is shown below (SEQ ID NO: 44) with the targeted sequence in upper case, underscored.

| atggcatctggacaaggaccaggtcccccgaaggtgggctgcgatga | |
|---|---|
| gtccccgtccccttctgaacagcaggttgcccaggacacagaggag | |
| gtctttcgaagctacgttttttacctccaccagcaggaacaggagac | |
| ccaggggcggccgcctgccaaccccgagatggacaacttgcccctg | |
| gaacccaacagcatcttgggtcaggtgggtcggcagcttgctctca | |
| tcggagatgatattaaccggcgctacgacacagagttccagaattt | |
| actagaacagcttcagcccacagccgggaaTGCCTACGAACTCTT | |
| CACCaagatcgcctccagcctatttaagagtggcatcagctggggc | |
| cgcgtggtggctctcctgggctttggctaccgtctggccctgtacg | |
| tctaccagcgtggtttgaccggcttcctgggccaggtgacctgctt | |
| tttggctgatatcatactgcatcattacatcgccagatggatcgca | |
| cagagaggcggttgggtggcagccctgaatttgcgtagagacc | |
| ccatcctgaccgtaatggtgatttttggtgtggttctgttgggccaa | |
| ttcgtggtacacagattcttcagatcatga | 637 |

The targeted sequence of Bak, TGCCTACGAACTCTTCACC is SEQ ID NO: 45

For Bax:

(SEQ ID NO: 46)

5′P-UAUGGAGCUGCAGAGGAUGdTdT-3′ (sense)

(SEQ ID NO: 47)

s5′P-CAUCCUCUGCAGCUCCAUAdTdT-3′ (antisense)

The nucleotide sequence encoding Bax (including the stop codon) (GenBank accession No. L22472 is shown below (SEQ ID NO: 48) with the targeted sequence shown in upper case and underscored

| atggacgggtccggggagcagcttgggagcggcgggcccaccagct | |
|---|---|
| ctgaacagatcatgaagacaggggcctttttgctacagggtttcatc | |
| caggatcgagcagggaggatggctggggagacacctgagctgacctt | |
| ggagcagccgccccaggatgcgtccaccaagaagctgagcgagtgt | |
| ctccggcgaattggagatgaactggatagcaaTATGGAGCTGCAGA | |
| GGATGattgctgacgtggacacggactccccccgagaggtcttcttc | |
| cgggtggcagctgacatgtttgctgatggcaacttcaactggggccg | |
| cgtggttgccctcttctactttgctagcaaactggtgctcaaggcc | |
| ctgtgcactaaagtgcccgagctgatcagaaccatcatgggctgga | |
| cactggacttcctccgtgagcggctgcttgtctggatccaagaccag | |
| ggtggctgggaaggcctcctctcctacttcgggacccccacatggca | |
| gacagtgaccatctttgtggctggagtcctcaccgcctcgctcacc | |
| atctggaagaagatgggctga | 589 |

The targeted sequence of Bax, TATGGAGCTGCAGAGGATG is SEQ ID NO: 49

In a preferred embodiment, the inhibitory molecule is a double stranded nucleic acid (preferably an RNA), used in a method of RNA interference. The following show the “paired” 19 nucleotide structures of the siRNA sequences shown above, where the symbol [symbol] :

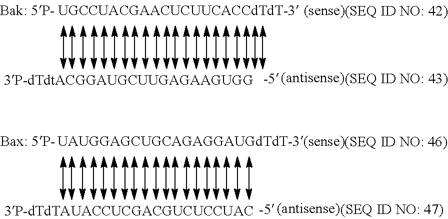

### Other Pro-Apoptotic Proteins to be Targeted

1. Caspase 8: The nucleotide sequence of human caspase-8 is shown below (SEQ ID NO: 50). GenBank Access. #NM_{—}001228. One target sequence for RNAi is underscored. Others may be identified using methods such as those described herein (and in reference cited herein, primarily Far et al., supra and Reynolds et al., supra).

| atg gac ttc agc aga aat ctt tat gat att ggg gaa caa ctg gac agt gaa gat ctg | |
|---|---|
| gcc tcc ctc aag ttc ctg agc ctg gac tac att ccg caa agg aag caa gaa ccc atc | |
| aag gat gcc ttg atg tta ttc cag aga ctc cag gaa aag aga atg ttg gag gaa agc | |
| aat ctg tcc ttc ctg aag gag ctg ctc ttc cga att aat aga ctg gat ttg ctg att | |
| acc tac cta aac act aga aag gag gag atg gaa agg gaa ctt cag aca cca ggc agg | |
| gct caa att tct gcc tac agg ttc cac ttc tgc cgc atg agc tgg gct gaa gca aac | |
| agc cag tgc cag aca cag tct gta cct ttc tgg cgg agg gtc gat cat cta tta ata | |
| agg gtc atg ctc tat cag att tca gaa gaa gtg agc aga tca gaa ttg agg tct ttt | |
| aag ttt ctt ttg caa gag gaa atc tcc aaa tgc aaa ctg gat gat gac atg aac ctg | |
| ctg gat att ttc ata gag atg gag aag agg gtc atc ctg gga gaa gga aag ttg gac | |
| atc ctg aaa aga gtc tgt gcc caa atc aac aag agc ctg ctg aag ata atc aac gac | |
| tat gaa gaa ttc agc aaa ggg gag gag ttg tgt ggg gta atg aca atc tcg gac tct | |
| cca aga gaa cag gat agt gaa tca cag act ttg gac aaa gtt tac caa atg aaa agc | |
| aaa cct cgg gga tac tgt ctg atc atc aac aat cac aat ttt gca aaa gca cgg gag | |
| aaa gtg ccc aaa ctt cac agc att agg gac agg aat gga aca cac ttg gat gca ggg | |
| gct ttg acc acg acc ttt gaa gag ctt cat ttt gag atc aag ccc cac gat gac tgc | |
| aca gta gag caa atc tat gag att ttg aaa atc tac caa ctc atg gac cac agt aac | |
| atg gac tgc ttc atc tgc tgt atc ctc tcc cat gga gac aag ggc atc atc tat ggc | |
| act gat gga cag gag gcc ccc atc tat gag ctg aca tct cag ttc act ggt ttg aag | |
| tgc cct tcc ctt gct gga aaa ccc aaa gtg ttt ttt att cag gct tgt cag ggg gat | |
| aac tac cag aaa ggt ata cct gtt gag act gat tca gag gag caa ccc tat tta gaa | |
| atg gat tta tca tca cct caa acg aga tat atc ccg gat gag gct gac ttt ctg ctg | |
| ggg atg gcc act gtg aat aac tgt gtt tcc tac cga aac cct gca gag gga acc tgg | |
| tac atc cag tca ctt tgc cag agc ctg aga gag cga tgt cct cga ggc gat gat att | |
| ctc acc atc ctg act gaa gtg aac tat gaa gta agc aac aag gat gac aag aaa aac | |
| atg ggg aaa cag atg cct cag cct act ttc aca cta aga aaa aaa ctt gtc ttc cct | |
| tct gat tga | 1491 |

The sequences of sense and antisense siRNA strands for targeting this sequence (including dTdT 3′ overhangs, are:

(SEQ ID NO: 51)

5′-AACCUCGGGGAUACUGUCUGAdTdT-3′ (sense)

(SEQ ID NO: 52)

5′-UCAGACAGUAUCCCCGAGGUUdTdT-3′ (antisense)

2. Caspase 9: The nucleotide sequence of human caspase-9 is shown below (SEQ ID NO: 53). See GenBank Access. #NM_{—}001229. The sequence below is of “variant α” which is longer than a second alternatively spliced variant β, which lacks the underscored part of the sequence shown below (and which is anti-apoptotic). Target sequences for RNAi, expected to fall in the underscored segment, are identified using known methods such as those described herein and in Far et al., supra and Reynolds et al., supra). and siNAs, such as siRNAs, are designed accordingly.

| atg gac gaa gcg gat cgg cgg ctc ctg cgg cgg tgc cgg ctg cgg ctg gtg gaa gag ctg | |
|---|---|
| cag gtg gac cag ctc tgg gac gcc ctg ctg agc cgc gag ctg ttc agg ccc cat atg atc | |
| gag gac atc cag cgg gca ggc tct gga tct cgg cgg gat cag gcc agg cag ctg atc ata | |
| gat ctg gag act cga ggg agt cag gct ctt cct ttg ttc atc tcc tgc tta gag gac aca | |
| ggc cag gac atg ctg gct tcg ttt ctg cga act aac agg caa gca gca aag ttg tcg aag | |
| cca acc cta gaa aac ctt acc cca gtg gtg ctc aga cca gag att cgc aaa cca gag gtt | |
| ctc aga ccg gaa aca ccc aga cca gtg gac att ggt tct gga gga ttt ggt gat gtc ggt | |
| gct ctt gag agt ttg agg gga aat gca gat ttg gct tac atc ctg agc atg gag ccc tgt | |
| ggc cac tgc ctc att atc aac aat gtg aac ttc tgc cgt gag tcc ggg ctc cgc acc cgc | |
| act ggc tcc aac atc gac tgt gag aag ttg cgg cgt cgc ttc tcc tcg ctg cat ttc atg | |
| gtg gag gtg aag ggc gac ctg act gcc aag aaa atg gtg ctg gct ttg ctg gag ctg gcg | |
| cag cag gac cac ggt gct ctg gac tgc tgc gtg gtg gtc att ctc tct cac ggc tgt cag | |
| gcc agc cac ctg cag ttc cca ggg gct gtc tac ggc aca gat gga tgc cct gtg tcg gtc | |
| gag aag att gtg aac atc ttc aat ggg acc agc tgc ccc agc ctg gga ggg aag ccc aag | |
| ctc ttt ttc atc cag gcc tgt ggt ggg gag cag aaa gac cat ggg ttt gag gtg gcc tcc | |
| act tcc cct gaa gac gag tcc cct ggc agt aac ccc gag cca gat gcc acc ccg ttc cag | |
| gaa ggt ttg agg acc ttc gac cag ctg gac gcc ata tct agt ttg ccc aca ccc agt gac | |
| atc ttt gtg tcc tac tct act ttc cca ggt ttt gtt tcc tgg agg gac ccc aag agt ggc | |
| tcc tgg tac gtt gag acc ctg gac gac atc ttt gag cag tgg gct cac tct gaa gac ctg | |
| cag tcc ctc ctg ctt agg gtc gct aat gct gtt tcg gtg aaa ggg att tat aaa cag atg | |
| cct ggt tgc ttt aat ttc ctc cgg aaa aaa ctt ttc ttt aaa aca tca taa | 1191 |

3. Caspase 3: The nucleotide sequence of human caspase-3 is shown below (SEQ ID NO: 54). See GenBank Access. #NM_{—}004346. The sequence below is of “variant α” which is the longer of two alternatively spliced variants, all of which encode the full protein. Target sequences for RNAi are identified using known methods such as those described herein and in Far et al., supra and Reynolds et al., supra) and siNAs, such as siRNAs, are designed accordingly.

| atg gag aac act gaa aac tca gtg gat tca aaa tcc att aaa aat ttg gaa cca aag atc | |
|---|---|
| ata cat gga agc gaa tca atg gac tct gga ata tcc ctg gac aac agt tat aaa atg gat | |
| tat cct gag atg ggt tta tgt ata ata att aat aat aag aat ttt cat aaa agc act gga | |
| atg aca tct cgg tct ggt aca gat gtc gat gca gca aac ctc agg gaa aca ttc aga aac | |
| ttg aaa tat gaa gtc agg aat aaa aat gat ctt aca cgt gaa gaa att gtg gaa ttg atg | |
| cgt gat gtt tct aaa gaa gat cac agc aaa agg agc agt ttt gtt tgt gtg ctt ctg agc | |
| cat ggt gaa gaa gga ata att ttt gga aca aat gga cct gtt gac ctg aaa aaa ata aca | |
| aac ttt ttc aga ggg gat cgt tgt aga agt cta act gga aaa ccc aaa ctt ttc att att | |
| cag gcc tgc cgt ggt aca gaa ctg gac tgt ggc att gag aca gac agt ggt gtt gat gat | |
| gac atg gcg tgt cat aaa ata cca gtg gag gcc gac ttc ttg tat gca tac tcc aca gca | |
| cct ggt tat tat tct tgg cga aat tca aag gat ggc tcc tgg ttc atc cag tcg ctt tgt | |
| gcc atg ctg aaa cag tat gcc gac aag ctt gaa ttt atg cac att ctt acc cgg gtt aac | |
| cga aag gtg gca aca gaa ttt gag tcc ttt tcc ttt gac gct act ttt cat gca aag aaa | |
| cag att cca tgt att gtt tcc atg ctc aca aaa gaa ctc tat ttt tat cac taa | 834 |

Long double stranded interfering RNAs, such a miRNAs, appear to tolerate mismatches more readily than do short double stranded RNAs. In addition, as used herein, the term RNAi is meant to be equivalent to other terms used to describe sequence specific RNA interference, such as post transcriptional gene silencing, or an epigenetic phenomenon. For example, siNA molecules of the invention can be used to epigenetically silence genes at both the post-transcriptional level or the pre-transcriptional level. In a non-limiting example, epigenetic regulation of gene expression by siNA molecules of the invention can result from siNA mediated modification of chromatin structure and thereby alter gene expression (see, for example, Allshire Science 297:1818-19, 2002; Volpe et al., Science 297:1833-37, 2002; Jenuwein, Science 297:2215-18, 2002; and Hall et al., Science 297, 2232-2237, 2002.)

An siNA can be designed to target any region of the coding or non-coding sequence of an mRNA. An siNA is a double-stranded polynucleotide molecule comprising self-complementary sense and antisense regions, wherein the antisense region comprises nucleotide sequence that is complementary to nucleotide sequence in a target nucleic acid molecule or a portion thereof and the sense region has a nucleotide sequence corresponding to the target nucleic acid sequence or a portion thereof. The siNA can be assembled from two separate oligonucleotides, where one strand is the sense strand and the other is the antisense strand, wherein the antisense and sense strands are self-complementary. The siNA can be assembled from a single oligonucleotide, where the self-complementary sense and antisense regions of the siNA are linked by means of a nucleic acid based or non-nucleic acid-based linker(s). The siNA can be a polynucleotide with a hairpin secondary structure, having self-complementary sense and antisense regions. The siNA can be a circular single-stranded polynucleotide having two or more loop structures and a stem comprising self-complementary sense and antisense regions, wherein the circular polynucleotide can be processed either in vivo or in vitro to generate an active siNA molecule capable of mediating RNAi. The siNA can also comprise a single stranded polynucleotide having nucleotide sequence complementary to nucleotide sequence in a target nucleic acid molecule or a portion thereof (or can be an siNA molecule that does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al. (2002) Cell 110, 563-574 and Schwarz et al. (2002) Molecular Cell 10, 537-568), or 5′,3′-diphosphate.

In certain embodiments, the siNA molecule of the invention comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, Van der Waal's interactions, hydrophobic interactions, and/or stacking interactions. Some preferred siRNAs are discussed above and in the Examples.

As used herein, siNA molecules need not be limited to those molecules containing only ribonucleotides but may also further encompass deoxyribonucleotides (as in the preferred siRNAs which each include a dTdT dinucleotide) chemically-modified nucleotides, and non-nucleotides. In certain embodiments, the siNA molecules of the invention lack 2′-hydroxy (2′-OH) containing nucleotides. In certain embodiments, siNAs do not require the presence of nucleotides having a 2′-hydroxy group for mediating RNAi and as such, siNAs of the invention optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions. If modified, the siNAs of the invention can also be referred to as “short interfering modified oligonucleotides” or “siMON.” Other chemical modifications, e.g., as described in Int'l Patent Publications WO 03/070918 and WO 03/074654, can be applied to any siNA sequence of the invention.

Preferably a molecule mediating RNAi has a 2 nucleotide 3′ overhang (dTdT in the preferred sequences disclosed herein). If the RNAi molecule is expressed in a cell from a construct, for example from a hairpin molecule or from an inverted repeat of the desired sequence, then the endogenous cellular machinery will create the overhangs.

Methods of making siRNAs are conventional. In vitro methods include processing the polyribonucleotide sequence in a cell-free system (e.g., digesting long dsRNAs with RNAse III or Dicer), transcribing recombinant double stranded DNA in vitro, and, preferably, chemical synthesis of nucleotide sequences homologous to Bak or Bax sequences. See, e.g., Tuschl et al., Genes & Dev. 13:3191-3197, 1999. In vivo methods include
(1) transfecting DNA vectors into a cell such that a substrate is converted into siRNA in vivo. See, for example, Kawasaki et al., Nucleic Acids Res 31:700-07, 2003; Miyagishi et al., Nature Biotechnol 20:497-500, 2003; Lee et al., Nature Biotechnol 20:500-05, 2002; Brummelkamp et al., Science 296:550-53, 2002; McManus et al., RNA 8:842-50, 2002; Paddison et al., Genes Dev 16:948-58, 2002; Paddison et al., Proc Natl Acad Sci USA 99:1443-48, 2002; Paul et al., Nature Biotechnol 20:505-08, 2002; Sui et al., Proc Natl Acad Sci USA 99:5515-20, 2002; Yu et al., Proc Natl Acad Sci USA 99:6047-52, 2002)
(2) expressing short hairpin RNAs from plasmid systems using RNA polymerase III (pol III) promoters. See, for example, Kawasaki et al., supra; Miyagishi et al., supra; Lee et al., supra; Brummelkamp et al., supra; McManus et al., supra), Paddison et al., supra (both); Paul et al., supra, Sui et al., supra; and Yu et al., supra; and/or
(3) expressing short RNA from tandem promoters. See, for example, Miyagishi et al., supra; Lee et al., supra).

When synthesized in vitro, a typical micromolar scale RNA synthesis provides about 1 mg of siRNA, which is sufficient for about 1000 transfection experiments using a 24-well tissue culture plate format. In general, to inhibit Bak or Bax expression in cells in culture, one or more siRNAs can be added to cells in culture media, typically at about 1 ng/ml to about 10 μg siRNA/ml.

For reviews and more general description of inhibitory RNAs, see Lau et al., Sci Amer August 2003: 34-41; McManus et al., Nature Rev Genetics 3, 737-47, 2002; and Dykxhoorn et al., Nature Rev Mol Cell Bio 4:457-467, 2003. For further guidance regarding methods of designing and preparing siRNAs, testing them for efficacy, and using them in methods of RNA interference (both in vitro and in vivo), see, e.g., Allshire, Science 297:1818-19, 2002; Volpe et al., Science 297:1833-37, 2002; Jenuwein, Science 297:2215-18, 2002; Hall et al., Science 297 2232-37, 2002; Hutvagner et al., Science 297:2056-60, 2002; McManus et al. RNA 8:842-850, 2002; Reinhart et al., Genes Dev. 16:1616-26, 2002; Reinhart et al., Science 297:1831, 2002; Fire et al. (1998) Nature 391:806-11, 2002; Moss, Curr Biol 11:R772-5, 2002:Brummelkamp et al., supra; Bass, Nature 411 428-9, 2001; Elbashir et al., Nature 411:494-8; U.S. Pat. No. 6,506,559; Published US Pat App. 20030206887; and PCT applications WO99/07409, WO99/32619, WO 00/01846, WO 00/44914, WO00/44895, WO01/29058, WO01/36646, WO01/75164, WO01/92513, WO 01/29058, WO01/89304, WO01/90401, WO02/16620, and WO02/29858.

Ribozymes and siNAs can take any of the forms, including modified versions, described for antisense nucleic acid molecules; and they can be introduced into cells as oligonucleotides (single or double stranded), or in the form of an expression vector.

In a preferred embodiment, an antisense nucleic acid, siNA (e.g., siRNA) or ribozyme comprises a single stranded polynucleotide comprising a sequence that is at least about 90% (e.g., at least about 93%, 95%, 97%, 98% or 99%) identical to a target segment (such as those indicted for Bak and Bax above) or a complement thereof. As used herein, a DNA and an RNA encoded by it are said to contain the same “sequence,” taking into account that the thymine bases in DNA are replaced by uracil bases in RNA.

Active variants (e.g., length variants, including fragments; and sequence variants) of the nucleic acid-based inhibitors discussed herein are also within the scope of the invention. An “active” variant is one that retains an activity of the inhibitor from which it is derived (preferably the ability to inhibit expression). It is routine to test a variant to determine for its activity using conventional procedures.

As for length variants, an antisense nucleic acid or siRNA may be of any length that is effective for inhibition of a gene of interest. Typically, an antisense nucleic acid is between about 6 and about 50 nucleotides (e.g., at least about 12, 15, 20, 25, 30, 35, 40, 45 or 50 nt), and may be as long as about 100 to about 200 nucleotides or more. Antisense nucleic acids having about the same length as the gene or coding sequence to be inhibited may be used. When referring to length, the terms bases and base pairs (bp) are used interchangeably, and will be understood to correspond to single stranded (ss) and double stranded (ds) nucleic acids. The length of an effective siNA is generally between about 15 by and about 29 by in length, preferably between about 19 and about 29 by (e.g., about 15, 17, 19, 21, 23, 25, 27 or 29 bp), with shorter and longer sequences being acceptable. Generally, siNAs are shorter than about 30 bases to prevent eliciting interferon effects. For example, an active variant of an siRNA having, for one of its strands, the 19 nucleotide sequence of any of SEQ ID NOs: 42, 43, 46, and 47 herein can lack base pairs from either, or both, of ends of the dsRNA; or can comprise additional base pairs at either, or both, ends of the ds RNA, provided that the total of length of the siRNA is between about 19 and about 29 bp, inclusive. One embodiment of the invention is an siRNA that “consists essentially of” sequences represented by SEQ ID NOs: 42, 43, 46, and 47 or complements of these sequence. The term “consists essentially of” is an intermediate transitional phrase, and in this case excludes, for example, sequences that are long enough to induce a significant interferon response. An siRNA of the invention may consist essentially of between about 19 and about 29 by in length.

As for sequence variants, it is generally preferred that an inhibitory nucleic acid, whether an antisense molecule, a ribozyme (the recognition sequences), or an siNA, comprise a strand that is complementary (100% identical in sequence) to a sequence of a gene that it is designed to inhibit. However, 100% sequence identity is not required to practice the present invention. Thus, the invention has the advantage of being able to tolerate naturally occurring sequence variations, for example, in human c-met, that might be expected due to genetic mutation, polymorphism, or evolutionary divergence. Alternatively, the variant sequences may be artificially generated. Nucleic acid sequences with small insertions, deletions, or single point mutations relative to the target sequence can be effective inhibitors.

The degree of sequence identity may be optimized by sequence comparison and alignment algorithms well-known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). At least about 90% sequence identity is preferred (e.g., at least about 92%, 95%, 98% or 99%), or even 100% sequence identity, between the inhibitory nucleic acid and the targeted sequence of targeted gene.

Alternatively, an active variant of an inhibitory nucleic acid of the invention is one that hybridizes to the sequence it is intended to inhibit under conditions of high stringency. For example, the duplex region of an siRNA may be defined functionally as a nucleotide sequence that is capable of hybridizing with a portion of the target gene transcript under high stringency conditions (e.g., 400 mM NaCl, 40 mM PIPES pH 6.4, 1 mM EDTA, 50° C. or 70° C., hybridization for 12-16 hours), followed generally by washing.

DC-1 cells or BM-DCs presenting a given antigen X, when not treated with the siRNAs of the invention, respond to sufficient numbers X-specific CD8+ CTL by apoptotic cell death. In contrast, the same cells transfected with the siRNA or infected with a viral vector encoding the present siRNA sequences survive better despite the delivery of killing signals.

Delivery and expression of the siRNA compositions of the present invention inhibit the death of DCs in vivo in the process of a developing T cell response, and thereby promote and stimulate the generation of an immune response induced by immunization with an antigen-encoding DNA vaccine vector. These capabilities have been exemplified by showing that:
(1) co-administration of DNA vaccines encoding HPV-16 E7 with siRNA targeted to Bak and Bax prolongs the lives of antigen-presenting DCs in the draining lymph nodes, thereby enhancing antigen-specific CD8⁺ T cell responses, and eliciting potent antitumor effects against an E7-expressing tumor in vaccinated subjects.
(2) DCs transfected with siRNA targeting Bak and Bax resist killing by T cells in vivo. E7-loaded DCs transfected with Bak/Bax siRNA so that Bak and Bax protein expression is down-regulated resist apoptotic death induced by T cells in vivo. When administered to subjects, these DCs generate stronger antigen-specific immune responses and manifest therapeutic effects (compared to DCs transfected with control siRNA).
   Thus, siRNA constructs are useful as a part of the nucleic acid vaccination and chemotherapy regimen described in this application.

### Potentiation of Immune Responses Using Anti-Apoptotic Proteins

Administration to a subject of a DNA vaccine and a chemotherapeutic drug may also be accompanied by administration of a nucleic acid encoding an anti-apoptotic protein, as described in WO2005/047501 and in U.S. Patent Application Publication No. 20070026076.

The present inventors have previously designed and disclosed an immunotherapeutic strategy that combines antigen-encoding DNA vaccine compositions with additional DNA vectors comprising anti-apoptotic genes including bcl-2, bc-1xL, XIAP, dominant negative mutants of caspase-8 and caspase-9, the products of which are known to inhibit apoptosis (Wu, et al. U.S. Patent Application Publication No. 20070026076). Serine protease inhibitor 6 (SPI-6) which inhibits granzyme B, may also be employed in compositions and methods to delay apoptotic cell death of DCs. The present inventors have shown that the harnessing of an additional biological mechanism, that of inhibiting apoptosis, significantly enhances T cell responses to DNA vaccines comprising antigen-coding sequences, as well as linked sequences encoding such IPPs.

Intradermal vaccination by gene gun efficiently delivers a DNA vaccine into DCs of the skin, resulting in the activation and priming of antigen-specific T cells in vivo. DCs, however, have a limited life span, hindering their long-term ability to prime antigen-specific T cells. According to the present invention, a strategy that combines combination therapy with methods to prolong the survival of DNA-transduced DCs enhances priming of antigen-specific T cells and thereby, increase DNA vaccine potency. Co-delivery of DNA encoding inhibitors of apoptosis (BCL-xL, BCL-2, XIAP, dominant negative caspase-9, or dominant negative caspase-8) with DNA encoding an antigen (exemplified as HPV-16 E7 protein) prolongs the survival of transduced DCs. More importantly, vaccinated subjects exhibited significant enhancement in antigen-specific CD8+ T cell immune responses, resulting in a potent antitumor effect against antigen-expressing tumors. Among these anti-apoptotic factors, BCL-XL demonstrated the greatest enhancement of both antigen-specific immune responses and antitumor effects. Thus, co-administration of a combination therapy including a DNA vaccine with one or more DNA constructs encoding anti-apoptotic proteins provides a way to enhance DNA vaccine potency.

Serine protease inhibitor 6 (SPI-6), also called Serpinb9, inhibits granzyme B, and may thereby delay apoptotic cell death in DCs. Intradermal co-administration of DNA encoding SPI-6 with DNA constructs encoding E7 linked to various IPPs significantly increased E7-specific CD8+ T cell and CD4+ Th1 cell responses and enhanced anti-tumor effects when compared to vaccination without SPI-6. Thus it is preferred to combine methods that enhance MHC class I and II antigen processing with delivery of SPI-6 to potentiate immunity

A similar approach employs DNA-based alphaviral RNA replicon vectors, also called suicidal DNA vectors. To enhance the immune response to an antigen, e.g., HPV E7, a DNA-based Semliki Forest virus vector, pSCA1, the antigen DNA is fused with DNA encoding an anti-apoptotic polypeptide such BCL-xL, a member of the BCL-2 family. pSCA1 encoding a fusion protein of an antigen polypeptide and/BCL-xL delays cell death in transfected DCs and generates significantly higher antigen-specific CD8+ T-cell-mediated immunity. The antiapoptotic function of BCL-xL is important for the enhancement of antigen-specific CD8+ T-cell responses. Thus, in one embodiment, delaying cell death induced by an otherwise desirable suicidal DNA vaccine enhances its potency.

Thus, the present invention is also directed to combination therapies including administering a chemotherapeutic drug with a nucleic acid composition useful as an immunogen, comprising a combination of: (a) first nucleic acid vector comprising a first sequence encoding an antigenic polypeptide or peptide, which first vector optionally comprises a second sequence linked to the first sequence, which second sequence encodes an immunogenicity-potentiating polypeptide (IPP); b) a second nucleic acid vector encoding an anti-apoptotic polypeptide, wherein, when the second vector is administered with the first vector to a subject, a T cell-mediated immune response to the antigenic polypeptide or peptide is induced that is greater in magnitude and/or duration than an immune response induced by administration of the first vector alone. The first vector above may comprises a promoter operatively linked the first and/or the second sequence.

In the above compositions the anti-apoptotic polypeptide is preferably selected from the group consisting of (a) BCL-xL, (b) BCL2, (c) XIAP, (d) FLICEc-s, (e) dominant-negative caspase-8, (f) dominant negative caspase-9, (g) SPI-6, and (h) a functional homologue or derivative of any of (a)-(g). The anti-apoptotic DNA may be physically linked to the antigen-encoding DNA. Examples of this are provided in U.S. Patent Application publication No. 20070026076, primarily in the form of suicidal DNA vaccine vectors. Alternatively, the anti-apoptotic DNA may be administered separately from, but in combination with the antigen-endcoding DNA molecule. Even more examples of the co-administration of these two types of vectors are provided in in U.S. patent application Ser. No. 10/546,810.

Exemplary nucleotide and amino acid sequences of anti-apoptotic and other proteins are provided in the sequence listing. Biologically active homologs of these proteins and constructs may also be used. Biologically active homologs is to be understood as described herein in the context of other proteins, e.g., IPPs.

The coding sequence for BCL-xL as present in the pcDNA3 vector of the present invention is SEQ ID NO:55; the amino acid sequence of BCL-xL is SEQ ID NO:56; the sequence pcDNA3-BCL-xL is SEQ ID NO:57 (the BCL-xL coding sequence corresponds to nucleotides 983 to 1732); a pcDNA3 vector combining E7 and BCL-xL, designated pcDNA3-E7/BCL-xL is SEQ ID NO:58 (the Eland BCL-xL sequences correspond to nucleotides 960 to 2009); the amino acid sequence of the E7-BCL-xL chimeric or fusion polypeptide is SEQ ID NO: 59; a mutant BCL-xL (“mtBCL-xL”) DNA sequence is SEQ ID NO:60; the amino acid sequence of mtBCL-xL is SEQ ID NO:61; the amino acid sequence of the E7-mtBCL-xL chimeric or fusion polypeptide is SEQ ID NO:62; in the pcDNA-mtBCL-xL [SEQ ID NO:63] vector, this mutant sequence is inserted in the same position that BCL-xL is inserted in SEQ ID NO:57 and in the pcDNA-E7/mtBCL-XL [SEQ ID NO:64], this sequence is inserted in the same position as the BCL-xL sequence is in SEQ ID NO:58; the sequence of the suicidal DNA vector pSCA1-BCL-xL is SEQ ID NO:65 (the BCL-xL sequence corresponds to nucleotides 7483 to 8232); the sequence of the “combined” vector, pSCA1-E7/BCL-xL is SEQ ID NO:66 (the sequence of E7 and BCL-xL corresponds to nucleotides 7461 to 8510); the sequence of pSCA1-mtBCL-xL [SEQ ID NO:67] is the same as that for the wild type BCL-xL except that the mtBCL-xL sequence is inserted in the same position as the wild type sequence in the pSCA1-mtBCL-xL vector; the sequence pSCA1-E7/mtBCL-xL [SEQ ID NO:68] is the same as that for the wild type pSCA1-E7/BCL-xL above, except that the mtBCL-xL sequence is inserted in the same position as the wild type sequence; the sequence of the vector pSG5-BCL-xL is SEQ ID NO:69 (the BCL-xL coding sequence corresponds to nucleotides 1061 to 1810); the sequenced of the vector pSG5-mtBCL-xL is SEQ ID NO:70 with the mutant BCL-xL sequence has the mtBCL-xL, shown above, inserted in the same location as for the wild type vector immediately above; the nucleotide sequence of the DNA encoding the XIAP anti-apoptotic protein is SEQ ID NO:71; the amino acid of the vector comprising the XIAP anti-apoptotic protein coding sequence is SEQ ID NO:72; the nucleotide sequence of the vector comprising the XIAP anti-apoptotic protein coding sequence, designated PSG5-XIAP is shown in SEQ ID NO:73 (with the XIAP corresponding to nucleotides 1055 to 2553); the sequence of DNA encoding the anti-apoptotic protein FLICEc-s is SEQ ID NO:74; the amino acid sequence of the anti-apoptotic protein FLICEc-s is SEQ ID NO:75; the PSG5 vector encoding the anti-apoptotic protein FLICEc-s, designated PSG5-FLICEc-s, has the sequence SEQ ID NO:76 (with the FLICEc-s sequence corresponding to nucleotides 1049 to 2443); the sequence of DNA encoding the anti-apoptotic protein Bcl2 is SEQ ID NO:77; the amino acid sequence of Bcl2 is SEQ ID NO:78; the PSG5 vector encoding Bcl2, designated PSG5-BCL2, has the sequence SEQ ID NO:79 (with the Bcl2 sequence corresponding to nucleotides 1061 to 1678); the pSG5-dn-caspase-8 vector is SEQ ID NO:80 (encoding the dominant-negative caspase-8 corresponding to nucleotides 1055 to 2449); the amino acid sequence of dn-caspase-8 is SEQ ID NO:81; the pSG5-dn-caspase-9 vector is SEQ ID NO:82 (encoding the dominant-negative caspase-9 as nucleotides 1055 to 2305); the amino acid sequence of dn-caspase-9 is SEQ ID NO:83); the nucleotide sequence of murine serine protease inhibitor 6 (SPI-6, deposited in GENEBANK as NM 009256) is SEQ ID NO:84; the amino acid sequence of the SPI-6 protein is SEQ ID NO:85; the nucleic acid sequence of the mutant SPI-6 (mtSPI6) is SEQ ID NO:86; the amino acid sequence of the mutant SPI-6 protein (mtSPI-6) is SEQ ID NO:87; the sequence of the pcDNA3-Spi6 vector is SEQ ID NO:88 (the SPI-6 sequence correponds to nucleotides 960 to 2081); and the sequence of the mutant vector pcDNA3-mtSpi6 vector [SEQ ID NO:89] is the same as that above, except that the mtSPI-6 sequence is inserted in the same location in place of the wild type SPI-6.

Biologically active homologs of these nucleic acids and proteins may be used. Biologically active homologs are to be understood as described in the context of other proteins, e.g., IPPs, herein. For example, a vector may encode an anti-apoptotic protein that is at least about 90%, 95%, 98% or 99% identical to that of a sequence set forth herein.

Also provided herein are compositions and kits comprising one or more DNA vaccines and one or more chemotherapeutic drugs, and optionally one or more other constructs described herein.

The present description is further illustrated by the following examples, which should not be construed as limiting in any way.

### EXAMPLES

### Example 1

### Epigallocatechin-3-Gallate Enhanhances CD8+ T Cell-Mediated Antitumor Immunity Induced by DNA Vaccination

### Abstract

Immunotherapy and chemotherapy are generally effective against small tumors in animal models of cancer. However, these treatment regimens are generally ineffective against large, bulky tumors. We have found that a multi-modality treatment regimen using DNA vaccination in combination with a chemotherapeutic agent, epigallocatechin-3-Gallate (EGCG), a compound found in green tea, is effective in inhibiting large tumor growth. EGCG was found to induce tumor cellular apoptosis in a dose-dependent manner. The combination of EGCG and DNA vaccination led to an enhanced tumor-specific T cell immune response as well as enhanced antitumor effects, resulting in a higher cure rate than either immunotherapy or EGCG alone. In addition, combined DNA vaccination and oral EGCG treatment provided long-term antitumor protection in cured mice. Cured animals rejected a challenge of E7-expressing tumors, such as TC-1 and B16E7, but not a challenge of B16 seven weeks after the combined treatment, demonstrating antigen specific immune responses. These results suggest that multi-modality treatment strategies such as combining immunotherapy with a tumor-killing cancer drug may be a more effective anti-cancer strategy than single modality treatments.

### Introduction

Multi-modality treatments which combine conventional cancer therapies with immunotherapy such as DNA vaccines have emerged as a potentially plausible approach in the fight against cancer (for reviews see (1, 2)). The present inventors have shown that the a multi-modality treatment regimen using DNA vaccination in combination with the chemotherapeutic agent EGCG is effective in inhibiting large tumor growth. The combination of EGCG and DNA vaccination led to an enhanced tumor-specific T cell immune response as well as enhanced antitumor effects, resulting in a higher cure rate than either immunotherapy or EGCG alone. In addition, combined DNA vaccination and oral EGCG treatment provided long-term antitumor protection in cured mice. Cured animals rejected a challenge of E7-expressing tumors, such as TC-1 and B16E7, but not a challenge of B16 seven weeks after the combined treatment, demonstrating antigen specific immune responses. This is shown in the Example below, as well as in other publications by the inventors (e.g., Wu et al., Cancer Res 2007, 67:802-811).

### Materials and Methods

Mice. Six- to eight-week-old female C57BL/6 mice were purchased from Daehan Biolink (Chungbuk, Korea). All animal procedures were performed according to approved protocols and in accordance with recommendations for the proper use and care of laboratory animals.
Tumor models. Three cell lines of H-2^{b} background, TC-1, B16 and B16E7, were used as murine tumor models. The HPV-16 E7-expressing murine tumor model, TC-1, has been described previously (29). In brief, HPV-16 E6, E7 and ras oncogene were used to transform primary C57BL/6 mice lung epithelial cells to generate the TC-1 cell line. The generation of a B16 melanoma cell line expressing HPV-16 E7 antigen, referred to as B16E7, has been previously described (30, 31). These cell lines were cultured in vitro in RPMI 1640 supplemented with 10% fetal bovine serum, 50 units/ml penicillin/streptomycin, 2 mM L-glutamine, 1 mM sodium pyruvate, and 2 mM nonessential amino acids, and grown at 37° with 5% CO₂.

### DNA Vaccination.

The generation and purification of pcDNA3-Sig/E7/LAMP-1 has been described previously (10). DNA-coated gold particles were prepared according to a previously described protocol (32). DNA-coated gold particles were delivered to the shaved abdominal region of mice using a helium-driven gene gun (BioRad, Hercules, Calif.) with a discharge pressure of 400 p.s.i. C57BL/6 mice were immunized with 2 μg of a plasmid encoding Sig/E7/LAMP-1 or a control plasmid with no insert. The mice received a booster with the same dose 7 days later.

Determination of apoptotic cells in tumors. C57BL/6 mice (five per group) were injected subcutaneously in the right leg with 5×10⁵ TC-1 tumor cells/mouse. Ten days later, EGCG (Sigma Chemical Co.) was administered in the drinking water at a concentration of 0, 0.1, 0.5 or 2.5 mg/ml for five days. After emulsifying the isolated tumors into single cell preparations, detection of apoptotic cells was performed using PE-conjugated Rabbit Anti-Active Caspase-3 Antibody (BD Bioscience, San Diego, Calif.) according to the manufacturer's instructions. To characterize the expression of HPV-16 E7 in TC-1 cells, single cell suspensions of isolated tumors were stained with E7-specific monoclonal antibody which was kindly provided by Dr. Ju-Hong Jeon (Seoul National University College of Medicine; ref (33)). The percent of apoptotic cells was analyzed using flow cytometry.
Activation of an E7-specific CD8⁺ T cell line by CD11c⁺-enriched cells from vaccinated mice. Ten days after tumor inoculation, tumor bearing mice were administered with EGCG in their drinking water at a concentration of 0 or 0.5 mg/ml for five days. Inguinal lymph nodes were then harvested from treated mice, and CD11c⁺ cells were enriched from a single cell suspension of isolated inguinal lymph nodes using CD11c (N418) microbeads (Miltenyi Biotec, Auburn, Calif.). Enriched CD11c⁺ cells were analyzed by forward and side scatter and gated around a population of cells with size and granular characteristics of dendritic cells (DCs). The isolated CD11c⁺DCs (2×10⁴) were incubated with 2×10⁶ E7-specific CD8⁺ T cells for 16 hours. Cells were then stained for both surface CD8 and intracellular IFN-γ and analyzed by flow-cytometry (10).
Intracellular cytokine staining and flow cytometry analysis. Splenocytes were harvested from the Sig/E7/LAMP-1 DNA and/or EGCG treated mice (five per group) seven days after the last vaccination. Prior to intracellular cytokine staining, 4×10⁶ pooled splenocytes from each vaccination group were incubated overnight with 1 μg/ml of E7 peptide containing either an MHC class I epitope (aa 49-57) for detecting E7-specific CD8⁺ T cell precursors, or 5 μg/ml of E7 peptide containing an MHC class II epitope (aa 30-67) for detecting E7-specific CD4⁺ T cell precursors (9). Intracellular IL-4 and IFN-γ staining and flow cytometric analysis were performed as described previously (32). Analyses were performed on a Becton-Dickinson FACScan with CELLQuest software (Becton Dickinson Immunocytometry System, Mountain View, Calif.).
In vivo tumor growth experiments. In vivo tumor growth experiments were performed in tumor challenged mice treated with EGCG at various concentrations. C57BL/6 mice (five per group) were injected subcutaneously in the right leg with 5×10⁵ TC-1 tumor cells/mouse. Ten days after tumor inoculation, EGCG was administered in the drinking water at a concentration of 0, 0.1, 0.5, or 2.5 mg/ml for five days. The TC-1 tumor-challenged mice were characterized for tumor growth by measuring the tumor volume 1 week after the termination of EGCG treatment.

For in vivo tumor protection experiments, C57BL/6 mice (five per group) were vaccinated and received a booster with the Sig/E7/LAMP-1 DNA or control DNA via gene gun and challenged with 5×10⁵ TC-1 tumor cells/mouse subcutaneously in the right leg three days after the initial vaccination. EGCG (Sigma Chemical Co.) was administered in the animals' drinking water at various concentrations (0, 0.02, 0.1, 0.5, or 2.5 mg/ml) at the time of tumor challenge and continued for 11 days. Mice were monitored for evidence of tumor growth by measuring the tumor volume at 14 days after tumor challenge. In another set of tumor protection experiments, EGCG was administered in the animals' drinking water at the concentration of 0.5 mg/ml at the time of tumor challenge and continued for 11 days. Treated mice were monitored for evidence of tumor growth by inspection and palpation twice a week.

For the characterization of the subsets of lymphocytes important for the anti-tumor effects, C57BL/6 mice (5 per group) were vaccinated and received a booster with the Sig/E7/LAMP-1 DNA via gene gun and were subsequently challenged with TC-1 tumor cells three days after initial vaccination. EGCG was provided in the drinking water at a concentration of 0.5 mg/ml at the time of tumor challenge and continued for 11 days. Antibody depletion of subsets of lymphocytes was initiated one week after the last immunization using the methods described previously (29). MAb GK1.5 was used for CD4 depletion, MAb 2.43 was used for CD8 depletion, and MAb PK136 was used for NK1.1 depletion. Depletion was terminated on day 40 after tumor challenge. Mice were monitored for evidence of tumor growth by inspection and palpation twice a week.

For long-term tumor protection experiments, C57BL/6 mice (five per group) were vaccinated and boostered with Sig/E7/LAMP-1 DNA via gene gun. Three days after the initial vaccination, the mice were subcutaneously challenged with 5×10⁵ TC-1 tumor cells/mouse in the right leg. EGCG (Sigma Chemical Co.) was administered in the animals' drinking water at a dose of 0.5 mg/ml at the time of tumor challenge and continued for 11 days. Seven weeks after the last vaccination, the mice were injected with TC-1, B-16 or B16-E7 at a dose of 5×10⁴ tumor cells/mouse via tail vein to simulate hematogenous spread of tumors and evaluate long-term protection. Mice were sacrificed 24 days after tumor challenge and assayed for tumor growth in the lung.

For the tumor treatment experiments, mice were challenged with 1×10⁴ TC-1 tumor cells/mouse subcutaneously. 3 days later, the mice were vaccinated with Sig/E7/LAMP-1 DNA and received a booster with the same DNA via gene gun one week later. EGCG was administered in the drinking water at a concentration of 0.5 mg/ml at the time of initial DNA treatment and continued for 14 days. Tumor volumes were measured and recorded twice a week for 78 days following tumor challenge. In vivo tumor experiments were performed three times to generate reproducible data.

Statistical analysis. All data are expressed as means±standard deviation (S.D.) and are representative of at least two separate experiments. Results for intracellular cytokine staining with flow cytometry analysis and tumor treatment experiments were evaluated by analysis of variance (ANOVA). Comparisons between individual data points were made using Student's t-test. In the tumor protection experiments, the principal outcome measure was time to tumor development. The event time distributions for different mice were compared using the Kaplan and Meier method and the log-rank statistic. All p values <0.05 were considered significant.

### Additional Materials & Methods

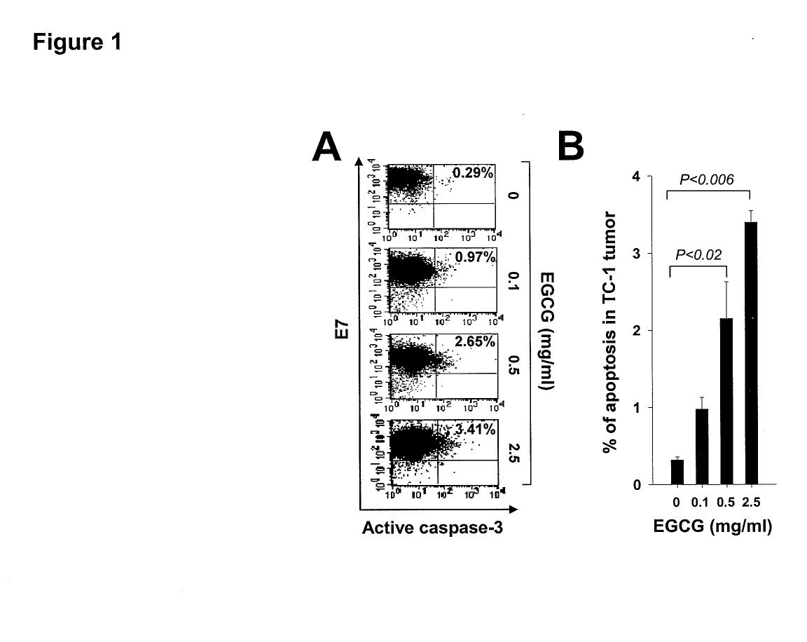

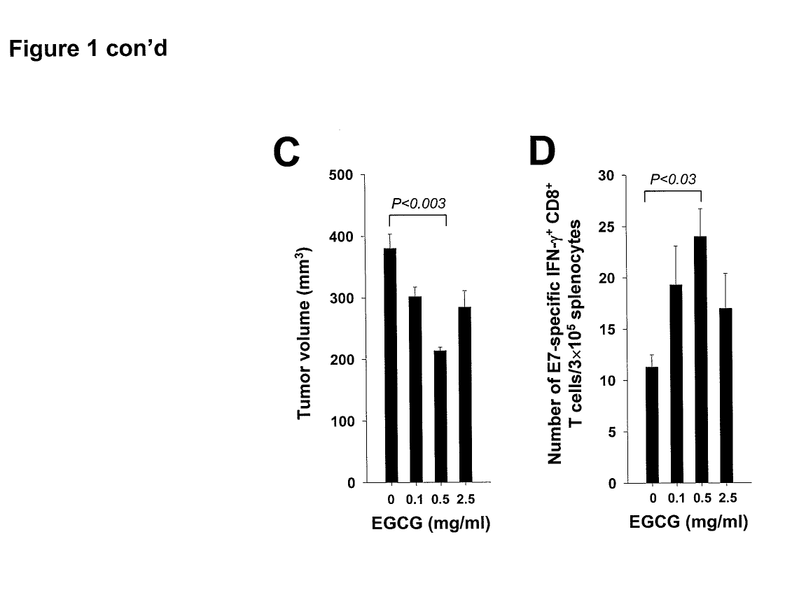

In FIG. 1, C57BL/6 mice (five per group) were injected subcutaneously in the right leg with 5×10⁵ TC-1 tumor cells/mouse. 10 days after tumor inoculation, EGCG was administered in the drinking water at a concentration of 0, 0.1, 0.5, or 2.5 mg/ml for five days. To characterize the expression of HPV-16 E7 protein in TC-1 tumor cells, single cell suspensions of isolated tumor were prepared and stained with E7 specific monoclonal antibody. Detection of apoptotic cells was performed using PE-conjugated Rabbit Anti-Active Caspase-3, a marker of apoptosis. The TC-1 tumor-challenged mice were characterized for tumor growth by measuring the tumor volume. The HPV-16 E7-specific CD8⁺ T cell immune responses in treated mice were characterized by intracellular cytokine staining for IFN-γ followed by flow cytometry analysis of splenocytes. Characterization of tumor volume and the number of E7-specific CD8 T⁺ cell were performed 1 week after the termination of ECGC treatment. A. Representative flow cytometry data. B. Bar graph of the percentage of apoptotic cells observed in TC-1 tumors (mean±SD). C. Bar graph of the volume of TC-1 tumors (mean±SD). D. Bar graph depicting the number of IFN-γ-secreting E7-specific CD8⁺ T cells/3×10⁵ splenocytes (mean±SD).

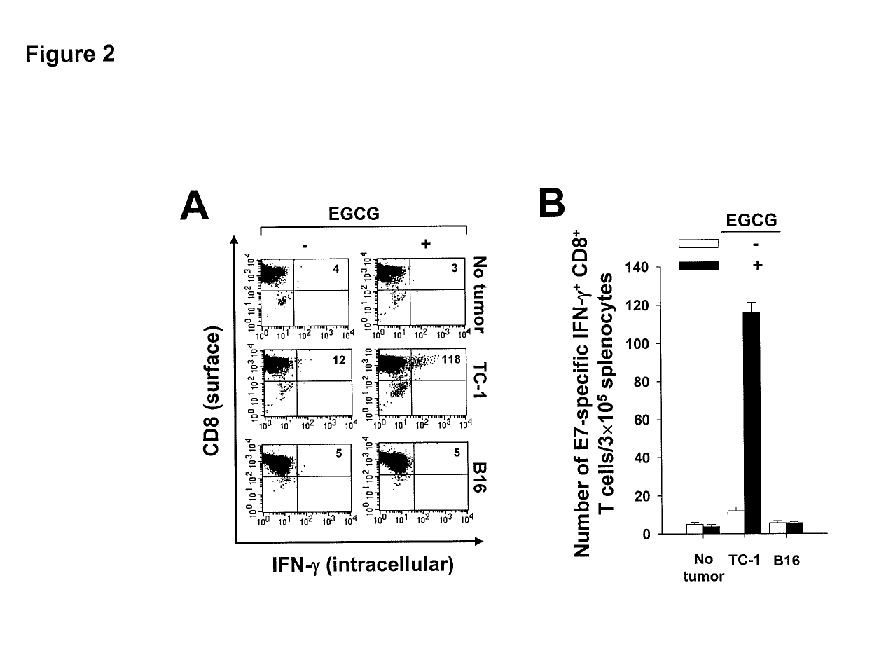

In FIG. 2, 10 days after tumor inoculation, tumor-bearing mice were given EGCG in their drinking water at a concentration of 0.5 mg/ml for five days. Inguinal lymph nodes were then harvested from the mice and CD11c⁺ cells were enriched from a single cell suspension of isolated inguinal lymph nodes using CD11c (N418) microbeads (Miltenyi Biotec, Auburn, Calif.). Enriched CD11c⁺ cells were analyzed by forward and side scatter and gated around a population of cells with size and granular characteristics of dendritic cells (DCs). 2×10⁴ isolated CD11c⁺ DC cells were incubated for 16 hours with 2×10⁶ E7-specific CD8⁺ T cells. Cells were then stained for both surface CD8 and intracellular IFN-γ and analyzed by flow cytometry. A. Representative flow cytometry data. B. Bar graph depicting the number of IFN-γ-secreting E7-specific CD8⁺ T cells/3×10⁵ cells (mean±SD). The data shown was from one representative experiment of three performed.

In FIG. 3, C57BL/6 mice (5 per group) were inoculated with TC-1 tumor cells (A & B) or 1×PBS (C) subcutaneously. Three days later, the mice were vaccinated with either the Sig/E7/LAMP-1 DNA vaccine or a control DNA containing no insert. Mice received a booster of Sig/E7/LAMP-1 DNA vaccine seven days after the first vaccination. For A and B, in the presence of tumor, oral EGCG treatment (0.5 mg/ml) was initiated at the time of vaccination and continued for 14 days. For C, in the absence of tumor, EGCG treatment was given at various concentrations (0, 0.1, 0.5 or 2.5 mg/ml) was initiated at the time of vaccination and continued for 14 days. Intracellular cytokine staining for IFN-γ was performed followed by flow cytometry analysis to characterize HPV-16 E7-specific CD8⁺ T cell immune responses in treated mice. A. Representative set of the flow cytometry data. B. & C. Bar graphs depicting the number of E7-specific IFN-γ-secreting CD8⁺ T cells/3×10⁵ splenocytes (mean±SD). The data shown was from one representative experiment of three performed.

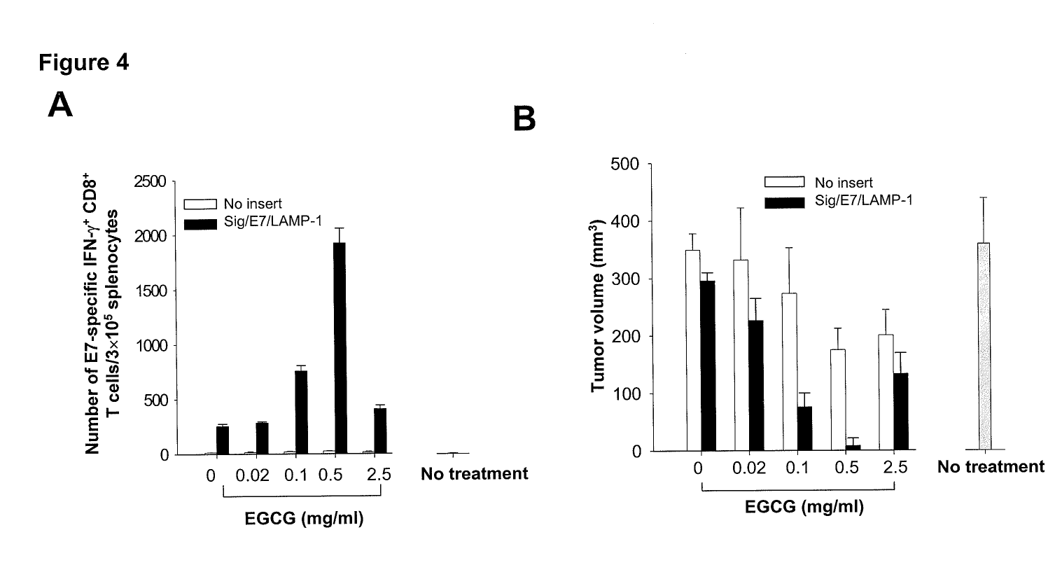

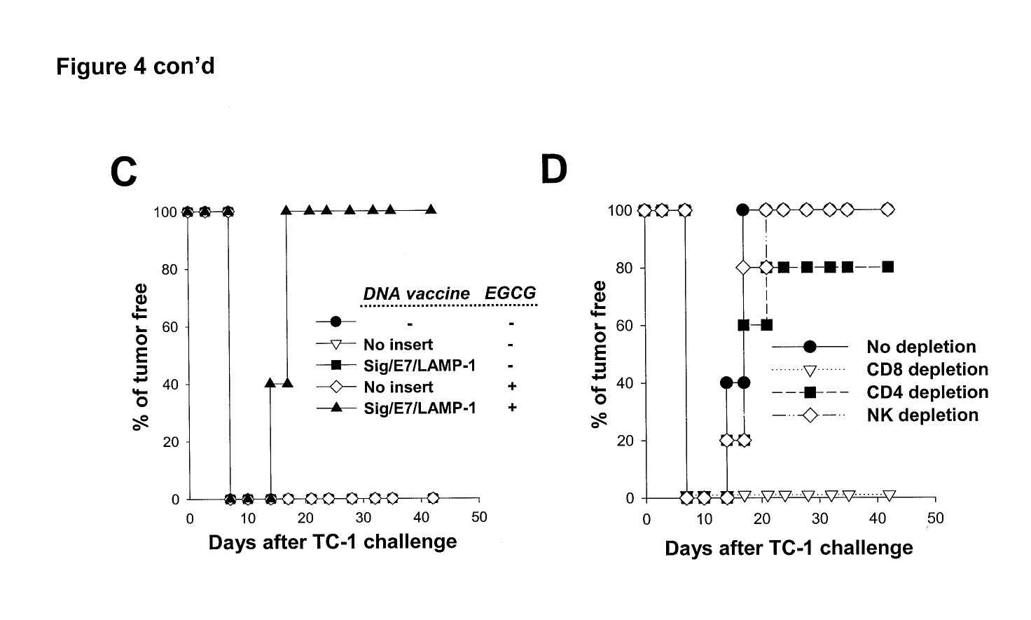

In FIG. 4, C57BL/6 mice (5 per group for all of the studies) were vaccinated and boostered with the Sig/E7/LAMP-1 DNA (solid bar) or a control DNA containing no insert (open bar) and were subsequently challenged with TC-1 tumor cells subcutaneously three days after initial vaccination. For A and B, EGCG of various concentrations was provided in the drinking water, ranging from 0 to 2.5 mg/ml at the time of tumor challenge and continued for 11 days. For C and D, EGCG was provided in the drinking water at the concentration of 0.5 mg/ml at the time of tumor challenge and continued for 11 days. A. Intracellular cytokine staining for IFN-γ followed by flow cytometry analysis was performed to characterize HPV-16 E7-specific CD8⁺ T cell immune responses in treated mice. Bar graph depicting the number of E7-specific IFN-γ-secreting CD8⁺ T cell precursors/3×10⁵ splenocytes (mean±SD). B. In vivo tumor growth experiments. TC-1 tumor-challenged mice were evaluated for tumor growth by measuring the tumor volume 14 days after TC-1 tumor challenge. C. In vivo tumor growth experiments. Tumor growth was monitored by inspection and palpation twice a week following subcutaneous TC-1 tumor challenge. D. In vivo antibody depletion experiment to characterize the subsets of lymphocytes important for the anti-tumor effects. Antibody depletion was initiated one week following the last immunization. Tumor growth was monitored by inspection and palpation twice a week.

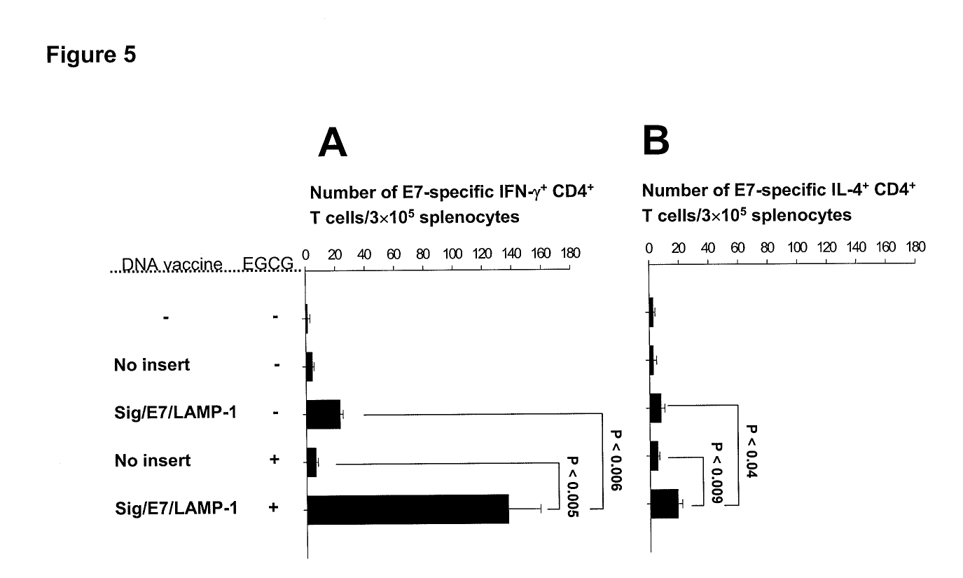

In FIG. 5, C57BL/6 mice (5 per group) were vaccinated with the Sig/E7/LAMP-1 DNA vaccine and treated with EGCG in the presence of established TC-1 tumor cells as described in FIG. 3. The presence of E7-specific CD4⁺ T cells in vaccinated mice were characterized by intracellular cytokine staining for IFN-γ (A. secreted by Th1 cells) or IL-4 (B. secreted by Th2 cells) using flow cytometric analysis of splenocytes derived from the treated mice.

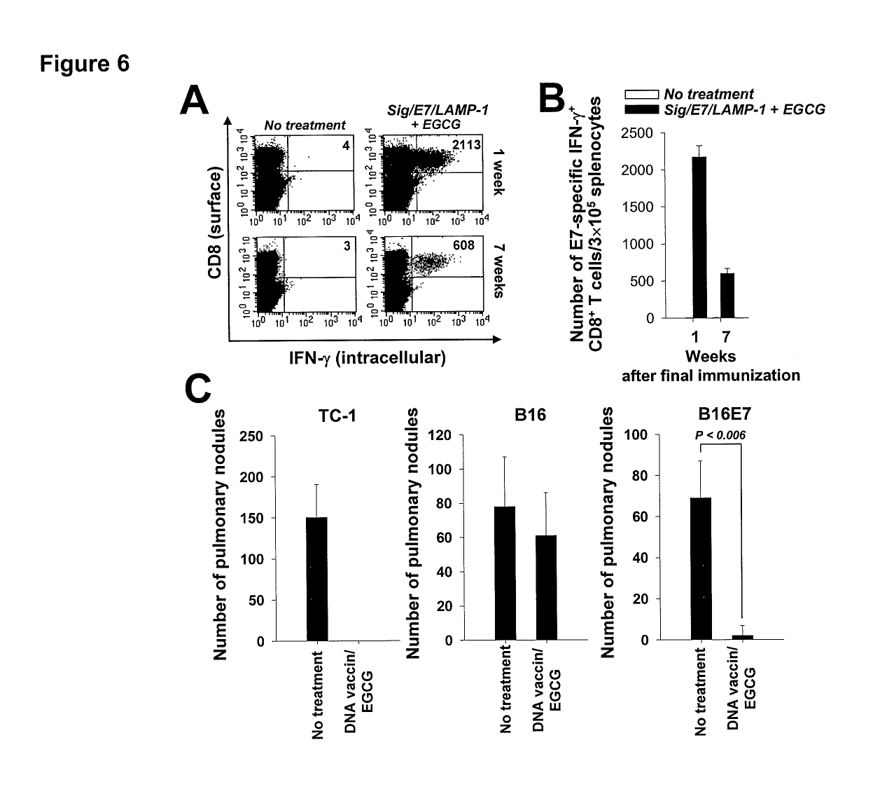

In FIG. 6, C57BL/6 mice (five per group) were vaccinated and boostered with the Sig/E7/LAMP-1 DNA vaccine and subsequently challenged with TC-1 tumor cells three days after initial vaccination. Mice were treated with EGCG provided in the drinking water at a dose of 0.5 mg/ml at the time of tumor challenge and continued for 11 days as described in FIG. 5. Intracellular cytokine staining followed by flow cytometric analysis was performed at week one and week seven after the last vaccination to characterize the levels of E7-specific CD8⁺ T cells generated in treated mice. A. Representative set of the flow cytometric analysis data. The data presented was from one representative experiment of three performed. B. Bar graph depicting the number of E7-specific IFN-γ-secreting CD8⁺ T cell precursors/3×10⁵ in splenocytes (mean±SD). C. Long term in vivo tumor protection experiments using TC-1, B-16 or B-16E7 tumor cells. To determine the long-term tumor protection ability of our vaccination strategy, tumor free mice were re-challenged with 5×10⁴ tumor cells/mouse of TC-1, B16 or B16E7 seven weeks after the last immunization.

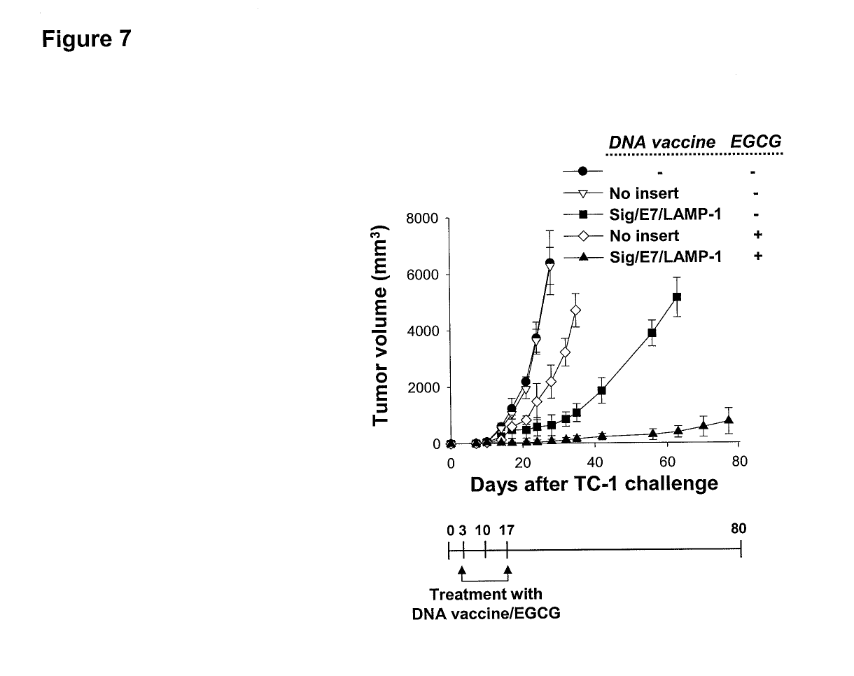

In FIG. 7, for the tumor treatment experiments, C57BL/6 mice (5 per group) were inoculated subcutaneously with 1×10⁴ TC-1 tumor cells/mouse. Three days after tumor inoculation, mice were vaccinated with Sig/E7/LAMP-1 DNA. Mice received a booster of Sig/E7/LAMP-1 DNA vaccine with the same dose and regimen 7 days after the first vaccination. EGCG was administered in the drinking water at a concentration of 0.5 mg/ml at the start of the vaccination and continued for 14 days. Tumor volumes were measured and recorded twice per week for eight weeks following immunization. Tumor treatment experiments were performed three times to generate reproducible data.

Tumor Treated with EGCG Induced Apoptotic Cell Death of Tumors, Generated HPV-16 E7-Specific CD8+ T Cells and Inhibited Tumor Growth of E7-Expressing Tumors

The percentage of apoptotic tumor cells and antigen presentation in the draining lymph nodes were quantified after EGCG administration in mice with established tumors. Mice were subcutaneously inoculated with 5×10⁵ TC-1 tumor cells/mouse. TC-1 is a previously described E7-expressing tumor model (29). Ten days after tumor inoculation, EGCG was administered for five days in the drinking water at a concentration of 0, 0.1, 0.5, or 2.5 mg/ml. After preparation of single cell suspensions of isolated tumors, detection of apoptotic cells was performed using PE-conjugated Rabbit Anti-Active Caspase-3 Antibody, according to the manufacturer's instructions. To identify TC-1 cells, single cell suspensions of the tumor were also stained with E7-specific monoclonal antibody. The percentage of apoptotic tumor cells was analyzed using flow cytometry. As shown in FIGS. 1 A and B, tumors of mice treated with EGCG demonstrated dose-dependent apoptosis. There was an increased percentage of tumor cell apoptosis in a dose-dependent manner of administered EGCG. In fact, there was a greater than 11 fold increase in the percentage of apoptosis in TC-1 tumors in mice treated with 2.5 mg/ml of EGCG in the drinking water compared to mice treated with 0 mg/ml of EGCG (3.41% vs. 0.29%). To determine whether EGCG induced-apoptosis leads to a decrease in the tumor volume, tumor-bearing mice were treated with EGCG as described above and tumor volume was measured lweek after the termination of ECGC treatment. As shown in FIG. 1C, there was a correlative decrease in tumor volume as EGCG concentrations increased from 0 to 0.5 mg/ml. However, at the highest dose of EGCG (2.5 mg/ml) there was a relative increase in tumor volume as compared to the 0.5 mg/ml dose. Further, the present inventors measured the E7-specific CD8⁺ T cell immune response in tumor-bearing mice treated with various concentrations of EGCG. As shown in FIG. 1D, there was an observed increase in the number of E7 specific CD8⁺ T cells in a dose-dependent manner of EGCG administered at doses ranging from 0 to 0.5 mg/ml. However, the number of E7 specific CD8⁺ T cell decreased when EGCG was administered at a concentration of 2.5 mg/ml which correlated with the increased tumor volume observed at this concentration as shown in FIG. 1C. These results indicate that tumor cell apoptosis occurs in a linear relationship with the dose of EGCG administered. Furthermore, immune cell responses and anti-tumor effects correlate with increasing doses of EGCG administered at a certain dose range (0 to 0.5 mg/ml). However, when EGCG is administered at the highest dose of 2.5 mg/ml there appears to be a decrease in E7-specific immune responses as well as a decrease in the observed anti-tumor effect. Our data suggest that at higher doses of EGCG, the enhancement of antigen-specific CD8⁺ T cell immune responses mediated by induced tumor cell apoptosis may be countered by the potential immunosuppressive effects of EGCG on the immune system.

Tumor Treated with EGCG Generated Higher Levels of Antigen-Loaded Dendritic Cells in the Draining Lymph Nodes of Tumor-Bearing Mice.

To determine whether apoptosis increased antigen cross-presentation in draining lymph nodes, tumor bearing mice were treated with EGCG in the drinking water at a concentration of 0.5 mg/ml, as described in FIGS. 1A and 1B. The selection of the EGCG dose at the concentration of 0.5 mg/ml was based on the observed findings from FIGS. 1C and 1D. After EGCG treatment, inguinal lymph nodes were harvested. CD11c⁺ cells were enriched from a single cell suspension of isolated inguinal lymph nodes and then incubated for 16 hours with an E7-specific CD8⁺ T cell line. Cells were then stained for both surface CD8 and intracellular IFN-γ and analyzed by flow cytometry to measure in vitro activation of E7-specific CD8⁺T cells(10). As shown in FIG. 2, CD11c⁺-enriched cells isolated from mice treated with 0.5 mg/ml EGCG were more effective in stimulating E7-specific CD8⁺ T cells to secrete IFN-γ, when compared with CD11c⁺-enriched cells from mice not treated with EGCG. These effects are antigen specific as demonstrated by the lack of response observed in mice bearing a non-E7 expressing tumor, B16. These results demonstrate that tumor-bearing mice treated with EGCG generate higher levels of antigen-loaded dendritic cells (DCs) in draining lymph nodes which are able to activate antigen-specific CD8⁺T cell immune responses.

### Combined DNA Vaccination and EGCG Treatment Generated an Enhanced E7-Specific CD8⁺ T Cell Immune Response as Compared to Monotherapy Alone.

The ability of a combined strategy of DNA vaccination and EGCG treatment to generate E7-specific CD8⁺ T cell immune responses was evaluated. Mice were inoculated with 1×10⁴ TC-1 tumor cells/mouse subcutaneously. Three days later, the mice were vaccinated with Sig/E7/LAMP-1 DNA or a control DNA without any insert. EGCG was administered in the drinking water at a concentration of 0.5 mg/ml at the time of vaccination and continued for 14 days. The E7-specific CD8⁺ T cell immune response in the mice treated as described above was assessed. As shown in FIGS. 3 A and B, the combination treatment with Sig/E7/LAMP-1 DNA and EGCG resulted in a robust increase in the number of IFN-γ-secreting E7-specific CD8⁺ T cell precursors as compared to single therapy with Sig/E7/LAMP-1 DNA alone (at least a 6.5 fold increase) or EGCG treatment alone. Thus, our data demonstrate that a combination of Sig/E7/LAMP-1 DNA vaccine with orally administered EGCG can significantly enhance tumor antigen-specific CD8⁺ T cell immune responses.

To determine whether EGCG treatment affects the generation of E7-specific CD8⁺T cell-mediated immunity in DNA vaccinated mice in the absence of tumor, C57BL/6 mice were vaccinated with the Sig/E7/LAMP-1 DNA intradermally and boostered with the same DNA vaccine at the same dose via gene gun one week later. EGCG was administered in the drinking water at various concentrations ranging from 0, 0.1, 0.5 or 2.5 mg/ml at the time of vaccination and continued for 14 days. HPV-16 E7-specific CD8⁺ T cell immune responses in treated mice were characterized by intracellular cytokine staining followed by flow cytometry analysis 14 days after DNA vaccination. As shown in FIG. 3C, in the absence of tumor, the HPV-16 E7-specific CD8⁺ T cell immune responses in vaccinated mice continued to decrease with the increasing amount of EGCG administered orally. Taken together, these data indicated that the enhanced antigen-specific CD8⁺ T cell immune responses observed by the DNA vaccine in combination with EGCG are only observed in the presence of tumor and are likely due to increased tumor cell apoptosis mediated by EGCG.

### The Levels of E7-Specific CD8⁺ T Cell Immune Responses and Anti-Tumor Effects Against E7-Expressing Tumors are Related to the Dose of EGCG Administered.

The present inventors further determined if the doses of EGCG treatment affects the generation of E7-specific CD8⁺T cell-mediated immunity and antitumor effects in tumor-challenged mice. C57BL/6 mice were vaccinated and boostered with the Sig/E7/LAMP-1 DNA or a DNA vector without insert, and were subsequently challenged with TC-1 tumor cells three days after initial vaccination. EGCG was provided at various concentrations, specifically 0, 0.02, 0.1, 0.5 or 2.5 mg/ml at the time of tumor challenge and continued for 11 days. Antigen-specific immune responses and tumor volume were measured 14 days after TC-1 challenge. As shown in FIG. 4A, the E7-specific CD8⁺ T cell immune responses increased in a dose-dependent manner with the concentration of EGCG, at a dose range of 0 to 0.5 mg/ml in mice immunized with Sig/E7/LAMP-1 DNA vaccine. However, EGCG treatment at 2.5 mg/ml dramatically decreased the number of E7-specific CD8⁺ T cells as compared to mice treated with EGCG at a dose of 0.5 mg/ml. Mice immunized with a DNA containing no insert failed to generate any significant levels of E7-specific CD8⁺ T cell immunity at any of the tested concentrations. Similarly, tumor volume decreased in a dose-dependent manner with the concentration of EGCG in mice vaccinated with Sig/E7/LAMP-1 DNA (FIG. 4B). However, the tumor volume of the DNA-vaccinated mice treated with 2.5 mg/ml of EGCG was significantly larger than those mice treated with 0.5 mg/ml of EGCG. Taken together, in the presence of tumor, the antigen specific immune responses and anti-tumor effects in DNA vaccinated, EGCG treated mice were enhanced at certain dose ranges of EGCG and, at higher doses of EGCG, the benefits of its anti-tumor effects may be countered by the potential immunosuppressive effects of EGCG on the immune system.

Antibody Depletion Experiments Demonstrated that CD8⁺ T Cells were Important for the Anti-Tumor Effects Generated by the Combined Therapy.

The anti-tumor effects generated by immunization with the Sig/E7/LAMP-1 DNA vaccine or an empty DNA vector in the presence or absence of EGCG administration at a concentration of 0.5 mg/ml were also characterized. Mice were vaccinated with the DNA vaccine and were subsequently challenged three days later with TC-1 tumor cells. Mice were then administered plain drinking water or drinking water containing EGCG at the time of tumor challenge and continued for 11 days. Tumor growth was monitored twice a week by inspection and palpation. As shown in FIG. 4C, only the mice receiving the combined therapy with DNA vaccine and EGCG had tumor regression within 20 days after tumor challenge. All of the mice receiving Sig/E7/LAMP-1 DNA in combination with EGCG remained tumor free 42 days after TC-1 tumor challenge. In contrast, all of the mice treated with Sig/E7/LAMP-1 or EGCG alone continued to demonstrate tumor growth.

To determine the subset of lymphocytes that are important for the anti-tumor effects generated by combined therapy, the present inventors performed in vivo antibody depletion experiments in mice that were challenged with TC-1 tumors and treated with Sig/E7/LAMP-1 DNA vaccine in combination with EGCG at a concentration of 0.5 mg/ml. As shown in FIG. 4D, all of the mice depleted of CD8⁺ T cells did not demonstrate tumor regression. In comparison, all of the mice depleted of NK cells demonstrated tumor regression similar to mice without antibody depletion. 80% of mice depleted of CD4 cells demonstrated tumor regression. These data suggest that CD8⁺T cells are essential for the anti-tumor effects generated by the combined therapy.

### Combined DNA Vaccination and EGCG Treatment Generated an Enhanced Th1 E7-Specific CD4⁺ T Cell Immune Response.

The ability of the Sig/E7/LAMP-1 targeting strategy to enhance antigen presentation to CD4⁺ T lymphocytes is achieved by targeting the expressed antigen to endosomal/lysosomal compartments and subsequently to the MHC class II antigen presentation pathway. To determine the nature of the E7-specific CD4⁺ T cell response to the combined treatment with Sig/E7/LAMP-1 DNA vaccination and oral EGCG administration, intracellular cytokine staining was performed for IFN-γ (secreted by Th1 cells) or IL-4 (secreted by Th2 cells) using flow cytometry analysis. Splenocytes derived from the mice were treated as previously described in FIG. 3. As shown in FIG. 5, vaccination with Sig/E7/LAMP-1 DNA combined with EGCG administration generated significantly higher levels of E7-specific Th1 CD4⁺ T lymphocytes than vaccination with Sig/E7/LAMP-1 alone or EGCG treatment alone. In contrast, there was only a slight increase in E7-specific Th2 CD4⁺ T lymphocytes. These data suggest that the combination of Sig/E7/LAMP-1 DNA vaccination with oral EGCG treatment may contribute to an enhanced E7-specific CD4⁺ Th1 cell response.

### Combined DNA Vaccination and EGCG Treatment Generated Significant Long-Term Immune Response and Antitumor Protection in Treated Mice.

Ideally, a successful cancer treatment must be capable of generating effective long-term protection. Therefore, the ability of our combined therapy to generate long-term E7-specific CD8⁺ T cell immune responses and protective antitumor effects was assessed. Intracellular cytokine staining was followed by flow cytometry analysis to identify E7-specific CD8⁺ T cells 1 week and 7 weeks after the last immunization of the mice which did not had evidence of tumor growth following the TC-1 tumor challenge. As shown in FIGS. 6A and 6B, significant levels of the E7-specific IFN-γ CD8⁺ T lymphocyte response generated by the combined therapy were still present up to 7 weeks post-immunization. All of the mice remained tumor-free.

To determine the long-term tumor protective ability of our vaccination strategy, the tumor-free mice were re-challenged intravenously with 5×10⁴ TC-1 tumor cells 7 weeks after the final immunization. As shown in FIG. 6C, the naïve mice exhibited 151.6±42.3 tumor nodules 42 days after TC-1 challenge, whereas the mice treated with the Sig/E7/LAMP-1 DNA vaccine and oral EGCG treatment exhibited no pulmonary tumor nodules. Thus, in a tumor protection experiment, the combined therapy successfully prevented tumor nodule formation up to seven weeks after vaccination. This long-term antitumor immunity was highly E7-specific because vaccinated mice were not protected from a non-E7 expressing tumor model, B16. In comparison, an E7 antigen-expressing B16 tumor cell line, B16E7, failed to form a high number of tumor nodules in the vaccinated mice. Taken together, these data indicate that DNA vaccination combined with oral EGCG treatment generates a strong long-term antigen-specific CD8⁺T cell immune response with excellent long-term protective anti-tumor effects.

Combined DNA Vaccination and EGCG Treatment Generated Synergistic Antitumor Therapeutic Effects than Monotherapy Alone.

For the tumor treatment experiments, mice were inoculated with 1×10⁴ TC-1 tumor cells/mouse subcutaneously. Three days later, mice were vaccinated with Sig/E7/LAMP-1 DNA. EGCG was administered in the drinking water at a concentration of 0.5 mg/ml at the start of the vaccination and continued for 14 days. Tumor volumes were measured and recorded twice per week for eight weeks following immunization. The present inventors found that the tumors in mice treated with the combined cancer therapy remained the smallest in size (FIG. 7). This indicates that the combined strategy of DNA vaccination and oral EGCG treatment results in greater loco-regional control of tumor than monotherapy alone in the TC-1 model.

### Discussion

Administration of highly cytotoxic cancer drugs has severe adverse side effects and causes discomfort for cancer patients. These highly toxic drugs also limit host immune reactions against cancers. In this study, the present inventors have demonstrated that oral administration of a low-toxic cancer drug, EGCG, resulted in complete tumor regression in mice vaccinated with Sig/E7/LAMP-1 DNA vaccine, without any severe systemic toxicity such as loss of hair, weight, or lymphopenia. Importantly, this combined therapeutic strategy generated stronger tumor-specific cytotoxic T cell immune responses, when compared to mice immunized with DNA vaccine alone. In addition, combined DNA vaccination and oral EGCG treatment generated a significant long-term immune response and protected mice from tumor growth upon repeated tumor challenges.

Immunotherapy and chemotherapy are generally rarely curative, even in small animal models of cancer, since many of these tumors rapidly grow to become large, bulky tumors, which present a challenge to either treatment regimen alone. At the start of this study, it was expected that EGCG might aid DNA vaccine-mediated antitumor effects by inhibiting tumor growth, thereby allowing time for a curative immune response to develop. Unexpectedly, however, a dramatic increase in E7-specific CD8⁺ T cell immunity was observed after combining DNA vaccination with oral administration of EGCG. This does not seem to be a direct adjuvant effect of EGCG on induction of E7-specific CD8⁺ T cell immunity, since oral administration of EGCG alone failed to increase the number of E7-specific CD8⁺ T cells generated by Sig/E7/LAMP-1 DNA vaccine in mice not bearing TC-1 tumors (see FIG. 3C). From these data, the present inventors propose that EGCG treatment may augment the antitumor immunity induced by genetic vaccination through enhanced tumor cell death, resulting in increased uptake of tumor antigens by antigen processing cells (APCs), such as dendritic cells, and enhanced antigen presentation in draining lymph nodes which can then activate CD8⁺ T cells (for review, see refs. (34), (35)). There is increasing evidence that the tumor antigens phagocytosed by bone marrow-derived DCs are introduced not only into the MHC class II but also the class I processing pathway in order to cross-prime naive T cells for development of potent immunity (36-38). Our data are consistent with this notion. Oral EGCG administration increased the percentage of apoptotic tumor cells and tumor-specific CD8⁺ T cell immunity in a dose-dependent manner up to certain level of EGCG concentration (0.5 mg/ml). Thus, these data provide direct evidence of how, after chemotherapy, the increased number of dying tumor cells led to more tumor antigen-loaded CD11c⁺ DCs in draining lymph nodes, resulting in increased tumor antigen-specific CD8⁺ T cells through cross-presentation.

Chemotherapy and immunotherapy have often been regarded as mutually exclusive. One of the reasons that contribute to this is lymphopaenia, a common side effect of most cancer drugs, which has been implicated as being detrimental to the antitumor immune response. It was shown that a high dose (2.5 mg/ml) of EGCG failed to enhance E7-specific CD8⁺ T cell immunity in mice with or without TC-1 tumors (see FIG. 4A and FIG. 3C) and, on the contrary, even decreased the anti-tumor effect in TC-1 tumor bearing mice (see FIG. 1C and FIG. 4B). This immune suppression may be related to an immune suppressive effect on T cells (39) and/or monocyte apoptosis (40) caused by high doses of EGCG, as has been reported by another group. Thus, in the presence of tumor, the antigen specific immune responses and anti-tumor effects at certain dose ranges of EGCG (0.1-0.5 mg/ml) are observed. However, at higher doses of EGCG (2.5 mg/ml), the benefits of its anti-tumor effects may be countered by the potential immunosuppressive effects of EGCG on the immune system.

Another possible reason that chemotherapy and immunotherapy have often been regarded as mutually exclusive is that chemotherapy induced apoptosis of cancer cells has been regarded as non-immunogenic, or even tolerogenic, in the absence of inflammatory molecules, called ‘danger signals’, which are necessary for the maturation of antigen presenting cells, such as DCs. The apoptotic death of a tumor cell, in the absence of inflammation, might appear as normal tissue turnover and generate immune ignorance or tolerance against a tumor cell (for review, see ref (41), (42), (43)). However, there is now increasing evidence that in appropriate immunological settings, cancer drug-induced apoptotic death of tumor cells can trigger the generation of effective antitumor immune responses (44-46). One such successful demonstration has been performed with cyclophosphamide. It is known that appropriate doses of cyclophosphamide help to generate strong immune priming after immunotherapy by depleting regulatory T cells from animals bearing tolerogenic tumors (47, 48).

Although sufficient numbers of tumor antigens are present within apoptotic tumor cells, their ability to induce a CTL response in the host may not be sufficient to cause rejection of the tumor as observed in our study using EGCG alone as a cancer drug. Under our experimental conditions, only weak E7-specific T cell immunity was demonstrated in mice bearing tumors that were treated with only EGCG, and dramatic regressions of the tumors did not occur (see FIG. 1). Only in the setting of combined DNA vaccination with EGCG treatment were enhanced E7-specific immune responses and anti-tumor therapeutic effects observed. One possible explanation for this observation is that EGCG induces tumor apoptosis, resulting in uptake of tumor antigen by professional antigen presenting cells, such as DCs and cross-presentation in tumor bearing mice. DCs play a critical role in priming as well as boosting adaptive immune responses. A number of investigators have demonstrated that DCs pulsed with tumor antigens induced cytokine production, enhanced proliferation of T cells in lymphoid tissues, and increased tumor infiltration by activated T cells (49-51). However, these strategies require ex vivo manipulation of DC and thus often are time and labor intensive. The combined therapy the present inventors propose in this study might be a promising approach for providing tumor specific antigens to DCs in draining lymph nodes for the enhancement of immune responses induced by vaccination.

The present inventors strongly believe that the results in the present study have great clinical implications. Since there are well-established effective chemotherapy protocols for controlling the rate of tumor growth and causing tumor cells to undergo apoptosis, immunotherapy might be used synergistically with chemotherapy for enhancing antitumor activity. On the basis of the fact that complete tumor regression and long-lasting tumor immunity was observed in this present study, the present inventors suggest that this same strategy could be applied to the treatment of other tumors using various immunotherapy models combined with effective cancer drugs. The present inventors have also tested a classic cytotoxic agent such as cisplatin in conjunction with DNA vaccination and have found that the combination of DNA vaccines with cisplatin also generated therapeutic effects in the control of TC-1 tumors as compared to monotherapy alone (Hung, et al., personal communication). The efficacy of immuno-chemotherapy for cancer has often been limited by the toxicity of the cancer drugs. The present inventors contemplate that local treatment of tumors using other efficient cancer treatments, such as radiotherapy (for review, see ref (52)), anti-angiogenesis agents (for review, see ref (53)), prodrug (for review, see ref (54)) strategies, or the use of drug delivery systems such as hydrogel-based systems (55), may be made more effective by increasing local toxic effects against tumors with minimal damage to host immune systems. Before undertaking such treatments, the routes and doses of drugs need to be optimized

The HPV DNA vaccine described in the current study is mainly for therapeutic purpose. The recently FDA-approved HPV vaccine is a preventive HPV vaccine using HPV virus-like particles (VLPs). While the HPV VLP vaccine is highly effective, it only includes four types of HPVs (HPV-6, -11, -16 and -18). Thus, the current preventive HPV vaccine can only prevent up to 70% of all cervical cancer. Furthermore, the preventive HPV vaccine cannot control existing HPV infections or HPV-associated lesions. A significant population of patients is currently suffering from HPV-associated morbidity or mortality. Thus, development of therapeutic vaccines such as the one reported here represents an important endeavor to complement the limitation of the FDA-approved preventive HPV vaccine.

In summary, our present study demonstrates that combined treatment with immune-modulating doses of chemotherapy can enhance the tumor-specific immune responses and antitumor effects induced by

DNA vaccines. These data provide an immunological rationale for testing various combinations of tumor vaccines with chemotherapy in patients with cancer. Many vaccine strategies and chemical drugs have been developed to control cancer. Considering that there are a multitude of possible combinations, a great deal of work could be forthcoming to evaluate combined therapy of tumor vaccines and chemotherapy for enhancing therapeutic effectiveness.

### Example 2

### The Vascular Disrupting Agent, 5,6 Di-methylxanthenone-4-acetic Acid enhances CD8+ T Cell-Mediated Antitumor Immunity Induced by DNA Vaccination

### Abstract

5,6-dimethylxanthenone-4-acetic acid (DMXAA), a small vascular disrupting agent (VDA) currently in advanced phase II clinical trials has been demonstrated potent ability to shutdown tumor blood flow and cause tumor necrosis. It has been shown that DMXAA efficiently activate tumor-associated macrophages to produce large amount of immunostimulatory cytokines and chemokines, such as TNF-alpha, inducing CD8+ T cell-dependent anti-tumor immune responses. More recently, DMXAA has been indicated to induce IFN-beta by potently and specifically activates TANK-binding kinase 1 (TBK1)-IFN regulatory factor 3 (IRF-3) signaling pathway. In the current study, we aim to investigate whether DMXAA can enhance the anti-tumor immunity induced by a DNA vaccine. We found that application of DMXAA is able to significantly enhance HPV 16 E6 and E7-specific CD8+ T cell responses induced by DNA vaccinations, although the time of DMXAA application significantly affect the outcome. Combination of DMXAA and DNA vaccination generated significantly better therapeutic anti-tumor effect in large, established tumor model. Therefore, combination of DMXAA, a chemotherapeutic agent with a therapeutic DNA vaccine provides a more effective immunotherapy against cancer.

### Results

### DMXAA Enhances HPV16 E7-Specific CD8+T Cell Response Induced by CRT/E7 DNA Vaccine in Vaccinated Mice

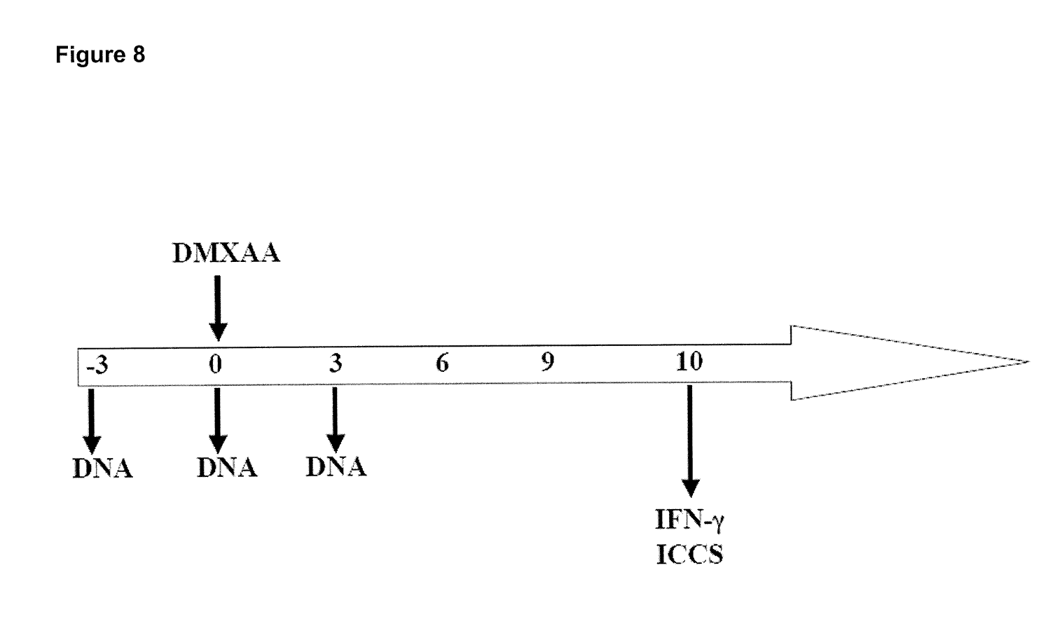

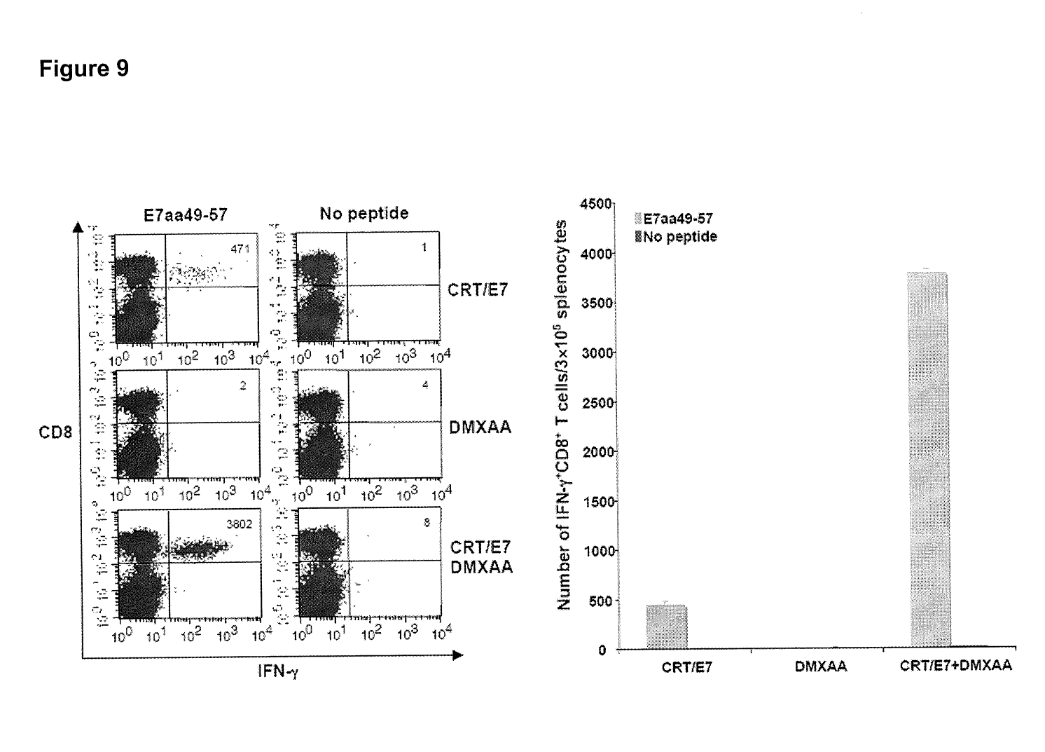

In order to determine the E7-specific CD8+ T cell immune response in mice treated with the various regimens, we treated the C57BL/6 mice (5 per group) with the DNA vaccine and/or DMXAA as illustrated in FIG. 8. Seven days after the last vaccination, we harvested splenocytes from vaccinated mice and characterized them for the presence of E7-specific CD8+ T cells using intracellular cytokine staining for IFN-γ followed by flow cytometry analysis. As shown in FIG. 9, mice that were administered DMXAA as well as CRT/E7 DNA generated significantly higher numbers of E7-specific CD8+ T cells compared to mice that were administered CRT/E7 DNA vaccine alone or DMXAA alone. Thus, our results suggest that treatment of mice with CRT/E7 DNA combined with DMXAA leads to the enhanced E7-specific CD8+ T cell immune response.

### DMXAA Enhances HPV16 E6-Specific CD8+ T Cell Response Induced by CRT/E6 DNA Vaccine in Vaccinated Mice

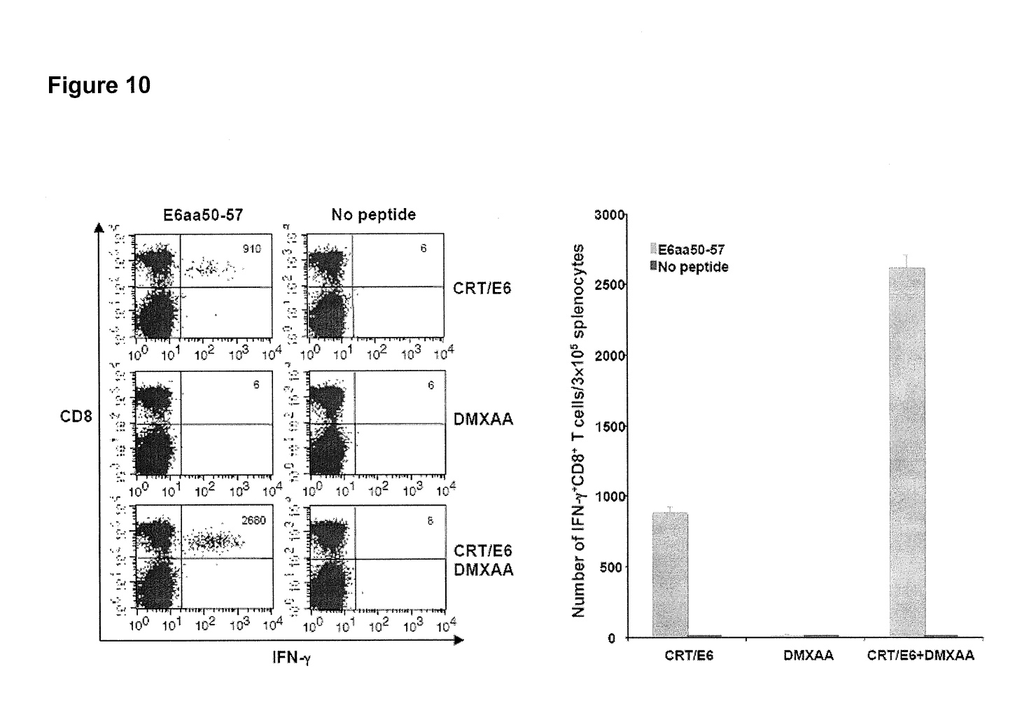

In order to determine the E7-specific CD8+ T cell immune response in mice treated with the various regimens, we treated C57BL/6 mice (5 per group) with the DNA vaccine and/or DMXAA as illustrated in FIG. 8. Seven days after the last vaccination, we harvested splenocytes from vaccinated mice and characterized them for the presence of E6-specific CD8+ T cells using intracellular cytokine staining for IFN-γ followed by flow cytometry analysis. As shown in FIG. 10, mice that were administered DMXAA as well as CRT/E6 DNA generated a significantly higher number of E6-specific CD8+ T cells compared to mice that were administered CRT/E6 DNA vaccine alone or DMXAA alone. Thus, our results suggest that treatment of mice with CRT/E6 DNA combined with DMXAA leads to an enhanced E6-specific CD8+ T cell immune response.

TC-1 Tumor Challenged Mice Treated with CRT/E7 DNA Combined with DMXAA Generate Highest Frequency of E7-Specific CD8+T Cells

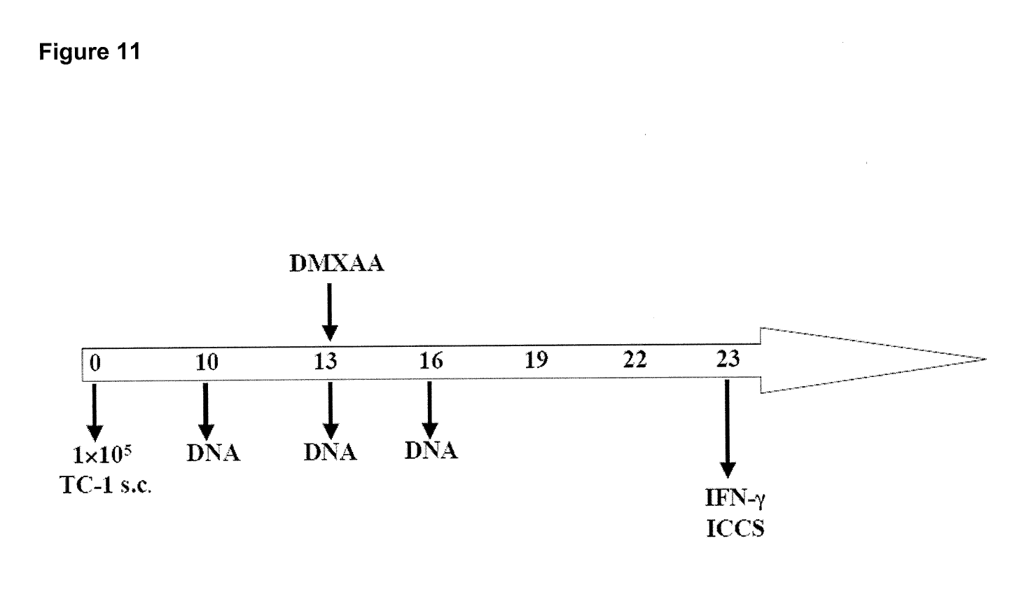

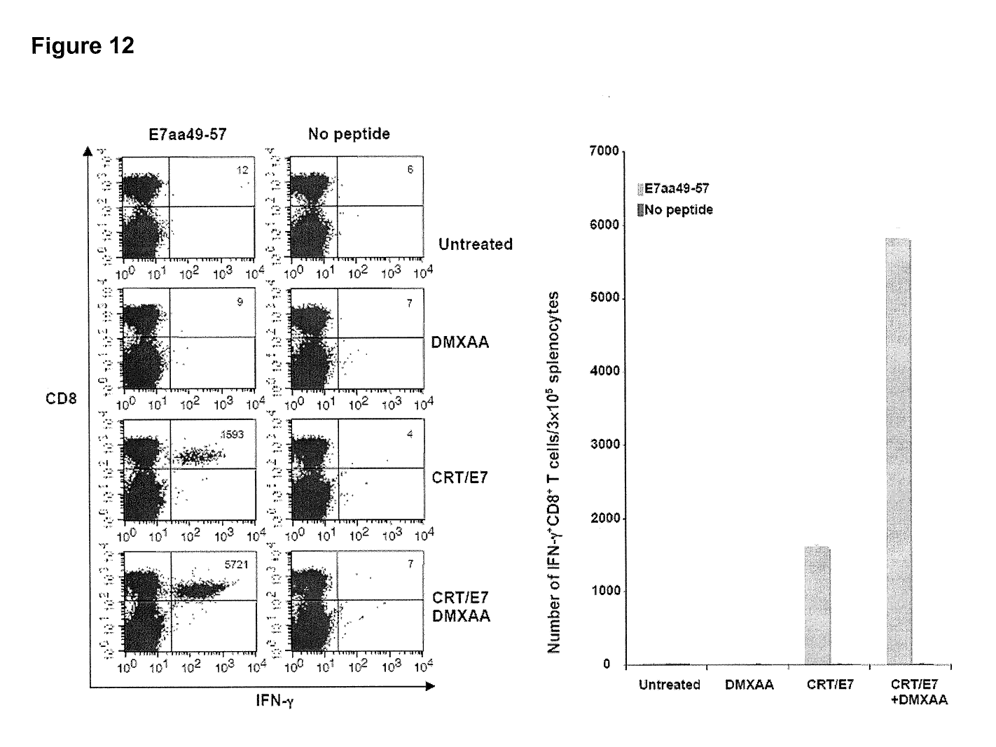

In order to determine the E7-specific CD8+ T cell immune responses in mice treated with the various regimens, we first challenged C57BL/6 mice (5 per group) with TC-1 tumor cells and then treated them with DNA vaccine alone, DNA vaccine combined with DMXAA or DMXAA alone as illustrated in FIG. 11. As a control, a group of tumor challenged C57BL/6 mice were left untreated for comparison. Seven days after the last treatment, we harvested splenocytes from tumor challenged mice and characterized them for the presence of E7-specific CD8+T cells using intracellular cytokine staining for IFN-γ followed by flow cytometry analysis. As shown in FIG. 12, tumor challenged mice that were administered CRT/E7 DNA combined with DMXAA generated significantly higher numbers of E7-specific CD8+ T cells compared to tumor challenged mice that were administered CRT/E7 DNA alone or DMXAA alone. Thus, our results suggest that treatment of tumor bearing mice with CRT/E7 DNA combined with DMXAA leads to an enhanced E7-specific CD8+ T cell immune response.

DMXAA Causes Extensive Tumor Necrosis and Infiltration of Inflammatory Cells into the Tumors of Mice Vaccinated with CRT/E7 DNA Vaccine

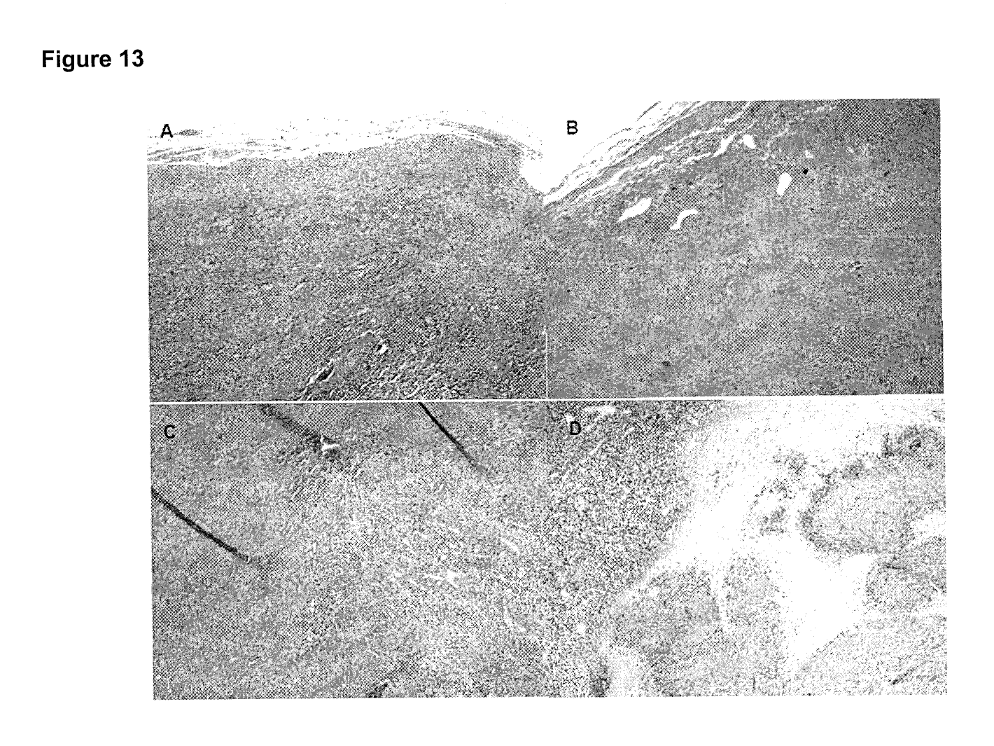

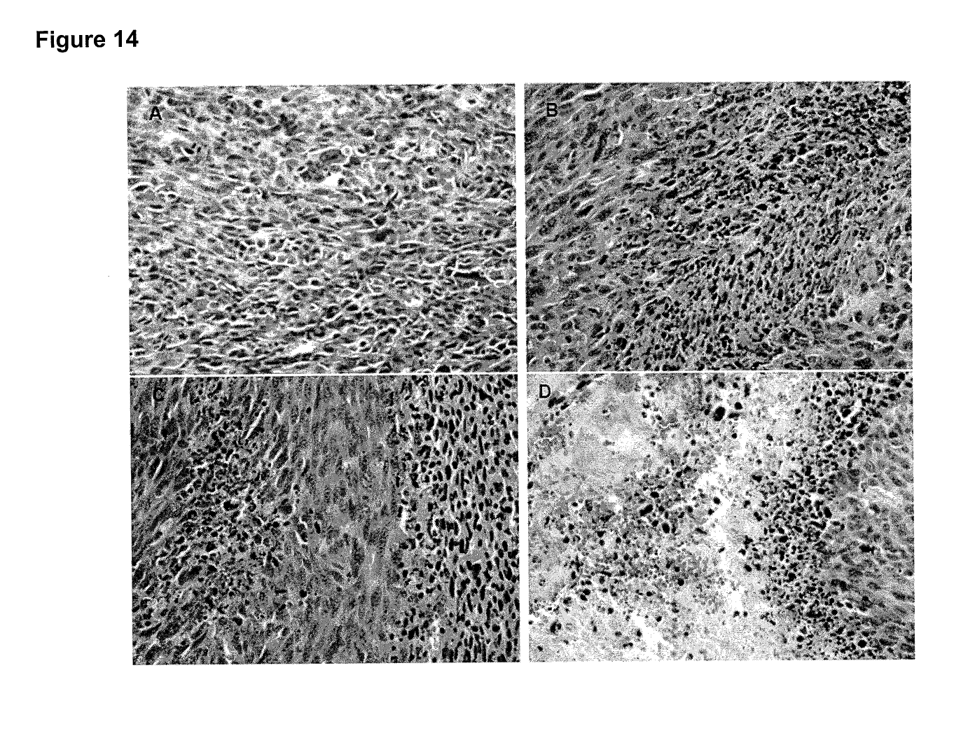

In order to determine the effect of DMXAA in the tumor microenvironment of vaccinated mice, we first challenged groups of C57BL/6 mice (5 per group) with TC-1 tumor cells and then treated them with DNA vaccine alone, DNA vaccine combined with DMXAA or DMXAA alone as illustrated in FIG. 11. As a control, a group of tumor challenged C57BL/6 mice were left untreated for comparison. Seven days after the last treatment, we extracted the tumors and performed immunohistochemistry analysis. As shown in FIG. 13, the tumors extracted from the tumor challenged mice that were administered CRT/E7 DNA combined with DMXAA showed extensive tumor cell necrosis compared to the tumors extracted from the tumor challenged mice that were administered CRT/E7 DNA alone or DMXAA alone. Furthermore, as shown in FIG. 14, the tumors extracted from the tumor challenged mice that were administered CRT/E7 DNA combined with DMXAA showed extensive infiltration of inflammatory cells compared to the tumors extracted from the tumor challenged mice that were administered CRT/E7 DNA alone or DMXAA alone. Thus, our results suggest that treatment of tumor bearing mice with CRT/E7 DNA combined with DMXAA leads to the enhanced tumor necrosis and infiltration of inflammatory cells into the tumors.

DMXAA Causes Extensive Infiltration of E7-Specific Tumor Infiltrating CD8+T Cells into the Tumors of Mice Vaccinated with CRT/E7 DNA Vaccine

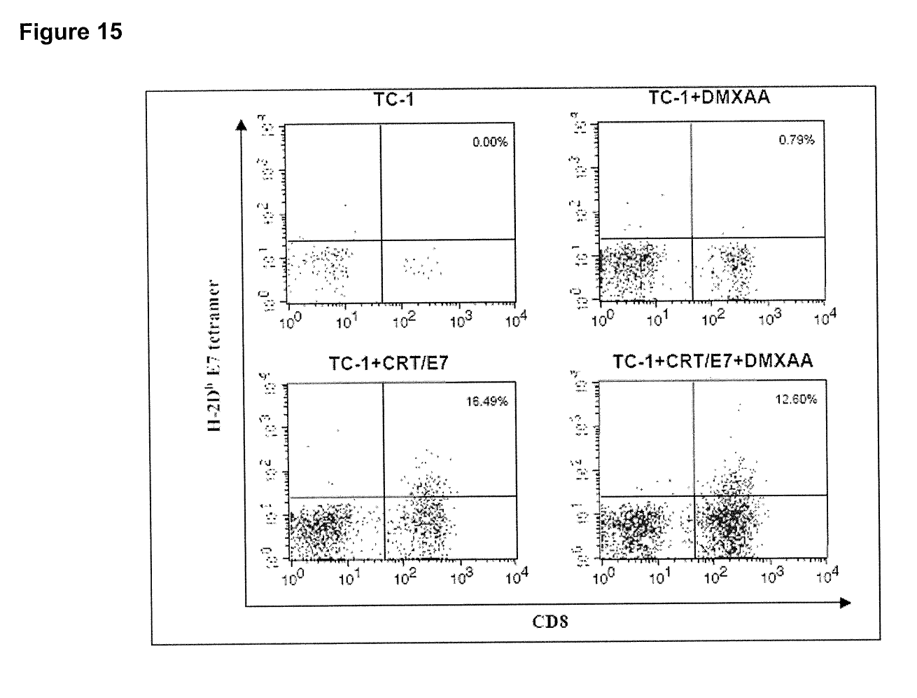

In order to determine the effect of DMXAA in the tumor microenvironment of vaccinated mice, we first challenged groups of C57BL/6 mice (5 per group) with TC-1 tumor cells and then treated them with DNA vaccine alone, DNA vaccine combined with DMXAA or DMXAA alone as illustrated in FIG. 11. As a control, a group of tumor challenged C57BL/6 mice were left untreated for comparison. Seven days after the last treatment, we performed E7 peptide-loaded MHC class I tetramer staining analysis. As shown in FIG. 15, tumor challenged mice that were administered CRT/E7 DNA combined with DMXAA generated significantly higher numbers of E7-specific tumor infiltrating CD8+ T cells compared to tumor challenged mice that were administered CRT/E7 DNA alone or DMXAA alone. Thus, our results suggest that treatment of tumor bearing mice with CRT/E7 DNA combined with DMXAA leads to the enhanced infiltration of E7-specific CD8+ T cells into the tumors.

Synergistic Antitumor Effects Generated by Combination of CRT/E7 DNA Vaccine with DMXAA

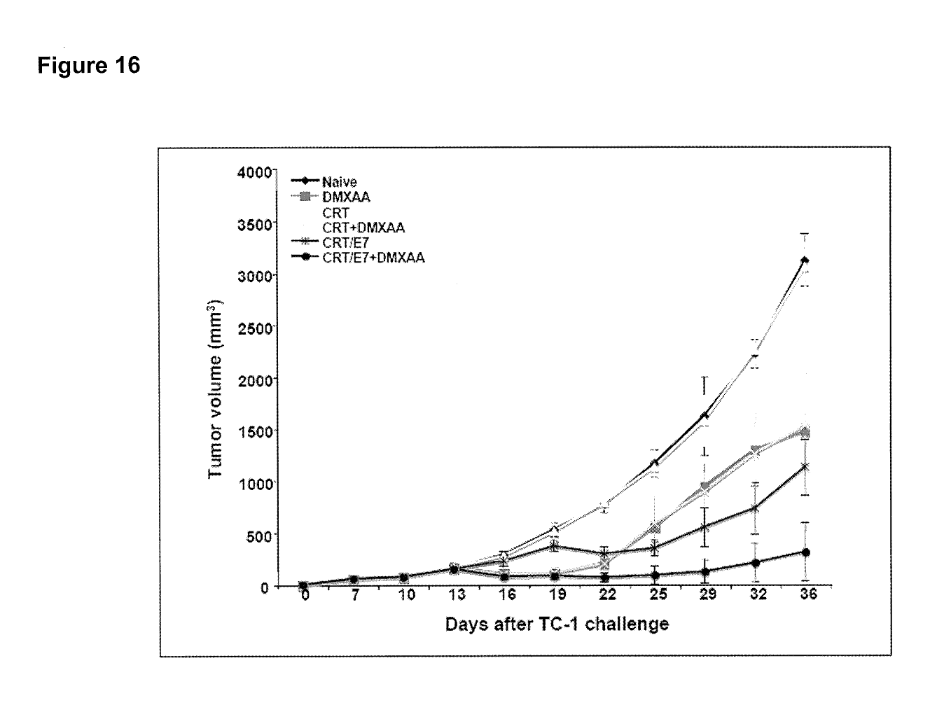

In order to determine the therapeutic antitumor effects of DMXAA in vaccinated mice, we first challenged groups of C57BL/6 mice (5 per group) with TC-1 tumor cells and then treated them with DNA vaccine alone, DNA vaccine combined with DMXAA or DMXAA alone as illustrated in FIG. 11. As a control, a group of tumor challenged C57BL/6 mice were left untreated for comparison. As shown in FIG. 16, tumor challenged mice treated with CRT/E7 DNA combined with DMXAA showed significantly lower tumor volumes over time as compared to challenged mice treated with the other treatment regimens. Furthermore, there was no statistical significance between tumor volumes in mice treated with CRT DNA and tumor volumes in mice treated with DMXAA alone. Thus, our data suggest that the treatment regimen using CRT/E7 DNA in combination with DMXAA produces the best therapeutic anti-tumor effects in TC-1 tumor bearing mice.

### Materials & Methods

In FIG. 8, C57BL/6 mice (5 per group) were vaccinated with 2 μg of CRT/E7 DNA three times with three-day intervals via gene gun delivery. A group of vaccinated mice was also injected with DMXAA (20 mg/kg, i.p injection) on the same day as the second DNA vaccination. Seven days after the last vaccination, splenocytes were harvested from mice for analysis.

In FIG. 9, C57BL/6 mice were vaccinated with CRT/E7 DNA vaccine and/or DMXAA as illustrated in FIG. 8. Seven days after last vaccination, pooled splenocytes were harvested and characterized for numbers of E7-specific IFN-γ+CD8+ T cells using intracellular IFN-γ staining followed by flow cytometry analysis. On the left, representative figure of the flow cytometry data. The numbers in the figure represent the numbers of E7-specific IFN-γ+CD8+ T cells out of 3×105 splenocytes. On the right, bar graph depicting the numbers of E7-specific IFN-γ-secreting CD8+ T cells per 3×105 pooled splenocytes (mean+s. d.).

In FIG. 10, C57BL/6 mice were vaccinated with CRT/E7 DNA vaccine and/or DMXAA as illustrated in FIG. 8. Pooled splenocytes were characterized for numbers of E6-specific IFN-γ+CD8+ T cells using intracellular IFN-γ staining followed by flow cytometry analysis. On the left, representative figure of the flow cytometry data. The numbers in the figure represent the number of E6-specific IFN-γ+CD8+ T cells out of 3×10⁵ splenocytes. On the right, bar graph depicting the numbers of E7-specific IFN-γ-secreting CD8+ T cells per 3×10⁵ pooled splenocytes (mean+s.d.).

In FIG. 11, C57BL/6 mice (5 per group) were challenged with 1×105 HPV16 E7-expressing TC-1 tumor cells subcutaneously. Ten days after tumor challenge, mice were treated with 2 μg of CRT/E7 DNA three times with three-day intervals via gene gun deliver. A group of vaccinated mice was also treated with DMXAA (20 mg/kg, i.p injection) on the same day as the second DNA vaccination. A control group of tumor challenged mice was left without treatment. Seven days after the last vaccination, splenocytes were harvested from mice for analysis.

In FIG. 12, C57BL/6 TC-1 tumor-bearing mice were treated with CRT/E7 DNA vaccine and/or DMXAA as illustrated in FIG. 11. Pooled splenocytes were characterized for numbers of E7-specific IFN-γ+CD8+ T cells using intracellular IFN-γ staining followed by flow cytometry analysis. were cytometry analysis. On the left, representative figure of the flow cytometry data. The numbers in the figure represent the numbers of E7-specific IFN-γ+CD8+ T cells out of 3×105 splenocytes. On the right, bar graph depicting the numbers of E7-specific IFN-γ-secreting CD8+ T cells per 3×105 pooled splenocytes (mean+s. d.).

In FIG. 13, C57BL/6 TC-1 tumor-bearing mice were treated with CRT/E7 DNA vaccine and/or DMXAA as illustrated in FIG. 11. Seven days after last vaccination, tumors were excised from the mice and histochemistry (H&E) staining was performed. Representative H&E stains showing tumor necrosis from tumor challenged mice (A) without treatment, (B) with CRT/E7 DNA treatment, (C) with DMXAA treatment and (D) with CRT/E7 DNA and DMXAA treatment.

In FIG. 14, C57BL/6 TC-1 tumor-bearing mice were treated with CRT/E7 DNA vaccine and/or DMXAA as illustrated in FIG. 11. Seven days after last vaccination, tumors were excised from the mice and histochemistry (H&E) staining was performed. Representative H&E stains showing tumor infiltration of inflammatory cells from tumor challenged mice (A) without treatment, (B) with CRT/E7 DNA treatment, (C) with DMXAA treatment and (D) with CRT/E7 DNA and DMXAA treatment.

In FIG. 15, C57BL/6 TC-1 tumor-bearing mice were treated with CRT/E7 DNA vaccine and/or DMXAA as illustrated in FIG. 11. Seven days after the last vaccination, tumors were excised from mice. Tumor infiltrating lymphocytes were isolated and characterized for numbers of E7-specific IFN-γ+CD8+ T cells using HPV-16 E7 peptide-loaded MHC class I tetramer and anti-mouse CD8 antibody staining, followed by flow cytometry analysis. On the left, representative figure of the flow cytometry data. The numbers in the figure represent the numbers of E7-specific IFN-γ+CD8+ T cells in relation to the total tumor infiltrating lymphocytes collected. On the right, bar graph depicting the numbers of E7-specific IFN-γ-secreting CD8+ T cells in relation to tumor inflitrating lymphoctes collected (mean+s.d.).

In FIG. 16, control groups of mice were treated with CRT DNA vaccine and/or DMXAA for comparison. Tumor size was measured twice every week with a caliper. Tumor volume was calculated using the formula: tumor volume (mm3)=3.14/6×[largest diameter×(perpendicular diameter)2]/6. Line graph depicting the tumor volume (mean+s.d.) in TC-1 tumor-bearing mice treated with the various combinations.

### Example 3

### Pretreatment with Cisplatin Enhances E7-Specific CD8+ T Cell-Mediated Antitumor Immunity Induced by DNA Vaccination

### Abstract

Immunotherapy has emerged as a potentially promising approach for the control of cancer. We have previously developed DNA vaccines targeting human papillomavirus type 16 (HPV-16) E7 antigen and identified calreticulin (CRT) as one of the most potent immunostimulatory molecules that is capable of improving E7 DNA vaccine potency. Since the combination of multiple modalities for cancer treatment is more likely to generate more potent therapeutic effects for the control of cancer, the current study has explored the combination of chemotherapy using cisplatin, which is routinely used in chemoradiation for advanced cervical cancer, with immunotherapy using DNA vaccines encoding CRT linked to HPV-16 E7 antigen (CRT/E7) in a preclinical model. Our results indicate that treatment of tumor challenged mice with chemo-immunotherapy combining cisplatin followed by CRT/E7 DNA generated the highest E7-specific CD8+ T cell immune response and produced the greatest anti-tumor effects as well as long-term survival compared to all the other treatment regimens. We also found that treatment of tumor cells with cisplatin and E7-specific CD8+ T cells from the spleens of immunized mice led to the highest cell-mediated lysis of E7-expressing tumor cells in vitro. Thus, our data suggest that chemo-immunotherapy using cisplatin followed by CRT/E7 DNA is an effective treatment against E7-expressing tumors.

### Introduction

Multimodality treatments that combine conventional cancer therapies with antigen-specific immunotherapy have emerged as promising approaches for the control of cancer (for reviews, see [Boyd, 2003 #19; Moniz, 2003 #20]). Antigen-specific immunotherapy is an attractive approach for the treatment of cancers since it has the potency to specifically eradicate systemic tumors and control metastases without damaging normal cells. A favorable approach to antigen-specific immunotherapy is the use of DNA vaccines based on their safety, stability and ease of preparation (for review, see [Gurunathan, 2000 #13]). However, DNA vaccines are poorly immunogenic. Thus, the potency of DNA vaccines needs to be enhanced by employing methods to target DNA to the professional APCs and by modifying the properties of antigen-expressing APCs in order to boost vaccine-elicited immune responses. A number of approaches have been developed to enhance DNA vaccine potency (For review see [Hung, 2003 #18; Tsen, 2007 #17]).

One particular approach involves the employment of intracellular targeting strategies to enhance MHC class I and class II antigen presentation in DCs. Our previous studies have explored the linkage of calreticulin (CRT), a Ca2+-binding protein located in the endoplasmic reticulum (ER) to a model tumor antigen, human papilloma virus type4 16 (HPV-16) E7, for the development of a DNA vaccine, CRT/E7 [Cheng, 2001 #6]. We have previously shown that mice vaccinated intradermally with CRT/E7 DNA exhibited a dramatic increase in E7-specific CD8+T cell immune response and an impressive antitumor effect against E7-expressing tumors [Cheng, 2001 #6]. This vaccine was also found to be the most effective of the HPV-16 E7 DNA vaccines employing intracellular targeting strategies tested [Kim, 2004 #1]. This study employed an attenuated (detox) versions of E7 that has been mutated at E7 position 24 and/or 26 which disrupts the Rb binding site of E7, abolishing the capacity of E7 to transform cells [Munger, 2001 #11]. This vaccine thus addresses the safety concerns regarding the potential for oncogenicity associated with administration of E7 as DNA vaccines into the body, thus making it suitable for clinical translation. These studies suggest that CRT is a highly potent candidate molecule to be used in DNA vaccines targeting HPV infections and HPVassociated lesions.

Antigen-specific DNA vaccines have been shown to be effective in preclinical models against small tumors. However, such immunotherapeutic strategies alone may not be capable of controlling bulky rapidly growing tumors. This challenge may be overcome by the employment of multimodality treatment regimens that combine immunotherapy with chemotherapy in order to generate a much stronger antitumor effect.

Chemotherapeutic reagents are generally used to treat cancer based on their inherent tendency to attack cells that rapidly proliferate and have a good blood supply. Furthermore, chemotherapeutic reagents travel in the blood system, which allows them to be used for cancers in multiple parts in the body. Cisplatin is one such chemotherapeutic drug that is commonly used to treat certain types of cancers including ovarian, breast and cervical cancers (for review, see [Sleijfer, 1985 #12]).

In the current study, we have utilized a combination strategy employing CRT/E7 DNA vaccine and cisplatin to generate an enhanced immune response and antitumor effect against E7-expressing tumors. We found that of treatment of tumor challenged mice with chemo-immunotherapy combining cisplatin followed by CRT/E7 DNA produced the greatest anti-tumor effects as well as long-term survival compared to all the other treatment regimens. Furthermore, immunization of mice with the same chemoimmunotherapy regimen generated the highest numbers of CD8+ T cells of all the treatment regimens tested. We also found that the treatment of tumor cells with cisplatin and E7-specific CD8+ T cells from the spleens of immunized mice led to the highest cellmediated lysis of E7-expressing tumor cells in vitro. Thus, our data suggest that the chemo-immunotherapy regimen of cisplatin followed by CRT/E7 DNA generates significant antitumor effects against E7-expressing tumors. The clinical implications of this treatment are discussed.

### Materials and Methods

Mice. Female C57BL/6 mice (5-8 weeks old) were purchased from the National Cancer Institute (Frederick, Md.) and kept in the oncology animal facility of the Johns Hopkins Hospital (Baltimore, Md.). All of the animal procedures were performed according to approved protocols and in accordance with recommendations for the proper use and care of laboratory animals.
Cell line. Briefly, TC-1 cells were obtained by co-transformation of primary C57BL/6 mouse lung epithelial cells with HPV-16 E6 and E7 and an activated ras oncogene as described previously [Lin, 1996 #2]. The expression of E7 in TC-1 cells has also been characterized previously by He et al [He, 2000 #3].
DNA Constructs. The generation of the DNA vaccine encoding CRT and E7(detox) was described previously [Kim, 2004 #11]. Briefly, pNGVL4a-CRT/E7(detox), was generated by PCR amplification of CRT by primers (5′-AAAGTCGACATGCTGCTATCCGTGCCGCTGC-3′ and 5′-GAATTCGTTGTCTGGC-CGCACAATCA-3′) using a human CRT plasmid as a template. The PCR product was cut with SalI/EcoRI and cloned into the SalI/EcoRI sites of pNGVL4a-E7(detox). The accuracy of DNA constructs was confirmed by DNA sequencing.
DNA Vaccination by gene gun. DNA-coated gold particles were prepared, and gene gun particle-mediated DNA vaccination was performed, according to a protocol described previously [Chen, 2000 #4]. Gold particles coated with DNA vaccines (1 μg DNA/bullet) were delivered to the shaved abdominal regions of mice by using a helium-driven gene gun (Bio-Rad Laboratories Inc., Hercules, Calif.) with a discharge pressure of 400 lb/in2. C57BL/6 mice (5 per group) were immunized with 2 μg of the DNA vaccine and received two boosters with the same dose at 4-day intervals. Splenocytes were harvested 30 days after tumor challenge.

### Cisplatin Treatment

C57BL/6 mice (5 per group) were intraperitoneally injected with 10 mg cisplatin/kg bodyweight twice with a 3-day interval. The administered doses were diluted with PBS solution to the required concentration and injected in volumes of 200 μl.

### In Vivo Tumor Treatment Experiment

For in vivo tumor treatment, 1×10⁵ TC-1 tumor cells/mouse were injected into 5-8 week-old C57BL/6 mice (5 per group) subcutaneously in the right leg. After 8 days, the mice were divided into five groups reflecting different treatment regimens: group 1 (5 per group) received only TC-1 tumor challenge, group 2 (5 per group) were injected with cisplatin as described above, group 3 (5 per group) were immunized with the DNA vaccine as described above, group 4 (5 per group) were injected with cisplatin and then immunized with the DNA vaccine 4 days later as described above and group 5 (5 per group) were immunized and then injected with cisplatin 4 days later as described above. Mice were monitored once a week by inspection and palpation.

### Intracellular Cytokine Staining and Flow Cytometery Analysis

Pooled splenocytes from tumor challenged and naïve mice that were treated with the various treatment regiments were harvested 7 days after the last treatment and incubated for 20 h with 1 μg/ml of E7 peptide containing an MHC class I epitope (aa49-57, RAHYNIVTF) in the presence of GolgiPlug (BD Pharmingen, San Diego, Calif., USA). The stimulated splenocytes were then washed once with FACScan buffer and stained with phycoerythrin-conjugated monoclonal rat anti-mouse CD8a (clone 53.6.7). Cells were subjected to intracellular cytokine staining using the Cytofix/Cytoperm kit according to the manufacturer's instruction (BD Pharmingen, San Diego, Calif., USA). Intracellular IFN-γ was stained with FITC-conjugated rat anti-mouse IFN-γ. All antibodies were purchased from BD Pharmingen. Flow cytometry analysis was performed using FACSCalibur with CELLQuest software (BD Biosciences, Mountain View, Calif., USA).

In Vitro CTL Assays after Ciplatin Treatment

Luciferase-expressing TC-1 cells in medium were seeded into a 24-well roundbottom plate (5×10⁴ cells/well). After sitting overnight, the medium was replaced with 1 ml of fresh medium containing 5 μg of cisplatin. The mixture of TC-1 tumor cells and cisplatin-containing medium was incubated in 5% CO2 for 24 h at 37° C. E7-specific cytotoxic T lymphocytes from the spleens of tumor challenged mice immunized with the DNA vaccine served as effector cells and were added in the amount of 1×10⁶ cells/well. TC-1 cells expressing luciferase were used as target cells. After incubation, D-luciferin (potassium salt; Xenogen Corp.) was added to each well at 150 μg/ml in media 7-8 min before imaging with the Xenogen IVIS 200 system.

### Additional Materials & Methods

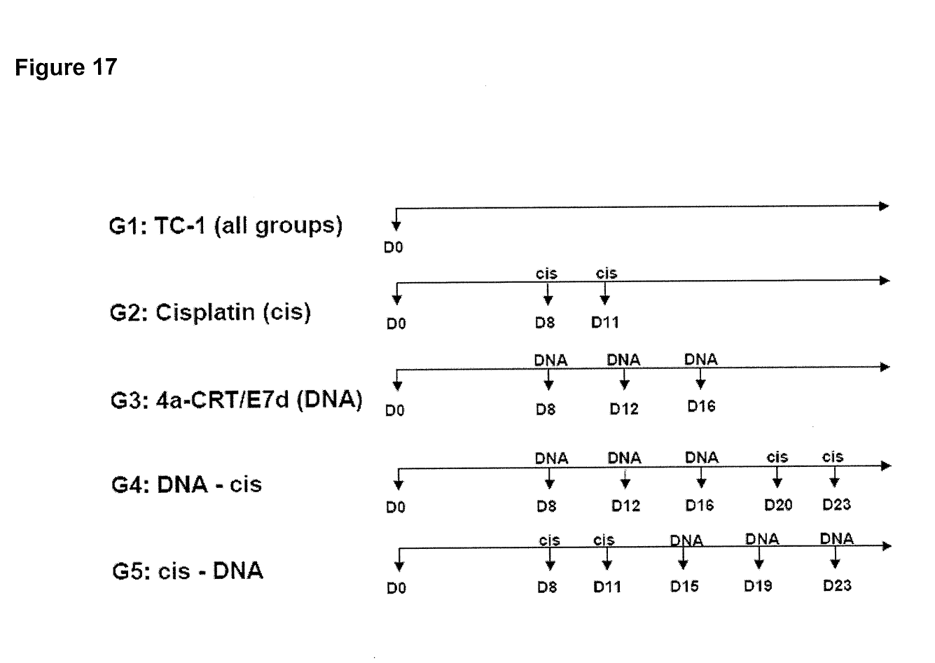

In FIG. 17, groups of C57BL/6 mice (5 per group) were subcutaneously challenged with 5×10⁴/mouse of TC-1 tumor cells on day 0. Tumor challenged mice were treated with cisplatin (cis) and/or

DNA encoding CRT/E7 (DNA) as indicated in the time line. Cisplatin was administered via intraperitoneal injection of 10 mg/kg bodyweight. DNA was administered via gene gun in the amount of 2 ug/mouse.

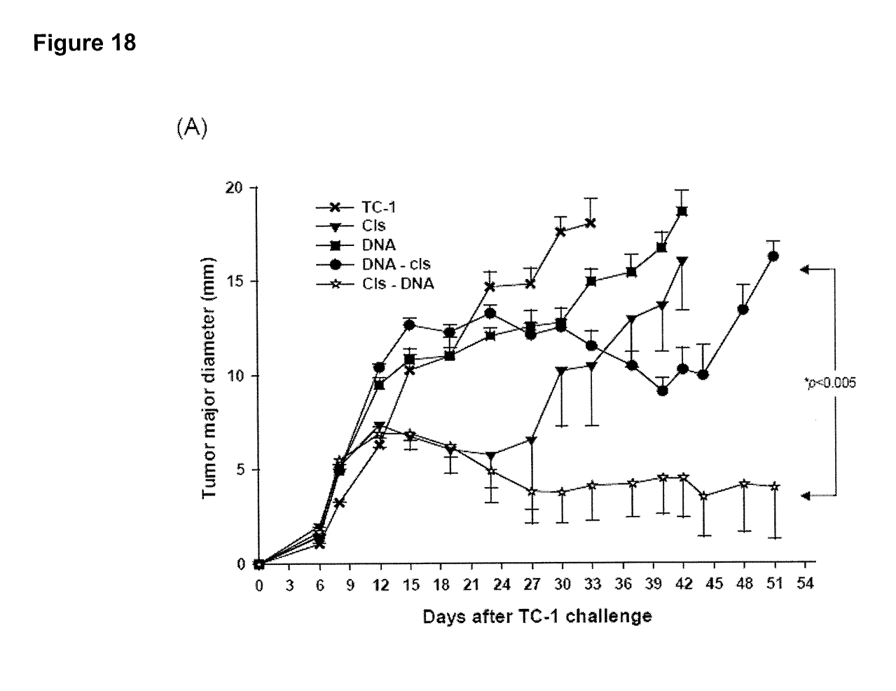

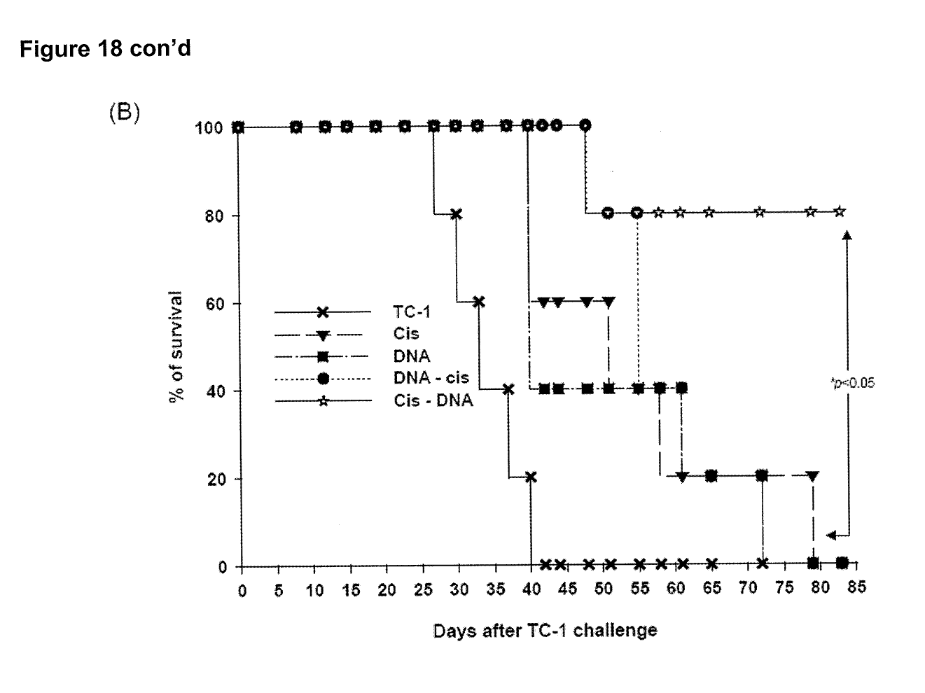

In FIG. 18, groups of C57BL/6 mice (5 per group) were challenged with TC-1 tumor cells and treated with cisplatin and/or DNA as illustrated in FIG. 1. (A) Line graph depicting the tumor volume in TC1 tumor bearing mice treated with the different treatment regimens (mean+s.d.). Note: The group of tumor challenged mice treated with cisplatin followed by the DNA vaccine had the best therapeutic antitumor effect over time as compared to challenged mice treated with the other treatment regimens (p<0.005). (B) Kaplan & Meier survival analysis of TC1 tumor challenged mice treated with the different treatment regimens. Note: The tumor challenged mice treated with cisplatin followed by DNA vaccine showed improved survival compared to challenged mice treated with the other treatment regimens (p<0.05).

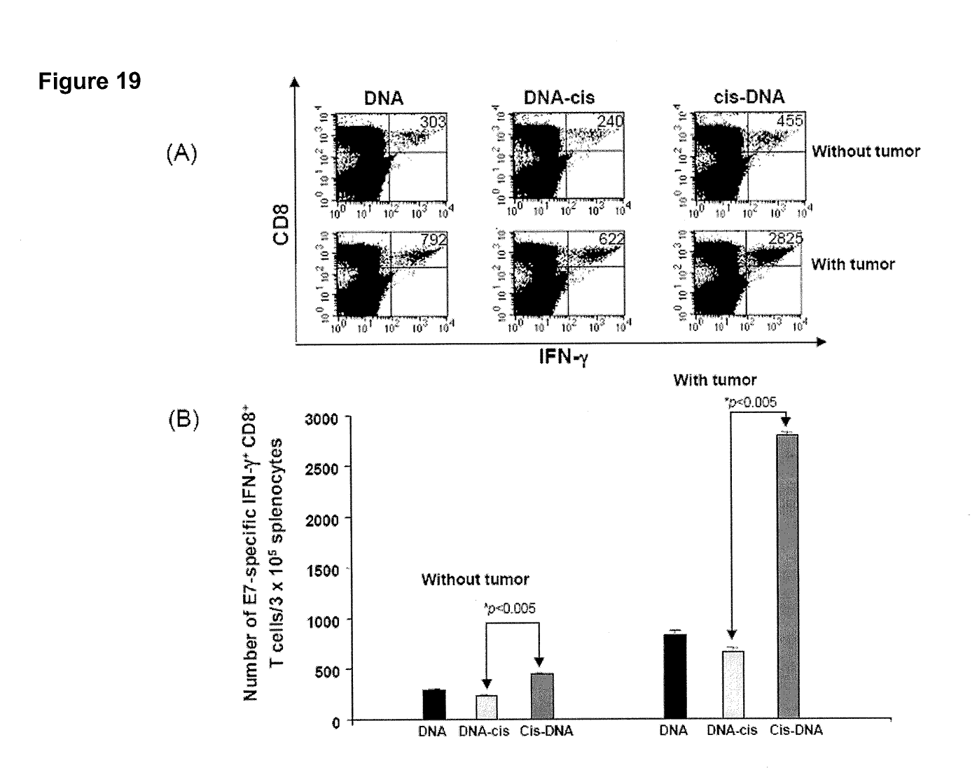

In FIG. 19, groups of C57BL/6 mice (5 per group) were challenged with TC-1 tumor cells and treated with cisplatin and/or DNA as illustrated in FIG. 1. Naive C57BL/6 mice (5 per group) were also administered cisplatin and/or DNA following the same regimen as tumor challenged mice for comparison. Thirty days after tumor challenge, splenocytes from mice with and without tumor challenge were harvested and stained for CD8 and intracellular IFN-γ and then characterized for E7-specific CD8+ T cells using intracellular IFN-γ staining followed by flow cytometry analysis. (A) Representative data of intracellular cytokine stain followed by flow cytometry analysis showing the number of E7-specific IFN γ+ CD8+ T cells in the various groups (right upper quadrant). (B) Bar graph depicting the numbers of E7-specific IFN-γ-secreting CD8+ T cells per 3×10⁵ pooled splenocytes (mean+s.d.).

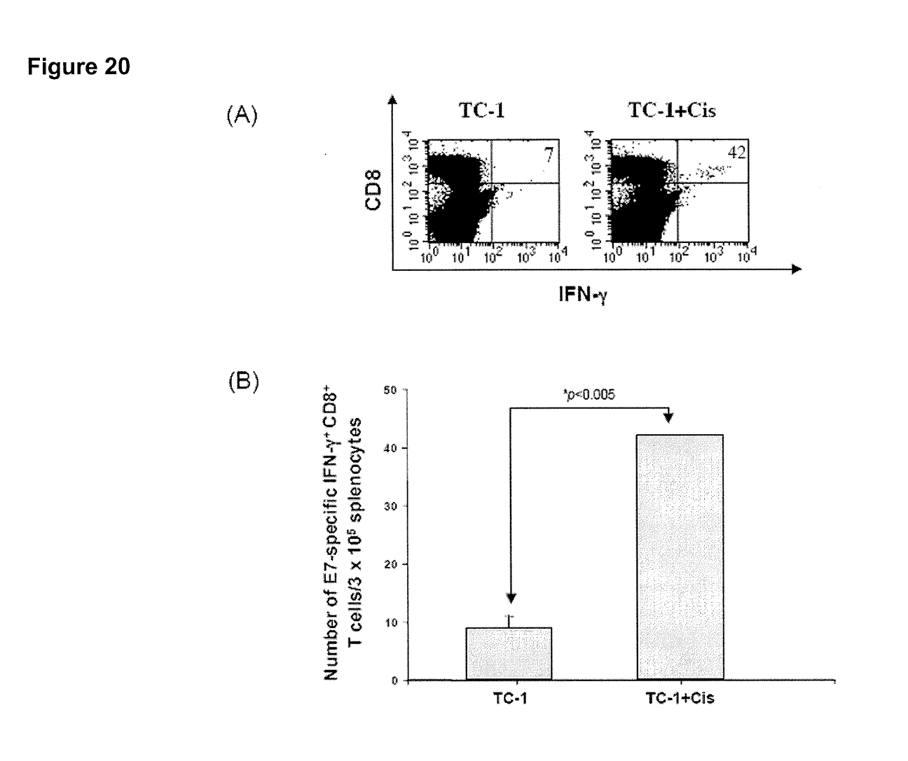

In FIG. 20, groups of C57BL/6 mice (5 per group) were challenged with TC-1 tumor cells and treated with or without cisplatin at the dose of 10 mg/kg bodyweight twice with a 3-day interval. Thirty days after tumor challenge, splenocytes from nontreated and treated mice were harvested and stained for CD8 and intracellular IFN-γ. The cells were then characterized for E7-specific CD8+ T cells using intracellular IFN-γ staining followed by flow cytometry analysis. (A) Representative data of intracellular cytokine stain followed by flow cytometry analysis showing the number of E7-specific IFNγ+CD8+ T cells in the different groups. (B) Bar graph depicting the numbers of E7-specific IFN-γ-secreting CD8+ T cells per 3×10⁵ pooled splenocytes (mean+s.d.). Note: TC-1 tumor-bearing mice treated with cisplatin showed significantly increased levels of E7-specific CD8+ T cells (p<0.005).

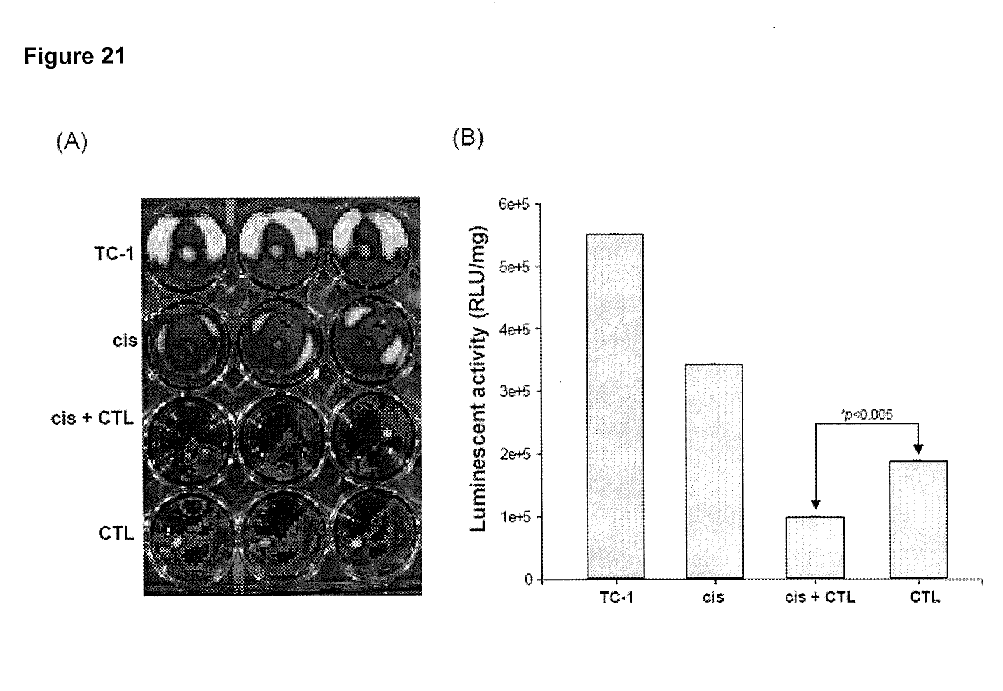

In FIG. 21, Luciferase-expressing TC-1 tumor cells were added to 24-well plates at a dose of 1×10⁶/well. TC-1 tumor cells were (a) untreated, (b) treated with 5 ug/ml of cisplatin (cis) alone, (c) treated with 5 ug/ml of cisplatin and 1×10⁶ E7-specific cytotoxic T cells (CTL), or (d) treated with 1×10⁶ E7-specific cytotoxic T cells (CTL) alone. The degree of CTL-mediated killing of the tumor cells was indicated by the decrease of luminescence activity using the IVIS luminescence imaging system series 200. Bioluminescence signals were acquired for one minute. A) Representative luminescence images of 24-well plates showing lysis of the tumor cells. B) Bar graph depicting the quantification of luminescence intensity in tumor cells treated with cisplatin and/or E7-specific cytotoxic T cells (mean+s.d.). Note: The TC-1 tumor cells treated with cisplatin and E7-specific cytotoxic T cells led to significant loss of luminescence intensity indicating enhanced lysis of tumor cells by the E7-specific CD8+T cells (p<0.005).

### Results

TC-1 Tumor Challenged Mice Treated with Cisplatin Followed by CRT/E7(Detox) DNA Generate the Best Therapeutic Anti-Tumor Effects

To determine the antitumor effect of chemo-immunotherapy combining cisplatin and DNA encoding CRT linked to the mutated form of E7 (CRT/E7(detox)), we first challenged groups of C57BL/6 mice (5 per group) with TC-1 tumor cells and then treated them with the different regimens as illustrated in FIG. 17. As shown in FIG. 18A, tumor challenged mice treated with cisplatin followed by CRT/E7(detox) DNA showed significantly lower tumor volumes over time as compared to challenged mice treated with the other treatment regimens (p<0.005). Furthermore, tumor challenged mice treated with cisplatin followed by CRT/E7(detox) DNA showed improved survival compared to challenged mice treated with the other treatment regimens (p<0.05) (FIG. 18B). Thus, our data suggest that the treatment regimen using cisplatin followed by CRT/E7(detox) DNA produces the best therapeutic anti-tumor effects and long-term survival in TC-1 tumor bearing mice.

TC-1 Tumor Challenged Mice Treated with Cisplatin Followed by CRT/E7(detox) DNA Generate Highest Frequency of E7-Specific CD8+ T Cells

In order to determine the E7-specific CD8+ T cell immune response in mice treated with the various regimens, we first challenged groups of C57BL/6 mice (5 per group) with TC-1 tumor cells and then treated them with DNA vaccine alone, DNA vaccine followed by cisplatin or cisplatin followed by DNA vaccine as illustrated in FIG. 17. As a control, a group of naïve C57BL/6 mice were also treated with similar regimens for comparison. Seven days after the last treatment, we harvested splenocytes from vaccinated mice and characterized them for the presence of E7-specific CD8+ T cells using intracellular cytokine staining for IFN-γ followed by flow cytometry analysis. As shown in FIG. 19, tumor challenged mice that were administered cisplatin followed by CRT/E7(detox) DNA generated a significantly higher number of E7-specific CD8+ T cells compared to tumor challenged mice that were administered CRT/E7(detox) DNA followed by cisplatin or DNA alone (p<0.005). Similarly, we also observed higher numbers of E7-specific CD8+ T cells in naïve mice treated with cisplatin followed by CRT/E7(detox) DNA compared to naïve mice treated with CRT/E7(detox) DNA followed by cisplatin or DNA alone (p<0.005). However, the enhancement of the E7-specific CD8+ T cells generated by treatment with cisplatin followed by CRT/E7(detox) DNA was more pronounced in tumor-bearing mice compared to naïve mice. Thus, our results suggest that treatment of tumor bearing mice with cisplatin followed by CRT/E7(detox) DNA leads to the strongest E7-specific CD8+ T cell immune response.

Treatment of Tumor Bearing Mice with Ciplatin Leads to Increased Number of E7-Specific CD8+ T Cell Precursors

In order to determine if the treatment of HPV-16 E7-expressing tumor bearing mice with cisplatin will lead to increased frequency of E7-specific CD8+ T cells, we treated TC-1 tumor-bearing C57BL/6 mice (5 per group) with or without cisplatin. Seven days after the cisplatin treatment, splenocytes were harvested and characterized for the presence of E7-specific CD8+ T cells using intracellular cytokine staining from IFN-γ followed by flow cytometry analysis. As shown in FIG. 20, TC-1 tumor-bearing mice treated with cisplatin showed significantly increased numbers of E7-specific CD8+ T cell precursors compared to tumor-bearing mice without cisplatin treatment (p<0.005). Thus, our data suggests that chemotherapy with cisplatin leads to an increase in the E7-specific CD8+ T cell response.

Treatment with Cisplatin Renders the TC-1 Tumor Cells More Susceptible to Lysis by E7-Specific CTLs

In order to determine if treatment of TC-1 tumor cells with cisplatin will render the tumor cell more susceptible to E7-specific T cell-mediated killing, we performed a cytotoxicity assay using luciferase-expressing TC-1 tumor cells. TC-1 tumor cells were treated with 5 μg/ml of cisplatin (cis) alone, treated with 5 ug/ml of cisplatin and 1×10⁶ E7-specific cytotoxic T cells (CTL) or treated with 1×10⁶ E7-specific cytotoxic T cells (CTL) alone. Untreated TC-1 tumor cells were used as a control. The CTL-mediated killing of the TC-1 tumor cells in each well was monitored using bioluminescent imaging systems. The degree of CTL-mediated killing of the tumor cells was indicated by the decrease of luminescence activity. As shown in FIG. 21, the lowest luciferase activity was observed in the wells incubated with cisplatin and E7-specific cytotoxic T cells as compared to the wells incubated with cisplatin alone or E7-specific cytotoxic T cells alone (p<0.005). Thus, our data suggests that the TC-1 tumor cells treated with cisplatin increased the susceptibility of the tumor cells for lysis by the E7-specific cytotoxic T cells.

### Discussion

In the current study, we tested the efficacy of chemo-immunotherapy employing CRT/E7 DNA vaccine and cisplatin. We found that treatment of tumor challenged mice with chemo-immunotherapy using cisplatin followed by CRT/E7 DNA generated the highest E7-specific CD8+ T cell immune response and produced the greatest anti-tumor effects as well as long-term survival compared to all the other treatment regimens. In addition, we showed that treatment of tumor cells with cisplatin and E7-specific CD8+ T cells from the spleens of immunized mice led to the highest cell-mediated lysis of E7-expressing tumor cells in vitro. Thus, our data suggest that chemo-immunotherapy using cisplatin followed by CRT/E7 DNA is an effective treatment against E7-expressing tumors.

Our results have shown that only the therapy using cisplatin followed by CRT/E7 DNA generated a strong immune response and antitumor effect compared to all the other treatment regimens. However, it is interesting to note that the reverse treatment involving administration of the DNA vaccine before cisplatin administration failed to result in a strong immune response against tumors. This is probably due to the mechanism of action of the chemotherapeutic drug, cisplatin. Cisplatin is known to induce cell death through apoptosis or necrosis (for review see [Cepeda, 2007 #21]). Specifically, cisplatin acts by crosslinking DNA in several different ways, making it impossible for rapidly dividing cells to duplicate their DNA for mitosis. The damaged DNA sets off DNA repair mechanisms, which activate apoptosis when repair proves impossible. Our hypothesis is that the apoptosis induced by cisplatin causes the antigen to be spread into the surrounding area. This could then potentially be taken up by the APC, which can activate more number of CD8+ T cells, thus leading to an enhanced immune response.

A recent study has been conducted that combines treatment modalities chemotherapy and immunotherapy using peptide-based vaccination. For example, Bae et al. performed a study using HPV E7-subunit vaccines in combination with cisplatin [Bae, 2007 #15]. They found that this combination improved the cure and recurrence rates of tumors as well as the long-term antitumor immunity compared to single therapy. This study involved simultaneous administration of cisplatin along with the E7 subunit vaccines.

In the future, it will be important to explore the effect of other chemotherapeutic agents in combination with various DNA vaccination strategies on the treatment of tumors. Thus, this study demonstrates the effectiveness and clinical feasibility of employing chemotherapy as a complement to immunotherapeutic strategies to enhance the antitumor immunity induced by DNA vaccination.

### Summary

Chemotherapeutic reagents are generally used to treat cancer based on their inherent tendency to attack cells that rapidly proliferate and have a good blood supply. Furthermore, chemotherapeutic reagents travel in the blood system, which allows them to be used for cancers in multiple parts in the body. Cisplatin is one such chemotherapeutic drug that is commonly used to treat certain types of cancers including ovarian, breast and cervical cancers. Our study specifically shows that treatment of HPV E7-expressing TC-1 tumor bearing mice with ciplatin will lead to apoptotic cell death of TC-1 tumor cells, leading to increased number of E7-specific CD8+ T cell precursors. Thus, TC-1 tumor challenged mice treated with cisplatin followed by vaccination with CRT/E7(detox) DNA show significantly enhanced HPV E7-specific CD8+ T cell immune responses, resulting in enhanced therapeutic anti-tumor effects against TC-1 tumors.

### Example 4

### Enhancing the Antitumor Effects Induced by DNA Vaccination by Combination with Agents that Generate Apoptotic Tumor Cell Death

### Abstract

Multimodality treatments that combine conventional cancer therapies with antigen-specific immunotherapy have emerged as promising approaches for the control of cancer. We have identified several agents that are capable of inducing apoptotic cell death of the tumor. These agents include doxorubicin, the death receptor 5 antibody MD5-1, the proteasome inhibitor bortezomib, the DNA methylation inhibitor 5-aza-2-deoxycytidin, the soyabean extract genistein, the Cox2 inhibitor celecoxib and the flavinoid apigenin. Our study has shown that the administration of these agents in combination with DNA vaccination generates significantly enhanced antitumor effects and increased survival in tumor-challenged mice. Thus, such combination strategies have significant potential for future clinical translation.

Although antigen-specific DNA vaccines may be effective against small tumors inpreclinical models, many tumors can grow rapidly resulting in bulky tumors, which present a challenge to immunotherapeutic strategies alone. Multi-modality treatments which combine conventional cancer therapies with immunotherapy such as DNA vaccines have emerged as a potentially plausible approach in the fight against cancer. Our invention combines immunotherapy such as DNA vaccination with various agents that are capable of inducing apoptotic tumor cell death and thus enhances the antitumor effects generated by DNA vaccination.

The agents included in this invention are doxorubicin, the death receptor 5 antibody MD5-1, the proteasome inhibitor bortezomib, the DNA methylation inhibitor 5-aza-2-deoxycytidin, the soyabean extract genistein, the Cox2 inhibitor Celecoxib and the flavinoid apigenin. All these agents are capable of inducing apoptotic cell death of the tumor and thus enhance the antitumor effects generated by DNA vaccination. Our study specifically shows that these agents are capable of increasing the survival of tumor-challenged mice and enhancing the antitumor effects induced by DNA vaccination.

### Results

Co-Administration of Doxorubicin with the CRT/E6 DNA Vaccine Generates Enhanced Antitumor Effects and Increased Survival in Treated Tumor-Challenged Mice

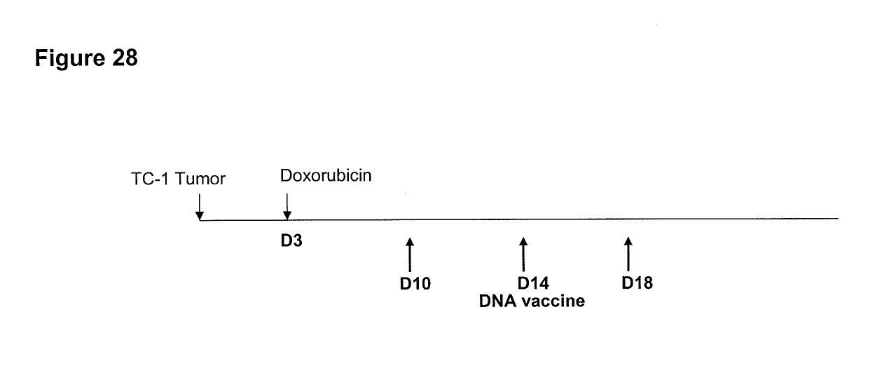

To determine the antitumor effect of chemo-immunotherapy combining doxorubicin and DNA encoding CRT linked to HPV-16 E6 (CRT/E6), we first challenged groups of C57BL/6 mice (5 per group) with TC-1 tumor cells and then treated them with the different regimens as illustrated in FIG. 28. Doxorubicin was used at 10 mg/kg body weight. Furthermore, tumor challenged mice treated with doxorubicin combined with CRT/E6 DNA showed improved survival compared to challenged mice treated with the DNA vaccine alone. Thus, our data suggest that the treatment regimen using doxorubicin combined with CRT/E6 DNA enhances the therapeutic anti-tumor effects and prolongs long-term survival in TC-1 tumor bearing mice.

Co-Administration of Mouse DR5 Antibody with the CRT/E7 DNA Vaccine Generates Enhanced Antitumor Effects and Increased Survival in Treated Tumor-Challenged Mice

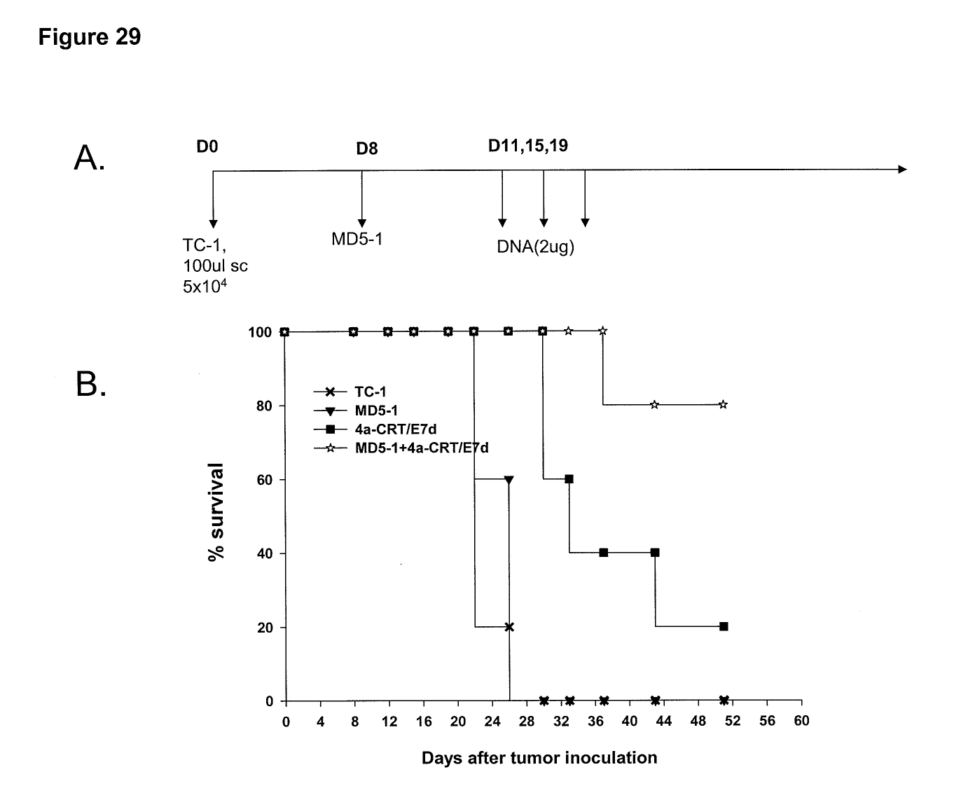

To determine the antitumor effect of chemo-immunotherapy combining mouse DR5 antibody and DNA encoding CRT linked to the mutated form of E7 (CRT/E7(detox)), we first challenged groups of C57BL/6 mice (5 per group) with TC-1 tumor cells and then treated them with the different regimens as illustrated in FIG. 29A. Furthermore, tumor challenged mice treated with mouse DR5 antibody combined with CRT/E7(detox) DNA showed improved survival compared to challenged mice treated with the DNA vaccine alone (FIG. 29B). Thus, our data suggest that the treatment regimen using mouse DR5 antibody combined with CRT/E7(detox) DNA enhances the therapeutic anti-tumor effects and prolongs long-term survival in TC-1 tumor bearing mice.

Co-administration of Bortezomib with the CRT/E7 DNA Vaccine Generates Enhanced Antitumor Effects in Treated Tumor-Challenged Mice

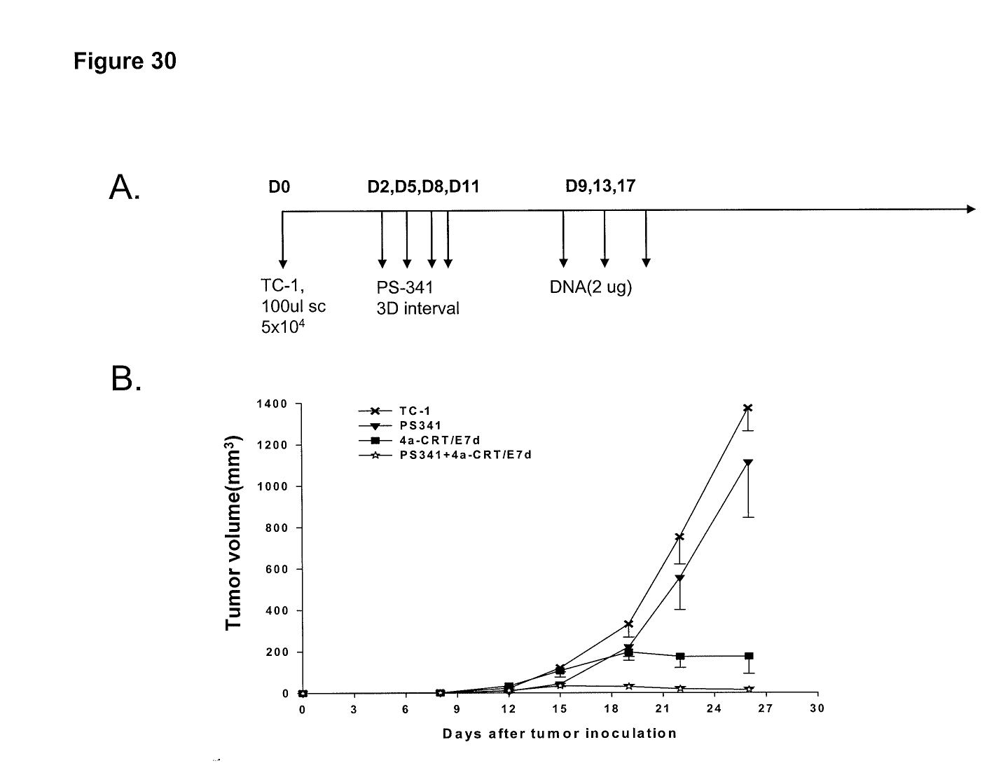

To determine the antitumor effect of chemo-immunotherapy combining bortezomib and DNA encoding CRT linked to the mutated form of E7 (CRT/E7(detox)), we first challenged groups of C57BL/6 mice (5 per group) with TC-1 tumor cells and then treated them with the different regimens as illustrated in FIG. 30A. As shown in FIG. 30B, tumor challenged mice treated with bortexomib followed by CRT/E7(detox) DNA showed significantly lower tumor volumes over time as compared to challenged mice treated with the other treatment regimens. Thus, our data suggest that the treatment regimen using bortezomib combined with CRT/E7(detox) DNA enhances the therapeutic anti-tumor effects in TC-1 tumor bearing mice.

Co-Administration of 5-aza-2-deoxycytidin with the CRT/E7 DNA Vaccine Generates Enhanced Antitumor Effects and Increased Survival in Treated Tumor-Challenged Mice

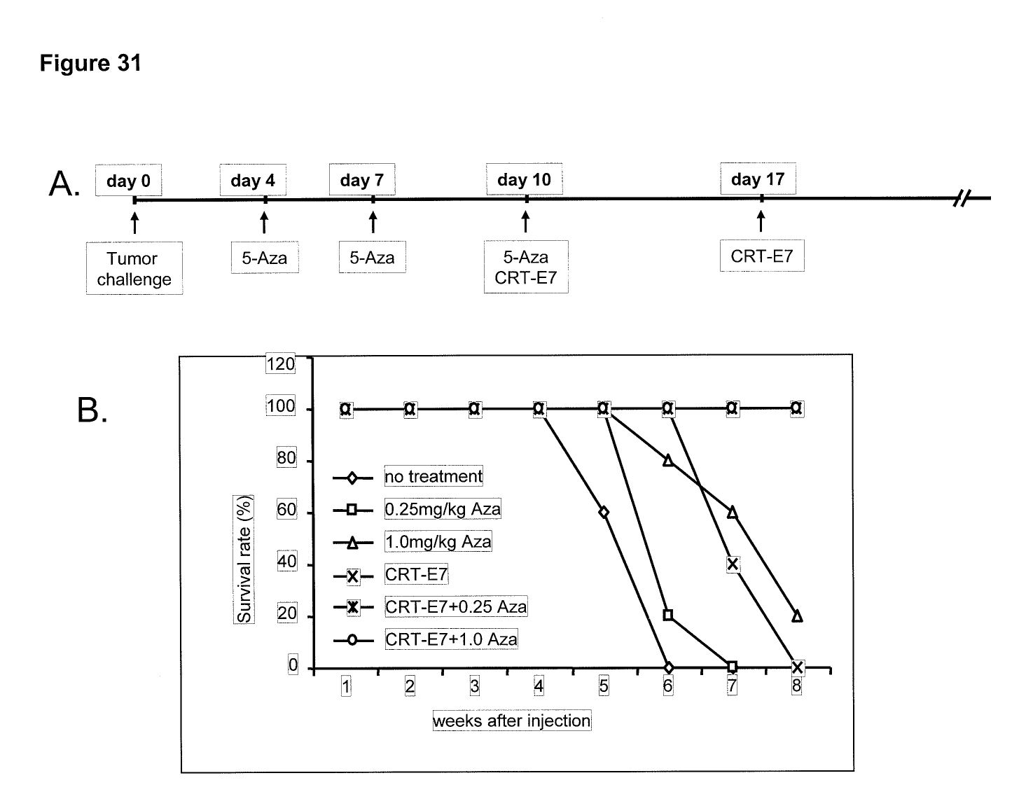

To determine the antitumor effect of chemo-immunotherapy combining 5-aza-2-deoxycytidin and DNA encoding CRT linked to the mutated form of E7 (CRT/E7(detox)), we first challenged groups of C57BL/6 mice (5 per group) with TC-1 tumor cells and then treated them with the different regimens as illustrated in FIG. 31A. Furthermore, tumor challenged mice treated with 5-aza-2-deoxycytidin combined with CRT/E7(detox) DNA showed improved survival compared to challenged mice treated with the DNA vaccine alone (FIG. 31B). Thus, our data suggest that the treatment regimen using 5-aza-2-deoxycytidin combined with CRT/E7(detox) DNA enhances the therapeutic anti-tumor effects and prolongs long-term survival in TC-1 tumor bearing mice.

Co-Administration of Genistein with the CRT/E7 DNA Vaccine Generates Enhanced Antitumor Effects and Increased Survival in Treated Tumor-Challenged Mice

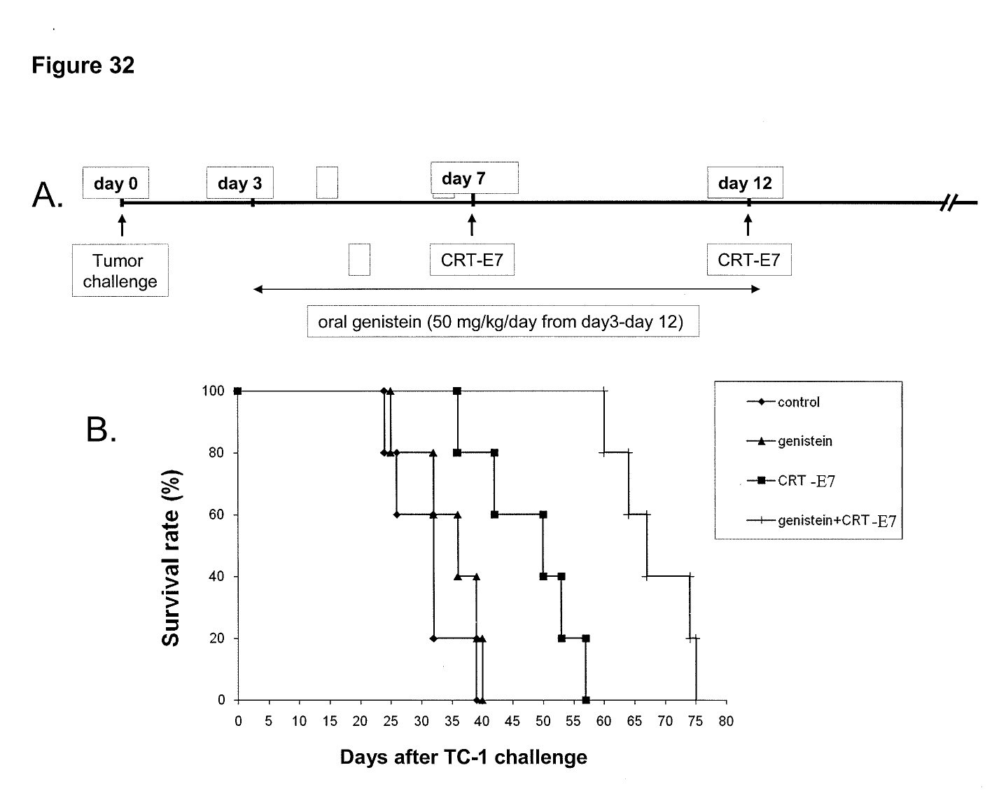

To determine the antitumor effect of chemo-immunotherapy combining genistein and DNA encoding CRT linked to the mutated form of E7 (CRT/E7(detox)), we first challenged groups of C57BL/6 mice (5 per group) with TC-1 tumor cells and then treated them with the different regimens as illustrated in FIG. 32A. Furthermore, tumor challenged mice treated with genistein combined with CRT/E7(detox) DNA showed improved survival compared to challenged mice treated with the DNA vaccine alone (FIG. 32B). Thus, our data suggest that the treatment regimen using genistein combined with CRT/E7(detox) DNA enhances the therapeutic anti-tumor effects and prolongs long-term survival in TC-1 tumor bearing mice.

Co-Administration of Celecoxib with the CRT/E7 DNA Vaccine Generates Enhanced Antitumor Effects and Increased Survival in Treated Tumor-Challenged Mice

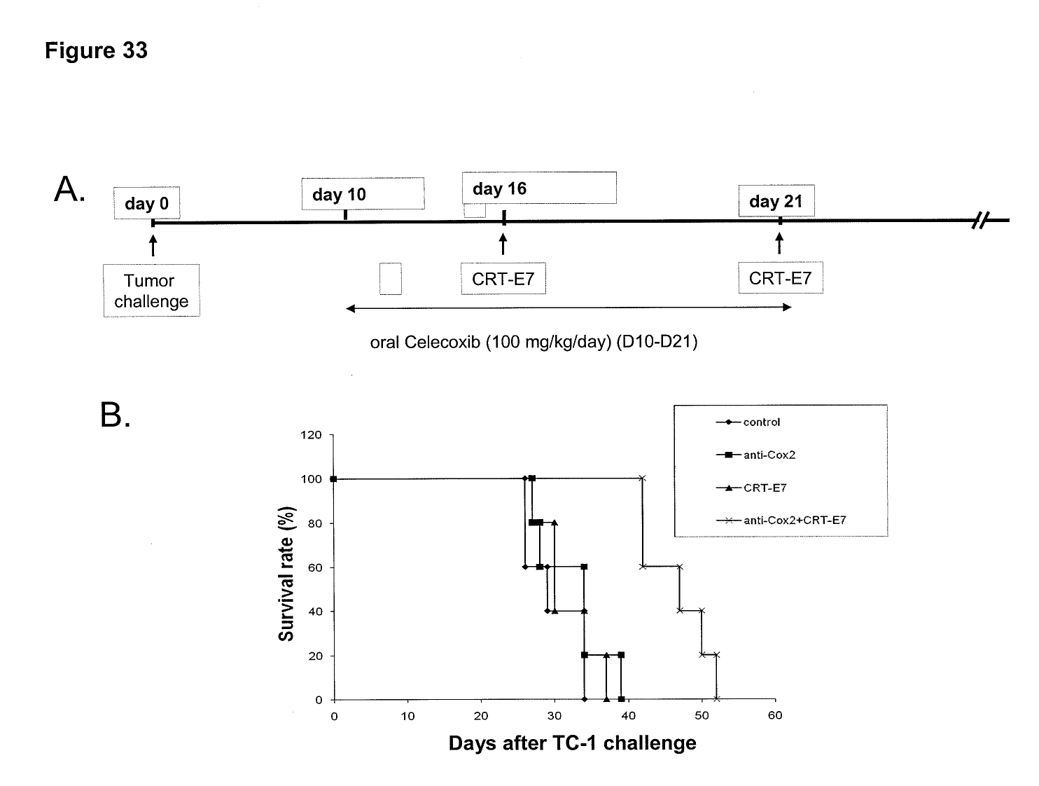

To determine the antitumor effect of chemo-immunotherapy combining celecoxib and DNA encoding CRT linked to the mutated form of E7 (CRT/E7(detox)), we first challenged groups of C57BL/6 mice (5 per group) with TC-1 tumor cells and then treated them with the different regimens as illustrated in FIG. 33A. Furthermore, tumor challenged mice treated with celecoxib combined with CRT/E7(detox) DNA showed improved survival compared to challenged mice treated with the DNA vaccine alone (FIG. 33B). Thus, our data suggest that the treatment regimen using celecoxib combined with CRT/E7(detox) DNA enhances the therapeutic anti-tumor effects and prolongs long-term survival in TC-1 tumor bearing mice.

Co-Administration of Apigenin with the E7-HSP70 DNA Vaccine Generates Enhanced Antitumor Effects and Increased Survival in Treated Tumor-Challenged Mice

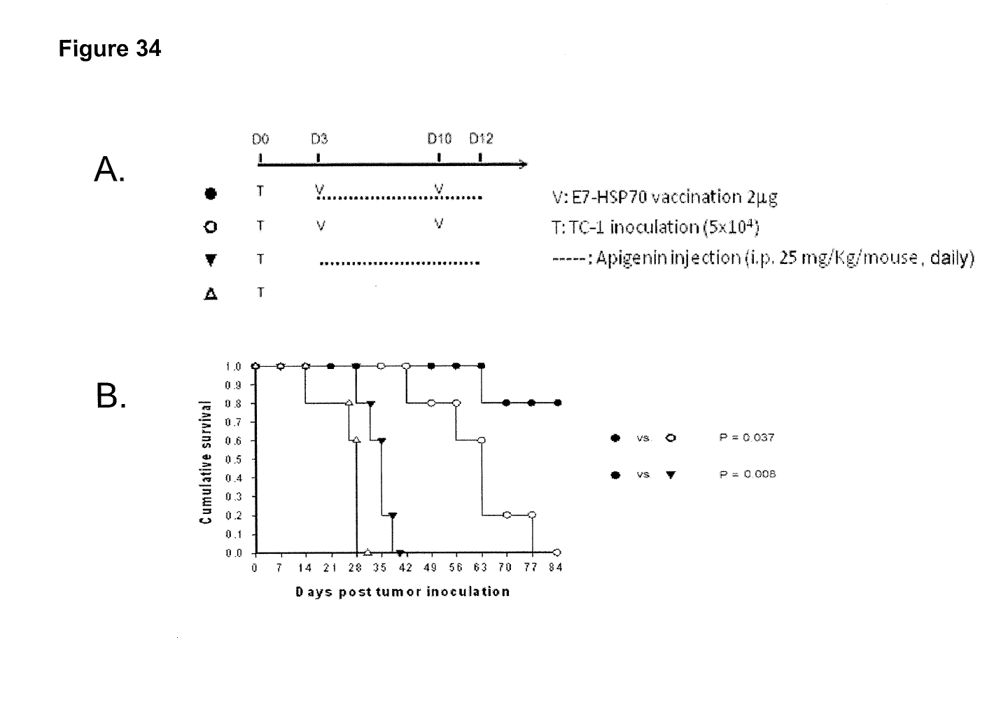

To determine the antitumor effect of chemo-immunotherapy combining apigenin and DNA encoding HSP70 linked to E7 (E7-HSP70) we first challenged groups of C57BL/6 mice (5 per group) with TC-1 tumor cells and then treated them with the different regimens as illustrated in FIG. 34A. Furthermore, tumor challenged mice treated with apigenin combined with E7-HSP70 DNA showed improved survival compared to challenged mice treated with the DNA vaccine alone (FIG. 34B). Thus, our data suggest that the treatment regimen using apigenin combined with E7-HSP70 DNA enhances the therapeutic anti-tumor effects and prolongs long-term survival in TC-1 tumor bearing mice.

### Additional Materials & Methods

In FIG. 29, C57BL/6 mice (5 per group) were challenged subcutaneously with 5×10⁴/mouse of TC-1 cells. Eight days later, the mice were treated with the mouse DR5 antibody (MD5-1) at a dose of 2.5 mg/ml. Eleven days after tumor challenge, mice were immunized via gene gun with 2 ug/mouse of the CRT/E7(detox) DNA vaccine three times at 3-day intervals. A. Treatment regimen B. Kaplan-Meier survival analysis of tumor-challenged mice treated with MD5-1 and/or the CRT/E7(detox) DNA vaccine.

In FIG. 30, C57BL/6 mice (5 per group) were challenged subcutaneously with 5×10⁴/mouse of TC-1 cells. Two days later, mice were treated intraperitoneally with bortezomib (PS341) at a dose of 0.1 ug/ul in a volume of 200 μl 4 times at 2-day intervals. Nine days after tumor challenge, mice were immunized via gene gun with 2 ug/mouse of the CRT/E7(detox) DNA vaccine three times at 3-day intervals. A. Treatment regimen B. Line graph depicting the tumor volume over time in TC-1 tumor-challenged mice treated with bortezomib and/or CRT/E7(detox) DNA vaccine.

In FIG. 31, C57BL/6 mice (5 per group) were challenged subcutaneously with 5×10⁴/mouse of TC-1 cells. Four days later, mice were treated with 5-aza-2-deoxycytidin at a dose of either 0.25 or 1 mg/kg 3 times at 2-day intervals. Ten days after tumor challenge, mice were immunized via gene gun with 2 ug/mouse of the CRT/E7(detox) DNA vaccine twice with a 1-week interval. A. Treatment regimen B. Kaplan-Meier survival analysis of tumor-challenged mice treated 5-aza-2-deoxycytidin and/or CRT/E7(detox) DNA vaccine.

In FIG. 32, C57BL/6 mice (5 per group) were challenged subcutaneously with 5×10⁴/mouse of TC-1 cells. Three days later, mice were treated with oral genistein (50 mg/kg/day) daily until day 12. Seven days after tumor challenge, mice were immunized via gene gun with 2 ug/mouse of the CRT/E7(detox) DNA vaccine twice with a 5-day interval. A. Treatment regimen B. Kaplan-Meier survival analysis of tumor-challenged mice treated with genistein and/or the CRT/E7(detox) DNA vaccine.

In FIG. 33, C57BL/6 mice (5 per group) were challenged subcutaneously with 5×10⁴/mouse of TC-1 cells. Ten days later, mice were treated with oral Celecoxib (100 mg/kg/day) daily until day 21. Sixteen days after tumor challenge, mice were immunized via gene gun with 2 ug/mouse of the CRT/E7(detox) DNA vaccine twice with a 5-day interval. A. Treatment regimen B. Kaplan-Meier survival analysis of tumor-challenged mice treated with celecoxib and the CRT/E7(detox) DNA vaccine.

In FIG. 34, C57BL/6 mice (5 per group) were challenged subcutaneously with 5×10⁴/mouse of TC-1 cells. Three days later, mice were treated intraperitoneally with apigenin daily (25 mg/kg/mouse) until day 12. Three days after tumor challenge, mice were immunized via gene gun with 2 ug/mouse of the E7-HSP70 DNA vaccine twice with 1-week interval. A. Treatment regimen B. Kaplan-Meier survival analysis of tumor-challenged mice treated with apigenin and/or the E7-HSP70 DNA vaccine.

All references cited above are all incorporated by reference herein, in their entirety, whether specifically incorporated or not. All publications, patents, patent applications, GenBank sequences and ATCC deposits, cited herein are hereby expressly incorporated by reference for all purposes. In case of conflict, the definitions within the instant application govern.

Having now fully described this invention, it will be appreciated by those skilled in the art that the same can be performed within a wide range of equivalent parameters, concentrations, and conditions without departing from the spirit and scope of the invention and without undue experimentation.

1. A method for treating cancer in a subject, comprising administering to a subject in need thereof a DNA vaccine encoding a tumor antigen or a biologically active homolog thereof and an apoptosis-inducing chemotherapeutic drug.

2. The method of claim 1, wherein the chemotherapeutic drug is selected from the group consisting of epigallocatechin-3-gallate (EGCG), 5,6 di-methylxanthenone-4-acetic acid (DMXAA), cisplatin, apigenin, doxorubicin, an anti-death receptor 5 antibody, a proteasome inhibitor, an inhibitor of DNA methylation, genistein, celecoxib and biologically active analogs thereof.

3. The method of claim 1, wherein the cancer is a head and neck cancer or cervical cancer.

4. The method of claim 1, wherein the tumor antigen is an antigen from a pathogenic organism.

5. The method of claim 4, wherein the tumor antigen is a viral antigen.

6. The method of claim 5, wherein the tumor antigen is an antigen from a human papilloma virus (HPV).

7. The method of claim 6, wherein the tumor antigen is E6 or E7.

8. The method of claim 7, wherein HPV is HPV-16.

9. The method of claim 1, wherein the tumor antigen is a protein that comprises an amino acid sequence that is at least about 90% identical to the amino acid sequence of an antigen from HPV or a biologically active fragment thereof.

10. The method of claim 9, wherein the tumor antigen is a protein that comprises an amino acid sequence that is at least about 90% identical to the amino acid sequence of a detox E6 or detox E7 protein and comprising the amino acid substitutions that are specific to detox E6 or E7, respectively, or a biologically active fragment thereof.

11. The method of claim 1, wherein the DNA vaccine comprises a nucleotide sequence encoding a fusion protein comprising the tumor antigen or a biologically active homolog thereof and an immunogenicity-potentiating polypeptide (IPP).

12. The method of claim 11, wherein the IPP comprises one or more of the translocation domain of a bacterial toxin, an endoplasmic reticulumn chaperone polypeptide, and an intercellular spreading protein or a biologically active homolog thereof.

13. The method of claim 12, wherein the IPP comprises ETA(dII), HSP70, calreticulin, LAMP-1 or VP22 or a biologically active homolog thereof.

14. The method of claim 11, wherein the fusion protein further comprises a linker linking the tumor antigen or the biologically active homolog thereof to the IPP.

15. The method of claim 1, wherein the chemotherapeutic drug is EGCG and wherein at least one dose of EGCG is administered before the first dose of the DNA vaccine.

16. The method of claim 1, wherein the chemotherapeutic drug is DMXAA and wherein at least one dose of the DNA vaccine is administered before the first dose of DMXAA.

17. The method of claim 1, wherein the chemotherapeutic drug is cisplatin and wherein at least one dose of cisplatin is administered before the first dose of DNA vaccine.

18. The method of claim 1, further comprising administering to the subject a nucleic acid that inhibits the expression of a pro-apoptotic protein and/or a nucleic acid that encoding an anti-apoptotic protein.

19. A composition comprising a DNA vaccine encoding a tumor antigen or a biologically active homolog thereof and an apoptosis-inducing chemotherapeutic drug.

20. A kit for treating cancer, comprising a DNA vaccine encoding a tumor antigen or a biologically active homolog thereof and an apoptosis-inducing chemotherapeutic drug.
